(19) United States
(12) Patent Application Publication
(10) Pub. No.: US 2020/0165257 A1
(43) Pub. Date: May 28, 2020

(54) INHIBITORS OF PHOSPHOINOSITIDE 3-KINASE AND HISTONE DEACETYLASE FOR TREATMENT OF CANCER

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Gurmit Grewal, Lexington, MA (US); Ashish Thakur, Gaithersburg, MD (US); Gregory James Tawa, Doylestown, PA (US); Marc Ferrer, Potomac, MD (US); Anton M. Simeonov, Bethesda, MD (US)

(21) Appl. No.: 16/621,290

(22) PCT Filed: Jun. 20, 2018
(86) PCT No.: PCT/US2018/038507

§ 371 Date: Dec. 11, 2019

Related U.S. Application Data
(60) Provisional application No. 62/523,390, filed on Jun. 22, 2017.

(51) Int. Cl. C07D 487/04; C07D 403/12; C07D 401/14; C07D 403/14; C07D 239/88; A61P 35/02

(52) U.S. Cl. CPC C07D 487/04; C07D 403/12; A61P 35/02; C07D 403/14; C07D 239/88; C07D 401/14

(57) ABSTRACT

The present invention is directed to a dual inhibitor of phosphoinositide 3-kinase (PI3K) and histone deacetylase (HDAC), including a core containing a quinazoline moiety or a quinazolin-4(3H)-one moiety, a kinase hinge binding moiety, and a histone deacetylase pharmacophore, a pharmaceutically acceptable salt thereof, a prodrug thereof, or solvate thereof. The present invention is also directed to a histone deacetylase inhibitor, including a core containing a quinazolin-4(3H)-one moiety and a histone deacetylase pharmacophore.

### CROSS-REFERENCE TO RELATED APPLICATION

This application claims priority to and the benefit of U.S. Provisional Application No. 62/523,390 filed on Jun. 22, 2017, which is hereby incorporated by reference in its entirety.

### BACKGROUND OF THE INVENTION

Histone deacetylases (HDACs) are key regulators of the cell cycle. They function by regulating expression of tumor suppressors (p21 and p27), c-Myc and cyclin Dl. Inhibition of HDACs causes cell cycle arrest and apoptosis. Dysregulation of HDACs is implicated in cancer initiation and proliferation. HDAC inhibition is an emerging therapeutic approach for the treatment of several cancers.

Dysregulated receptor tyrosine kinase (RTK) signaling is also linked to many cancers. Activation of epidermal growth factor (EFG) and human epidermal growth factor receptor 2 (HER2) pathways causes reduced activity of p21 and p27 and increased expression of c-Myc and cyclin Dl, which in turn promote cell proliferation, survival and angiogenesis. Often, activation of these pathways is driven by the activation of their downstream kinases. Inhibition of these kinases is an established pathway for cancer treatment. In many human cancers, phosphoinositide 3-kinase (PI3K) is activated, causing upregulation of the EGFR pathway. Simultaneous inhibition of both HDAC and RTK pathways may synergistically inhibit tumor growth.

PI3K and HDAC inhibitors are important cancer therapeutics. Several of them have been approved. But both classes of inhibitors suffer from two major limitations, insufficient efficacy and developed resistance. There is strong evidence in the literature that, simultaneous inhibition of both PI3K and HDAC would address both these limitations, giving better efficacy, and a better therapeutic window than single inhibitors, while avoiding developed resistance. Panobinostat and SAHA (suberanilohydroxamic acid, a.k.a. Vorinostat), while resulting in modulation of the acetylation status of a wide range of protein targets leading to a therapeutic response, also lead to undesired toxic effects, including hematological, gastrointestinal and cardiac toxicity. SAHA monotherapy is approved by the Food and Drug Administration (FDA) for the treatment of cutaneous T-cell lymphoma, however it has been demonstrated to have little activity. Pan PI3K inhibitors also suffer from toxicity and smaller therapeutic window. Selective inhibitors of specific isoforms of HDAC (such as HDAC6) and PI3K (such as PI3K6) potentially would have better toxicity profile and therefore bigger therapeutic window. In this context, CURIS is developing an HDAC-PI3K dual inhibitor, CUDC-907 for the treatment of lymphoma and multiple myeloma. With its integrated HDAC and PI3K inhibitory activity, CUDC-907 may thus offer improved therapeutic benefit through simultaneous suppression of cancer cell proliferation and perturbation of their protective microenvironment. However, CUDC-907 is not selective to any specific isoform of HDAC or PI3K and exhibits pan-HDAC and pan-PI3K inhibition, which might contribute to toxicity and low tolerability.

Thus, there remains an unmet need for new dual inhibitors having high potency and selectivity.

### SUMMARY OF THE INVENTION

In an embodiment, a dual inhibitor of phosphoinositide 3-kinase (PI3K) and histone deacetylase (HDAC), a pharmaceutically acceptable salt thereof, a prodrug thereof, or solvate thereof are provided. The dual inhibitor includes a core containing a quinazoline moiety or a quinazolin-4(3H)-one moiety, a kinase hinge binding moiety, and a histone deacetylase pharmacophore.

In another embodiment, an inhibitor of histone deacetylase (HDAC), a pharmaceutically acceptable salt thereof, a prodrug thereof, or solvate thereof are provided. The HDAC inhibitor includes a core containing a quinazolin-4(3H)-one moiety and a histone deacetylase pharmacophore.

In still another embodiment, a method for treating or diagnosing cancer in a mammal is provided. The method includes administering to the mammal a pharmaceutical composition including an effective amount of an active agent, wherein the active agent is the dual inhibitor of phosphoinositide 3-kinase (PI3K) and histone deacetylase (HDAC), a pharmaceutically acceptable salt thereof, a prodrug thereof, or solvate thereof.

In yet another embodiment, a method for treating or diagnosing cancer in a mammal is provided. The method includes administering to the mammal a pharmaceutical composition including an effective amount of an active agent, wherein the active agent is the inhibitor of histone deacetylase, a pharmaceutically acceptable salt thereof, a prodrug thereof, or solvate thereof.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

Formulae 1, 2, 3, 4, 5, 6, 7, 8, 9, and 10 include all pharmaceutically acceptable salts of Formulae 1, 2, 3, 4, 5, 6, 7, 8, 9, and 10.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“−”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The term C₁-C₅alkyl as used herein indicates an alkyl group having from 1, 2, 3, 4, or 5 carbon atoms.

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Pharmaceutical compositions” means compositions comprising at least one active agent, such as a compound or salt of Formula 3, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “mammal” means a human or non-human animal. In some embodiments the mammal is a human.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of Formulae 1, 2, 3, 4, 5, 6, 7, 8, 9, and 10 may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill “Dictionary of Chemical Terms” (1984) McGraw-Hill Book Company, New York; and Eliel, E. and Wilen, S., “Stereochemistry of Organic Compounds” (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, iso-propanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

### Embodiments

In an embodiment, a dual inhibitor of phosphoinositide 3-kinase (PI3K) and histone deacetylase (HDAC), a pharmaceutically acceptable salt thereof, a prodrug thereof, or solvate thereof are provided. The dual inhibitor may include a core containing a quinazoline moiety or a quinazolin-4(3H)-one moiety, a kinase hinge binding moiety, and a histone deacetylase pharmacophore.

In an embodiment, the histone deacetylase pharmacophore may include:

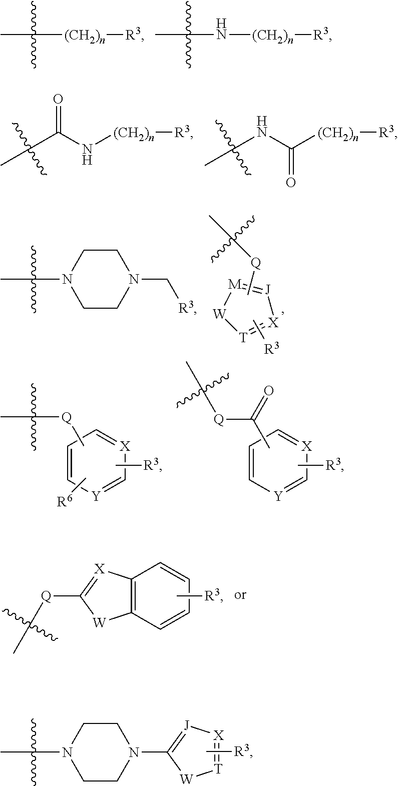

but is not limited thereto.

In the above formulae,
at least one non-adjacent —CH₂— group may be optionally replaced with —O—;
n may be 1, 2, 3, 4, and 5;
J may be CH or N;
M may be CH or N;
W may be N, O, or S;
X may be CH or N;
T may be CH or N;
Q may be —(CH₂)ₚ—, —(CH₂)ₚNH(CH₂)ᵣ—, —NH(CH₂)ₚ— or —(CH₂)ₚNH—, wherein p and r may each independently be 0, 1, 2, 3, or 5;
Y may be CH or N;
R³ may be

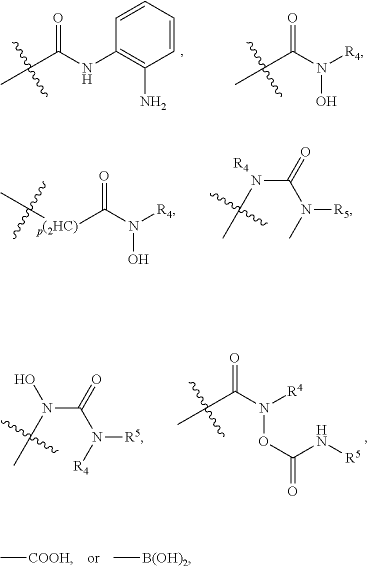

wherein R⁴ and R⁵ may each independently be H or a C₁-C₅ alkyl group;
R⁶ is H or a C₁-C₄ alkyl group.

The kinase hinge binding moiety may include, but is not limited thereto:

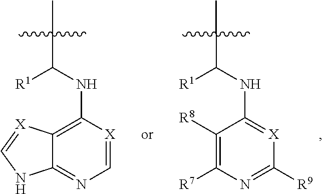

wherein R¹ may be a C₁-C₅ alkyl group;

R⁷ may be H, a C₁-C₅ alkyl group, a C₁-C₅ alkyl containing 1-5 fluorine atoms, a C₁-C₅ alkyl containing 1-5 deuterium atoms, or NH₂;

R⁸ may be H, a C₁-C₅ alkyl group, Cl, CONH₂, or CN;

R⁹ may be H, a C₁-C₅ alkyl group, a C₁-C₅ alkyl containing 1-5 fluorine atoms, a C₁-C₅ alkyl containing 1-5 deuterium atoms, or NH₂; and

X may be CH or N.

In an embodiment, the core of the dual inhibitor may be represented by Formula 1:

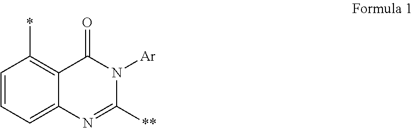

wherein Ar is an aryl or heteroaryl group unsubstituted or substituted with 1-3 C₁-C₆ alkyl groups,

“*” indicates a binding site to the histone deacetylase pharmacophore, and

“**′” indicates a binding site to the kinase hinge binding moiety.

For example, the histone deacetylase pharmacophore may be:

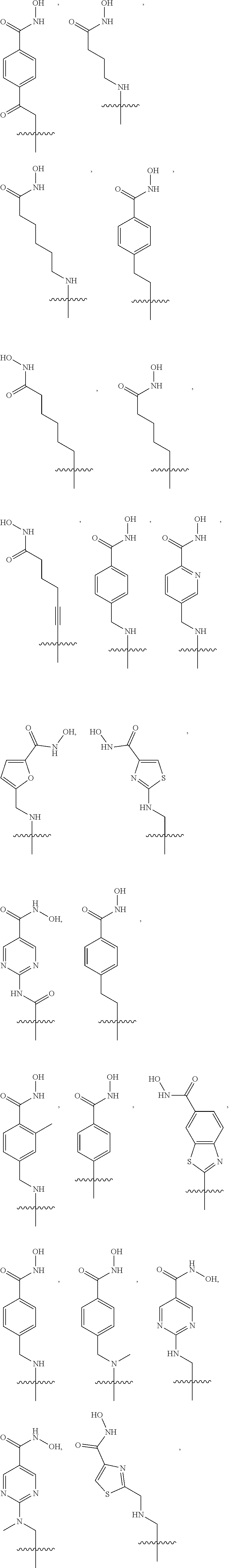

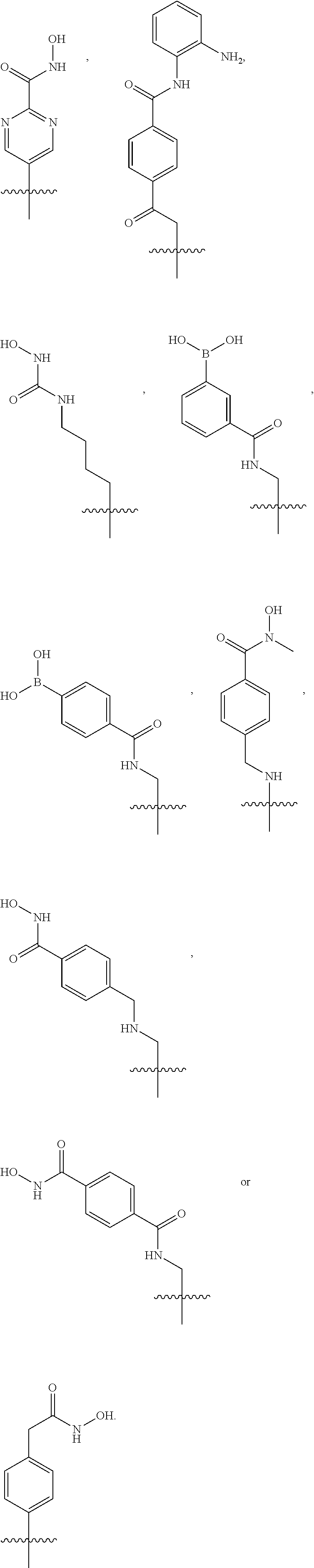

For example, the kinase hinge binding moiety may be:

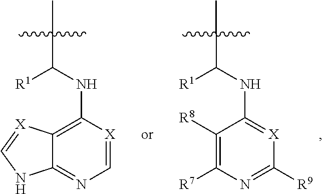

wherein R¹ may be a C₁-C₅ alkyl group;

R⁷ may be H, a C₁-C₅ alkyl group, a C₁-C₅ alkyl containing 1-5 fluorine atoms, a C₁-C₅ alkyl containing 1-5 deuterium atoms, or NH₂;

R⁸ may be H, a C₁-C₅ alkyl group, Cl, CONH₂, or CN;

R⁹ may be H, a C₁-C₅ alkyl group, a C₁-C₅ alkyl containing 1-5 fluorine atoms, a C₁-C₅ alkyl containing 1-5 deuterium atoms, or NH₂; and

X may be CH or N.

In another embodiment, the core of the dual inhibitor may be represented by Formula 2, but is not limited thereto:

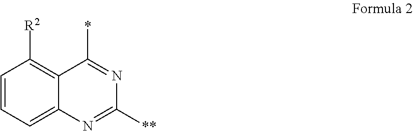

wherein

R² may be hydrogen, a halogen, or a C₁-C₅ alkyl group.

“*” indicates a binding site to the histone deacetylase pharmacophore, and

“**′” indicates a binding site to the kinase hinge binding moiety.

For example, the histone deacetylase pharmacophore may be:

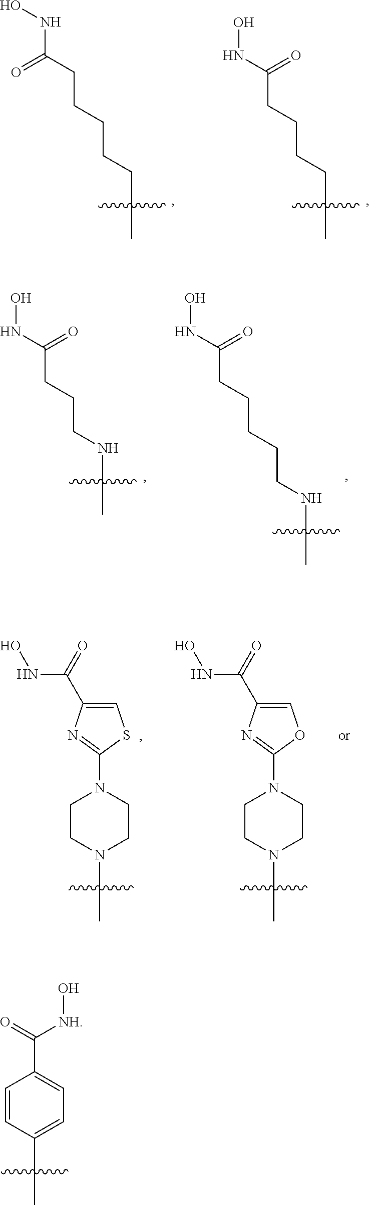

For example, the kinase hinge binding moiety may be:

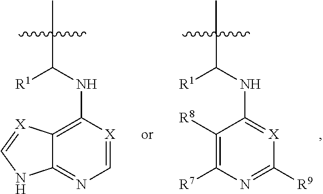

wherein R¹ may be a C₁-C₅ alkyl group;

R⁷ may be H, a C₁-C₅ alkyl group, a C₁-C₅ alkyl containing 1-5 fluorine atoms, a C₁-C₅ alkyl containing 1-5 deuterium atoms, or NH₂;

R⁸ may be H, a C₁-C₅ alkyl group, Cl, CONH₂, or CN;

R⁹ may be H, a C₁-C₅ alkyl group, a C₁-C₅ alkyl containing 1-5 fluorine atoms, a C₁-C₅ alkyl containing 1-5 deuterium atoms, or NH₂; and

X may be CH or N.

In an embodiment, the dual inhibitor may be represented by Formula 3:

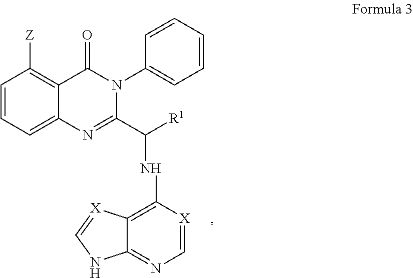

In Formula 3,
R¹ may be a C₁-C₅ alkyl group,
X may be CH or N, and
Z may be:

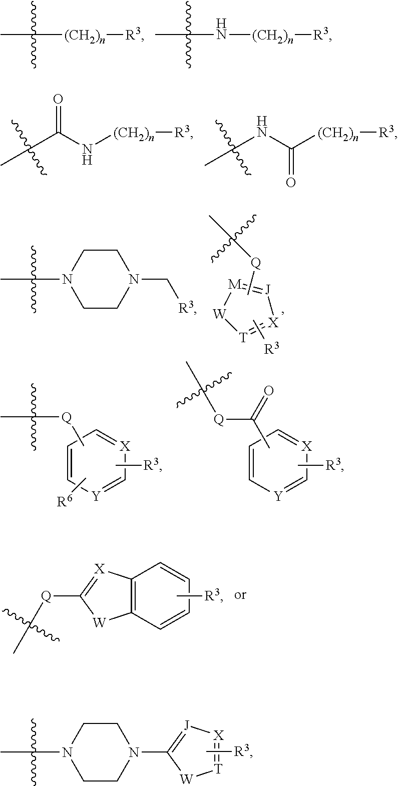

but is not limited thereto,
wherein in the above formulae,
at least one non-adjacent —CH₂— group may be optionally replaced with —O—;
n may be 1, 2, 3, 4, and 5;
J may be CH or N;
M may be CH or N;
W may be N, O, or S;
X may be CH or N;
T may be CH or N;
Q may be —(CH₂)ₚ—, —(CH₂)ₚNH(CH₂)ᵣ—, —NH(CH₂)ₚ— or —(CH₂)ₚNH—, wherein p and r may each independently be 0, 1, 2, 3, or 5;
Y may be CH or N;
R³ may be

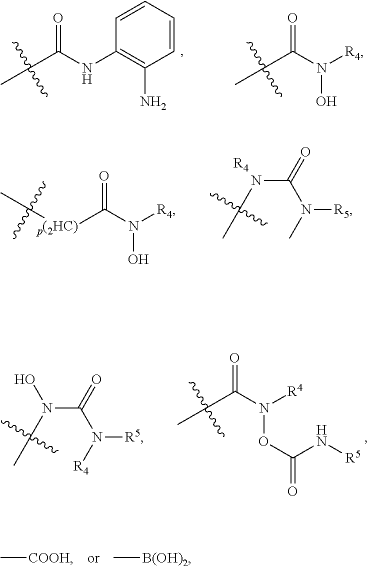

wherein R⁴ and R⁵ may each independently be H or a C₁-C₅ alkyl group;

R⁶ is H or a C₁-C₄ alkyl group.

In an embodiment, the dual inhibitor may be represented by Formula 4:

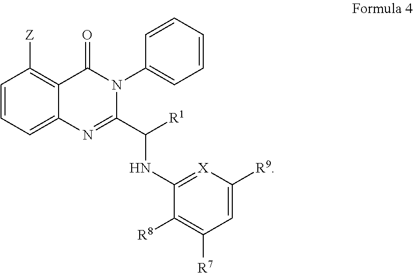

In Formula 4,

R¹ may be a C₁-C₅ alkyl group;

R⁷ may be H, a C₁-C₅ alkyl group, a C₁-C₅ alkyl containing 1-5 fluorine atoms, a C₁-C₅ alkyl containing 1-5 deuterium atoms, or NH₂;

R⁸ may be H, a C₁-C₅ alkyl group, Cl, CONH₂, or CN;

R⁹ may be H, a C₁-C₅ alkyl group, a C₁-C₅ alkyl containing 1-5 fluorine atoms, a C₁-C₅ alkyl containing 1-5 deuterium atoms, or NH₂; and

X may be CH or N; and

Z may be

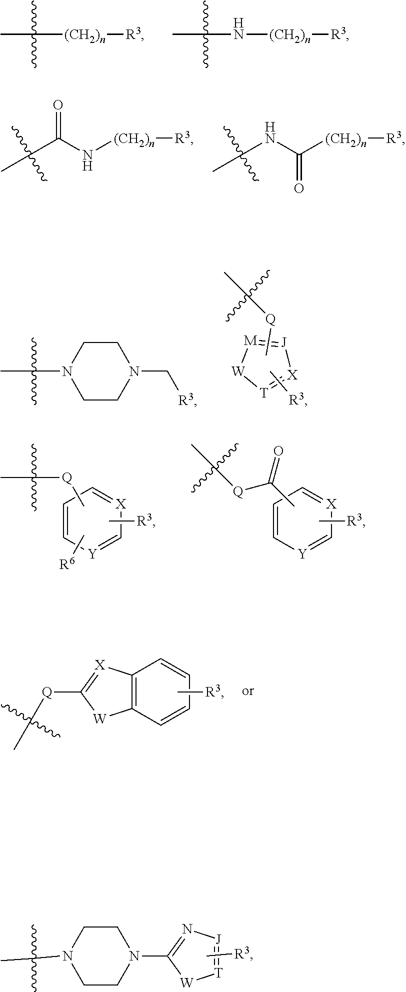

but is not limited thereto,

wherein in the above formulae,
at least one non-adjacent —CH₂— group may be optionally replaced with —O—;
n may be 1, 2, 3, 4, and 5;
J may be CH or N;
M may be CH or N;
W may be N, O, or S;
X may be CH or N;
T may be CH or N;
Q may be —(CH₂)ₚ—, —(CH₂)ₚNH(CH₂)ᵣ—, —NH(CH₂)ₚ— or —(CH₂)ₚNH—, wherein p and r may each independently be 0, 1, 2, 3, or 5;
Y may be CH or N;
R³ may be

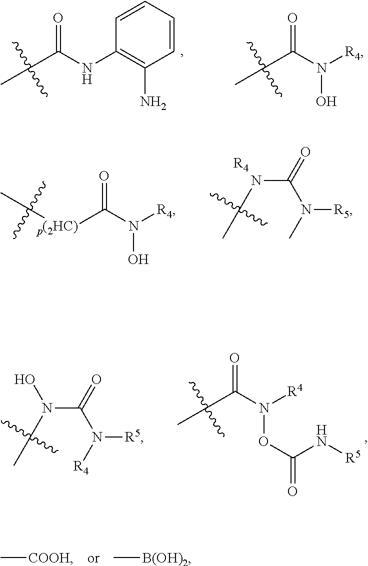

wherein R⁴ and R⁵ may each independently be H or a C₁-C₅ alkyl group;
R⁶ is H or a C₁-C₄ alkyl group;

In another embodiment, the dual inhibitor may be represented by Formula 5:

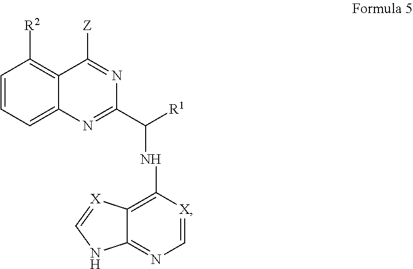

In Formula 5,

R¹ may be a C₁-C₅ alkyl group,

R² may be hydrogen, a halogen, or a C₁-C₅ alkyl group,

X may be CH or N, and

Z may be

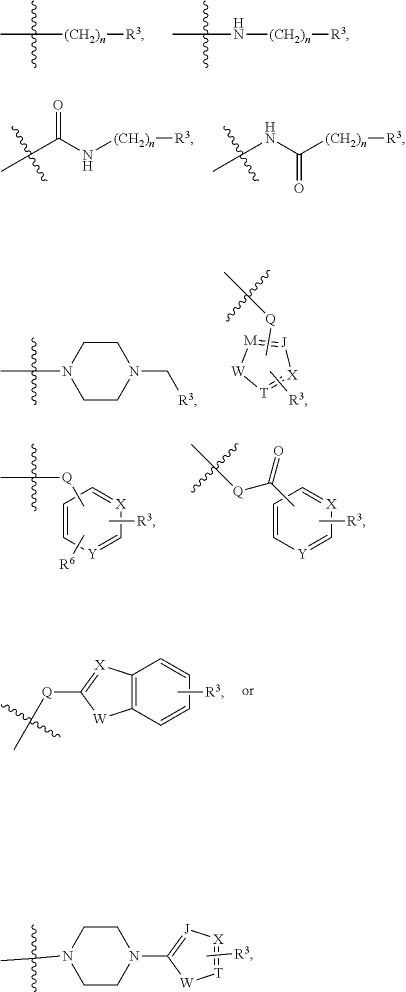

but is not limited thereto,
wherein in the above formulae,
at least one non-adjacent —CH₂— group may be optionally replaced with —O—;
n may be 1, 2, 3, 4, and 5;
J may be CH or N;
M may be CH or N;
W may be N, O, or S;
X may be CH or N;
T may be CH or N;
Q may be —(CH₂)—, —(CH₂)ₚNH(CH₂)ᵣ—, —NH(CH₂)ₚ—, or —(CH₂)ₚNH—, wherein p and r may each independently be 0, 1, 2, 3, or 5;
Y may be CH or N;
R³ may be

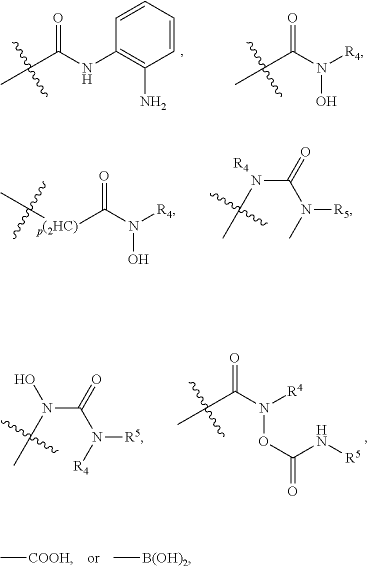

wherein R⁴ and R⁵ may each independently be a C₁-C₅ alkyl group;
R⁶ may be H or a C₁-C₄ alkyl group.

In another embodiment, the dual inhibitor may be represented by Formula 6:

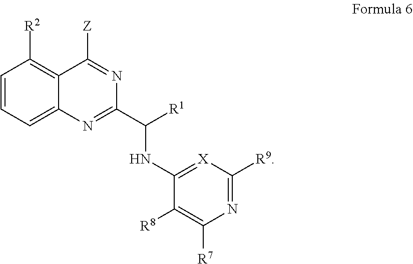

In Formula 6,

R¹ may be a C₁-C₅ alkyl group;

R² may be hydrogen, a halogen, or a C₁-C₅ alkyl group;

R⁷ may be H, a C₁-C₅ alkyl group, a C₁-C₅ alkyl containing 1-5 fluorine atoms, a C₁-C₅ alkyl containing 1-5 deuterium atoms, or NH₂;

R⁸ may be H, a C₁-C₅ alkyl group, Cl, CONH₂, or CN;

R⁹ may be H, a C₁-C₅ alkyl group, a C₁-C₅ alkyl containing 1-5 fluorine atoms, a C₁-C₅ alkyl containing 1-5 deuterium atoms, or NH₂; and
X may be CH or N; and
Z may be

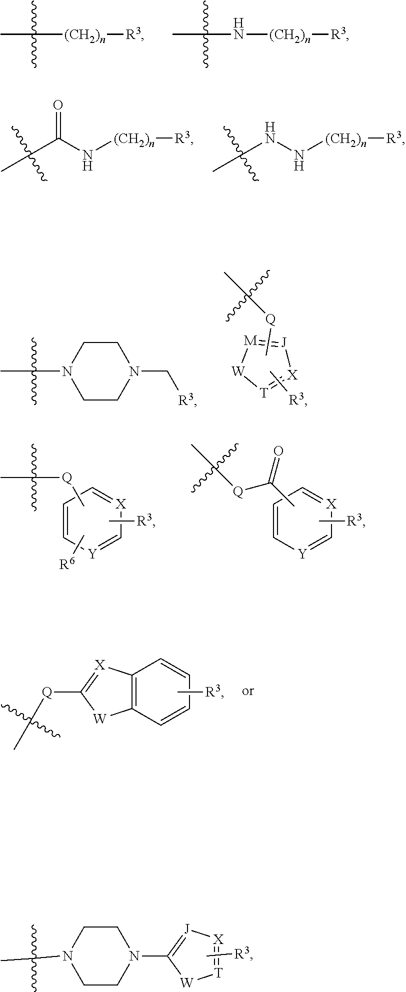

but is not limited thereto,
wherein in the above formulae,
at least one non-adjacent —CH₂— group may be optionally replaced with —O—;
n may be 1, 2, 3, 4, and 5;
J may be CH or N;
M may be CH or N;
W may be N, O, or S;
X may be CH or N;
T may be CH or N;
Q may be —(CH₂)—, —(CH₂)ₚNH(CH₂)ᵣ—, —NH(CH₂)ₚ— or —(CH₂)ₚNH—, wherein p and r may each independently be 0, 1, 2, 3, or 5;
Y may be CH or N;
R³ may be

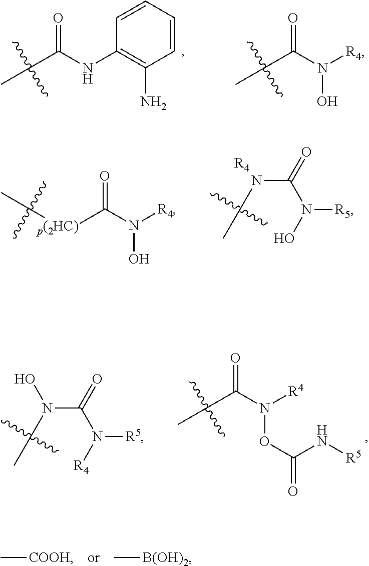

wherein R⁴ and R⁵ may each independently be H or a C₁-C₅ alkyl group;
R⁶ is H or a C₁-C₄ alkyl group;

The dual inhibitor may be represented by one of the following compounds:

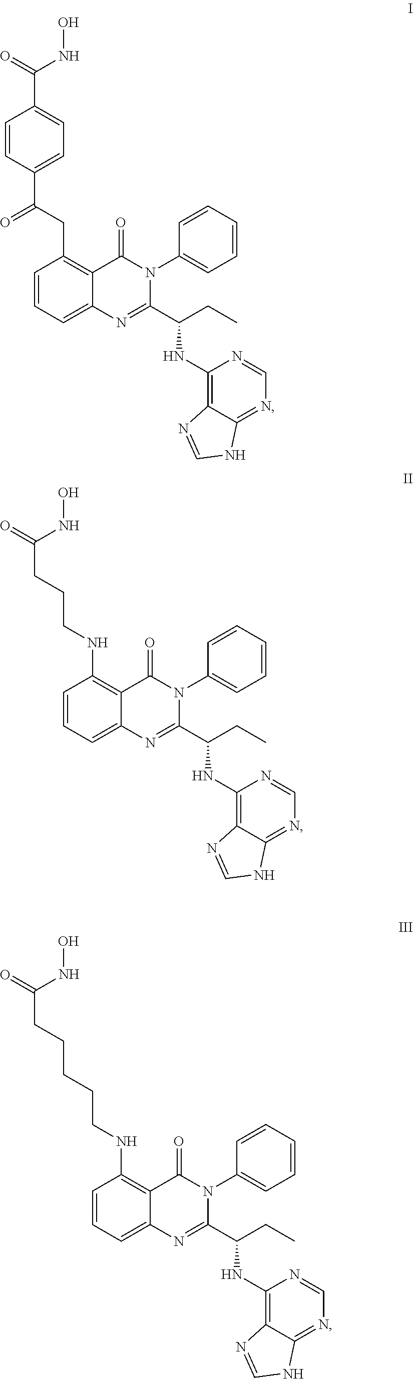 [SMILES: CC[C@H](Nc1ncnc2[nH]cnc12)c1nc2cccc(CC(=O)c3ccc(C(=O)NO)cc3)c2c(=O)n1-c1ccccc1 ; CC[C@H](Nc1ncnc2[nH]cnc12)c1nc2cccc(NCCCC(=O)NO)c2c(=O)n1-c1ccccc1 ; CC[C@H](Nc1ncnc2[nH]cnc12)c1nc2cccc(NCCCCCC(=O)NO)c2c(=O)n1-c1ccccc1]

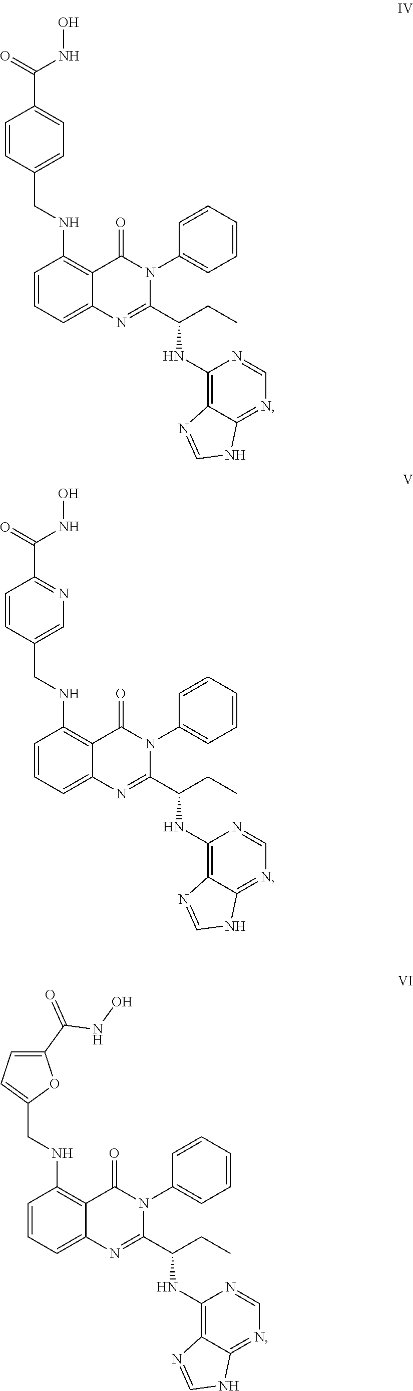 [SMILES: CC[C@H](Nc1ncnc2[nH]cnc12)c1nc2cccc(NCc3ccc(C(=O)NO)cc3)c2c(=O)n1-c1ccccc1 ; CC[C@H](Nc1ncnc2[nH]cnc12)c1nc2cccc(NCc3ccc(C(=O)NO)nc3)c2c(=O)n1-c1ccccc1 ; CC[C@H](Nc1ncnc2[nH]cnc12)c1nc2cccc(NCc3ccc(C(=O)NO)o3)c2c(=O)n1-c1ccccc1]

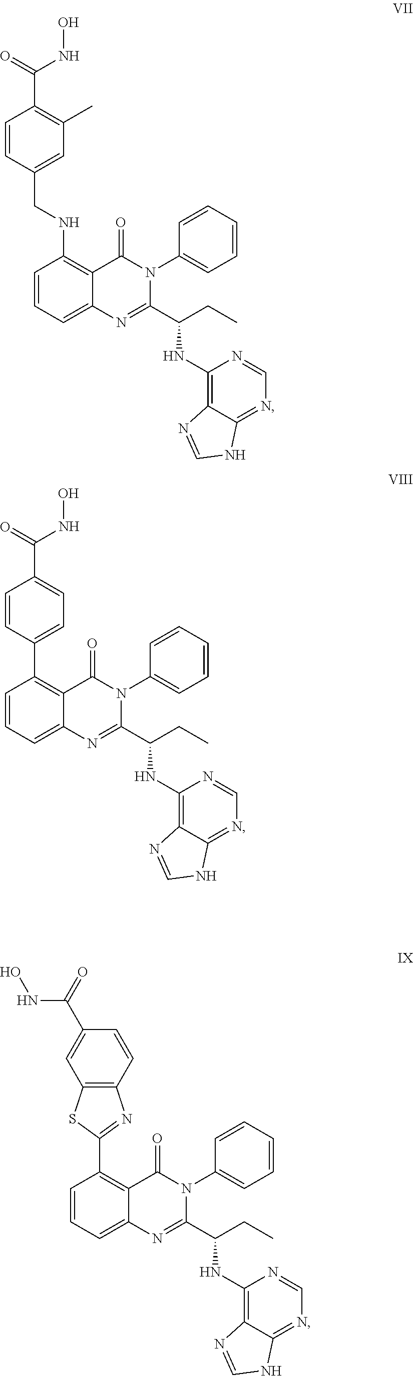 [SMILES: CC[C@H](Nc1ncnc2[nH]cnc12)c1nc2cccc(NCc3ccc(C(=O)NO)c(C)c3)c2c(=O)n1-c1ccccc1 ; CC[C@H](Nc1ncnc2[nH]cnc12)c1nc2cccc(-c3ccc(C(=O)NO)cc3)c2c(=O)n1-c1ccccc1 ; CC[C@H](Nc1ncnc2[nH]cnc12)c1nc2cccc(-c3nc4ccc(C(=O)NO)cc4s3)c2c(=O)n1-c1ccccc1]

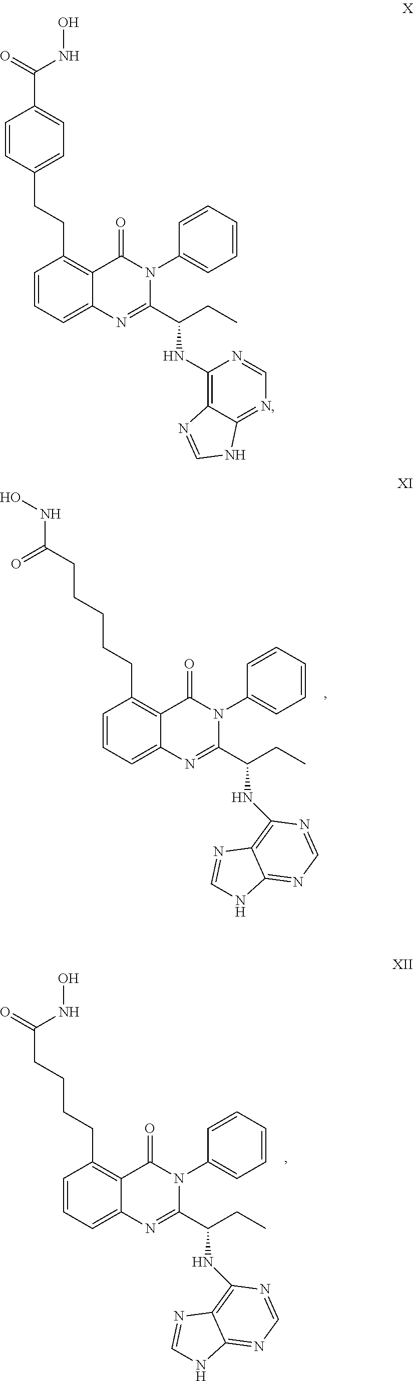 [SMILES: CC[C@H](Nc1ncnc2[nH]cnc12)c1nc2cccc(CCc3ccc(C(=O)NO)cc3)c2c(=O)n1-c1ccccc1 ; CC[C@H](Nc1ncnc2[nH]cnc12)c1nc2cccc(CCCCCC(=O)NO)c2c(=O)n1-c1ccccc1 ; CC[C@H](Nc1ncnc2[nH]cnc12)c1nc2cccc(CCCCC(=O)NO)c2c(=O)n1-c1ccccc1]

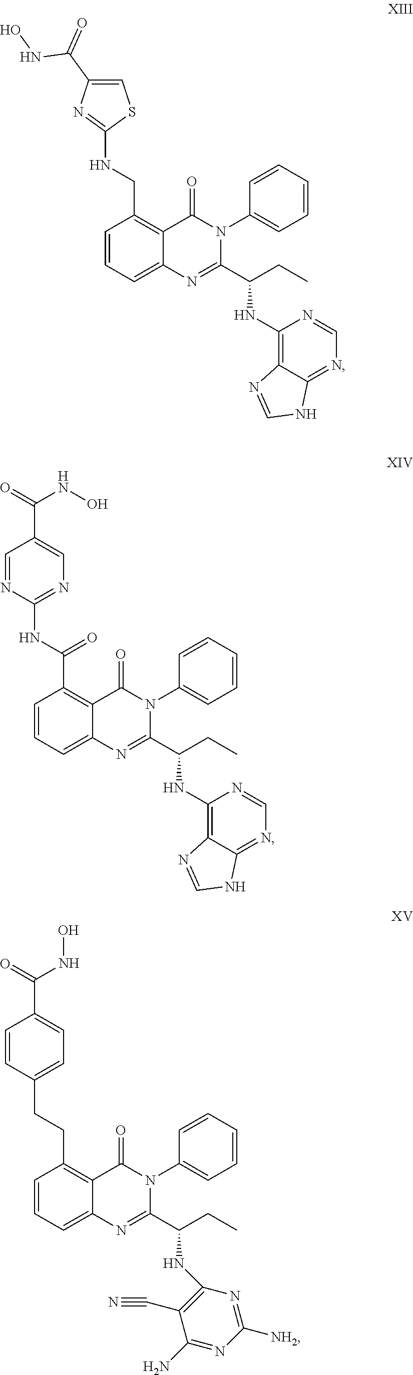 [SMILES: CC[C@H](Nc1ncnc2[nH]cnc12)c1nc2cccc(CNc3nc(C(=O)NO)cs3)c2c(=O)n1-c1ccccc1 ; CC[C@H](Nc1ncnc2[nH]cnc12)c1nc2cccc(C(=O)Nc3ncc(C(=O)NO)cn3)c2c(=O)n1-c1ccccc1 ; CC[C@H](Nc1nc(N)nc(N)c1C#N)c1nc2cccc(CCc3ccc(C(=O)NO)cc3)c2c(=O)n1-c1ccccc1]

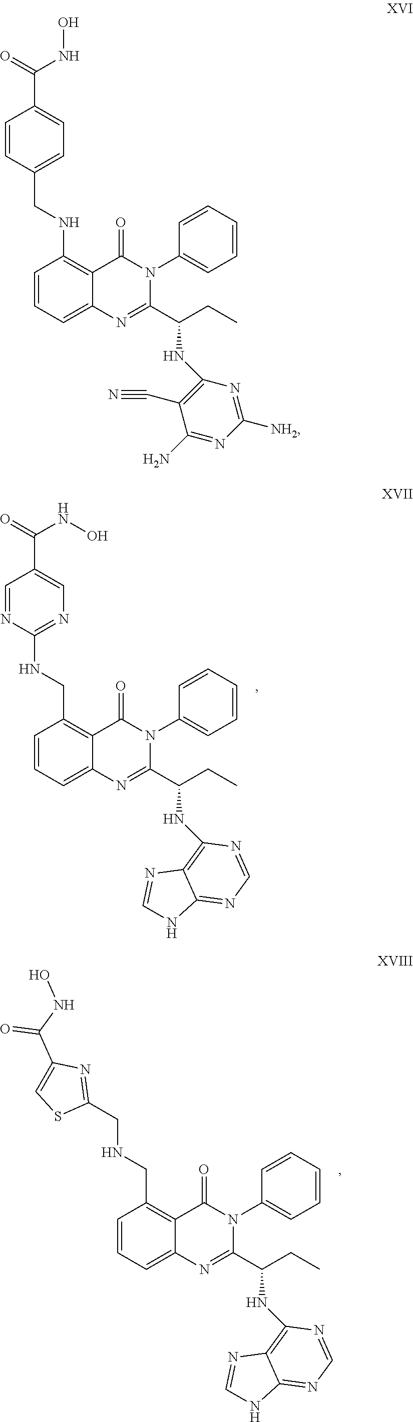 [SMILES: CC[C@H](Nc1nc(N)nc(N)c1C#N)c1nc2cccc(NCc3ccc(C(=O)NO)cc3)c2c(=O)n1-c1ccccc1 ; CC[C@H](Nc1ncnc2[nH]cnc12)c1nc2cccc(CNc3ncc(C(=O)NO)cn3)c2c(=O)n1-c1ccccc1 ; CC[C@H](Nc1ncnc2[nH]cnc12)c1nc2cccc(CNCc3nc(C(=O)NO)cs3)c2c(=O)n1-c1ccccc1]

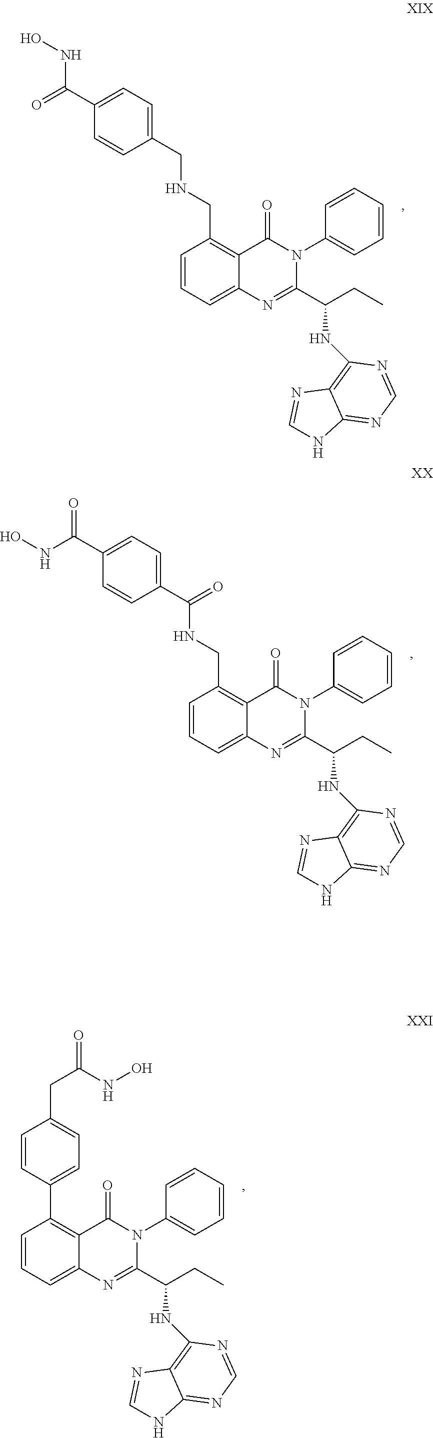 [SMILES: CC[C@H](Nc1ncnc2[nH]cnc12)c1nc2cccc(CNCc3ccc(C(=O)NO)cc3)c2c(=O)n1-c1ccccc1 ; CC[C@H](Nc1ncnc2[nH]cnc12)c1nc2cccc(CNC(=O)c3ccc(C(=O)NO)cc3)c2c(=O)n1-c1ccccc1 ; CC[C@H](Nc1ncnc2[nH]cnc12)c1nc2cccc(-c3ccc(CC(=O)NO)cc3)c2c(=O)n1-c1ccccc1]

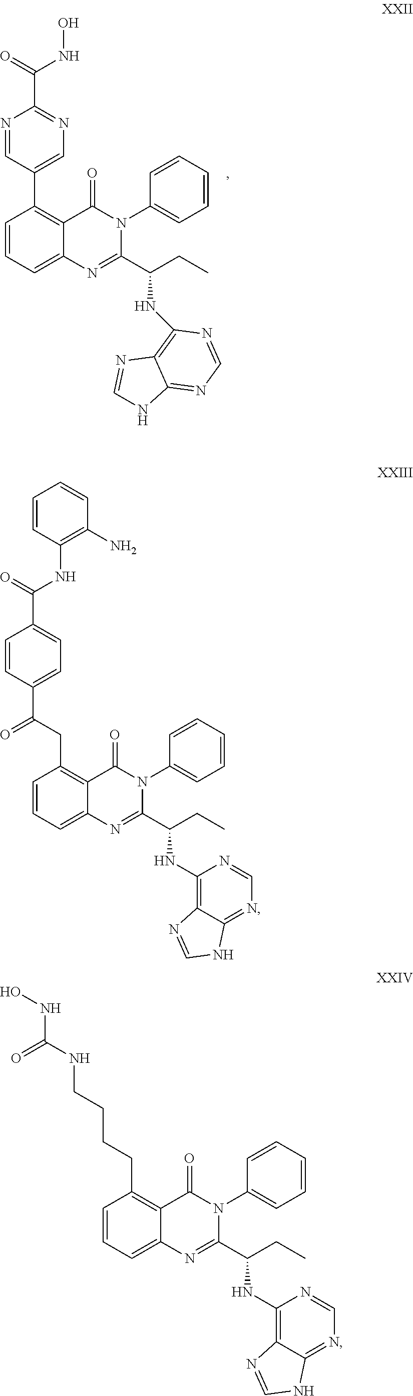 [SMILES: CC[C@H](Nc1ncnc2[nH]cnc12)c1nc2cccc(-c3cnc(C(=O)NO)nc3)c2c(=O)n1-c1ccccc1 ; CC[C@H](Nc1ncnc2[nH]cnc12)c1nc2cccc(CC(=O)c3ccc(C(=O)Nc4ccccc4N)cc3)c2c(=O)n1-c1ccccc1 ; CC[C@H](Nc1ncnc2[nH]cnc12)c1nc2cccc(CCCCNC(=O)NO)c2c(=O)n1-c1ccccc1]

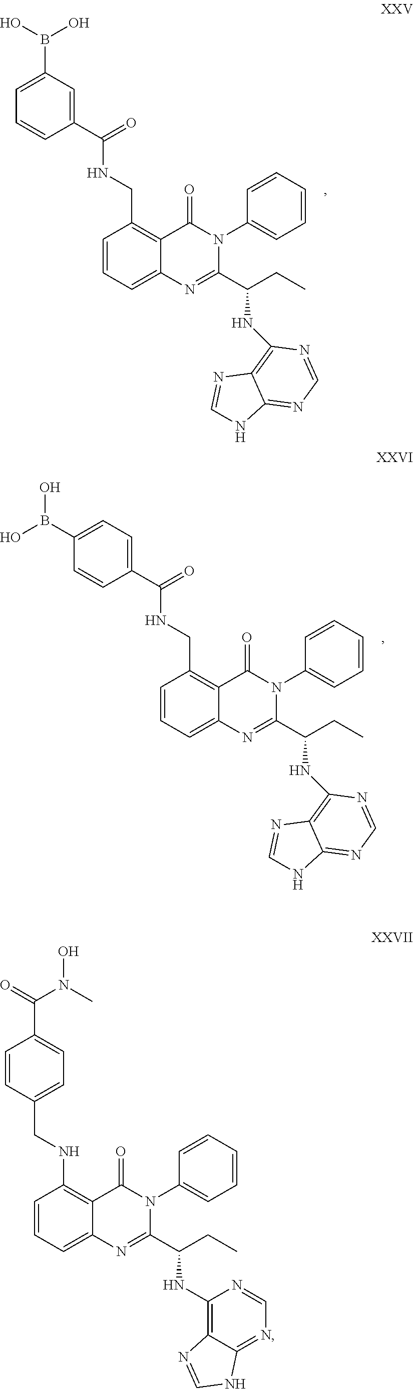 [SMILES: CC[C@H](Nc1ncnc2[nH]cnc12)c1nc2cccc(CNC(=O)c3cccc(B(O)O)c3)c2c(=O)n1-c1ccccc1 ; CC[C@H](Nc1ncnc2[nH]cnc12)c1nc2cccc(CNC(=O)c3ccc(B(O)O)cc3)c2c(=O)n1-c1ccccc1 ; CC[C@H](Nc1ncnc2[nH]cnc12)c1nc2cccc(NCc3ccc(C(=O)N(C)O)cc3)c2c(=O)n1-c1ccccc1]

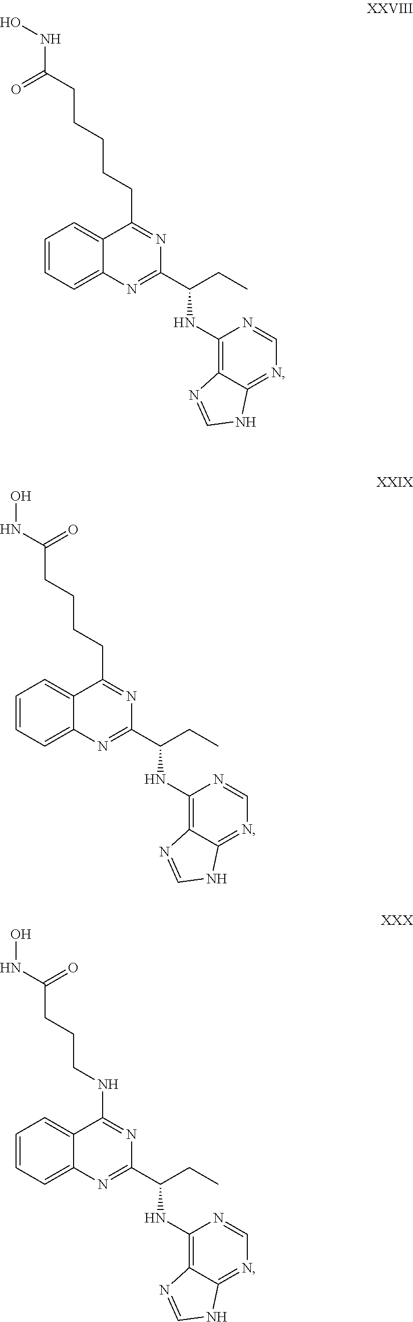 [SMILES: CC[C@H](Nc1ncnc2[nH]cnc12)c1nc(CCCCCC(=O)NO)c2ccccc2n1 ; CC[C@H](Nc1ncnc2[nH]cnc12)c1nc(CCCCC(=O)NO)c2ccccc2n1 ; CC[C@H](Nc1ncnc2[nH]cnc12)c1nc(NCCCC(=O)NO)c2ccccc2n1]

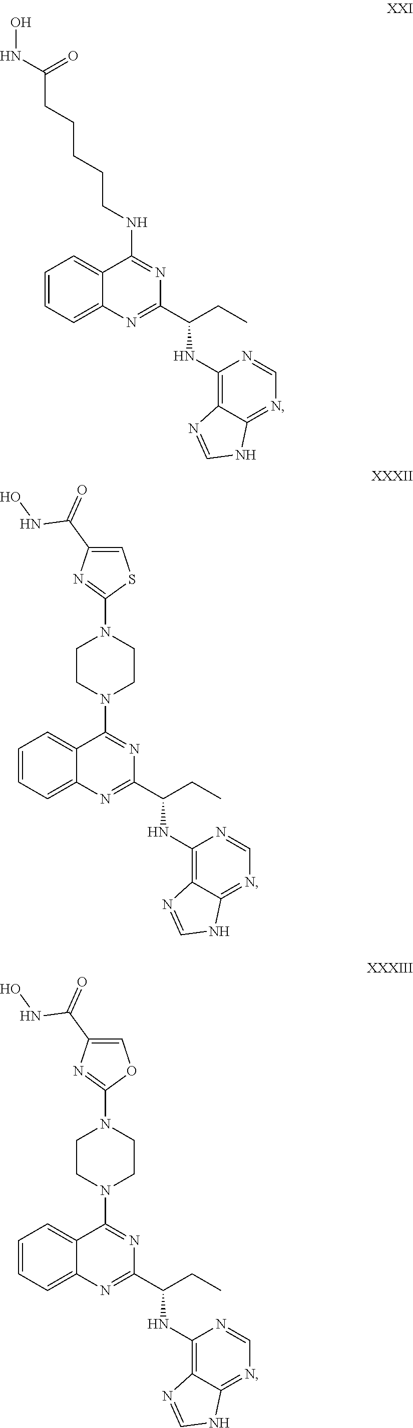 [SMILES: CC[C@H](Nc1ncnc2[nH]cnc12)c1nc(NCCCCCC(=O)NO)c2ccccc2n1 ; CC[C@H](Nc1ncnc2[nH]cnc12)c1nc(N2CCN(c3nc(C(=O)NO)cs3)CC2)c2ccccc2n1 ; CC[C@H](Nc1ncnc2[nH]cnc12)c1nc(N2CCN(c3nc(C(=O)NO)co3)CC2)c2ccccc2n1]

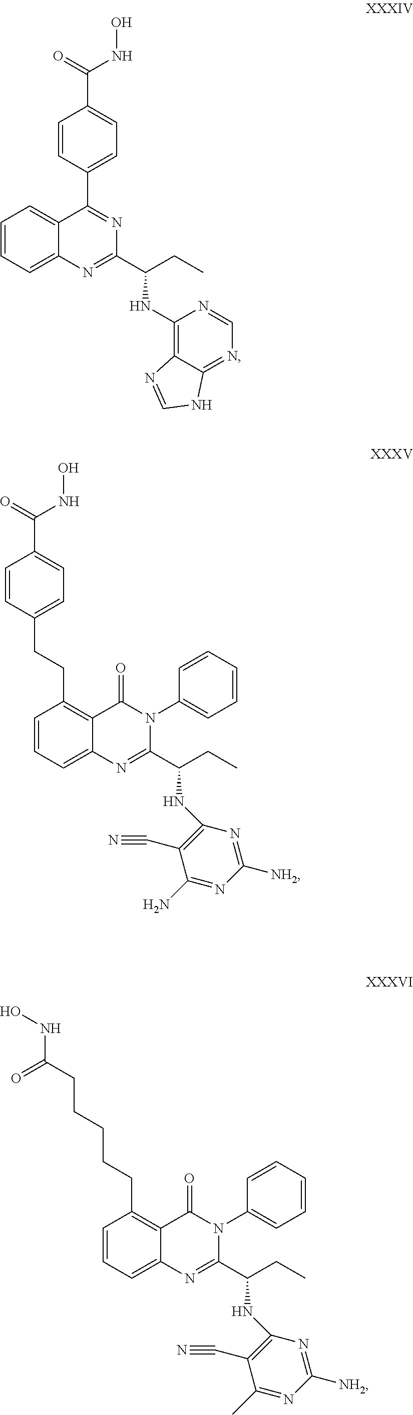 [SMILES: CC[C@H](Nc1ncnc2[nH]cnc12)c1nc(-c2ccc(C(=O)NO)cc2)c2ccccc2n1 ; CC[C@H](Nc1nc(N)nc(N)c1C#N)c1nc2cccc(CCc3ccc(C(=O)NO)cc3)c2c(=O)n1-c1ccccc1 ; CC[C@H](Nc1nc(N)nc(C)c1C#N)c1nc2cccc(CCCCCC(=O)NO)c2c(=O)n1-c1ccccc1]

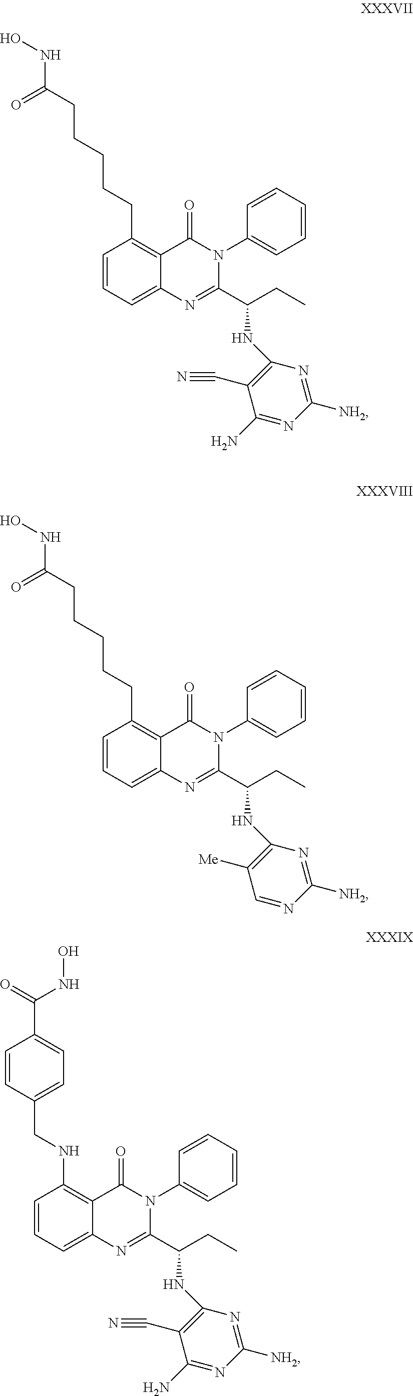 [SMILES: CC[C@H](Nc1nc(N)nc(N)c1C#N)c1nc2cccc(CCCCCC(=O)NO)c2c(=O)n1-c1ccccc1 ; CC[C@H](Nc1nc(N)ncc1C)c1nc2cccc(CCCCCC(=O)NO)c2c(=O)n1-c1ccccc1 ; CC[C@H](Nc1nc(N)nc(N)c1C#N)c1nc2cccc(NCc3ccc(C(=O)NO)cc3)c2c(=O)n1-c1ccccc1]

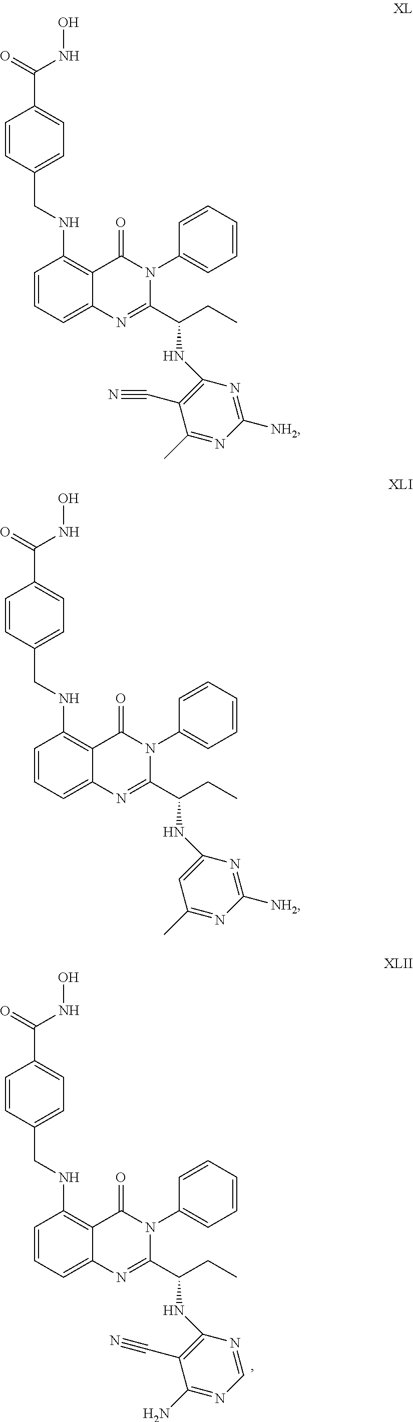 [SMILES: CC[C@H](Nc1nc(N)nc(C)c1C#N)c1nc2cccc(NCc3ccc(C(=O)NO)cc3)c2c(=O)n1-c1ccccc1 ; CC[C@H](Nc1cc(C)nc(N)n1)c1nc2cccc(NCc3ccc(C(=O)NO)cc3)c2c(=O)n1-c1ccccc1 ; CC[C@H](Nc1ncnc(N)c1C#N)c1nc2cccc(NCc3ccc(C(=O)NO)cc3)c2c(=O)n1-c1ccccc1]

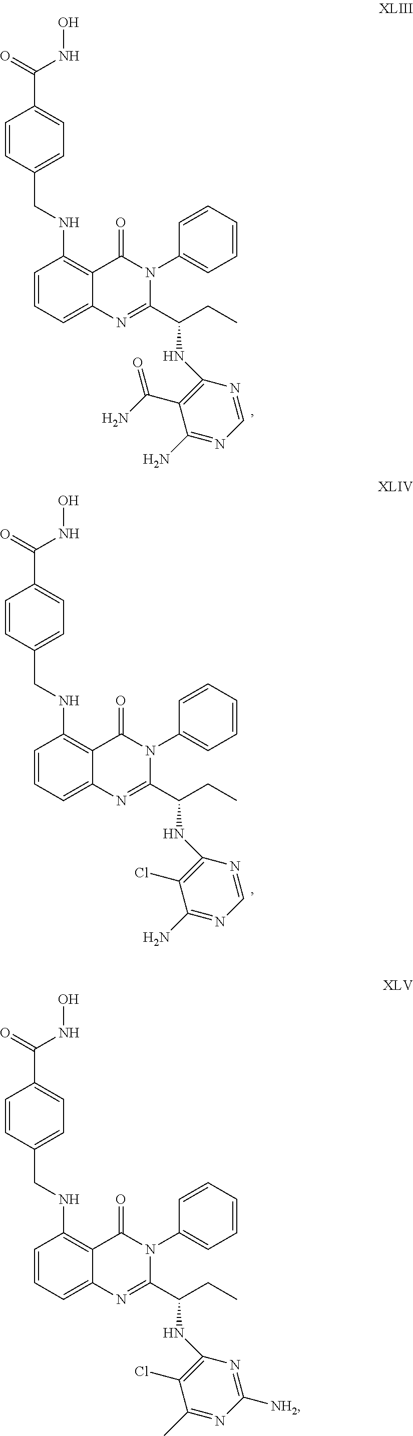 [SMILES: CC[C@H](Nc1ncnc(N)c1C(N)=O)c1nc2cccc(NCc3ccc(C(=O)NO)cc3)c2c(=O)n1-c1ccccc1 ; CC[C@H](Nc1ncnc(N)c1Cl)c1nc2cccc(NCc3ccc(C(=O)NO)cc3)c2c(=O)n1-c1ccccc1 ; CC[C@H](Nc1nc(N)nc(C)c1Cl)c1nc2cccc(NCc3ccc(C(=O)NO)cc3)c2c(=O)n1-c1ccccc1]

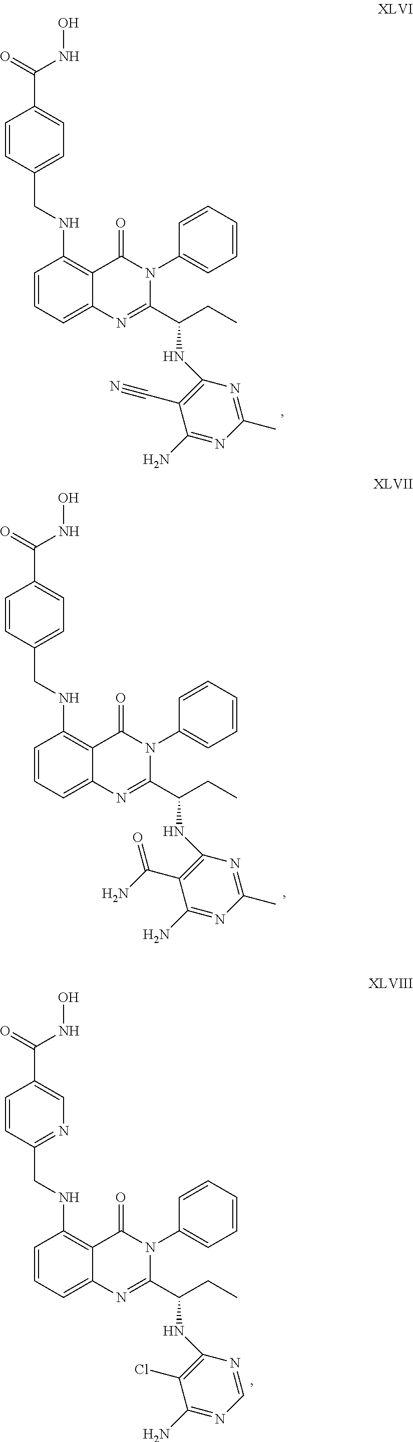 [SMILES: CC[C@H](Nc1nc(C)nc(N)c1C#N)c1nc2cccc(NCc3ccc(C(=O)NO)cc3)c2c(=O)n1-c1ccccc1 ; CC[C@H](Nc1nc(C)nc(N)c1C(N)=O)c1nc2cccc(NCc3ccc(C(=O)NO)cc3)c2c(=O)n1-c1ccccc1 ; CC[C@H](Nc1ncnc(N)c1Cl)c1nc2cccc(NCc3ccc(C(=O)NO)cn3)c2c(=O)n1-c1ccccc1]

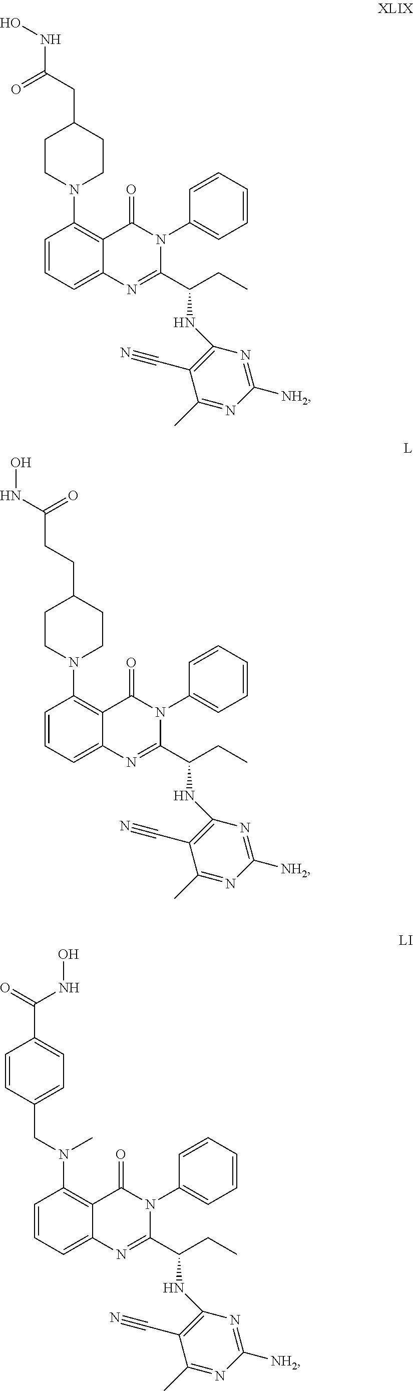 [SMILES: CC[C@H](Nc1nc(N)nc(C)c1C#N)c1nc2cccc(N3CCC(CC(=O)NO)CC3)c2c(=O)n1-c1ccccc1 ; CC[C@H](Nc1nc(N)nc(C)c1C#N)c1nc2cccc(N3CCC(CCC(=O)NO)CC3)c2c(=O)n1-c1ccccc1 ; CC[C@H](Nc1nc(N)nc(C)c1C#N)c1nc2cccc(N(C)Cc3ccc(C(=O)NO)cc3)c2c(=O)n1-c1ccccc1]

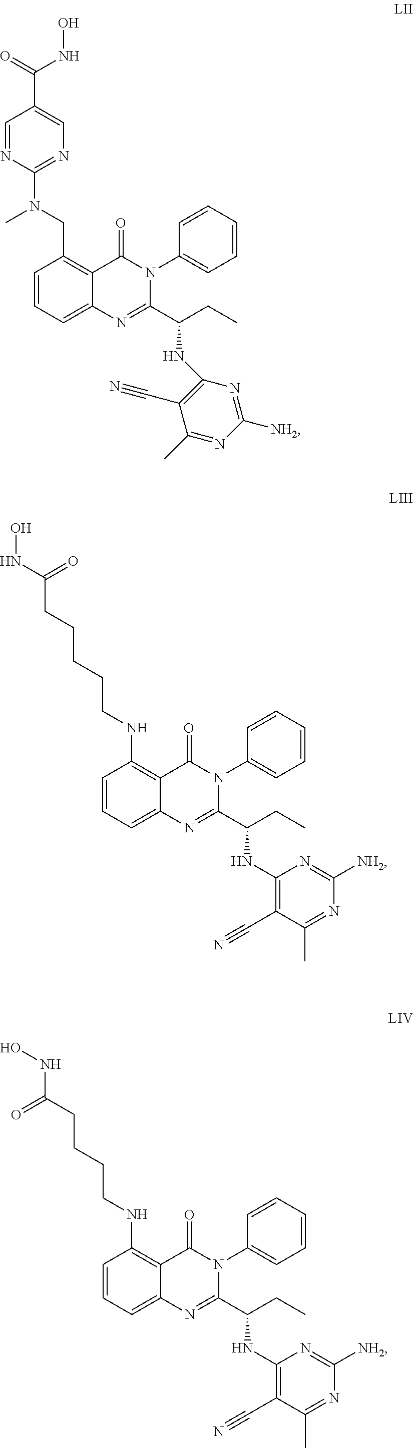 [SMILES: CC[C@H](Nc1nc(N)nc(C)c1C#N)c1nc2cccc(CN(C)c3ncc(C(=O)NO)cn3)c2c(=O)n1-c1ccccc1 ; CC[C@H](Nc1nc(N)nc(C)c1C#N)c1nc2cccc(NCCCCCC(=O)NO)c2c(=O)n1-c1ccccc1 ; CC[C@H](Nc1nc(N)nc(C)c1C#N)c1nc2cccc(NCCCCC(=O)NO)c2c(=O)n1-c1ccccc1]

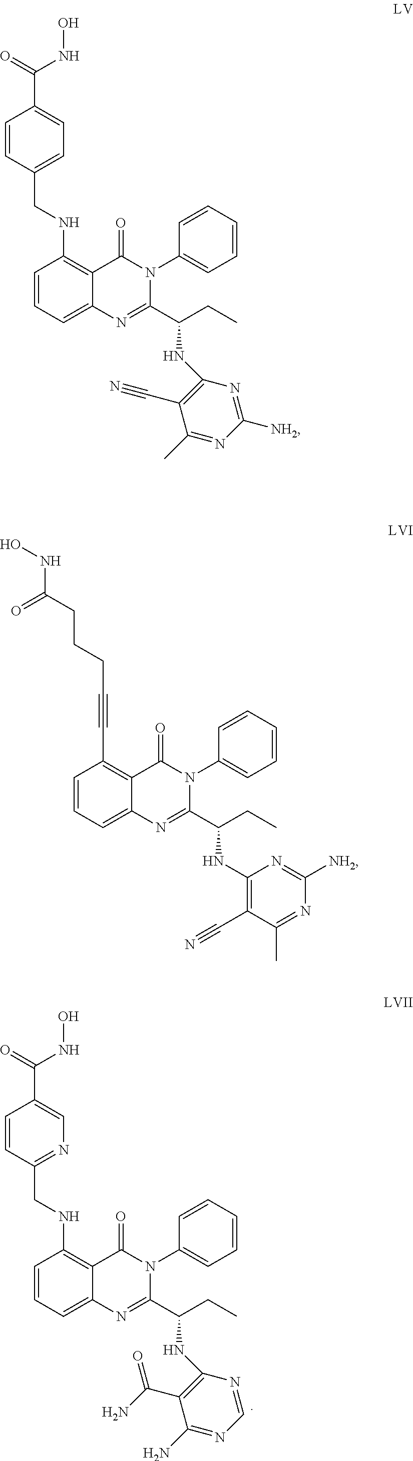 [SMILES: CC[C@H](Nc1nc(N)nc(C)c1C#N)c1nc2cccc(NCc3ccc(C(=O)NO)cc3)c2c(=O)n1-c1ccccc1 ; CC[C@H](Nc1nc(N)nc(C)c1C#N)c1nc2cccc(C#CCCCC(=O)NO)c2c(=O)n1-c1ccccc1 ; CC[C@H](Nc1ncnc(N)c1C(N)=O)c1nc2cccc(NCc3ccc(C(=O)NO)cn3)c2c(=O)n1-c1ccccc1]

The kinase may be a phosphoinositide 3-kinase (PI3K).

In an embodiment, a dual inhibitor of phosphoinositide 3-kinase (PI3K) and histone deacetylase (HDAC) represented by Formula 7 or Formula 8 is provided:

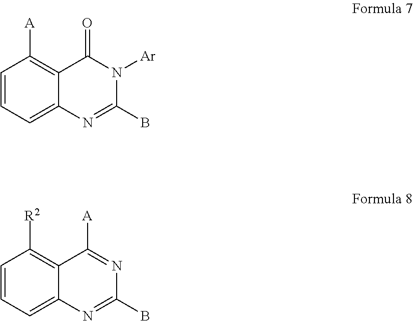

In Formulae 7 and 8, Ar is an aryl or heteroaryl group unsubstituted or substituted with 1-3 C₁-C₆ alkyl groups, R² is hydrogen, a halogen, or a C₁-C₅ alkyl group, A is histone deacetylase pharmacophore, and B is a kinase hinge binding moiety described in detail above.

In another embodiment, a pharmaceutically acceptable salt, a prodrug, or solvate of the dual inhibitor represented by Formulae 7 and 8 is provided.

In another embodiment, a method for treating or diagnosing cancer in a mammal is provided. The method includes administering to the mammal a pharmaceutical composition including an effective amount of an active agent, wherein the active agent is the dual inhibitor of phosphoinositide 3-kinase (PI3K) and histone deacetylase (HDAC), a pharmaceutically acceptable salt thereof, a prodrug thereof, or solvate thereof.

In another embodiment, an inhibitor of histone deacetylase (HDAC), a pharmaceutically acceptable salt thereof, a prodrug thereof, or solvate thereof are provided. The HDAC inhibitor may include a core containing a quinazolin-4(3H)-one moiety and a histone deacetylase pharmacophore.

The HDAC inhibitor may be represented by Formula 9, but is not limited thereto:

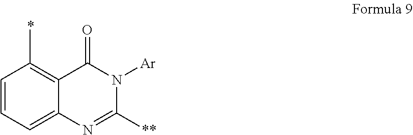

wherein Ar may be an aryl or heteroaryl group unsubstituted or substituted with 1-3 C₁-C₆ alkyl groups,
“*” may be

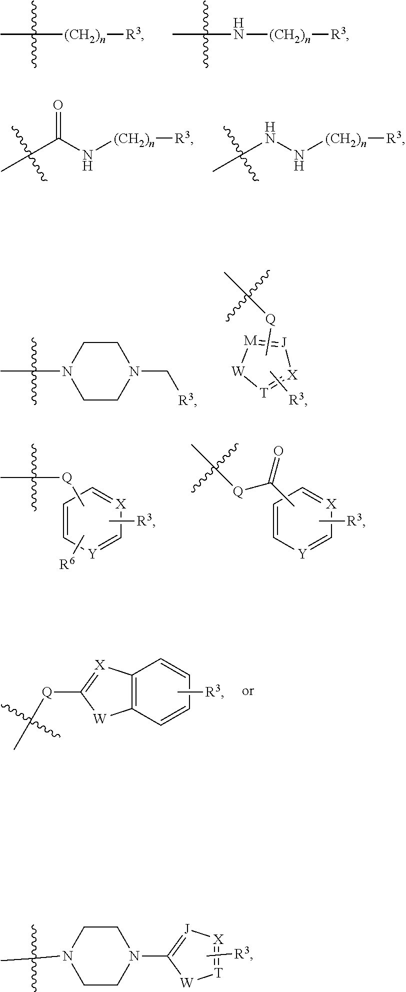

wherein in the above formulae,
at least one non-adjacent —CH₂— group may be optionally replaced with —O—;
n may be 1, 2, 3, 4, and 5;
J may be CH or N;
M may be CH or N;
W may be N, O, or S;
X may be CH or N;
T may be CH or N;
Q may be —(CH₂)—, —(CH₂)ₚNH(CH₂)ᵣ—, —NH(CH₂)ₚ— or —(CH₂)ₚNH—, wherein p and r may each independently be 0, 1, 2, 3, or 5;
Y may be CH or N;
R³ may be

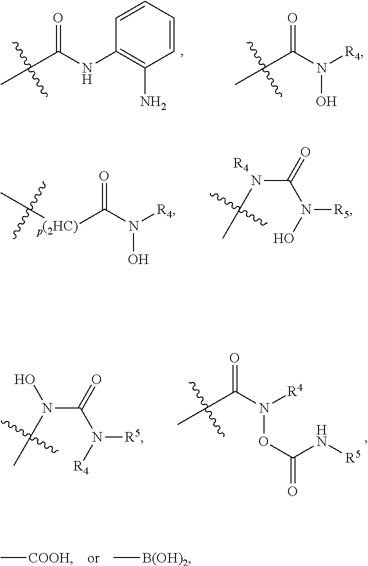

wherein R⁴ and R⁵ may each independently be H or a C₁-C₅ alkyl group; and
R⁶ may be H or a C₁-C₄ alkyl group, and
“**′” may be H, C₁-C₆ alkyl, C₃-C₆ cycloalkyl, or aryl.

The HDAC inhibitor may be represented by one of the following compounds:

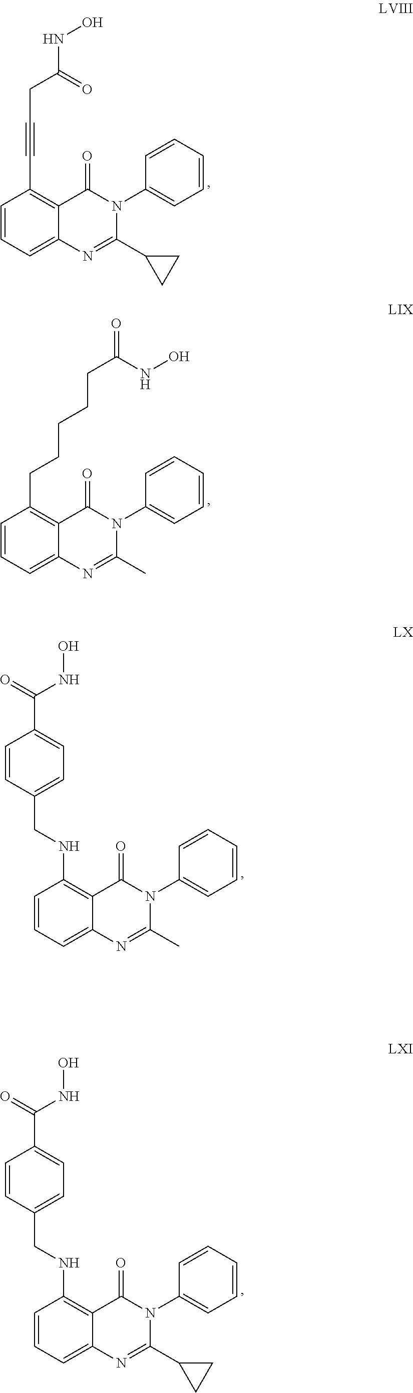 [SMILES: O=C(CC#Cc1cccc2nc(C3CC3)n(-c3ccccc3)c(=O)c12)NO ; Cc1nc2cccc(CCCCCC(=O)NO)c2c(=O)n1-c1ccccc1 ; Cc1nc2cccc(NCc3ccc(C(=O)NO)cc3)c2c(=O)n1-c1ccccc1 ; O=C(NO)c1ccc(CNc2cccc3nc(C4CC4)n(-c4ccccc4)c(=O)c23)cc1]

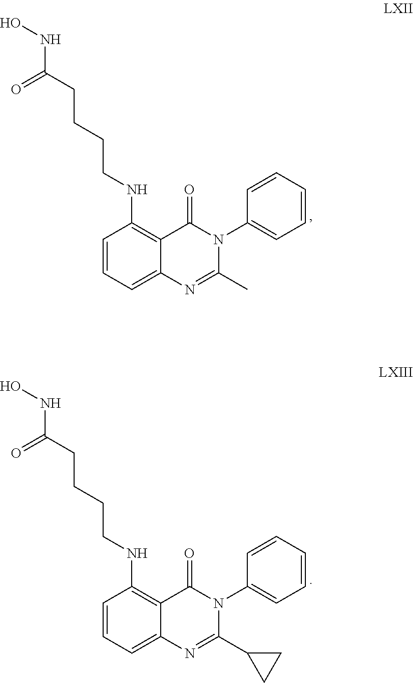 [SMILES: Cc1nc2cccc(NCCCCC(=O)NO)c2c(=O)n1-c1ccccc1 ; O=C(CCCCNc1cccc2nc(C3CC3)n(-c3ccccc3)c(=O)c12)NO]

In an embodiment, an inhibitor of histone deacetylase (HDAC) represented by Formula 10 is provided:

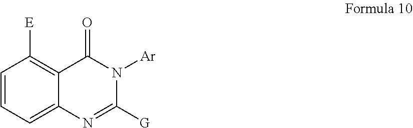

In Formula 10,

Ar is an aryl or heteroaryl group unsubstituted or substituted with 1-3 C₁-C₆ alkyl group,

E is histone deacetylase pharmacophore, and

G is H, C₁-C₆ alkyl, C₃-C₆ cycloalkyl or aryl.

In another embodiment, a pharmaceutically acceptable salt, a prodrug, or solvate of the HDAC inhibitor represented by Formula 10 is provided.

In another embodiment, a method for treating or diagnosing cancer in a mammal is provided. The method includes administering to the mammal a pharmaceutical composition including an effective amount of an active agent, wherein the active agent is the HDAC inhibitor, a pharmaceutically acceptable salt thereof, a prodrug thereof, or solvate thereof.

The cancer to be treated may be blood cancer, lung cancer, colon cancer, central nervous system (CNS) cancer, melanoma cancer, ovarian cancer, renal cancer, prostate cancer, and breast cancer.

Treatment of the blood cancer may include Leukemias represented by cell lines selected from the group consisting of CCRF-CEM, HL-60(TB), K-562, MOLT-4, RPMI-8226, and SR.

Treatment of the lung cancer may include Non-Small Cell Lung Cancer represented by cell lines selected from the group consisting of A549/ATCC, EKVX, HOP-62, HOP-92, NCI-H226, NCI-H23, NCI-H322M, NCI-H460, and NCI-H522.

Treatment of the colon cancer may include colon cancers represented by cell lines selected from the group consisting of COLO 205, HCC-2998, HCT-116, HCT-15, HT29, KM-12, and SW-620.

Treatment of the CNS cancer may include CNS Cancers represented by cell lines selected from the group consisting of SF-268, SF-295, SF-539, SNB-19, SNB-75, and U251.

Treatment of the melanoma cancer may include Melanomas represented by cell lines selected from the group consisting of LOX IMVI, MALME-3M, M14, MDA-MB-435, SK-MEL-2, SK-MEL-28, SK-MEL-5, UACC-257, and UACC-62.

Treatment of the ovarian cancer may include Ovarian Cancers represented by cell lines selected from the group consisting of IGROV1, OVCAR-3, OVCAR-4, OVCAR-5, OVCAR-8, NCI/ADR-RES, and SK-OV-3.

Treatment of the renal cancer may include Renal Cancers represented by cell lines selected from the group consisting of 786-0, A498, ACHN, CAKI-1, RXF 393, SN12C, TK-10, and UO-31.

Treatment of the prostate cancer may include prostate cancer represented by PC-3 and DU-145 cell lines.

Treatment of the breast cancer may Breast Cancer represented by cell lines selected from the group consisting of MCF7, MDA-MB-231/ATCC, HS 578T, BT-549, T-47D, and MDA-MB-468.

### EXAMPLES

### Compound Synthesis

### General Chemical Methods

All air or moisture sensitive reactions were performed under positive pressure of nitrogen with oven-dried glassware. Chemical reagents and anhydrous solvents were obtained from commercial sources and used as is. Preparative purification was performed on a Waters semi-preparative HPLC instrument. The column used was a Phenomenex Luna C18 (5 μm, 30 mm×75 mm) at a flow rate of 45 mL/min. The mobile phase consisted of acetonitrile and water (each containing 0.1% trifluoroacetic acid). A gradient from 10% to 50% acetonitrile over 8 min was used during the purification. Fraction collection was triggered by UV detection (220 nm). Alternately, flash chromatography on silica gel was performed using forced flow (liquid) of the indicated solvent system on Biotage KP-Sil pre-packed cartridges and using the Biotage SP-1 automated chromatography system.

Analytical analysis for purity was determined by two different methods denoted as final QC methods 1 and 2.

Method 1. Analysis was performed on an Agilent 1290 Infinity series HPLC instrument. UHPLC long gradient equivalent from 4% to 100% acetonitrile (0.05% trifluoroacetic acid) in water over 3 min run time of 4.5 min with a flow rate of 0.8 mL/min. A Phenomenex Luna C18 column (3 m, 3 mm×75 mm) was used at a temperature of 50° C.
Method 2. Analysis was performed on an Agilent 1260 with a 7 min gradient from 4% to 100% acetonitrile (containing 0.025% trifluoroacetic acid) in water (containing 0.05% trifluoroacetic acid) over 8 min run time at a flow rate of 1 mL/min. A Phenomenex Luna C18 column (3 m, 3 mm×75 mm) was used at a temperature of 50° C.

Purity determination was performed using an Agilent diode array detector for both method 1 and method 2. Mass determination was performed using an Agilent 6130 mass spectrometer with electrospray ionization in the positive mode. All of the analogs for assay have purity greater than 95% based on both analytical methods. ¹H NMR spectra were recorded on Varian 400 MHz spectrometers. All proton spectra are referenced relative to the deuterated solvent peak: 7.27 ppm for CDCl₃, 2.50 ppm (center line signal) for DMSO-d⁶. High resolution mass spectrometry results were recorded on Agilent 6210 time-of-flight LC/MS system.

### Synthetic Procedures

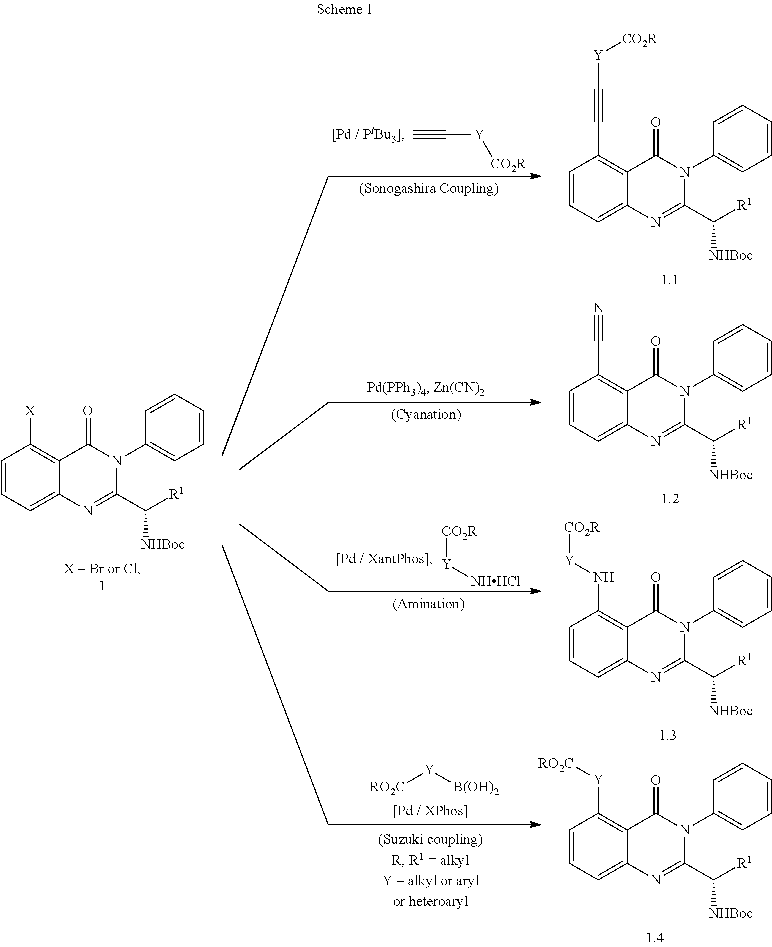

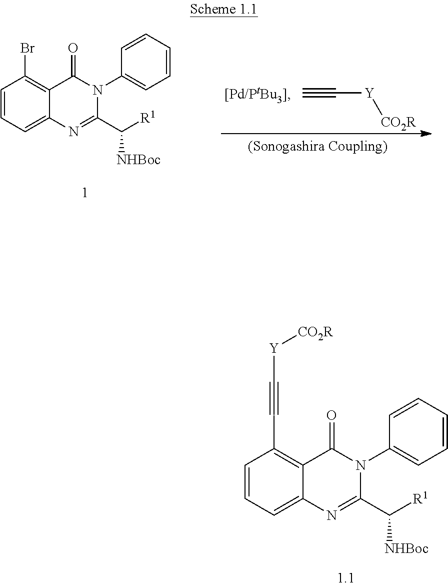

### Scheme 1.1

The substituted aryl bromide 1 (1 equiv, Wei, M. et al. Eur. J. Med. Chem. 2017, 125, 1156), Allylpalladium(II) chloride dimer (0.05 equiv), Tri-tert-butylphosphonium tetrafluoroborate (0.20 equiv) and alkyne (1.2 equiv) [if solid at room temperature] were weighed and added to a MW vial equipped with a stir bar. The vial was covered with a rubber septum and placed under nitrogen atmosphere. In a separate scintillation vial, DABCO was weighed and dissolved in dry 1,4-dioxane (5 ml/mmol of aryl bromide). This DABCO solution and alkyne [if liquid at room temperature] were added to the MW vial via syringe and the resulting mixture is bubbled with nitrogen for 5 min followed by stirring for 16 hours at room temperature under nitrogen atmosphere. After 16 hours, the crude reaction mixture is filtered through a short pad of celite and concentrated in vacuo. The remaining residue was purified by flash chromatography on silica using forced flow of ethyl acetate/hexanes system on Biotage KP-Sil pre-packed cartridges and using the Biotage SP-1 automated chromatography system to afford the coupled product 1.1.

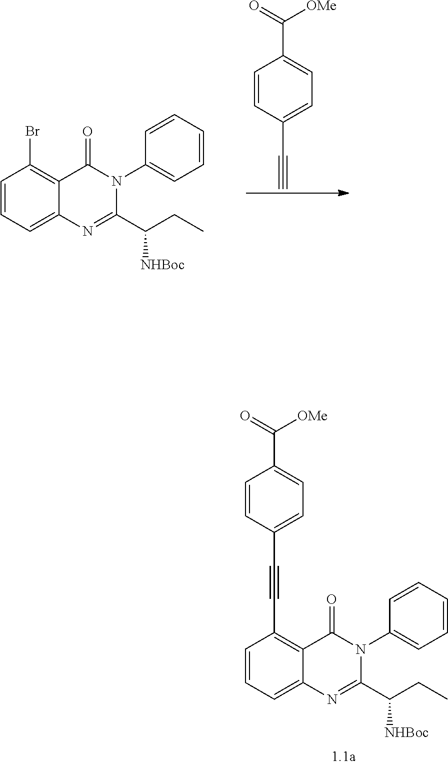 [SMILES: CC[C@H](NC(=O)OC(C)(C)C)c1nc2cccc(Br)c2c(=O)n1-c1ccccc1 ; C#Cc1ccc(C(=O)OC)cc1 ; CC[C@H](NC(=O)OC(C)(C)C)c1nc2cccc(C#Cc3ccc(C(=O)OC)cc3)c2c(=O)n1-c1ccccc1]

The procedure mentioned in Scheme 1.1 was used with ((S)-tert-butyl (1-(5-bromo-4-oxo-3-phenyl-3,4-dihydroquinazolin-2-yl)propyl)carbamate (183.0 mg, 0.40 mmol), Allylpalladium(II) chloride dimer (7.2 mg, 0.02 mmol), Tri-tert-butylphosphonium tetrafluoroborate (12.0 mg, 0.04 mmol), methyl 4-ethynylbenzoate (77.0 mg, 0.48 mmol) and DABCO (90.0 mg, 0.80 mmol) in 2.0 ml of dry 1,4-dioxane. The resulting mixture was stirred at room temperature for 16 hours and concentrated in vacuo. The remaining residue was purified by flash chromatography on silica using 0-25% ethyl acetate/hexanes to afford the product methyl (S)-4-((2-(1-((tert-butoxycarbonyl)amino)propyl)-4-oxo-3-phenyl-3,4-dihydroquinazolin-5-yl)ethynyl)benzoate 1.1a (200.0 mg, 0.37 mmol) as a yellow solid in 93% yield. LC-MS (method 1): t_{R}=3.78 min, m/z (M+H)⁺=538.3. ¹H NMR (400 MHz, Chloroform-d) δ 8.00-7.95 (m, 2H), 7.74-7.69 (m, 3H), 7.66-7.49 (m, 5H), 7.40 (d, J=7.9 Hz, 1H), 7.36-7.30 (m, 1H), 5.49 (d, J=9.0 Hz, 1H), 4.40 (s, 1H), 3.91 (s, 3H), 1.75 (ddd, J=13.9, 7.3, 4.6 Hz, 1H), 1.55-1.48 (m, 1H), 1.43 (s, 9H), 0.77 (t, J=7.4 Hz, 3H).

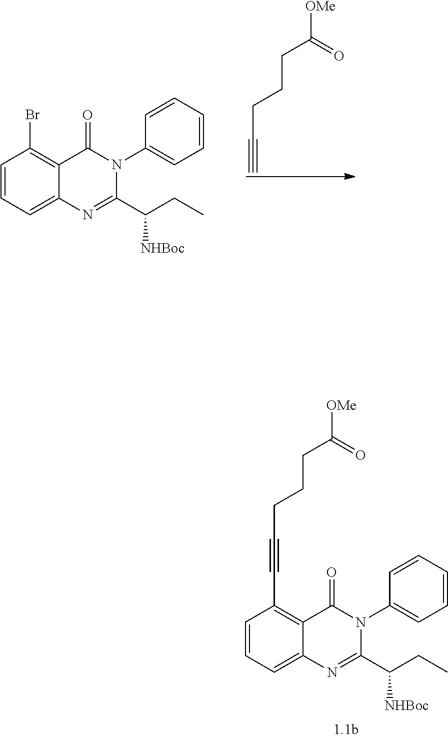

The procedure mentioned in Scheme 1.1 was used with ((S)-tert-butyl (1-(5-bromo-4-oxo-3-phenyl-3,4-dihydroquinazolin-2-yl)propyl)carbamate (234.0 mg, 0.51 mmol), Allylpalladium(II) chloride dimer (9.3 mg, 0.03 mmol), Tri-tert-butylphosphonium tetrafluoroborate (15.0 mg, 0.05 mmol), methyl hex-5-ynoate (77.0 mg, 0613 mmol) and DABCO (115.0 mg, 1.02 mmol) in 2.5 ml of dry 1,4-dioxane. The resulting mixture was stirred at room temperature for 16 hours and concentrated in vacuo. The remaining residue was purified by flash chromatography on silica using 0-30% ethyl acetate/hexanes to afford the product methyl (S)-6-(2-(1-((tert-butoxycarbonyl)amino)propyl)-4-oxo-3-phenyl-3,4-dihydroquinazolin-5-yl)hex-5-ynoate 1.1b (162.0 mg, 0.322 mmol) as a yellow oil in 63% yield. LC-MS (method 1): t_{R}=3.60 min, m/z (M+H)⁺=504.3. ¹H NMR (400 MHz, Chloroform-d) δ 7.67-7.47 (m, 6H), 7.39-7.28 (m, 2H), 5.49 (d, J=9.0 Hz, 1H), 4.38 (s, 1H), 3.65 (s, 3H), 2.53 (dt, J=15.0, 7.2 Hz, 4H), 1.94 (p, J=7.2 Hz, 2H), 1.79-1.66 (m, 1H), 1.57-1.45 (m, 2H), 1.43 (s, 9H), 0.75 (t, J=7.4 Hz, 3H).

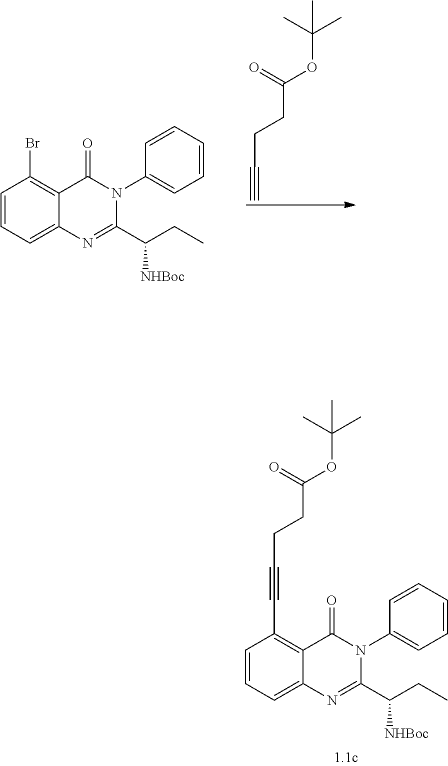 [SMILES: CC[C@H](NC(=O)OC(C)(C)C)c1nc2cccc(Br)c2c(=O)n1-c1ccccc1 ; C#CCCC(=O)OC(C)(C)C ; CC[C@H](NC(=O)OC(C)(C)C)c1nc2cccc(C#CCCC(=O)OC(C)(C)C)c2c(=O)n1-c1ccccc1]

The procedure mentioned in Scheme 1.1 was used with ((S)-tert-butyl (1-(5-bromo-4-oxo-3-phenyl-3,4-dihydroquinazolin-2-yl)propyl)carbamate (150.0 mg, 0.33 mmol), Allylpalladium(II) chloride dimer (5.9 mg, 0.02 mmol), Tri-tert-butylphosphonium tetrafluoroborate (9.5 mg, 0.03 mmol), tert-butyl pent-4-yn-oate (60.6 mg, 0.39 mmol) and DABCO (73.4 mg, 0.66 mmol) in 2.5 ml of dry 1,4-dioxane. The resulting mixture was stirred at room temperature for 16 hours and concentrated in vacuo. The remaining residue was purified by flash chromatography on silica using 0-25% ethyl acetate/hexanes to afford the product methyl tert-butyl (S)-5-(2-(1-((tert-butoxycarbonyl)amino)propyl)-4-oxo-3-phenyl-3,4-dihydroquinazolin-5-yl)pent-4-ynoate 1.1c (130.0 mg, 0.245 mmol) as a yellow oil in 75% yield. LC-MS (method 1): t_{R}=3.90 min, m/z (M+H)⁺=532.4. 1H NMR (400 MHz, Chloroform-d) δ 7.67-7.48 (m, 6H), 7.37 (d, J=8.0 Hz, 1H), 7.32-7.28 (m, 1H), 5.55 (s, 1H), 4.38 (s, 1H), 2.74 (dd, J=8.4, 6.7 Hz, 2H), 2.55 (dd, J=8.3, 6.7 Hz, 2H), 1.47-1.40 (m, 18H), 0.76 (t, J=7.4 Hz, 3H).

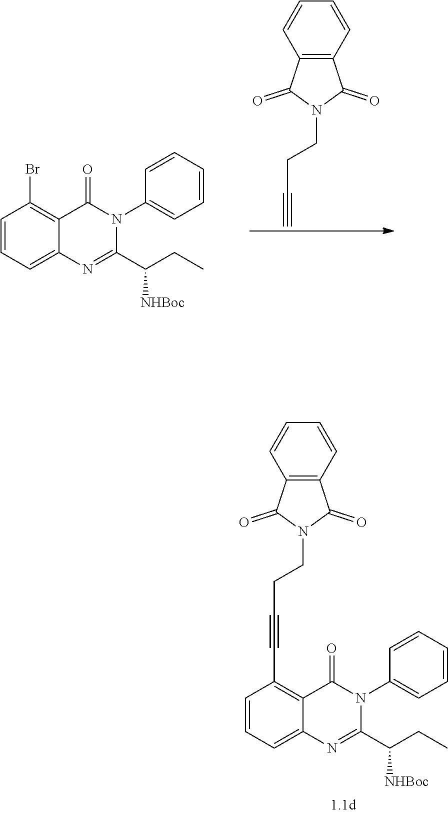 [SMILES: CC[C@H](NC(=O)OC(C)(C)C)c1nc2cccc(Br)c2c(=O)n1-c1ccccc1 ; C#CCCN1C(=O)c2ccccc2C1=O ; CC[C@H](NC(=O)OC(C)(C)C)c1nc2cccc(C#CCCN3C(=O)c4ccccc4C3=O)c2c(=O)n1-c1ccccc1]

The procedure mentioned in Scheme 1.1 was used with ((S)-tert-butyl (1-(5-bromo-4-oxo-3-phenyl-3,4-dihydroquinazolin-2-yl)propyl)carbamate (190.0 mg, 0.42 mmol), Allylpalladium(II) chloride dimer (7.5 mg, 0.02 mmol), Tri-tert-butylphosphonium tetrafluoroborate (12.0 mg, 0.04 mmol), 2-(but-3-yn-1-yl)isoindoline-1,3-dione (99.0 mg, 0.50 mmol) and DABCO (93.0 mg, 0.83 mmol) in 2.0 ml of dry 1,4-dioxane. The resulting mixture was stirred at room temperature for 16 hours and concentrated in vacuo. The remaining residue was purified by flash chromatography on silica using 0-45% ethyl acetate/hexanes to afford the product methyl tert-butyl (S)-(1-(5-(4-(1,3-dioxoisoindolin-2-yl)but-1-yn-1-yl)-4-oxo-3-phenyl-3,4-dihydroquinazolin-2-yl)propyl)carbamate 1.1d (125.0 mg, 0.22 mmol) in 52% yield. LC-MS (method 1): t_{R}=3.72 min, m/z (M+H)⁺=577.3.

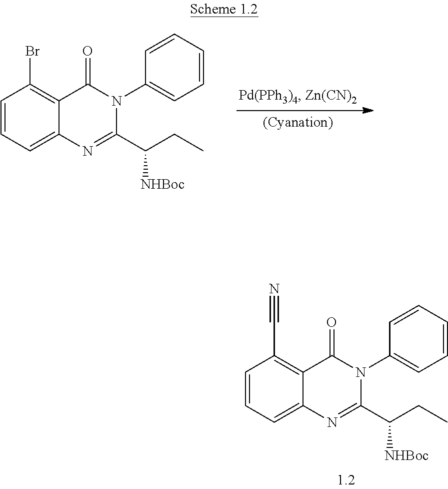 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(C#N)c2c(=O)n1-c1ccccc1]

### Scheme 1.2

((S)-tert-butyl (1-(5-bromo-4-oxo-3-phenyl-3,4-dihydroquinazolin-2-yl)propyl)carbamate (181.0 mg, 0.40 mmol), zinc cyanide (58.0 mg, 0.49 mmol), and tetrakis(triphenylphosphine)Pd(0) (23.0 mg, 0.02 mmol) in dry DMF (2.0 ml) in a MW vial equipped with a stir bar under nitrogen atmosphere. The mixture was bubbled with N₂ gas for 2 minutes, sealed and heated at 100° C. for 16 hours. After 16 hours, the crude reaction mixture is filtered through a short pad of celite and concentrated in vacuo. The remaining residue was purified by flash chromatography on silica using 0-45% ethyl acetate/hexanes to afford the product tert-butyl (S)-(1-(5-cyano-4-oxo-3-phenyl-3,4-dihydroquinazolin-2-yl)propyl)carbamate 1.2 (154.0 mg, 0.38 mmol) as a colorless solid in 96% yield. LC-MS (method 1): t_{R}=3.64 min, m/z (M+H)⁺=405.2. 1H NMR (400 MHz, Chloroform-d) δ 7.99-7.92 (m, 1H), 7.91-7.79 (m, 2H), 7.65-7.50 (m, 3H), 7.42 (d, J=7.8 Hz, 1H), 7.30 (d, J=7.1 Hz, 1H), 5.39 (d, J=9.0 Hz, 1H), 4.46 (s, 1H), 1.75 (ddd, J=12.2, 7.3, 4.6 Hz, 1H), 1.55-1.49 (m, 1H), 1.43 (s, 9H), 0.78 (t, J=7.4 Hz, 3H).

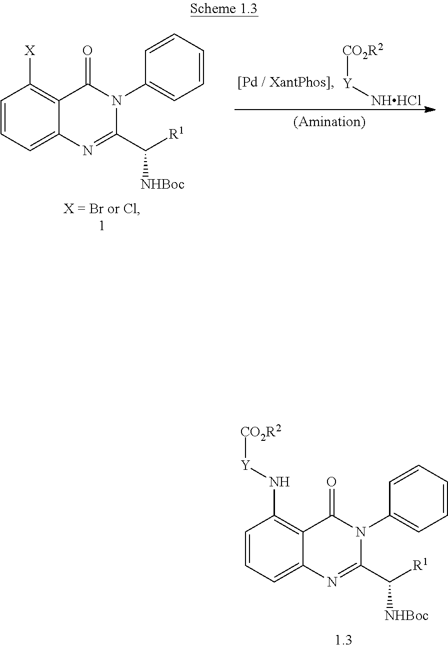

### Scheme 1.3

The substituted aryl bromide 1 (1 equiv), Methanesulfonato[9,9-dimethyl-4,5-bis(diphenylphosphino)xanthene](2′-methylamino-1,1′-biphenyl-2-yl)palladium(II) XantPhos Palladacycle (Methanesulfonato[9,9-dimethyl-4,5-bis(diphenylphosphino)xanthene](2′-methylamino-1,1′-biphenyl-2-yl)palladium(II), Strem Chemicals Inc.) (0.025-0.05 equiv) and amine [if solid] (1.3 equiv) were weighed and added to a microwave vial equipped with a stir bar. The vial was covered with a rubber septum, evacuated and then filled with nitrogen. Dry toluene or 1,4-dioxane (0.2 M) and alkyne [if oil at room temperature] (1.3 equiv) were added to the vial followed by the addition of Cs₂CO₃ (3.0 equiv) under nitrogen bubbling through the solvent. The microwave vial is sealed and heated at 110° C. for 20 hours. After 20 hours, the crude reaction mixture is filtered through a short pad of celite and concentrated in vacuo. The remaining residue was purified by flash chromatography on silica using forced flow of ethyl acetate/hexanes system on Biotage KP-Sil pre-packed cartridges and using the Biotage SP-1 automated chromatography system to afford the coupled product 1.3.

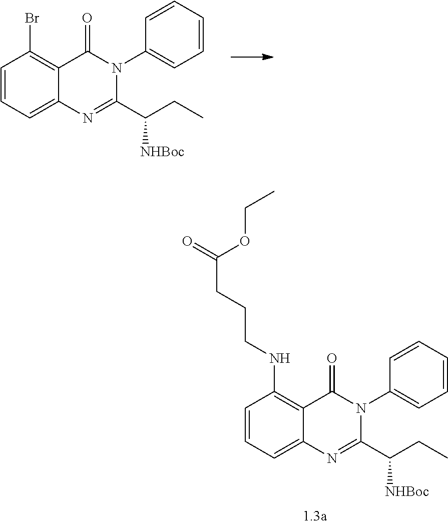 [SMILES: CC[C@H](NC(=O)OC(C)(C)C)c1nc2cccc(Br)c2c(=O)n1-c1ccccc1 ; CCOC(=O)CCCNc1cccc2nc([C@H](CC)NC(=O)OC(C)(C)C)n(-c3ccccc3)c(=O)c12]

The procedure mentioned in Scheme 1.3 was used with ((S)-tert-butyl (1-(5-bromo-4-oxo-3-phenyl-3,4-dihydroquinazolin-2-yl)propyl)carbamate (75.2 mg, 0.16 mmol), [XantPhos Palladacycle] (3.9 mg, 4.10 μmol), Cs₂CO₃ (160.0 mg, 0.49 mmol) and 4-ethoxy-4-oxobutan-1-aminium chloride (35.8 mg, 0.21 mmol) were combined in dry toluene (0.8 ml). The resulting mixture was heated at 110° C. for 20 hours and concentrated in vacuo. The remaining residue was purified by flash chromatography on silica using 0-30% ethyl acetate/hexanes to afford the product ethyl (S)-4-((2-(1-((tert-butoxycarbonyl)amino)propyl)-4-oxo-3-phenyl-3,4-dihydroquinazolin-5-yl)amino)butanoate 1.3a (58.4 mg, 0.115 mmol) in 70% yield. LC-MS (method 1): t_{R}=3.77 min, m/z (M+H)⁺=509.4. ¹H NMR (400 MHz, Chloroform-d) δ 8.49 (s, 1H), 7.62 (d, J=7.1 Hz, 1H), 7.55 (q, J=8.0, 7.6 Hz, 3H), 7.40 (s, 1H), 6.93 (d, J=27.4 Hz, 1H), 6.56 (d, J=8.4 Hz, 1H), 5.57 (s, 1H), 4.39-4.32 (m, 1H), 4.12 (q, J=7.1 Hz, 2H), 3.25 (q, J=6.5 Hz, 2H), 2.41 (t, J=7.3 Hz, 2H), 1.97 (p, J=7.2 Hz, 2H), 1.73 (s, 2H), 1.43 (s, 9H), 1.24 (t, J=7.1 Hz, 3H), 0.77 (t, J=7.4 Hz, 3H).

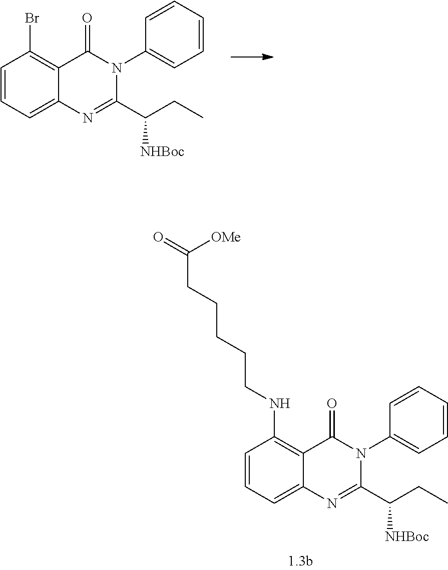 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(NCCCCCC(=O)OC)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (121.2 mg, 0.26 mmol), [XantPhos Palladacycle] (6.3 mg, 6.61 μmol), Cs₂CO₃ (258.0 mg, 0.79 mmol) and 6-methoxy-6-oxohexan-1-aminium chloride (62.4 mg, 0.34 mmol) were combined in dry toluene (1.3 ml). The resulting mixture was heated at 110° C. for 20 hours and concentrated in vacuo. The remaining residue was purified by flash chromatography on silica using 0-20% ethyl acetate/hexanes to afford the product ethyl (S)-methyl 6-((2-(1-((tert-butoxycarbonyl)amino)propyl)-4-oxo-3-phenyl-3,4-dihydroquinazolin-5-yl)amino)hexanoate 1.3b (126.0 mg, 0.24 mmol) in 91% yield. LC-MS (method 1): t_{R}=3.83 min, m/z (M+H)⁺=523.3. 1H NMR (400 MHz, Chloroform-d) δ 8.44 (d, J=5.3 Hz, 1H), 7.66-7.48 (m, 4H), 7.37 (d, J=7.9 Hz, 1H), 6.84 (d, J=7.8 Hz, 1H), 6.50 (d, J=8.4 Hz, 1H), 4.40-4.30 (m, 1H), 3.65 (s, 3H), 3.17 (td, J=6.9, 5.1 Hz, 2H), 2.31 (t, J=7.5 Hz, 2H), 1.67 (ddt, J=17.5, 15.2, 7.6 Hz, 6H), 1.58-1.45 (m, 2H), 1.43 (s, 9H), 0.76 (t, J=7.4 Hz, 3H).

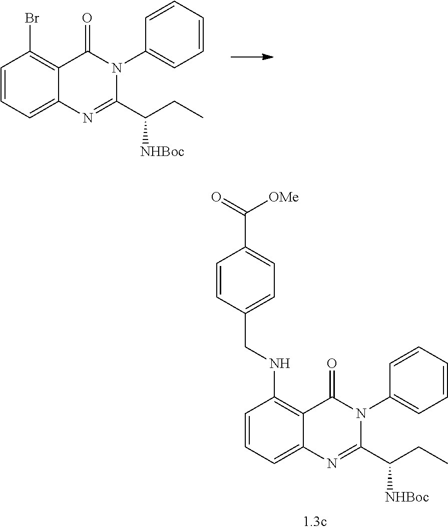 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(NCc3ccc(C(=O)OC)cc3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (95.2 mg, 0.21 mmol), [XantPhos Palladacycle] (4.9 mg, 5.19 μmol), Cs₂CO₃ (203.0 mg, 0.62 mmol) and methyl 4-(aminomethyl)benzoate hydrochloride (54.3 mg, 1.3 mmol) were combined in dry toluene (1.0 ml). The resulting mixture was heated at 110° C. for 20 hours and concentrated in vacuo. The remaining residue was purified by flash chromatography on silica using 0-30% ethyl acetate/hexanes to afford the product methyl (S)-4-(((2-(1-((tert-butoxycarbonyl)amino)propyl)-4-oxo-3-phenyl-3,4-dihydroquinazolin-5-yl)amino)methyl)benzoate 1.3c (100.0 mg, 0.184 mmol) as an off-white solid in 89% yield. LC-MS (method 1): t_{R}=3.82 min, m/z (M+H)⁺=542.3. ¹H NMR (400 MHz, Chloroform-d) δ 9.03 (t, J=5.6 Hz, 1H), 8.02-7.95 (m, 2H), 7.57 (td, J=17.2, 14.9, 8.1 Hz, 3H), 7.49-7.37 (m, 4H), 7.31-7.28 (m, 1H), 6.91 (d, J=7.8 Hz, 1H), 6.40 (d, J=8.4 Hz, 1H), 5.54 (s, 1H), 4.49 (d, J=5.8 Hz, 2H), 4.43-4.33 (m, 1H), 3.90 (s, 3H), 1.79-1.70 (m, 1H), 1.64-1.56 (m, 1H), 1.43 (s, 9H), 0.77 (t, J=7.4 Hz, 3H).

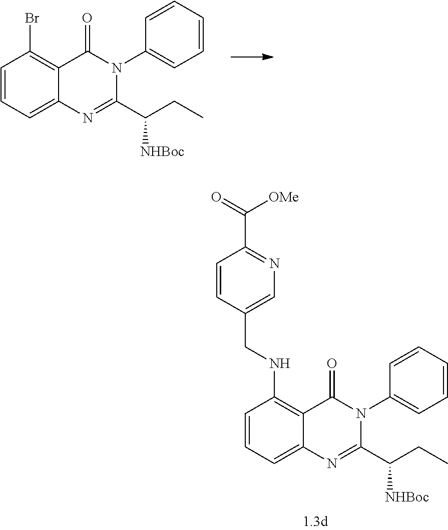 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(NCc3ccc(C(=O)OC)nc3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (96.5 mg, 0.21 mmol), [XantPhos Palladacycle] (5.0 mg, 5.26 μmol), Cs₂CO₃ (206.0 mg, 0.63 mmol) and methyl 5-(aminomethyl)picolinate dihydrochloride (54.3 mg, 1.3 mmol) were combined in dry toluene (1.0 ml). The resulting mixture was heated at 110° C. for 20 hours and concentrated in vacuo. The remaining residue was purified by flash chromatography on silica using 0-80% ethyl acetate/hexanes to afford the product methyl (S)-5-(((2-(1-((tert-butoxycarbonyl)amino)propyl)-4-oxo-3-phenyl-3,4-dihydroquinazolin-5-yl)amino)methyl)picolinate 1.3d (67.5 mg, 0.124 mmol) in 59% yield. LC-MS (method 1): t_{R}=3.56 min, m/z (M+H)⁺=544.3. 1H NMR (400 MHz, Chloroform-d) δ 9.07 (s, 1H), 8.73 (d, J=2.1 Hz, 1H), 8.08 (d, J=8.0 Hz, 1H), 7.82 (dd, J=8.1, 2.2 Hz, 1H), 7.67-7.51 (m, 3H), 7.47 (t, J=8.1 Hz, 1H), 7.41 (d, J=8.2 Hz, 1H), 7.29 (s, 1H), 6.97 (s, 1H), 6.38 (d, J=8.4 Hz, 1H), 4.54 (d, J=5.8 Hz, 2H), 4.38 (s, 1H), 4.01 (d, J=1.2 Hz, 3H), 1.78-1.72 (m, 1H), 1.63-1.53 (m, 1H) 1.43 (s, 9H), 0.78 (t, J=7.3 Hz, 3H).

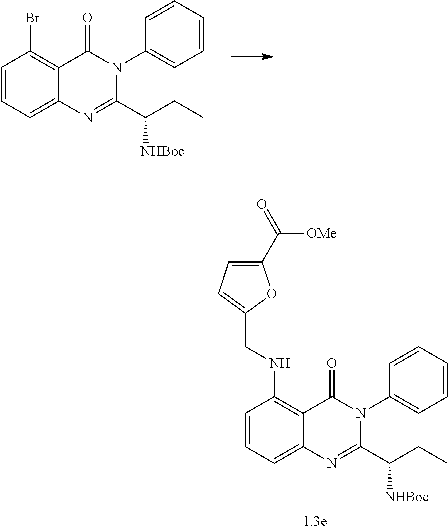 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(NCc3ccc(C(=O)OC)o3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (56.6 mg, 0.12 mmol), [XantPhos Palladacycle] (2.9 mg, 3.09 μmol), Cs₂CO₃ (121.0 mg, 0.37 mmol) and methyl 5-(aminomethyl)furan-2-carboxylate hydrochloride (35.5 mg, 0.18 mmol) were combined in dry toluene (0.6 ml). The resulting mixture was heated at 110° C. for 20 hours and concentrated in vacuo. The remaining residue was purified by flash chromatography on silica using 0-55% ethyl acetate/hexanes to afford the product methyl (S)-5-(((2-(1-((tert-butoxycarbonyl)amino)propyl)-4-oxo-3-phenyl-3,4-dihydroquinazolin-5-yl)amino)methyl)furan-2-carboxylate 1.3e (58.0 mg, 0.11 mmol) in 88% yield. LC-MS (method 1): t_{R}=3.71 min, m/z (M+H)⁺=533.3. 1H NMR (400 MHz, Chloroform-d) δ 8.94 (t, J=5.9 Hz, 1H), 7.66-7.48 (m, 4H), 7.38 (d, J=7.9 Hz, 1H), 7.09 (d, J=3.5 Hz, 1H), 6.94 (d, J=7.9 Hz, 1H), 6.51 (d, J=8.4 Hz, 1H), 6.34 (d, J=3.3 Hz, 1H), 4.48 (d, J=5.8 Hz, 2H), 4.39 (d, J=11.2 Hz, 1H), 3.88 (s, 3H), 1.74 (ddd, J=14.3, 7.4, 4.8 Hz, 1H), 1.55 (dt, J=14.0, 7.1 Hz, 1H), 1.43 (s, 9H), 0.77 (t, J=7.4 Hz, 3H).

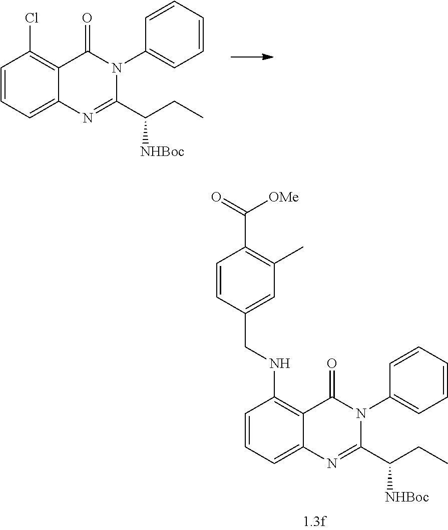 [SMILES: CC[C@H](NC(=O)OC(C)(C)C)c1nc2cccc(Cl)c2c(=O)n1-c1ccccc1 ; CC[C@H](NC(=O)OC(C)(C)C)c1nc2cccc(NCc3ccc(C(=O)OC)c(C)c3)c2c(=O)n1-c1ccccc1]

The procedure mentioned in Scheme 1.3 was used with ((S)-tert-butyl (1-(5-chloro-4-oxo-3-phenyl-3,4-dihydroquinazolin-2-yl)propyl)carbamate (170.0 mg, 0.411 mmol, Castro, A. C. et al. WO 2015/061204 A1), [XantPhos Palladacycle] (20.0 mg, 0.021 mmol), Cs₂CO₃ (401.0 mg, 1.23 mmol) and methyl 5-(aminomethyl)picolinate dihydrochloride (88.0 mg, 0.493 mmol) were combined in dry toluene (2.0 ml). The resulting mixture was heated at 110° C. for 20 hours and concentrated in vacuo. The remaining residue was purified by flash chromatography on silica using 0-50% ethyl acetate/hexanes to afford the product methyl (S)-4-(((2-(1-((tert-butoxycarbonyl)amino)propyl)-4-oxo-3-phenyl-3,4-dihydroquinazolin-5-yl)amino)methyl)-2-methylbenzoate 1.3f (175.0 mg, 0.31 mmol) in 88% yield. LC-MS (method 1): t_{R}=3.88 min, m/z (M+H)⁺=557.3.

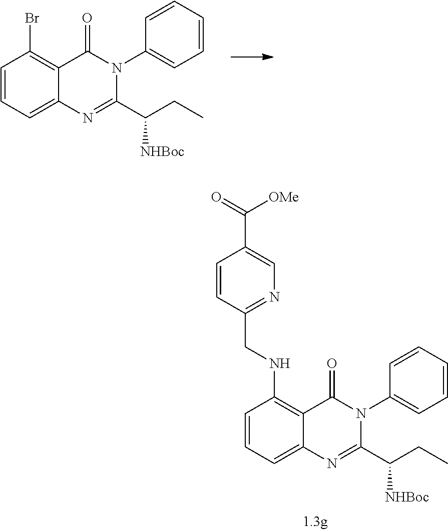 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(NCc3ccc(C(=O)OC)cn3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (109.0 mg, 0.24 mmol), [XantPhos Palladacycle] (6.8 mg, 0.007 mmol), Cs₂CO₃ (232.0 mg, 0.71 mmol) and methyl 5-(aminomethyl)picolinate dihydrochloride (58.0 mg, 0.29 mmol) were combined in dry toluene (1.2 ml). The resulting mixture was heated at 110° C. for 20 hours and concentrated in vacuo. The remaining residue was purified by flash chromatography on silica using 0-50% ethyl acetate/hexanes to afford the product methyl (S)-6-(((2-(1-((tert-butoxycarbonyl)amino)propyl)-4-oxo-3-phenyl-3,4-dihydroquinazolin-5-yl)amino)methyl)nicotinate 1.3g (100.0 mg, 0.18 mmol) in 77% yield. LC-MS (method 1): t_{R}=3.58 min, m/z (M+H)⁺=544.3. ¹H NMR (400 MHz, Chloroform-d) δ 9.28-9.15 (m, 2H), 8.20 (dd, J=8.2, 2.2 Hz, 1H), 7.67-7.50 (m, 3H), 7.44 (dt, J=13.8, 9.0 Hz, 3H), 7.33-7.28 (m, 1H), 6.91 (d, J=7.9 Hz, 1H), 6.37 (d, J=8.3 Hz, 1H), 5.56-5.51 (m, 1H), 4.64 (d, J=6.0 Hz, 2H), 4.39 (s, 1H), 3.94 (d, J=1.6 Hz, 3H), 1.78-1.70 (m, 1H), 1.60-1.50 (m, 1H), 1.40 (s, 9H), 0.77 (t, J=7.4 Hz, 3H).

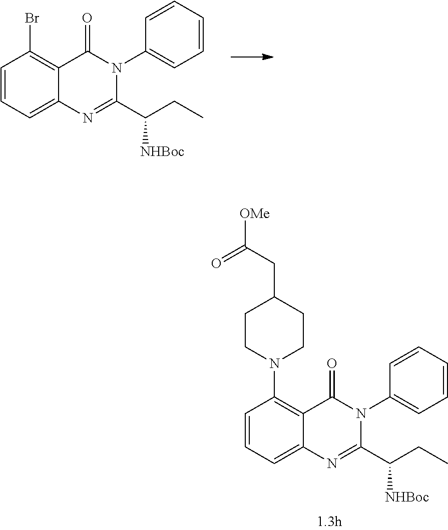 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(N3CCC(CC(=O)OC)CC3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (130.0 mg, 0.28 mmol), [XantPhos Palladacycle] (8.1 mg, 0.009 mmol), Cs₂CO₃ (277.0 mg, 0.85 mmol) and methyl 2-(piperidin-4-yl)acetate hydrochloride (67.0 mg, 0.34 mmol) were combined in dry 1,4-dioxane (1.4 ml). The resulting mixture was heated at 110° C. for 20 hours and concentrated in vacuo. The remaining residue was purified by flash chromatography on silica using 0-50% ethyl acetate/hexanes to afford the product methyl (S)-2-(1-(2-(1-((tert-butoxycarbonyl)amino)propyl)-4-oxo-3-phenyl-3,4-dihydroquinazolin-5-yl)piperidin-4-yl)acetate 1.3h (46.1 mg, 0.09 mmol) in 30% yield. LC-MS (method 1): t_{R}=3.18 min, m/z (M+H)⁺=535.3. ¹H NMR (400 MHz, Chloroform-d) δ 7.54 (dq, J=32.3, 7.7, 7.3 Hz, 4H), 7.32 (d, J=7.9 Hz, 1H), 7.27-7.23 (m, 2H), 6.96 (d, J=8.1 Hz, 1H), 5.55 (d, J=9.1 Hz, 1H), 4.32 (td, J=8.7, 4.5 Hz, 1H), 3.66 (s, 3H), 3.49-3.38 (m, 2H), 2.74 (d, J=13.1 Hz, 2H), 2.27 (d, J=7.1 Hz, 2H), 1.91 (d, J=22.8 Hz, 1H), 1.81-1.50 (m, 6H), 1.43 (s, 9H), 0.74 (t, J=7.3 Hz, 3H).

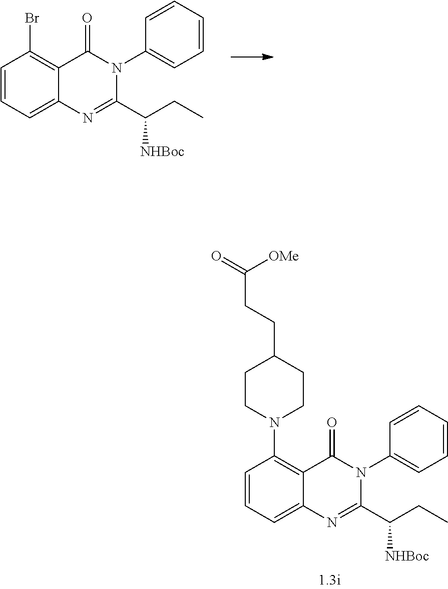 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(N3CCC(CCC(=O)OC)CC3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (118.5 mg, 0.26 mmol), [XantPhos Palladacycle] (7.4 mg, 0.008 mmol), Cs₂CO₃ (253.0 mg, 0.78 mmol) and methyl 3-(piperidin-4-yl)propanoate hydrochloride (68.0 mg, 0.31 mmol) were combined in dry 1,4-dioxane (1.3 ml). The resulting mixture was heated at 110° C. for 20 hours and concentrated in vacuo. The remaining residue was purified by flash chromatography on silica using 0-50% ethyl acetate/hexanes to afford the product methyl (S)-3-(1-(2-(1-((tert-butoxycarbonyl)amino)propyl)-4-oxo-3-phenyl-3,4-dihydroquinazolin-5-yl)piperidin-4-yl)propanoate 1.3i (32.0 mg, 0.06 mmol) in 23% yield. LC-MS (method 1): t_{R}=3.19 min, m/z (M+H)⁺=549.3.

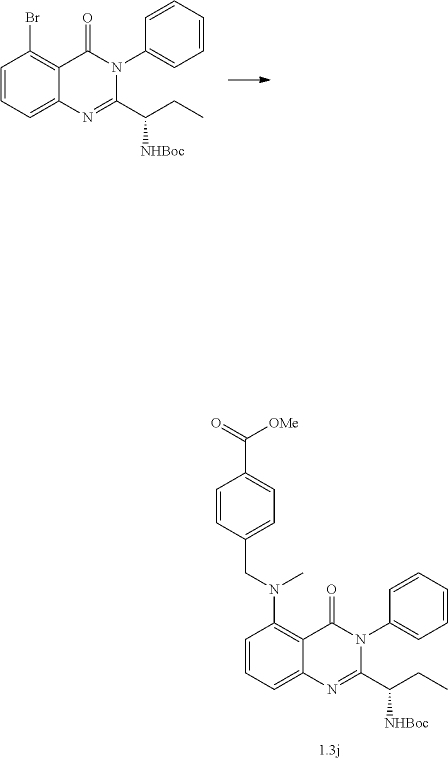 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(N(C)Cc3ccc(C(=O)OC)cc3)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (222.0 mg, 0.48 mmol), [XantPhos Palladacycle] (14.0 mg, 0.015 mmol), Cs₂CO₃ (473.0 mg, 1.45 mmol) and methyl 4-((methylamino)methyl)benzoate hydrochloride (125.0 mg, 0.58 mmol) were combined in dry 1,4-dioxane (2.4 ml). The resulting mixture was heated at 110° C. for 20 hours and concentrated in vacuo. The remaining residue was purified by flash chromatography on silica using 0-50% ethyl acetate/hexanes to afford the product methyl (S)-4-(((2-(1-((tert-butoxycarbonyl)amino)propyl)-4-oxo-3-phenyl-3,4-dihydroquinazolin-5-yl)(methyl)amino)methyl)benzoate 1.3j (58.0 mg, 0.10 mmol) in 22% yield. LC-MS (method 1): t_{R}=3.20 min, m/z (M+H)⁺=557.3.

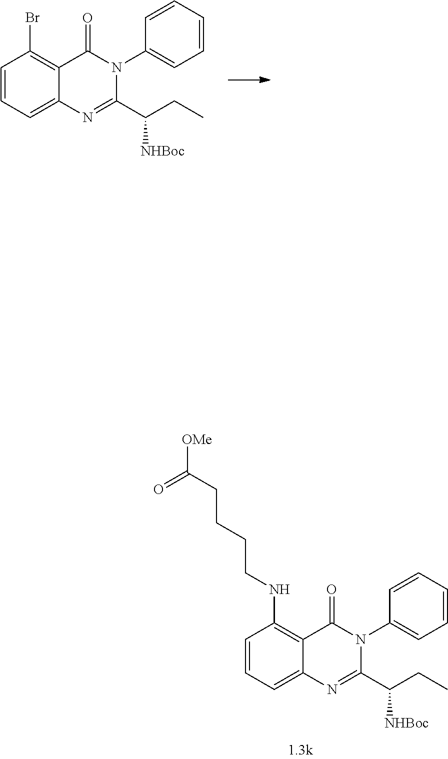 [SMILES: CC[C@H](NC(=O)OC(C)(C)C)c1nc2cccc(Br)c2c(=O)n1-c1ccccc1 ; CC[C@H](NC(=O)OC(C)(C)C)c1nc2cccc(NCCCCC(=O)OC)c2c(=O)n1-c1ccccc1]

The procedure mentioned in Scheme 1.3 was used with ((S)-tert-butyl (1-(5-bromo-4-oxo-3-phenyl-3,4-dihydroquinazolin-2-yl)propyl)carbamate (218.4 mg, 0.48 mmol), [XantPhos Palladacycle] (13.7 mg, 14.0 mol), Cs₂CO₃ (466.0 mg, 1.43 mmol) and 5-methoxy-5-oxopentan-1-aminium chloride (96.0 mg, 0.57 mmol) were combined in dry toluene (2.4 ml). The resulting mixture was heated at 110° C. for 20 hours and concentrated in vacuo. The remaining residue was purified by flash chromatography on silica using 0-50% ethyl acetate/hexanes to afford the product ethyl (S)-methyl 6-((2-(1-((tert-butoxycarbonyl)amino)propyl)-4-oxo-3-phenyl-3,4-dihydroquinazolin-5-yl)amino)hexanoate 1.3k (153.0 mg, 0.30 mmol) in 63% yield. LC-MS (method 1): t_{R}=3.75 min, m/z (M+H)⁺=509.3. 1H NMR (400 MHz, Chloroform-d) δ 8.42 (s, 1H), 7.57 (td, J=18.2, 14.9, 7.7 Hz, 4H), 7.41 (s, 1H), 7.26 (s, 1H), 6.53 (d, J=8.4 Hz, 1H), 4.35 (s, 1H), 3.66 (s, 3H), 3.20 (q, J=6.2 Hz, 2H), 2.34 (t, J=6.9 Hz, 2H), 1.79-1.64 (m, 6H), 1.43 (s, 9H), 0.77 (t, J=7.3 Hz, 3H).

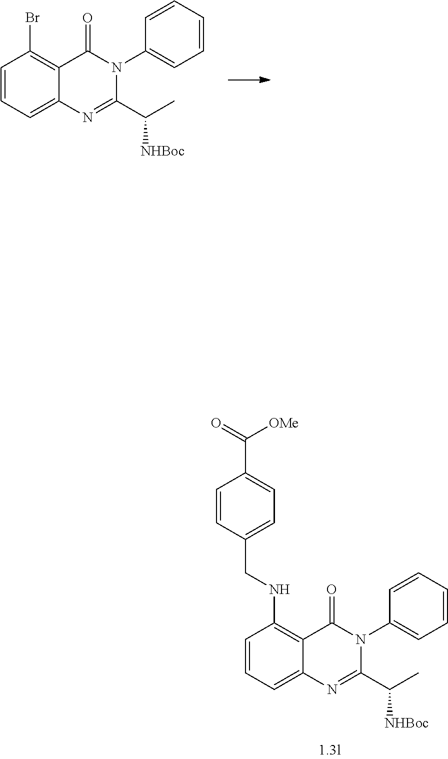 [SMILES: C[C@H](NC(=O)OC(C)(C)C)c1nc2cccc(Br)c2c(=O)n1-c1ccccc1 ; COC(=O)c1ccc(CNc2cccc3nc([C@H](C)NC(=O)OC(C)(C)C)n(-c4ccccc4)c(=O)c23)cc1]

The procedure mentioned in Scheme 1.3 was used with tert-butyl (S)-(1-(5-bromo-4-oxo-3-phenyl-3,4-dihydroquinazolin-2-yl)ethyl)carbamate (300.0 mg, 0.675 mmol), [XantPhos Palladacycle] (19.0 mg, 0.02 mmol), Cs₂CO₃ (660.0 mg, 2.03 mmol) and methyl 4-(aminomethyl)benzoate hydrochloride (177.0 mg, 0.88 mmol) were combined in dry toluene (3.4 ml). The resulting mixture was heated at 110° C. for 20 hours and concentrated in vacuo. The remaining residue was purified by flash chromatography on silica using 0-50% ethyl acetate/hexanes to afford the product methyl (S)-4-(((2-(1-((tert-butoxycarbonyl)amino)ethyl)-4-oxo-3-phenyl-3,4-dihydroquinazolin-5-yl)amino)methyl)benzoate 1.31 (274.0 mg, 0.52 mmol) as an off-white solid in 77% yield. LC-MS (method 1): t_{R}=3.53 min, m/z (M+H)⁺=529.3. 1H NMR (400 MHz, Chloroform-d) δ 8.98 (s, 1H), 8.02-7.96 (m, 2H), 7.69-7.37 (m, 8H), 7.30 (d, J=7.3 Hz, 1H), 6.42 (d, J=8.4 Hz, 1H), 4.52 (m, 1H), 4.49 (d, J=5.9 Hz, 2H), 3.91 (d, J=1.2 Hz, 3H), 1.43 (s, 9H), 1.32 (m, 3H).

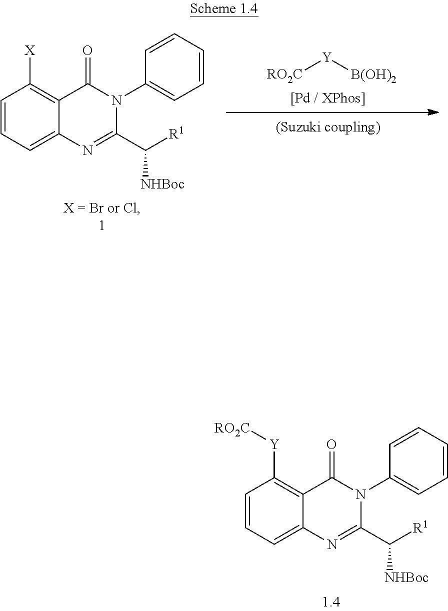

### Scheme 1.4

The substituted aryl chloride 1 (1 equiv), chloro(crotyl)(2-dicyclohexylphosphino-2′,4′,6′-triisopropylbiphenyl)palladium(II) [Pd-170] (0.05 equiv) and boronic acid (1.2 equiv) were suspended in dioxane/water (0.2 M, 4:1 by vol) in a MW vial equipped with a stir bar under N₂ atmosphere and potassium phosphate (3.0 equiv) was added to it. The MW vial was sealed and heated at 100° C. for 1 h in a MW reactor. The reaction mixture was allowed to cool to RT, quenched with water, and then extracted 3 times with ethyl acetate. The combined organic fractions were dried over MgSO₄ and then concentrated in vacuo. The remaining residue was purified by flash chromatography on silica using forced flow of ethyl acetate/hexanes system on Biotage KP-Sil pre-packed cartridges and using the Biotage SP-1 automated chromatography system to afford the coupled product 1.4.

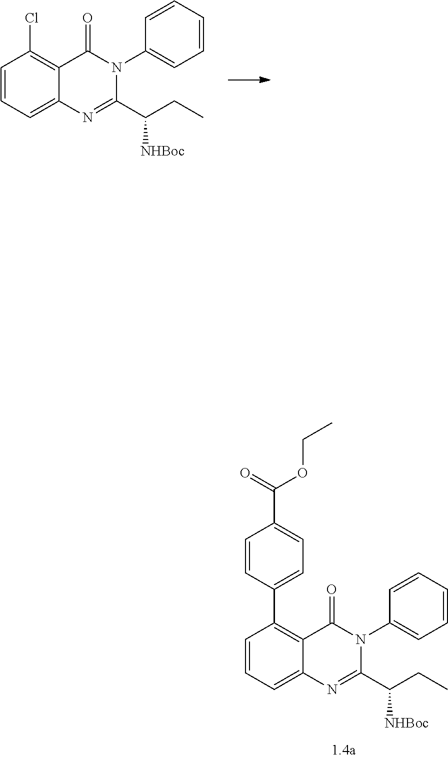 [SMILES: CC[C@H](NC(=O)OC(C)(C)C)c1nc2cccc(Cl)c2c(=O)n1-c1ccccc1 ; CCOC(=O)c1ccc(-c2cccc3nc([C@H](CC)NC(=O)OC(C)(C)C)n(-c4ccccc4)c(=O)c23)cc1]

The procedure mentioned in Scheme 1.4 was used with ((S)-tert-butyl (1-(5-chloro-4-oxo-3-phenyl-3,4-dihydroquinazolin-2-yl)propyl)carbamate (261.0 mg, 0.63 mmol), [Pd-170] (21.0 mg, 0.03 mmol), (4-(ethoxycarbonyl)phenyl)boronic acid (147.0 mg, 0.76 mmol) and potassium phosphate (402.0 mg, 1.89 mmol) in 1,4-dioxane/water (2.0 ml, 4:1). The resulting mixture was heated at 100° C. for 1 hour in MW and concentrated in vacuo. The remaining residue was purified by flash chromatography on silica using 0-35% ethyl acetate/hexanes to afford the product ethyl (S)-4-(2-(1-((tert-butoxycarbonyl)amino)propyl)-4-oxo-3-phenyl-3,4-dihydroquinazolin-5-yl)benzoate 1.4a (320.0 mg, 0.61 mmol) as a colorless solid in 96% yield. LC-MS (method 1): t_{R}=3.82 min, m/z (M+H)⁺=528.3. ¹H NMR (400 MHz, Chloroform-d) δ 8.05-8.00 (m, 2H), 7.80-7.76 (m, 2H), 7.56-7.40 (m, 3H), 7.39-7.30 (m, 3H), 7.27 (t, J=4.4 Hz, 1H), 7.22-7.16 (m, 1H), 5.57 (d, J=9.1 Hz, 1H), 4.41 (d, J=8.6 Hz, 1H), 4.36 (q, J=7.1 Hz, 2H), 1.76 (ddd, J=14.2, 7.4, 4.6 Hz, 1H), 1.62-1.53 (m, 1H), 1.45 (s, 9H), 0.78 (t, J=7.4 Hz, 3H).

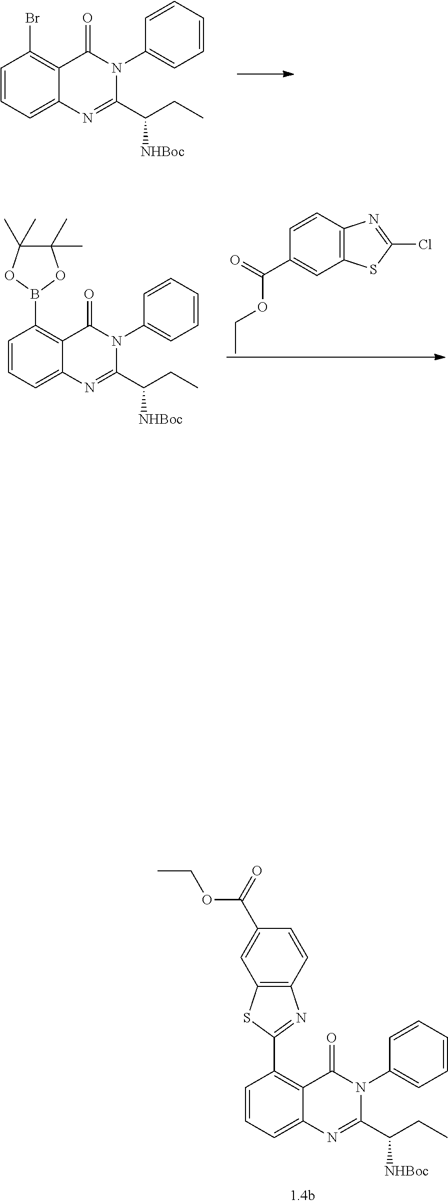

To a mixture of ((S)-tert-butyl (1-(5-bromo-4-oxo-3-phenyl-3,4-dihydroquinazolin-2-yl)propyl)carbamate (116.0 mg, 0.25 mmol) and 4,4,4′,4′,5,5,5′,5′-octamethyl-2,2′-bi(1,3,2-dioxaborolane) [BPin]₂ (77.0 mg, 0.30 mmol) in 1,4-dioxane (1.0 ml) in a sealed tube, Pd(dppf)Cl₂ (9.3 mg, 13.0 μmol) and potassium acetate (74.5 mg, 0.76 mmol) were added under N₂ bubbling through the solvent. The resulting mixture was stirred at 100° C. for 16 hours. After completion of the reaction, the crude reaction mixture is filtered into a MW vial equipped with a stir bar and ethyl 2-bromobenzo[d]thiazole-6-carboxylate (60.0 mg, 0.21 mmol) and 0.1 ml of water were added to it. Added [Pd-170] (10.6 mg, 7.1 μmol) and potassium phosphate (134.0 mg, 0.63 mmol) to this mixture under nitrogen atmosphere. The MW vial was sealed and heated at 100° C. for 10 hours. The reaction mixture was allowed to cool to room temperature, quenched with water, and then extracted 3 times with ethyl acetate. The combined organic fractions were dried over MgSO₄ and then concentrated in vacuo. The remaining residue was purified by flash chromatography on silica using 0-45% EtOAc/Hexanes to afford the coupled product ethyl (S)-2-(2-(1-((tert-butoxycarbonyl)amino)propyl)-4-oxo-3-phenyl-3,4-dihydroquinazolin-5-yl)benzo[d]thiazole-6-carboxylate (87.0 mg, 0.15 mmol) 1.4b in 70% yield. LC-MS (method 1): t_{R}=3.80 min, m/z (M+H)⁺=585.2. ¹H NMR (400 MHz, Chloroform-d) δ 8.59 (d, J=1.6 Hz, 1H), 8.14 (dd, J=8.6, 1.7 Hz, 1H), 8.05 (d, J=8.6 Hz, 1H), 7.94 (d, J=8.3 Hz, 1H), 7.90-7.82 (m, 1H), 7.63-7.40 (m, 4H), 7.36 (d, J=8.0 Hz, 1H), 7.23 (d, J=7.3 Hz, 1H), 5.50 (d, J=9.1 Hz, 1H), 4.42 (q, J=7.1 Hz, 3H), 1.75 (dt, J=12.6, 6.9 Hz, 1H), 1.58-1.52 (m, 1H), 1.44 (m, 9H), 0.78 (t, J=7.4 Hz, 3H).

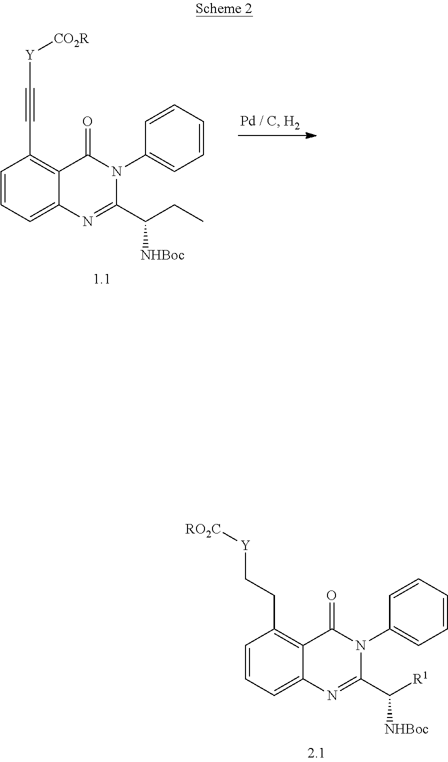

The internal alkyne 1.1 and 10 wt % Pd/C were added to a round-bottomed flask fitted with a rubber septum. The reaction flask is evacuated followed by the addition of dry EtOAc (0.1 M). The vacuum is removed and the reaction flask is kept under an atmosphere of hydrogen using a balloon and was stirred for 20 h. After completion of reaction (by LC MS), the crude reaction mixture is filtered using celite, concentrated in vacuo to afford the product.

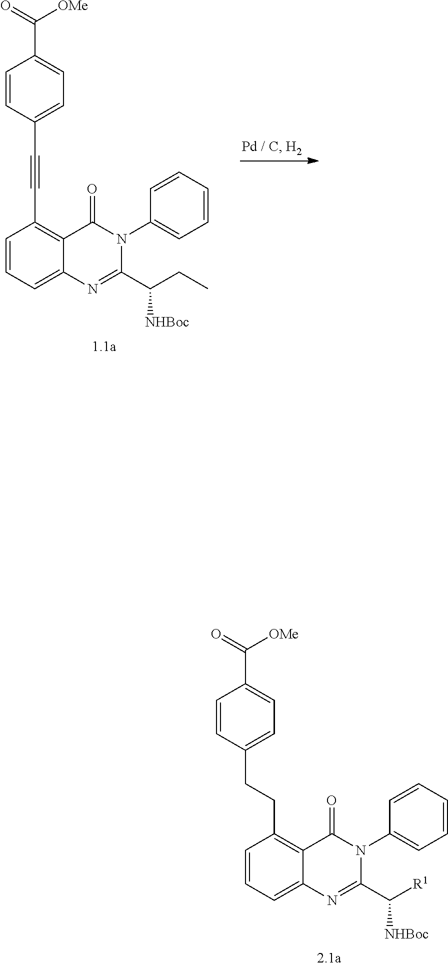

The procedure mentioned in Scheme 2 was used with (S)-methyl 4-((2-(1-((tert-butoxycarbonyl)amino)propyl)-4-oxo-3-phenyl-3,4-dihydroquinazolin-5-yl)ethynyl)benzoate (68.0 mg, 0.13 mmol) and 10% Pd/C (7.0 mg) in EtOAc (1.3 ml). The resulting suspension was stirred under hydrogen atmosphere for 20 hours, filtered through celite and concentrated in vacuo to afford the product (S)-methyl 4-(2-(2-(1-((tert-butoxycarbonyl)amino)propyl)-4-oxo-3-phenyl-3,4-dihydroquinazolin-5-yl)ethyl)benzoate 2.1a. LC-MS (method 1): t_{R}=3.92 min, m/z (M+H)⁺=542.3.

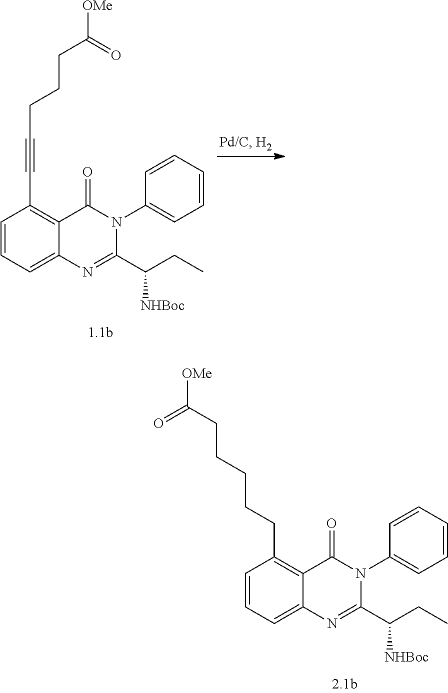 [SMILES: CC[C@H](NC(=O)OC(C)(C)C)c1nc2cccc(C#CCCCC(=O)OC)c2c(=O)n1-c1ccccc1 ; CC[C@H](NC(=O)OC(C)(C)C)c1nc2cccc(CCCCCC(=O)OC)c2c(=O)n1-c1ccccc1]

The procedure mentioned in Scheme 2 was used with (S)-methyl 6-(2-(1-((tert-butoxycarbonyl)amino)propyl)-4-oxo-3-phenyl-3,4-dihydroquinazolin-5-yl)hex-5-ynoate (160.0 mg, 0.32 mmol) and 10% Pd/C (16.0 mg) in EtOAc (3.2 ml). The resulting suspension was stirred under hydrogen atmosphere for 20 hours, filtered through celite and concentrated in vacuo to afford the product (S)-methyl 6-(2-(1-((tert-butoxycarbonyl)amino)propyl)-4-oxo-3-phenyl-3,4-dihydroquinazolin-5-yl)hexanoate 2.1b. LC-MS (method 1): t_{R}=3.85 min, m/z (M+H)⁺=508.4.

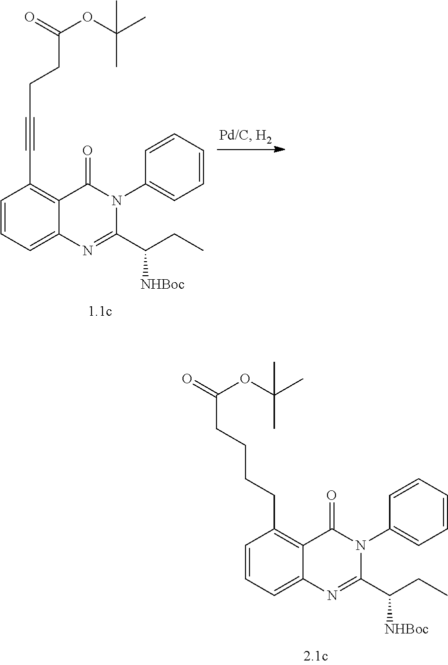 [SMILES: CC[C@H](NC(=O)OC(C)(C)C)c1nc2cccc(C#CCCC(=O)OC(C)(C)C)c2c(=O)n1-c1ccccc1 ; CC[C@H](NC(=O)OC(C)(C)C)c1nc2cccc(CCCCC(=O)OC(C)(C)C)c2c(=O)n1-c1ccccc1]

The procedure mentioned in Scheme 2 was used with tert-butyl (S)-5-(2-(1-((tert-butoxycarbonyl)amino)propyl)-4-oxo-3-phenyl-3,4-dihydroquinazolin-5-yl)pent-4-ynoate (102.0 mg, 0.19 mmol) and 10% Pd/C (10.0 mg) in EtOAc (1.9 ml). The resulting suspension was stirred under hydrogen atmosphere for 20 hours, filtered through celite and concentrated in vacuo to afford the product tert-butyl (S)-5-(2-(1-((tert-butoxycarbonyl)amino)propyl)-4-oxo-3-phenyl-3,4-dihydroquinazolin-5-yl)pentanoate 2.1c. LC-MS (method 1): t_{R}=4.04 min, m/z (M+H)⁺=536.3.

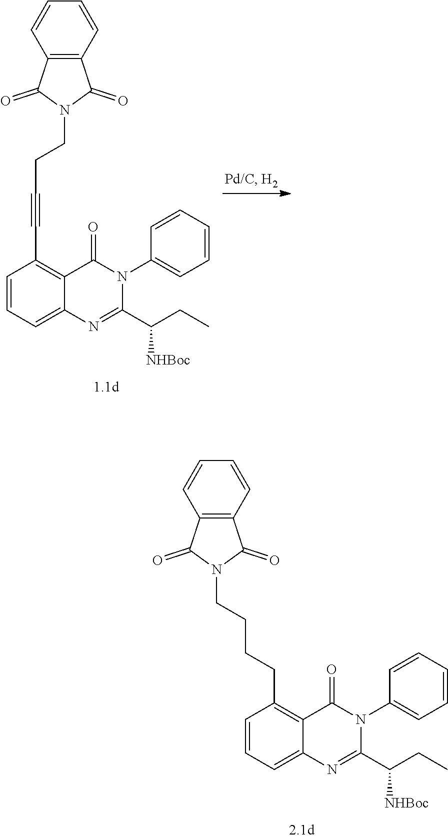 [SMILES: CC[C@H](NC(=O)OC(C)(C)C)c1nc2cccc(C#CCCN3C(=O)c4ccccc4C3=O)c2c(=O)n1-c1ccccc1 ; CC[C@H](NC(=O)OC(C)(C)C)c1nc2cccc(CCCCN3C(=O)c4ccccc4C3=O)c2c(=O)n1-c1ccccc1]

The procedure mentioned in Scheme 2 was used with tert-butyl (S)-(1-(5-(4-(1,3-dioxoisoindolin-2-yl)but-1-yn-1-yl)-4-oxo-3-phenyl-3,4-dihydroquinazolin-2-yl)propyl)carbamate (125.0 mg, 0.22 mmol) and 10% Pd/C (12.5 mg) in EtOAc (2.2 ml). The resulting suspension was stirred under hydrogen atmosphere for 20 hours, filtered through celite and concentrated in vacuo to afford the product tert-butyl (S)-(1-(5-(4-(1,3-dioxoisoindolin-2-yl)butyl)-4-oxo-3-phenyl-3,4-dihydroquinazolin-2-yl)propyl)carbamate 2.1d. LC-MS (method 1): t_{R}=3.85 min, m/z (M+H)⁺=581.3.

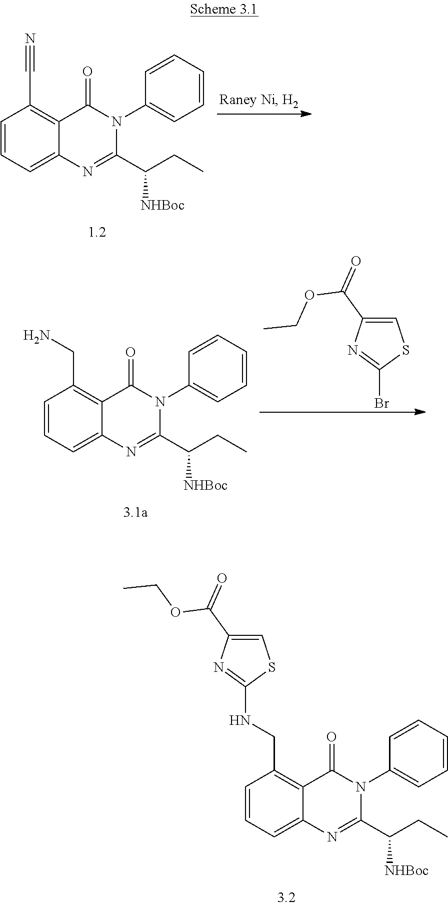

### Scheme 3.1

Dissolved tert-butyl (S)-(1-(5-cyano-4-oxo-3-phenyl-3,4-dihydroquinazolin-2-yl)propyl)carbamate (180.0 mg, 0.445 mmol) in ammonia (2.2 mL, 7N in MeOH) in a 20 ml scintillation vial and added Raney Ni (20.0 mg (approx.)) to it. The reaction vial is evacuated and then kept under hydrogen atmosphere using a balloon. The resulting suspension was stirred at room temperature for 20 hours. After completion of reaction (by LC-MS), the crude reaction mixture is carefully filtered under nitrogen and concentrated in vacuo to afford the product tert-butyl (S)-(1-(5-(aminomethyl)-4-oxo-3-phenyl-3,4-dihydroquinazolin-2-yl)propyl)carbamate 3.1a (177.6 mg, 0.435 mmol) in 98% yield. LC-MS (method 1): t_{R}=2.85 min, m/z (M+H)⁺=409.3.

Tert-butyl (S)-(1-(5-(aminomethyl)-4-oxo-3-phenyl-3,4-dihydroquinazolin-2-yl)propyl)carbamate 3.1a (102.0 mg, 0.25 mmol) was dissolved in dry DMF (0.6 ml) in a microwave vial and ethyl 2-bromothiazole-4-carboxylate (118.0 mg, 0.5 mmol) and N-ethyl-N-isopropylpropan-2-amine [DIPEA] (129.0 mg, 1.00 mmol) were added to it. The microwave vial was sealed and the resulting mixture was heated at 180° C. for 30 min in a microwave. After completion of the reaction, the reaction mixture is concentrated in vacuo and the remaining residue was purified using 0-5% MeOH/DCM to afford the product ethyl (S)-2-(((2-(1-((tert-butoxycarbonyl)amino)propyl)-4-oxo-3-phenyl-3,4-dihydroquinazolin-5-yl)methyl)amino)thiazole-4-carboxylate 3.2 (52.8 mg, 0.094 mmol) in 38% yield. LC-MS (method 1): t_{R}=3.55 min, m/z (M+H)⁺=564.3.

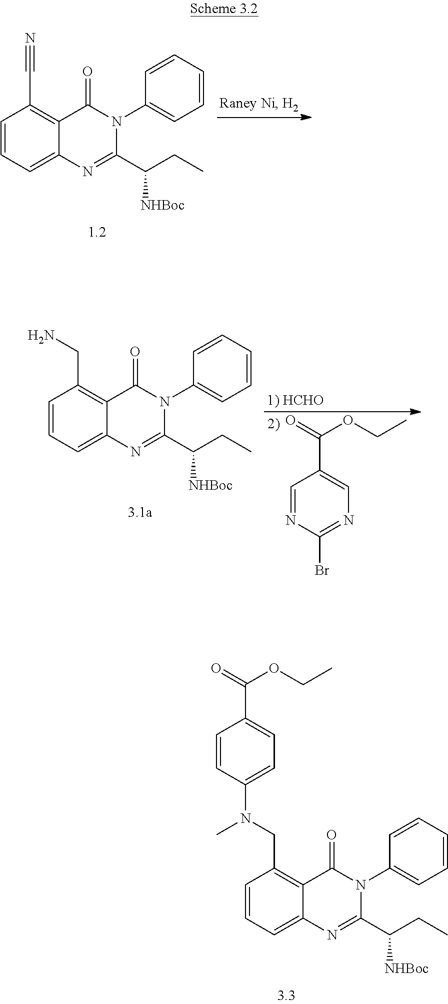 [SMILES: CC[C@H](NC(=O)OC(C)(C)C)c1nc2cccc(C#N)c2c(=O)n1-c1ccccc1 ; CC[C@H](NC(=O)OC(C)(C)C)c1nc2cccc(CN)c2c(=O)n1-c1ccccc1 ; CCOC(=O)c1cnc(Br)nc1 ; CCOC(=O)c1ccc(N(C)Cc2cccc3nc([C@H](CC)NC(=O)OC(C)(C)C)n(-c4ccccc4)c(=O)c23)cc1]

### Scheme 3.2

Dissolved tert-butyl (S)-(1-(5-cyano-4-oxo-3-phenyl-3,4-dihydroquinazolin-2-yl)propyl)carbamate (180.0 mg, 0.445 mmol) in ammonia (2.2 mL, 7N in MeOH) in a 20 ml scintillation vial and added Raney Ni (20.0 mg (approx.)) to it. The reaction vial is evacuated and then kept under hydrogen atmosphere using a balloon. The resulting suspension was stirred at room temperature for 20 hours. After completion of reaction (by LC-MS), the crude reaction mixture is carefully filtered under nitrogen and concentrated in vacuo to afford the product tert-butyl (S)-(1-(5-(aminomethyl)-4-oxo-3-phenyl-3,4-dihydroquinazolin-2-yl)propyl)carbamate 3.1a (177.6 mg, 0.435 mmol) in 98% yield. LC-MS (method 1): t_{R}=2.85 min, m/z (M+H)⁺=409.3.

Tert-butyl (S)-(1-(5-(aminomethyl)-4-oxo-3-phenyl-3,4-dihydroquinazolin-2-yl)propyl)carbamate 3.1a (90.0 mg, 0.22 mmol) was dissolved in dry DCM (2.0 ml) in a vial and paraformaldehyde (6.9 mg, 0.22 mmol) followed by a drop of acetic acid were added to it. The resulting mixture was stirred at room temperature for 2 h followed by the addition of sodium triacetoxy borohydride (93.0 mg, 0.44 mmol). The reaction mixture was stirred at room temperature for another 2 h. After completion of the reaction by LC-MS, a saturated aqueous solution of sodium bicarbonate was added. The product was extracted three times with CH₂Cl₂. The combined organic layers were washed dried over MgSO₄, filtered and concentrated. After completion of the reaction, the reaction mixture is concentrated in vacuo and the remaining residue was purified using 0-15% MeOH (0.1% TEA/DCM to afford the product tert-butyl (S)-(1-(5-((methylamino)methyl)-4-oxo-3-phenyl-3,4-dihydroquinazolin-2-yl)propyl)carbamate (19.5 mg, 0.046 mmol) in 21% yield. LC-MS (method 1): t_{R}=2.90 min, m/z (M+H)⁺=423.3.

(S)-(1-(5-((methylamino)methyl)-4-oxo-3-phenyl-3,4-dihydroquinazolin-2-yl)propyl)carbamate (19.5 mg, 0.046 mmol) and ethyl 2-chloropyrimidine-5-carboxylate (12.9 mg, 0.069 mmol) were suspended in butanol (0.5 ml) in a 5 ml microwave vial and DIPEA (17.9 mg, 0.14 mmol, 24 μl) was added to it. The resulting mixture was heated for 2 hours at 130° C. in a microwave. After completion of reaction, the crude reaction mixture is concentrated in vacuo and purified by silica gel column chromatography using 0-40% EtOAc/Hexanes to afford the product, ethyl (S)-2-(((2-(1-((tert-butoxycarbonyl)amino)propyl)-4-oxo-3-phenyl-3,4-dihydroquinazolin-5-yl)methyl)(methyl)amino)pyrimidine-5-carboxylate 3.3 (22.0 mg, 0.038 mmol) in 83% yield. LC-MS (method 1): t_{R}=3.84 min, m/z (M+H)⁺=573.3.

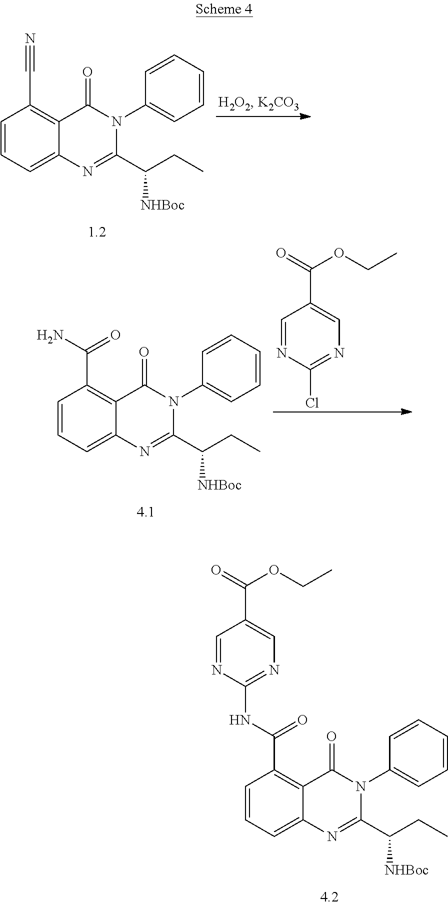

### Scheme 4

Suspended tert-butyl (S)-(1-(5-cyano-4-oxo-3-phenyl-3,4-dihydroquinazolin-2-yl)propyl)carbamate (60.0 mg, 0.148 mmol) in ethanol/water in a microwave vial containing a stir bar (4.0 ml, 1:1) and added potassium carbonate (205.0 mg, 1.48 mmol) and 50% aqueous hydrogen peroxide solution (101.0 mg, 1.48 mmol, 84 μl) to it. The reaction mixture wad stirred at room temperature for 18 hours and concentrated in vacuo. The remaining residue is dissolved in DCM and extracted 3 times with water. The organic phases were separated, dried over sodium sulfate and evaporated to dryness in vacuo. Chromatographic purification on silica using 0-5% MeOH/DCM afforded the product tert-butyl (S)-(1-(5-carbamoyl-4-oxo-3-phenyl-3,4-dihydroquinazolin-2-yl)propyl)carbamate 4.1 (37.4 mg, 0.089 mmol) in 60% yield. LC-MS (method 1): t_{R}=3.05 min, m/z (M+H)⁺=423.2.

Tert-butyl (S)-(1-(5-carbamoyl-4-oxo-3-phenyl-3,4-dihydroquinazolin-2-yl)propyl)carbamate 4.1 (37.4 mg, 0.089 mmol), [XantPhos Palladacycle] (2.1 mg, 0.025 mmol) and ethyl 2-chloropyrimidine-5-carboxylate (21.5 mg, 0.12 mmol) were weighed and added to a microwave vial equipped with a stir bar. The vial was covered with a rubber septum, evacuated and then filled with nitrogen. Dry toluene (0.3 ml) was added to the vial followed by the addition of Cs₂CO₃ (3.0 equiv) under nitrogen bubbling through the solvent. The microwave vial is sealed and heated reflux for 20 hours. The crude product is filtered through a short pad of celite, concentrated in vacuo and purified by column chromatography on silica using 0-5% MeOH/DCM to afford the product ethyl (S)-2-(2-(1-((tert-butoxycarbonyl)amino)propyl)-4-oxo-3-phenyl-3,4-dihydroquinazoline-5-carboxamido)pyrimidine-5-carboxylate 4.2 (24.3 mg, 0.042 mmol) in 48% yield. LC-MS (method 1): t_{R}=3.42 min, m/z (M+H)⁺=573.3.

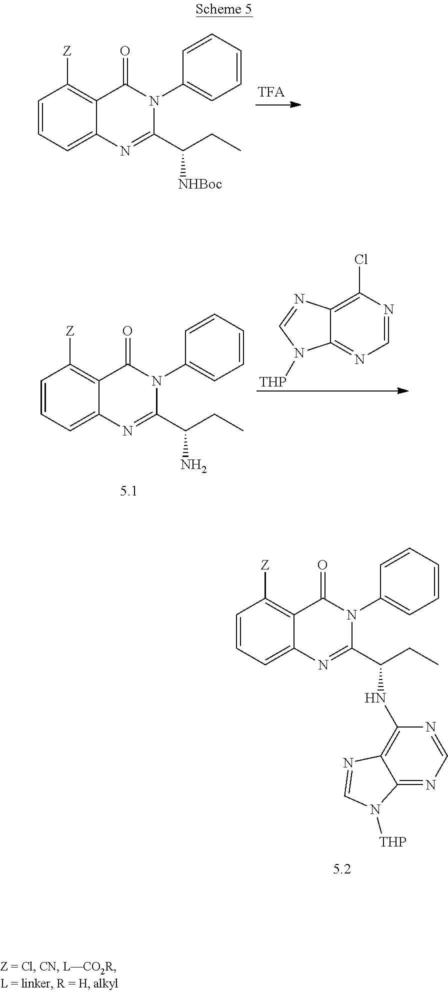

### Scheme 5

The Boc-protected amine (1 equiv) was dissolved in DCM (0.1 M) in a vial and trifluoroacetic acid (20 equiv) was added dropwise to it. The resulting mixture was stirred at room temperature for 3 hours. After completion of reaction (by LC-MS) the reaction mixture is worked-up by either of the following two methods:

Method A: The crude reaction is quenched with aqueous saturated NaHCO₃ solution and extracted three times with DCM. The combined organic layers were dried over anhydrous MgSO₄, filtered and concentrated in vacuo to afford the free amine 5.1.

Method B: The crude reaction mixture is concentrated in vacuo, re-dissolved in 1-2 ml of DCM, passed through pre-conditioned PL-HCO₃ MP SPE device and washed with 2 ml of DCM. The filtrate was concentrated in vacuo to afford the free amine 5.1.

The free amine 5.1 was dissolved in ethanol (0.4 M) in a microwave vial equipped with a stir bar followed by the addition of 6-chloro-9-(tetrahydro-2H-pyran-2-yl)-9H-purine (2.0 equiv) and triethylamine (4.0 equiv) to it. The vial was sealed and heated for 4 hours at 100° C. in a microwave. After completion of reaction (by LC-MS), the reaction mixture was concentrated in vacuo and the remaining residue was purified by flash chromatography on silica gel using forced flow of indicated solvent system on Biotage KP-Sil pre-packed cartridges and using the Biotage SP-1 automated chromatography system to afford the coupled product 5.2.

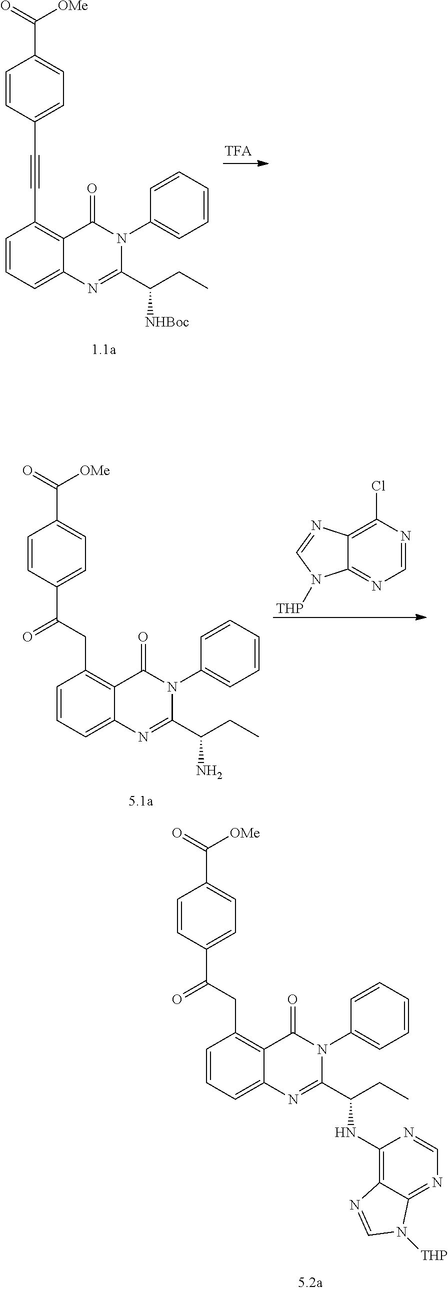

The procedure mentioned in Scheme 5 was used with (S)-methyl 4-((2-(1-((tert-butoxycarbonyl)amino)propyl)-4-oxo-3-phenyl-3,4-dihydroquinazolin-5-yl)ethynyl)benzoate 1.1a (205.0 mg, 0.38 mmol) and trifluoroacetic acid (870.0 mg, 7.63 mmol, 0.58 ml) in dichloromethane (3.8 ml). The resulting mixture was stirred at room temperature for 3 hours and worked-up (Method A) to afford methyl (S)-4-(2-(2-(1-aminopropyl)-4-oxo-3-phenyl-3,4-dihydroquinazolin-5-yl)acetyl)benzoate 5.1a (Note: the alkyne in 1.1a hydrolyzed to ketone in product 5.1a under the reaction conditions). This free amine 5.1a was used with 6-chloro-9-(tetrahydro-2H-pyran-2-yl)-9H-purine (182.0 mg, 0.76 mmol) and triethylamine (154.0 mg, 1.52 mmol, 0.21 ml) in ethanol (1.0 ml). The remaining residue was purified by flash chromatography on silica gel using 0-5% MeOH/DCM to afford the product methyl 4-(2-(4-oxo-3-phenyl-2-((1S)-1-((9-(tetrahydro-2H-pyran-2-yl)-9H-purin-6-yl)amino)propyl)-3,4-dihydroquinazolin-5-yl)acetyl)benzoate 5.2a (236.0 mg, 0.36 mmol) as a light-brown solid in 94% yield. LC-MS (method 1): t_{R}=3.43 min, m/z (M+H)⁺=658.2.

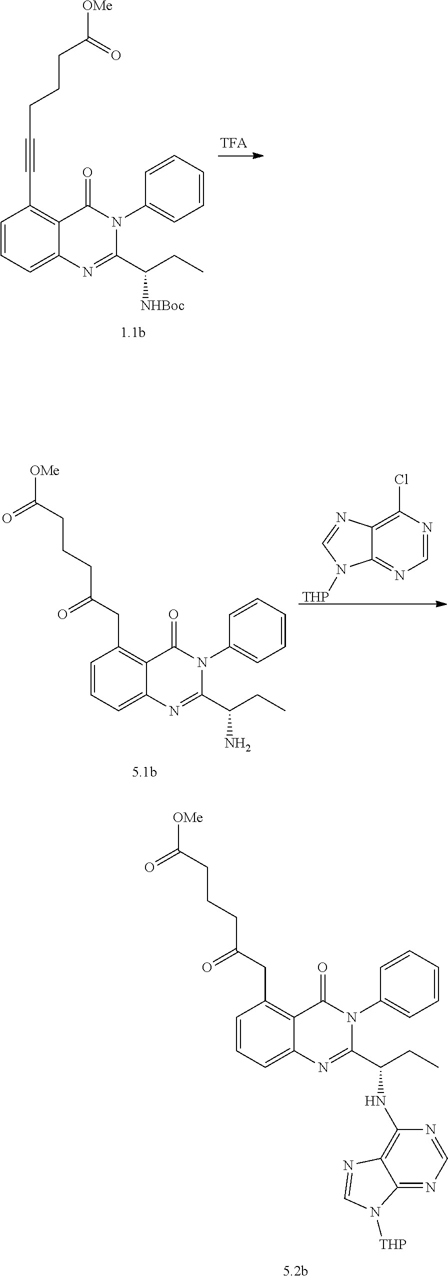

The procedure mentioned in Scheme 5 was used with (S)-methyl 6-(2-(1-((tert-butoxycarbonyl)amino)propyl)-4-oxo-3-phenyl-3,4-dihydroquinazolin-5-yl)hex-5-ynoate 1.1b (150.0 mg, 0.30 mmol) and trifluoroacetic acid (679.0 mg, 5.96 mmol, 0.46 ml) in dichloromethane (3.0 ml). The resulting mixture was stirred at room temperature for 3 hours and worked-up (Method A) to afford methyl (S)-6-(2-(1-aminopropyl)-4-oxo-3-phenyl-3,4-dihydroquinazolin-5-yl)-5-oxohexanoate 5.1b (Note: the alkyne hydrolyzed to ketone in product 5.1 b under the reaction conditions). This free amine 5.1b was used with 6-chloro-9-(tetrahydro-2H-pyran-2-yl)-9H-purine (142.0 mg, 0.60 mmol) and triethylamine (121.0 mg, 1.19 mmol, 0.17 ml) in ethanol (0.7 ml). The remaining residue was purified by flash chromatography on silica gel using 0-5% MeOH/DCM to afford the product methyl 5-oxo-6-(4-oxo-3-phenyl-2-((1S)-1-((9-(tetrahydro-2H-pyran-2-yl)-9H-purin-6-yl)amino)propyl)-3,4-dihydroquinazolin-5-yl)hexanoate 5.2b (140.0 mg, 0.22 mmol) as a light-brown solid in 75% yield. LC-MS (method 1): t_{R}=3.29 min, m/z (M+H)⁺=624.2.

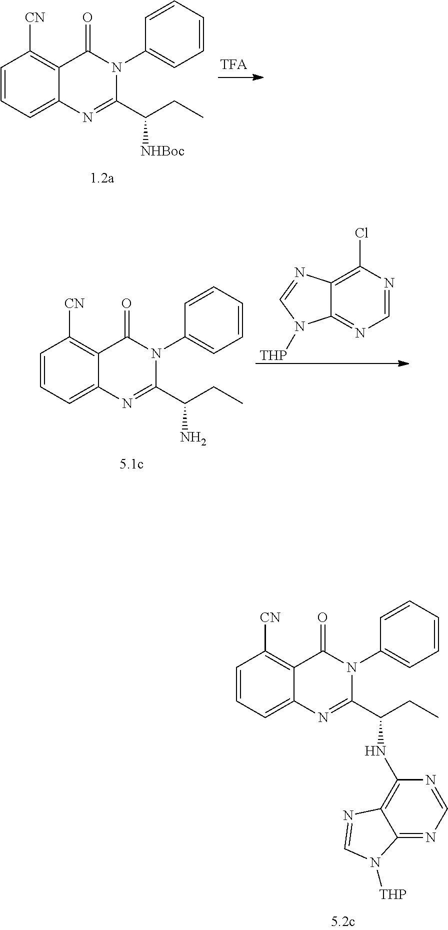

The procedure mentioned in Scheme 5 was used with tert-butyl (S)-(1-(5-cyano-4-oxo-3-phenyl-3,4-dihydroquinazolin-2-yl)propyl)carbamate 1.2a (83.0 mg, 0.21 mmol) and trifluoroacetic acid (468.0 mg, 4.10 mmol, 0.32 ml) in dichloromethane (2.0 ml). The resulting mixture was stirred at room temperature for 3 hours and worked-up (Method A) to afford (S)-2-(1-aminopropyl)-4-oxo-3-phenyl-3,4-dihydroquinazoline-5-carbonitrile 5.1c. This free amine 5.1c was used with 6-chloro-9-(tetrahydro-2H-pyran-2-yl)-9H-purine (98.0 mg, 0.41 mmol) and triethylamine (83.0 mg, 0.82 mmol, 114.0 μl) in ethanol (0.5 ml). The remaining residue was purified by flash chromatography on silica gel using 0-5% MeOH/DCM to afford the product 4-oxo-3-phenyl-2-((1S)-1-((9-(tetrahydro-2H-pyran-2-yl)-9H-purin-6-yl)amino)propyl)-3,4-dihydroquinazoline-5-carbonitrile 5.2c (84.2 mg, 0.17 mmol) in 81% yield. LC-MS (method 1): t_{R}=3.18 min, m/z (M+H)⁺=507.3.

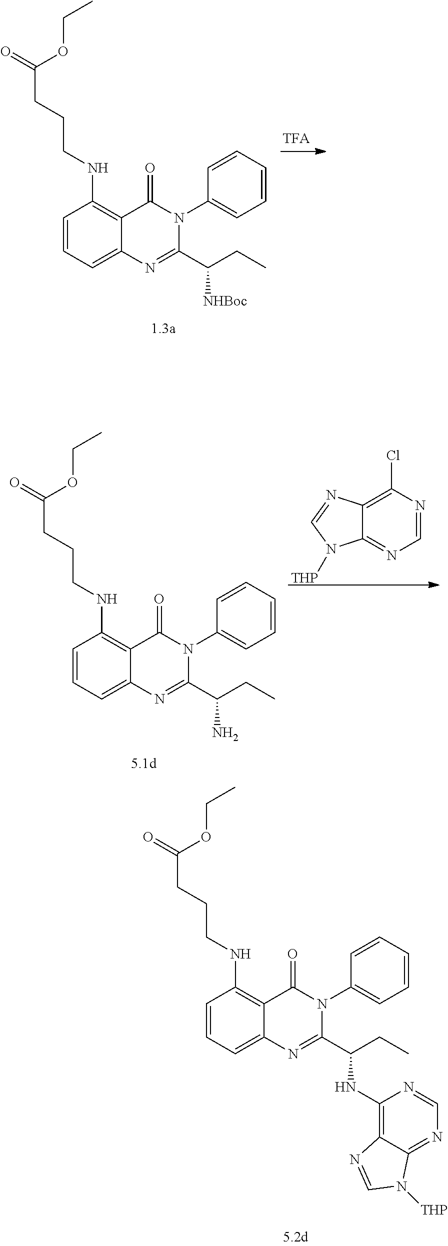

The procedure mentioned in Scheme 5 was used with (S)-ethyl 4-((2-(1-((tert-butoxycarbonyl)amino)propyl)-4-oxo-3-phenyl-3,4-dihydroquinazolin-5-yl)amino)butanoate 1.3a (83.0 mg, 0.16 mmol) and trifluoroacetic acid (374.0 mg, 3.28 mmol, 0.25 ml) in dichloromethane (1.6 ml). The resulting mixture was stirred at room temperature for 3 hours and worked-up (Method A) to afford (S)-ethyl 4-((2-(1-aminopropyl)-4-oxo-3-phenyl-3,4-dihydroquinazolin-5-yl)amino)butanoate 5.1d. This free amine 5.1d was used with 6-chloro-9-(tetrahydro-2H-pyran-2-yl)-9H-purine (78.0 mg, 0.33 mmol) and triethylamine (66.0 mg, 0.66 mmol, 91.0 μl) in ethanol (0.5 ml). The remaining residue was purified by flash chromatography on silica gel using 0-5% MeOH/DCM to afford the product ethyl 4-((4-oxo-3-phenyl-2-((1S)-1-((9-(tetrahydro-2H-pyran-2-yl)-9H-purin-6-yl)amino)propyl)-3,4-dihydroquinazolin-5-yl)amino)butanoate 5.2d (53.0 mg, 0.09 mmol) in 53% yield. LC-MS (method 1): t_{R}=3.46 min, m/z (M+H)⁺=611.4.

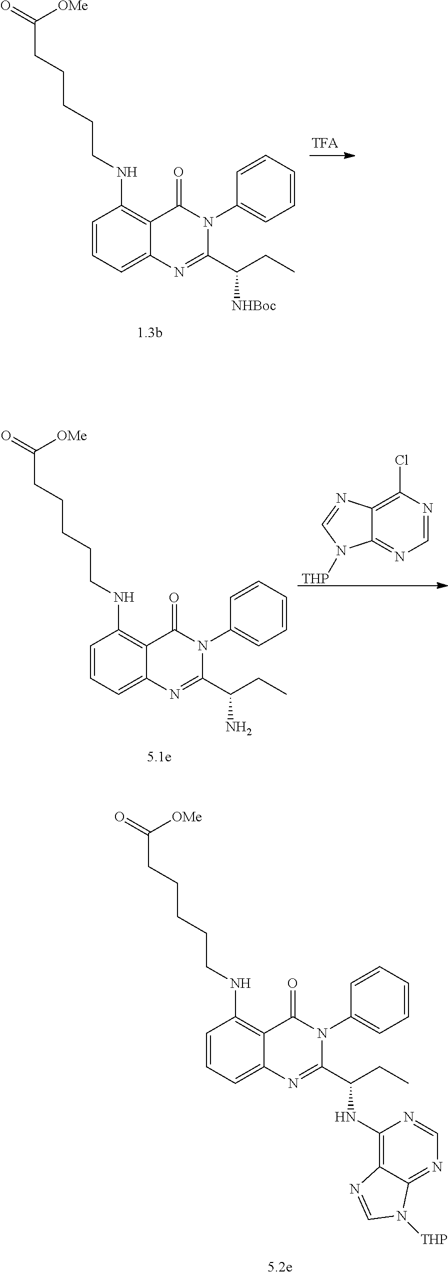

The procedure mentioned in Scheme 5 was used with (S)-methyl 6-((2-(1-((tert-butoxycarbonyl)amino)propyl)-4-oxo-3-phenyl-3,4-dihydroquinazolin-5-yl)amino)hexanoate 1.3b (126.0 mg, 0.24 mmol) and trifluoroacetic acid (550.0 mg, 4.82 mmol, 0.37 ml) in dichloromethane (2.4 ml). The resulting mixture was stirred at room temperature for 3 hours and worked-up (Method A) to form (S)-methyl 6-((2-(1-aminopropyl)-4-oxo-3-phenyl-3,4-dihydroquinazolin-5-yl)amino)hexanoate 5.1e. This free amine 5.1e was used with 6-chloro-9-(tetrahydro-2H-pyran-2-yl)-9H-purine (115.0 mg, 0.48 mmol) and triethylamine (98.0 mg, 0.96 mmol, 134.0 μl) in ethanol (0.9 ml). The remaining residue was purified by flash chromatography on silica gel using 0-5% MeOH/DCM to afford the product methyl 6-((4-oxo-3-phenyl-2-((1S)-1-((9-(tetrahydro-2H-pyran-2-yl)-9H-purin-6-yl)amino)propyl)-3,4-dihydroquinazolin-5-yl)amino)hexanoate 5.2e (134.9 mg, 0.22 mmol) in 90% yield. LC-MS (method 1): t_{R}=3.52 min, m/z (M+H)⁺=625.4.

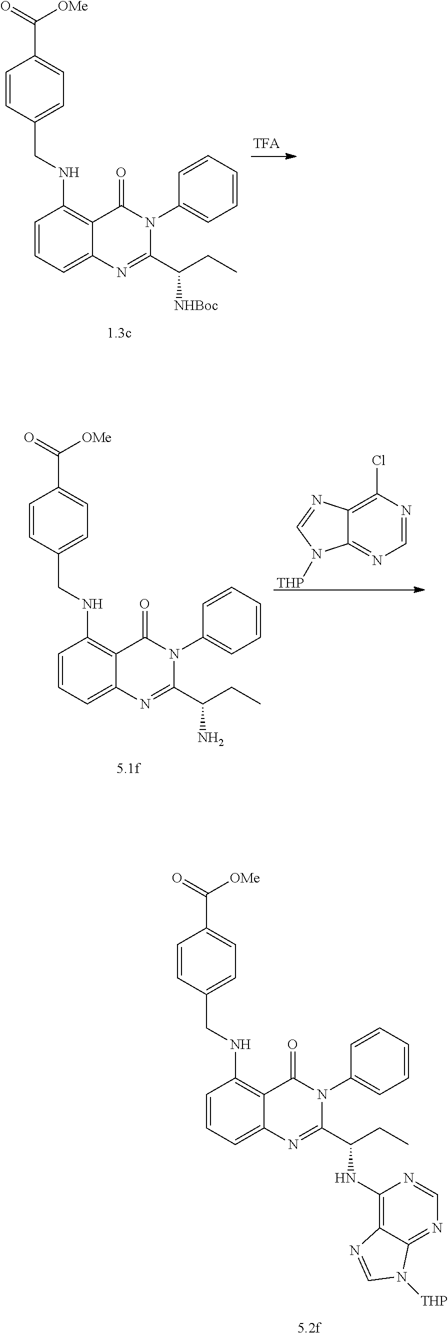

The procedure mentioned in Scheme 5 was used with methyl (S)-4-(((2-(1-((tert-butoxycarbonyl)amino)propyl)-4-oxo-3-phenyl-3,4-dihydroquinazolin-5-yl)amino)methyl)benzoate 1.3c (100.0 mg, 0.18 mmol) and trifluoroacetic acid (420.0 mg, 3.69 mmol, 0.28 ml) in dichloromethane (1.8 ml). The resulting mixture was stirred at room temperature for 3 hours and worked-up (Method A) to afford methyl (S)-4-(((2-(1-aminopropyl)-4-oxo-3-phenyl-3,4-dihydroquinazolin-5-yl)amino)methyl)benzoate 5.1f. This free amine 5.1f was used with 6-chloro-9-(tetrahydro-2H-pyran-2-yl)-9H-purine (88.0 mg, 0.37 mmol) and triethylamine (74.5 mg, 0.74 mmol, 103.0 μl) in ethanol (0.4 ml). The remaining residue was purified by flash chromatography on silica gel using 0-100% EtOAc/Hexanes to afford the product methyl 4-(((4-oxo-3-phenyl-2-((1S)-1-((9-(tetrahydro-2H-pyran-2-yl)-9H-purin-6-yl)amino)propyl)-3,4-dihydroquinazolin-5-yl)amino)methyl)benzoate 5.2f (94.5 mg, 0.15 mmol) in 80% yield. LC-MS (method 1): t_{R}=3.54 min, m/z (M+H)⁺=645.3.

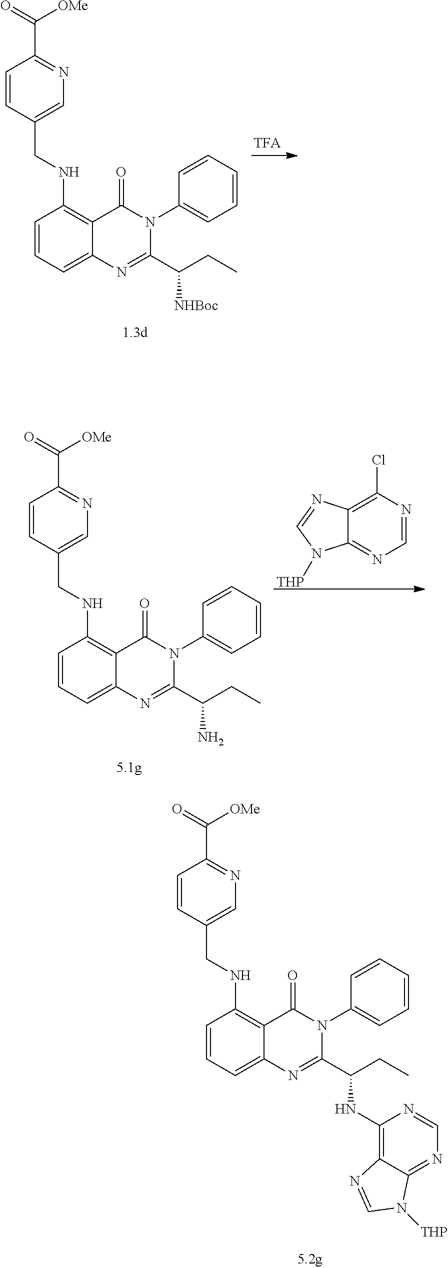

The procedure mentioned in Scheme 5 was used with methyl (S)-5-(((2-(1-((tert-butoxycarbonyl)amino)propyl)-4-oxo-3-phenyl-3,4-dihydroquinazolin-5-yl)amino)methyl)picolinate 1.3d (67.5 mg, 0.124 mmol) and trifluoroacetic acid (283.0 mg, 2.48 mmol, 0.19 ml) in dichloromethane (1.2 ml). The resulting mixture was stirred at room temperature for 3 hours and worked-up (Method A) to afford methyl (S)-5-(((2-(1-aminopropyl)-4-oxo-3-phenyl-3,4-dihydroquinazolin-5-yl)amino)methyl)picolinate 5.1g. This free amine 5.1g was used with 6-chloro-9-(tetrahydro-2H-pyran-2-yl)-9H-purine (59.2 mg, 0.25 mmol) and triethylamine (50.0 mg, 0.50 mmol, 69.2 μl) in ethanol (0.3 ml). The remaining residue was purified by flash chromatography on silica gel using 0-5% MeOH/DCM to afford the product methyl 5-(((4-oxo-3-phenyl-2-((1S)-1-((9-(tetrahydro-2H-pyran-2-yl)-9H-purin-6-yl)amino)propyl)-3,4-dihydroquinazolin-5-yl)amino)methyl)picolinate 5.2g (60.5 mg, 0.094 mmol) in 76% yield. LC-MS (method 1): t_{R}=3.27 min, m/z (M+H)⁺=646.3.

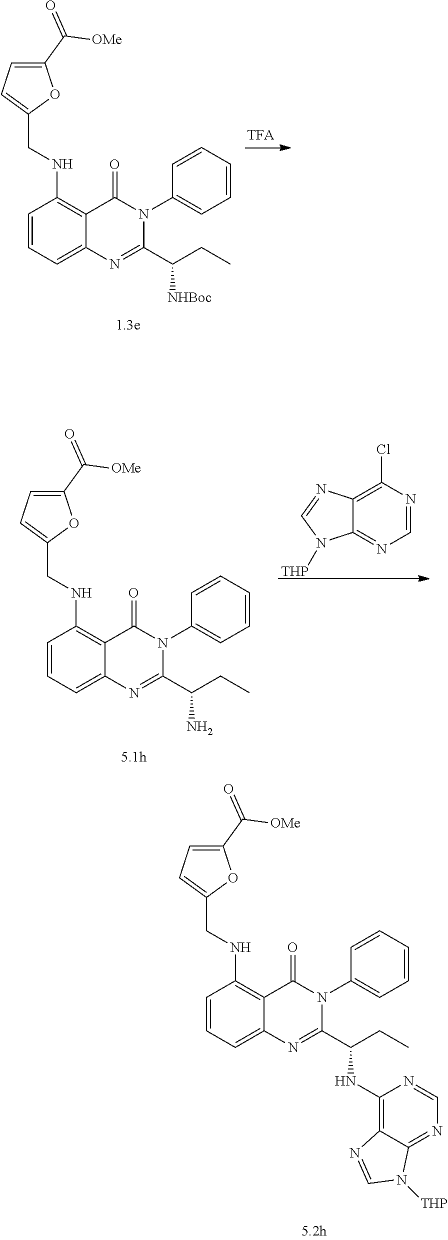

The procedure mentioned in Scheme 5 was used with methyl (S)-5-(((2-(1-((tert-butoxycarbonyl)amino)propyl)-4-oxo-3-phenyl-3,4-dihydroquinazolin-5-yl)amino)methyl)furan-2-carboxylate 1.3e (58.0 mg, 0.109 mmol) and trifluoroacetic acid (248.0 mg, 2.18 mmol, 0.17 ml) in dichloromethane (1.1 ml). The resulting mixture was stirred at room temperature for 3 hours and worked-up (Method A) to afford methyl (S)-5-(((2-(1-aminopropyl)-4-oxo-3-phenyl-3,4-dihydroquinazolin-5-yl)amino)methyl)furan-2-carboxylate 5.1h. This free amine 5.1h was used with 6-chloro-9-(tetrahydro-2H-pyran-2-yl)-9H-purine (52.0 mg, 0.22 mmol) and triethylamine (44.1 mg, 0.44 mmol, 61.0 μl) in ethanol (0.5 ml). The remaining residue was purified by flash chromatography on silica gel using 0-100% EtOAc/Hexanes to afford the product methyl 5-(((4-oxo-3-phenyl-2-((1S)-1-((9-(tetrahydro-2H-pyran-2-yl)-9H-purin-6-yl)amino)propyl)-3,4-dihydroquinazolin-5-yl)amino)methyl)furan-2-carboxylate 5.2h (50.0 mg, 0.079 mmol) in 72% yield. LC-MS (method 1): t_{R}=3.41 min, m/z (M+H)⁺=635.3.

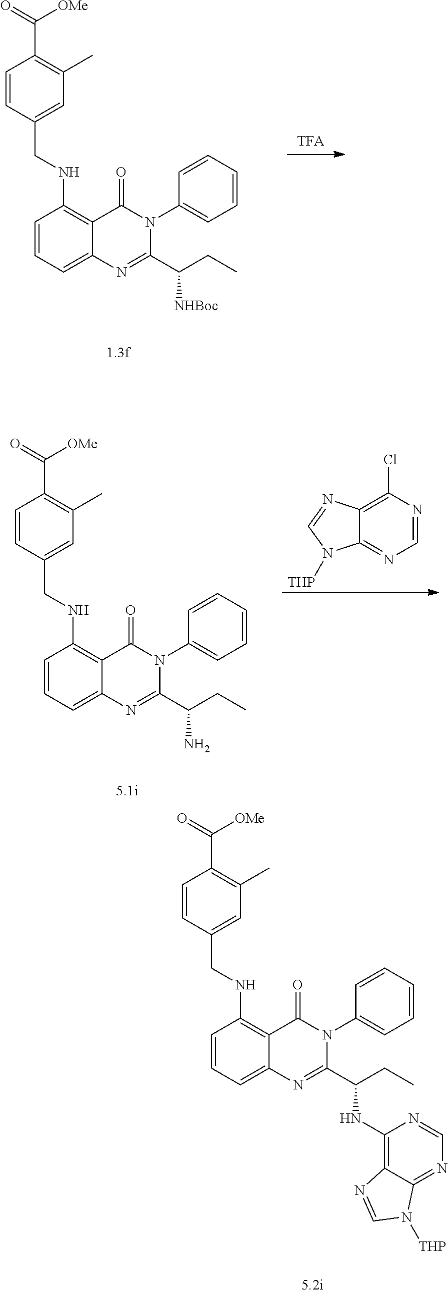

The procedure mentioned in Scheme 5 was used with methyl (S)-4-(((2-(1-((tert-butoxycarbonyl)amino)propyl)-4-oxo-3-phenyl-3,4-dihydroquinazolin-5-yl)amino)methyl)-2-methylbenzoate 1.3f (175.0 mg, 0.314 mmol) and trifluoroacetic acid (717.0 mg, 6.29 mmol, 0.48 ml) in dichloromethane (3.1 ml). The resulting mixture was stirred at room temperature for 3 hours and worked-up (Method A) to afford methyl (S)-4-(((2-(1-aminopropyl)-4-oxo-3-phenyl-3,4-dihydroquinazolin-5-yl)amino)methyl)-2-methylbenzoate 5.1i. This free amine 5.1i was used with 6-chloro-9-(tetrahydro-2H-pyran-2-yl)-9H-purine (151.0 mg, 0.63 mmol) and triethylamine (128.0 mg, 1.26 mmol, 176 μl) in ethanol (0.7 ml). The remaining residue was purified by flash chromatography on silica gel using 0-100% EtOAc/Hexanes to afford the product methyl 2-methyl-4-(((4-oxo-3-phenyl-2-((1S)-1-((9-(tetrahydro-2H-pyran-2-yl)-9H-purin-6-yl)amino)propyl)-3,4-dihydroquinazolin-5-yl)amino)methyl)benzoate 5.2i (189.0 mg, 0.29 mmol) in 91% yield. LC-MS (method 1): t_{R}=3.66 min, m/z (M+H)⁺=659.3.

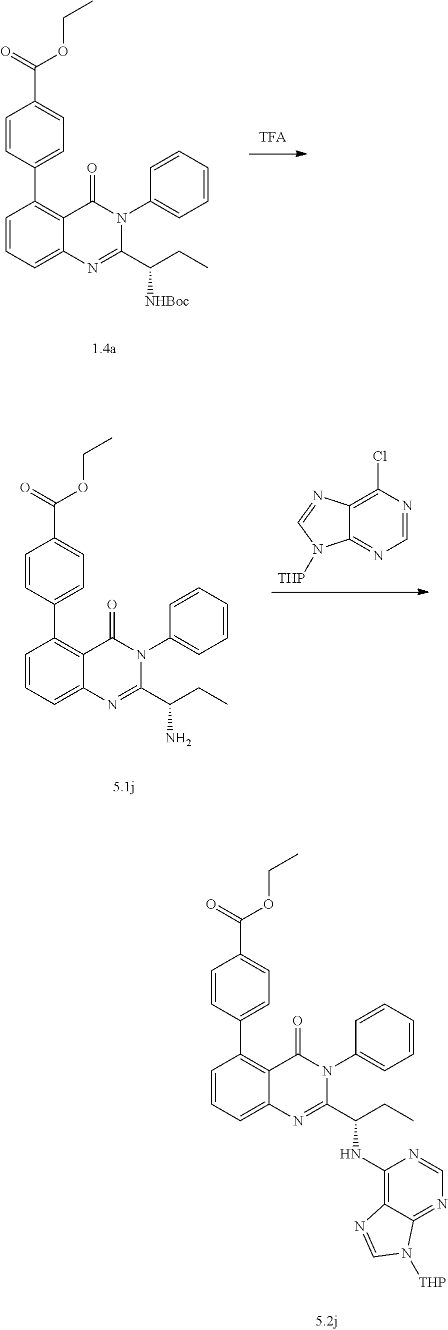

The procedure mentioned in Scheme 5 was used with ethyl (S)-4-(2-(1-((tert-butoxycarbonyl)amino)propyl)-4-oxo-3-phenyl-3,4-dihydroquinazolin-5-yl)benzoate 1.4a (320.0 mg, 0.61 mmol) and trifluoroacetic acid (1.38 g, 12.13 mmol, 0.93 ml) in dichloromethane (6.0 ml). The resulting mixture was stirred at room temperature for 3 hours and worked-up (Method A) to afford ethyl (S)-4-(2-(1-aminopropyl)-4-oxo-3-phenyl-3,4-dihydroquinazolin-5-yl)benzoate 5.1j. This free amine 5.1j was used with 6-chloro-9-(tetrahydro-2H-pyran-2-yl)-9H-purine (289.0 mg, 1.21 mmol) and triethylamine (245 mg, 2.42 mmol, 0.34 ml) in ethanol (1.2 ml). The remaining residue was purified by flash chromatography on silica gel using 0-5% MeOH/DCM to afford the product ethyl 4-(4-oxo-3-phenyl-2-((1S)-1-((9-(tetrahydro-2H-pyran-2-yl)-9H-purin-6-yl)amino)propyl)-3,4-dihydroquinazolin-5-yl)benzoate 5.2j (348.0 mg, 0.55 mmol) in 91% yield. LC-MS (method 1): t_{R}=3.56 min, m/z (M+H)⁺=630.3.

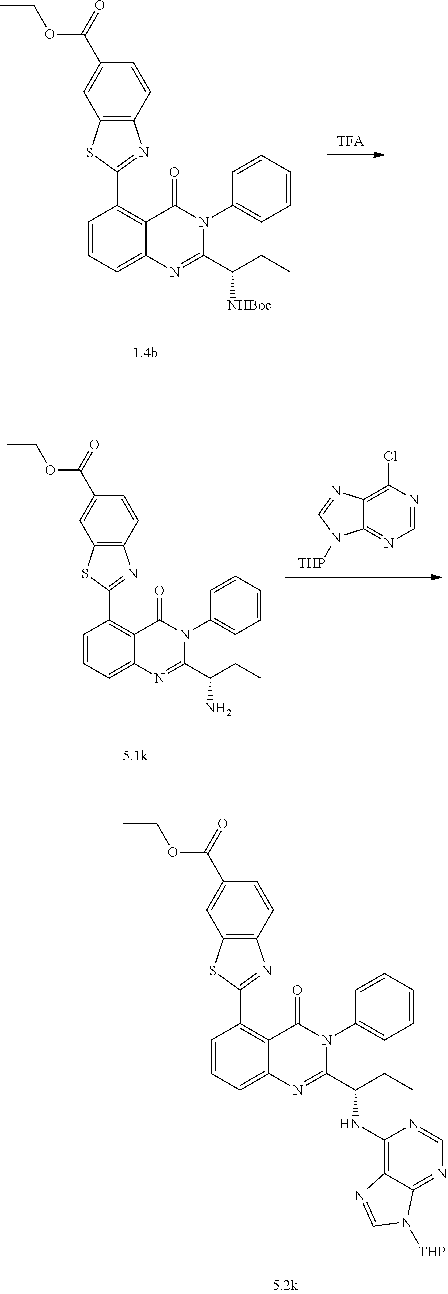

The procedure mentioned in Scheme 5 was used with ethyl (S)-2-(2-(1-((tert-butoxycarbonyl)amino)propyl)-4-oxo-3-phenyl-3,4-dihydroquinazolin-5-yl)benzo[d]thiazole-6-carboxylate 1.4b (41.0 mg, 0.07 mmol) and trifluoroacetic acid (160.0 mg, 1.40 mmol, 0.11 ml) in dichloromethane (0.7 ml). The resulting mixture was stirred at room temperature for 3 hours and worked-up (Method A) to afford ethyl (S)-2-(2-(1-aminopropyl)-4-oxo-3-phenyl-3,4-dihydroquinazolin-5-yl)benzo[d]thiazole-6-carboxylate 5.1k. This free amine 5.1k was used with 6-chloro-9-(tetrahydro-2H-pyran-2-yl)-9H-purine (33.0 mg, 0.14 mmol) and triethylamine (28.0 mg, 0.28 mmol, 39.0 μl) in ethanol (0.2 ml). The remaining residue was purified by flash chromatography on silica gel using 0-5% MeOH/DCM to afford the product ethyl 2-(4-oxo-3-phenyl-2-((1S)-1-((9-(tetrahydro-2H-pyran-2-yl)-9H-purin-6-yl)amino)propyl)-3,4-dihydroquinazolin-5-yl)benzo[d]thiazole-6-carboxylate 5.2k (23.0 mg, 0.033 mmol) in 48% yield. LC-MS (method 1): t_{R}=3.64 min, m/z (M+H)⁺=687.2.

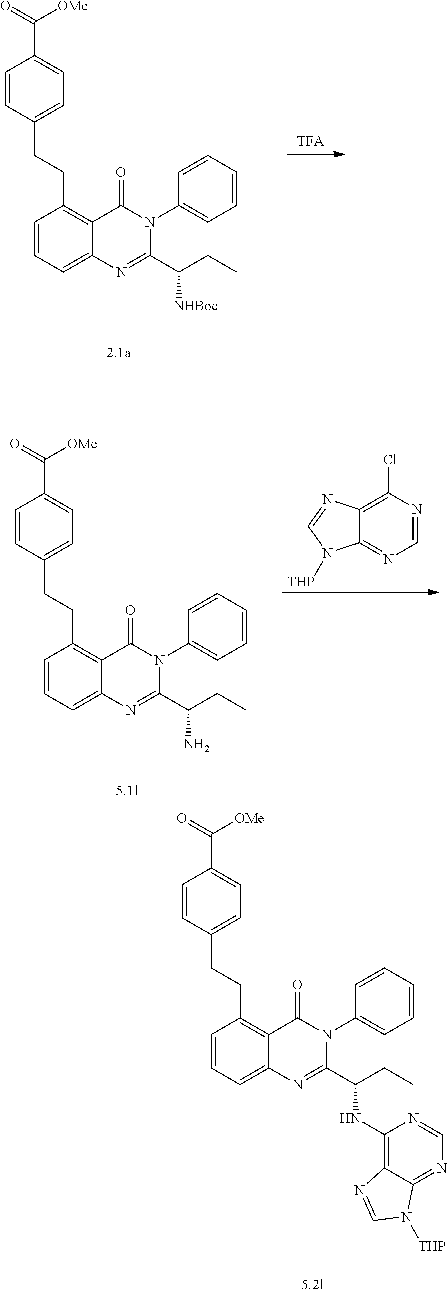

The procedure mentioned in Scheme 5 was used with (S)-methyl 4-(2-(2-(1-((tert-butoxycarbonyl)amino)propyl)-4-oxo-3-phenyl-3,4-dihydroquinazolin-5-yl)ethyl)benzoate 2.1a (68.0 mg, 0.13 mmol) and trifluoroacetic acid (287.0 mg, 2.52 mmol, 0.19 ml) in dichloromethane (1.3 ml) The resulting mixture was stirred at room temperature for 3 hours and worked-up (Method A) to afford (S)-methyl 4-(2-(2-(1-aminopropyl)-4-oxo-3-phenyl-3,4-dihydroquinazolin-5-yl)ethyl)benzoate 5.11. This free amine 5.11 was used with 6-chloro-9-(tetrahydro-2H-pyran-2-yl)-9H-purine (60.0 mg, 0.25 mmol) and triethylamine (51.0 mg, 0.50 mmol, 70 μl) in ethanol (0.4 ml). The remaining residue was purified by flash chromatography on silica gel using 0-5% MeOH/DCM to afford the product methyl 4-(2-(4-oxo-3-phenyl-2-((1S)-1-((9-(tetrahydro-2H-pyran-2-yl)-9H-purin-6-yl)amino)propyl)-3,4-dihydroquinazolin-5-yl)ethyl)benzoate 5.21 (60.0 mg, 0.093 mmol) in 74% yield. LC-MS (method 1): t_{R}=3.62 min, m/z (M+H)⁺=644.3.

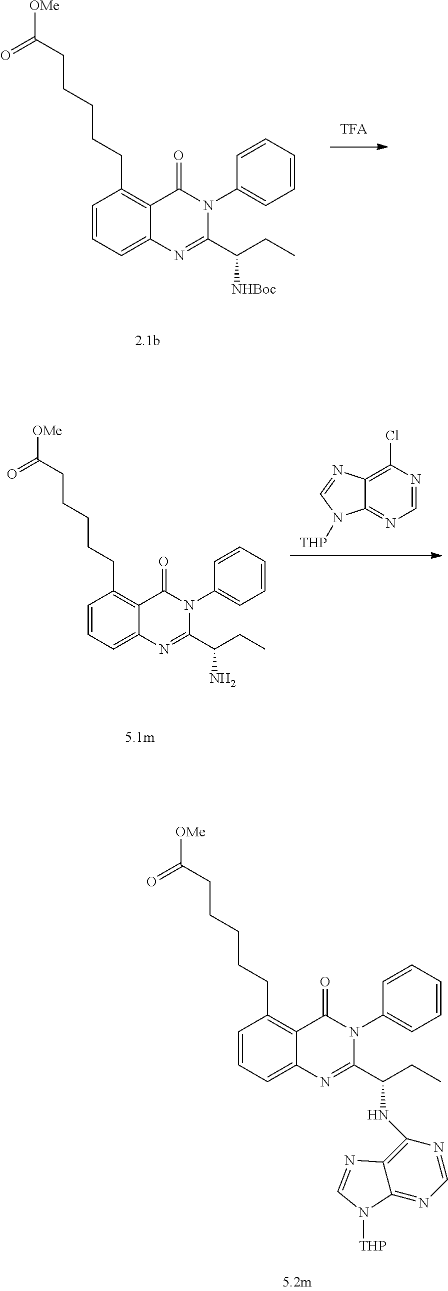

The procedure mentioned in Scheme 5 was used with (S)-methyl 6-(2-(1-((tert-butoxycarbonyl)amino)propyl)-4-oxo-3-phenyl-3,4-dihydroquinazolin-5-yl)hexanoate 2.1b (111.0 mg, 0.22 mmol) and trifluoroacetic acid (497.0 mg, 4.36 mmol, 0.34 ml) in dichloromethane (2.2 ml) The resulting mixture was stirred at room temperature for 3 hours and worked-up (Method A) to afford (S)-methyl 6-(2-(1-aminopropyl)-4-oxo-3-phenyl-3,4-dihydroquinazolin-5-yl)hexanoate 5.1m. This free amine 5.1m was used with 6-chloro-9-(tetrahydro-2H-pyran-2-yl)-9H-purine (104.0 mg, 0.44 mmol) and triethylamine (88.0 mg, 0.87 mmol, 122 μl) in ethanol (0.5 ml). The remaining residue was purified by flash chromatography on silica gel using 0-5% MeOH/DCM to afford the product methyl 6-(4-oxo-3-phenyl-2-((1S)-1-((9-(tetrahydro-2H-pyran-2-yl)-9H-purin-6-yl)amino)propyl)-3,4-dihydroquinazolin-5-yl)hexanoate 5.2m (122.0 mg, 0.2 mmol) in 92% yield. LC-MS (method 1): t_{R}=3.53 min, m/z (M+H)⁺=610.4.

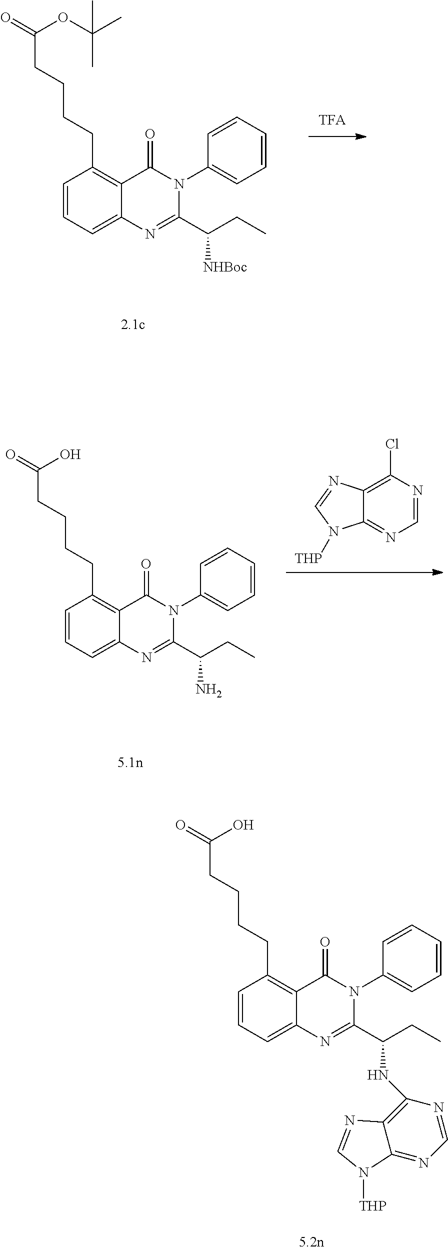

The procedure mentioned in Scheme 5 was used with (S)-tert-butyl 5-(2-(1-((tert-butoxycarbonyl)amino)propyl)-4-oxo-3-phenyl-3,4-dihydroquinazolin-5-yl)pentanoate 2.1c (103.0 mg, 0.19 mmol) and trifluoroacetic acid [TFA] (438.0 mg, 3.84 mmol, 0.29 ml) in dichloromethane (1.9 ml). The resulting mixture was stirred at room temperature for 3 hours and worked-up (Method B) to afford (S)-5-(2-(1-aminopropyl)-4-oxo-3-phenyl-3,4-dihydroquinazolin-5-yl)pentanoic acid 5.1n. This free amine 5.1n was used with 6-chloro-9-(tetrahydro-2H-pyran-2-yl)-9H-purine (92.0 mg, 0.38 mmol) and triethylamine (78.0 mg, 0.77 mmol, 107 μl) in ethanol (0.5 ml). The remaining residue was purified by flash chromatography on silica gel using 0-100% EtOAc (0.5% AcOH by vol)/hexanes to afford the product 5-(4-oxo-3-phenyl-2-((1S)-1-((9-(tetrahydro-2H-pyran-2-yl)-9H-purin-6-yl)amino)propyl)-3,4-dihydroquinazolin-5-yl)pentanoic acid 5.2n (60.0 mg, 0.103 mmol) in 54% yield. LC-MS (method 1): t_{R}=3.17 min, m/z (M+H)⁺=582.4.

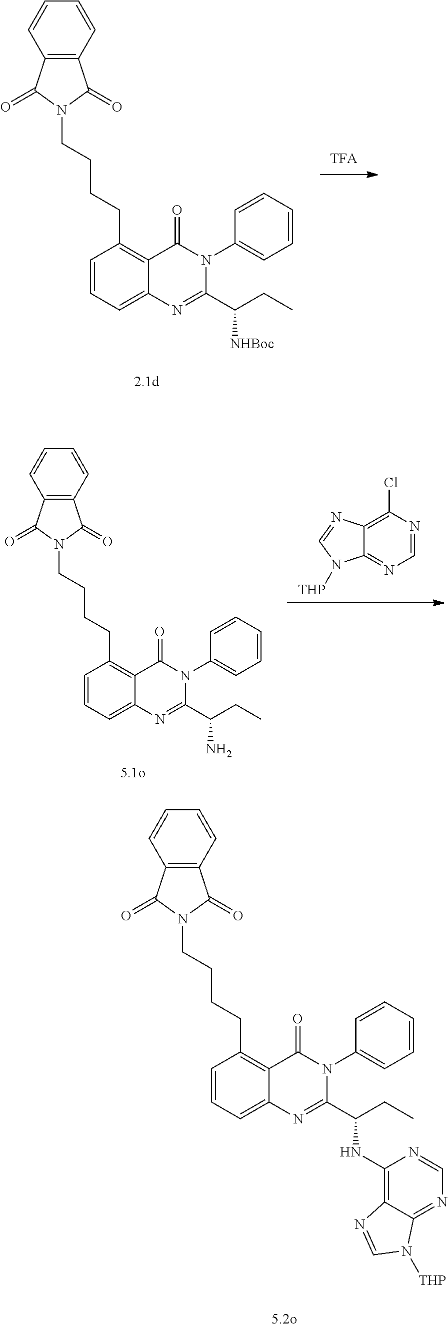

The procedure mentioned in Scheme 5 was used with (S)-tert-butyl (1-(5-(4-(1,3-dioxoisoindolin-2-yl)butyl)-4-oxo-3-phenyl-3,4-dihydroquinazolin-2-yl)propyl)carbamate 2.1d (126.0 mg, 0.22 mmol) and trifluoroacetic acid [TFA] (495.0 mg, 4.34 mmol, 0.34 ml) in dichloromethane (2.2 ml) The resulting mixture was stirred at room temperature for 3 hours and worked-up (Method A) to afford (S)-2-(4-(2-(1-aminopropyl)-4-oxo-3-phenyl-3,4-dihydroquinazolin-5-yl)butyl)isoindoline-1,3-dione 5.1o. This free amine 5.1o was used with 6-chloro-9-(tetrahydro-2H-pyran-2-yl)-9H-purine (104.0 mg, 0.43 mmol) and triethylamine (88.0 mg, 0.87 mmol, 122 μl) in ethanol (0.5 ml). The remaining residue was purified by flash chromatography on silica gel using 0-10% MeOH/DCM to afford the product 2-(4-(4-oxo-3-phenyl-2-((1S)-1-((9-(tetrahydro-2H-pyran-2-yl)-9H-purin-6-yl)amino)propyl)-3,4-dihydroquinazolin-5-yl)butyl)isoindoline-1,3-dione 5.2o (127.5 mg, 0.19 mmol) in 86% yield. LC-MS (method 1): t_{R}=3.55 min, m/z (M+H)⁺=683.4.

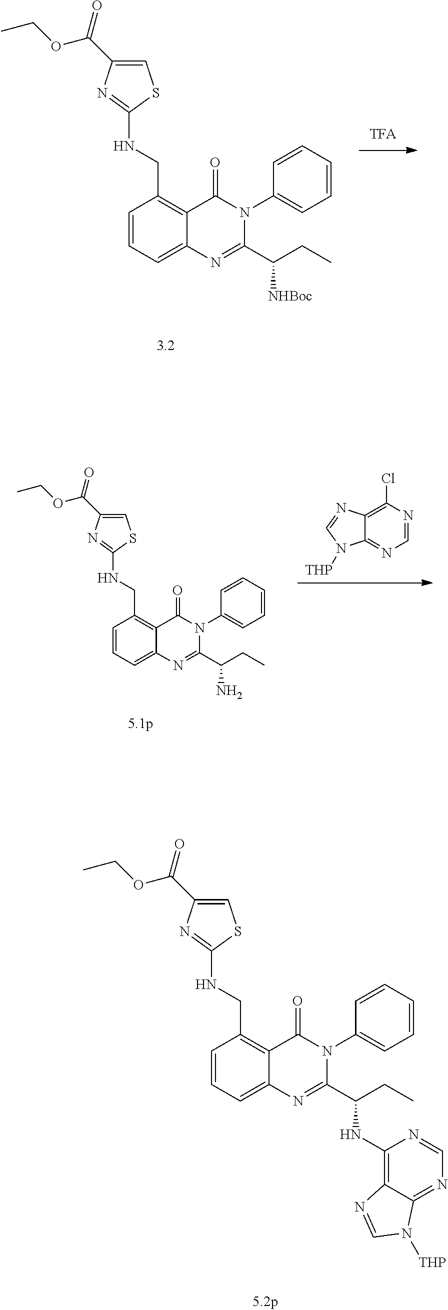

The procedure mentioned in Scheme 5 was used with ethyl (S)-2-(((2-(1-((tert-butoxycarbonyl)amino)propyl)-4-oxo-3-phenyl-3,4-dihydroquinazolin-5-yl)methyl)amino)thiazole-4-carboxylate 3.2 (78.8 mg, 0.14 mmol) and trifluoroacetic acid (319.0 mg, 2.80 mmol, 0.21 ml) in dichloromethane (1.4 ml). The resulting mixture was stirred at room temperature for 3 hours and worked-up (Method A) to afford ethyl (S)-2-(((2-(1-aminopropyl)-4-oxo-3-phenyl-3,4-dihydroquinazolin-5-yl)methyl)amino)thiazole-4-carboxylate 5.1p. This free amine 5.1p was used with 6-chloro-9-(tetrahydro-2H-pyran-2-yl)-9H-purine (66.8 mg, 0.28 mmol) and triethylamine (56.7 mg, 0.56 mmol, 78.0 μl) in ethanol (0.3 ml). The remaining residue was purified by flash chromatography on silica gel using 0-5% MeOH/DCM to afford the product ethyl 2-(((4-oxo-3-phenyl-2-((1S)-1-((9-(tetrahydro-2H-pyran-2-yl)-9H-purin-6-yl)amino)propyl)-3,4-dihydroquinazolin-5-yl)methyl)amino)thiazole-4-carboxylate 5.2p (68.1 mg, 0.102 mmol) in 73% yield. LC-MS (method 1): t_{R}=3.35 min, m/z (M+H)⁺=666.3.

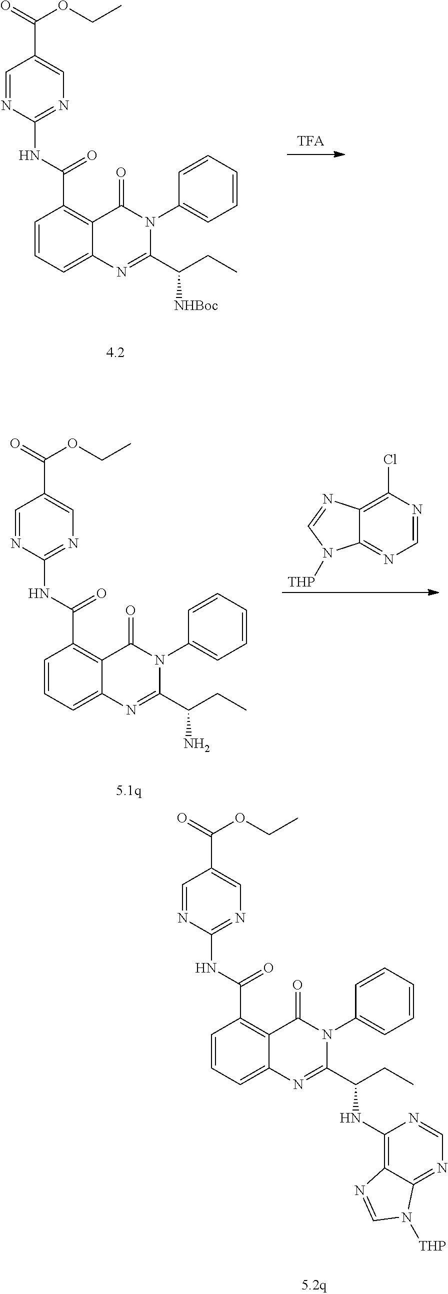

The procedure mentioned in Scheme 5 was used with (S)-ethyl 2-(2-(1-((tert-butoxycarbonyl)amino)propyl)-4-oxo-3-phenyl-3,4-dihydroquinazoline-5-carboxamido)pyrimidine-5-carboxylate 4.2 (37.0 mg, 0.064 mmol) and trifluoroacetic acid (146.0 mg, 1.28 mmol, 0.10 ml) in dichloromethane (0.6 ml). The resulting mixture was stirred at room temperature for 3 hours and worked-up (Method A) to afford (S)-ethyl 2-(2-(1-aminopropyl)-4-oxo-3-phenyl-3,4-dihydroquinazoline-5-carboxamido)pyrimidine-5-carboxylate 5.1q. This free amine 5.1q was used with 6-chloro-9-(tetrahydro-2H-pyran-2-yl)-9H-purine (31.0 mg, 0.13 mmol) and triethylamine (26.0 mg, 0.26 mmol, 36.0 μl) in ethanol (0.2 ml). The remaining residue was purified by flash chromatography on silica gel using 0-5% MeOH/DCM to afford the product ethyl 2-(4-oxo-3-phenyl-2-((1S)-1-((9-(tetrahydro-2H-pyran-2-yl)-9H-purin-6-yl)amino)propyl)-3,4-dihydroquinazoline-5-carboxamido)pyrimidine-5-carboxylate 5.2q (30.0 mg, 0.044 mmol) in 69% yield. LC-MS (method 1): t_{R}=3.14 min, m/z (M+H)⁺=675.3.

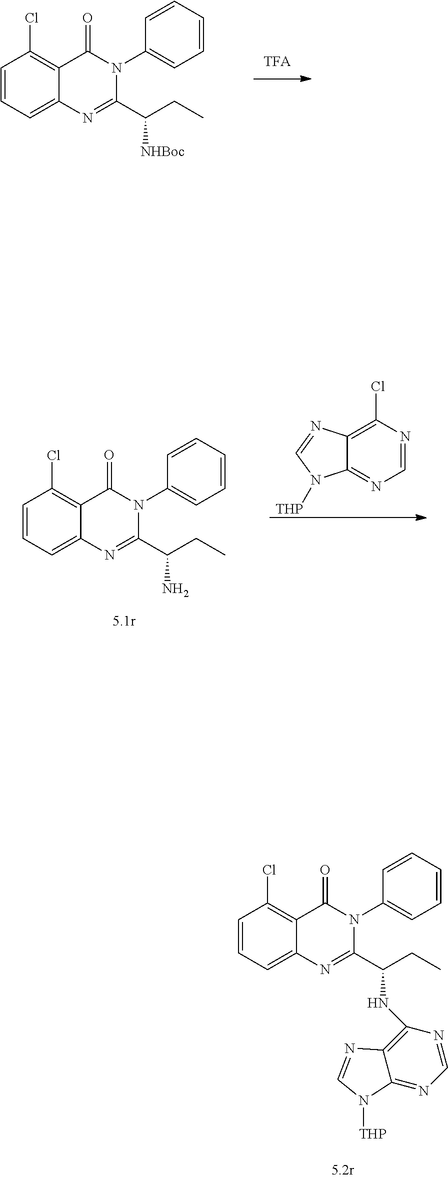

The procedure mentioned in Scheme 5 was used with tert-butyl (S)-(1-(5-chloro-4-oxo-3-phenyl-3,4-dihydroquinazolin-2-yl)propyl)carbamate (212.0 mg, 0.51 mmol) and trifluoroacetic acid (1.17 g, 10.24 mmol, 0.78 ml) in dichloromethane (5.0 ml). The resulting mixture was stirred at room temperature for 3 hours and worked-up (Method A) to afford (S)-2-(1-aminopropyl)-5-chloro-3-phenylquinazolin-4(3H)-one 5.1r. This free amine 5.1r was used with 6-chloro-9-(tetrahydro-2H-pyran-2-yl)-9H-purine (244.0 mg, 1.02 mmol) and triethylamine (207.0 mg, 2.05 mmol, 0.29 ml) in ethanol (1.2 ml). The remaining residue was purified by flash chromatography on silica gel using 0-5% MeOH/DCM to afford the product 5-chloro-3-phenyl-2-((1S)-1-((9-(tetrahydro-2H-pyran-2-yl)-9H-purin-6-yl)amino)propyl)quinazolin-4(3H)-one 5.2r (250.0 mg, 0.48 mmol) in 95% yield. LC-MS (method 1): t_{R}=3.33 min, m/z (M)⁺=516.2.

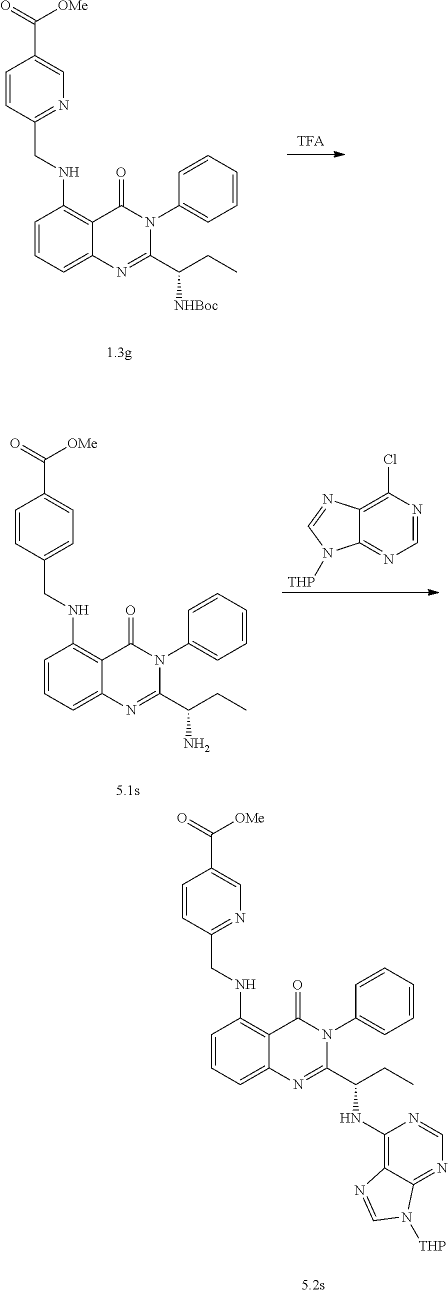

The procedure mentioned in Scheme 5 was used with methyl (S)-6-(((2-(1-((tert-butoxycarbonyl)amino)propyl)-4-oxo-3-phenyl-3,4-dihydroquinazolin-5-yl)amino)methyl)nicotinate (100.0 mg, 0.18 mmol) and trifluoroacetic acid (419.0 mg, 3.68 mmol, 0.28 ml) in dichloromethane (1.8 ml). The resulting mixture was stirred at room temperature for 3 hours and worked-up (Method A) to afford methyl (S)-6-(((2-(1-aminopropyl)-4-oxo-3-phenyl-3,4-dihydroquinazolin-5-yl)amino)methyl)nicotinate 5.1s. This free amine 5.1s was used with 6-chloro-9-(tetrahydro-2H-pyran-2-yl)-9H-purine (66.0 mg, 0.28 mmol) and triethylamine (56.0 mg, 0.55 mmol, 0.08 ml) in ethanol (0.6 ml). The remaining residue was purified by flash chromatography on silica gel using 0-100% EtOAc/hexanes to afford the product methyl 6-(((4-oxo-3-phenyl-2-((1S)-1-((9-(tetrahydro-2H-pyran-2-yl)-9H-purin-6-yl)amino)propyl)-3,4-dihydroquinazolin-5-yl)amino)methyl)nicotinate 5.2s (79.1 mg, 0.12 mmol) in 67% yield. LC-MS (method 1): t_{R}=3.34 min, m/z (M+H)⁺=646.3.

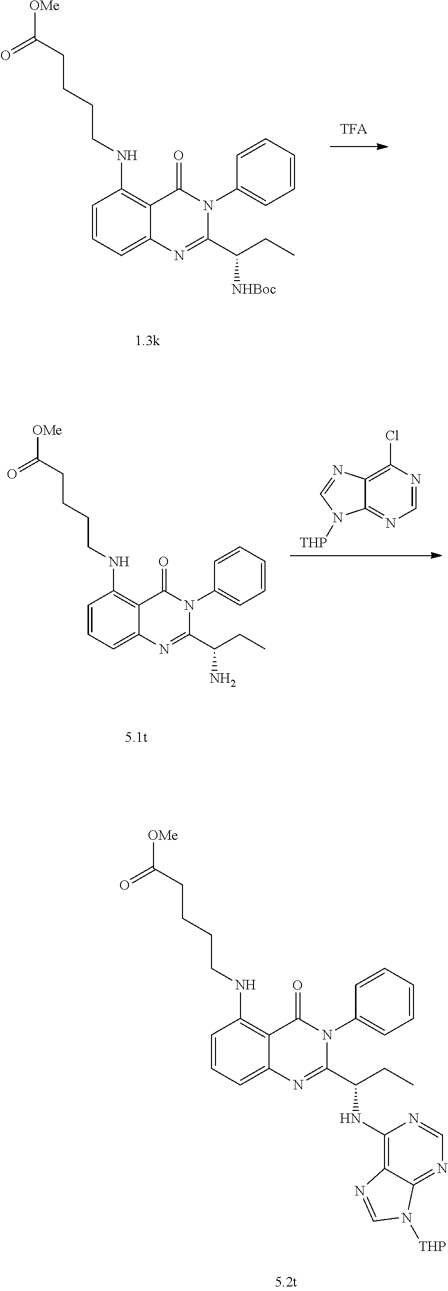

The procedure mentioned in Scheme 5 was used with methyl (S)-5-((2-(1-((tert-butoxycarbonyl)amino)propyl)-4-oxo-3-phenyl-3,4-dihydroquinazolin-5-yl)amino)pentanoate 1.3k (70.0 mg, 0.14 mmol) and trifluoroacetic acid (314.0 mg, 2.75 mmol, 211 μl) in dichloromethane (1.4 ml). The resulting mixture was stirred at room temperature for 3 hours and worked-up (Method B) to form methyl (S)-5-((2-(1-aminopropyl)-4-oxo-3-phenyl-3,4-dihydroquinazolin-5-yl)amino)pentanoate 5.1t. This free amine 5.1t was used with 6-chloro-9-(tetrahydro-2H-pyran-2-yl)-9H-purine (49.0 mg, 0.21 mmol) and triethylamine (42.0 mg, 0.41 mmol, 58.0 μl) in ethanol (0.7 ml). The remaining residue was purified by flash chromatography on silica gel using 0-5% MeOH/DCM to afford the product methyl 5-((4-oxo-3-phenyl-2-((1S)-1-((9-(tetrahydro-2H-pyran-2-yl)-9H-purin-6-yl)amino)propyl)-3,4-dihydroquinazolin-5-yl)amino)pentanoate 5.2t (68.5 mg, 0.112 mmol) in 81% yield. LC-MS (method 1): t_{R}=3.32 min, m/z (M+H)⁺=611.3.

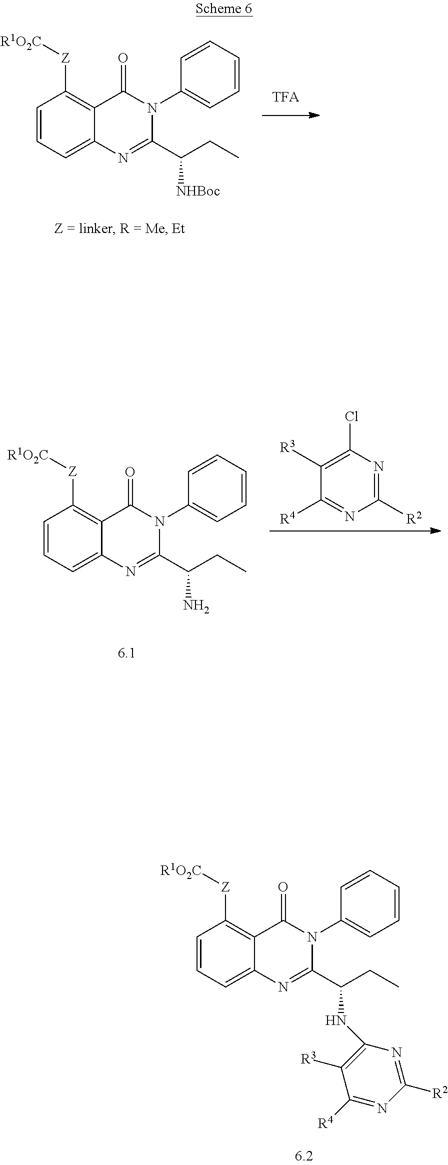

### Scheme 6

The Boc-protected amine (1 equiv) was dissolved in DCM (0.1 M) in a vial and trifluoroacetic acid (20 equiv) was added dropwise to it. The resulting mixture was stirred at room temperature for 3 hours. After completion of reaction (by LC-MS) the reaction mixture is worked-up by either of the following two methods:

Method A: The crude reaction is quenched with aqueous saturated NaHCO₃ solution and extracted three times with DCM. The combined organic layers were dried over anhydrous MgSO₄, filtered and concentrated in vacuo to afford the free amine 6.1.

Method B: The crude reaction mixture is concentrated in vacuo, re-dissolved in 1-2 ml of DCM, passed through pre-conditioned PL-HCO₃ MP SPE device and washed with 2 ml of DCM. The filtrate was concentrated in vacuo to afford the free amine 6.1.

The free amine 6.1 was dissolved in n-butanol or isopropanol (0.4 M) in a microwave vial equipped with a stir bar followed by the addition of substituted chloro-pyrimidine (1.5-2.0 equiv) and diisopropylethylamine (3.0-4.0 equiv) to it. The vial was sealed and heated for 1-24 hours at 130° C. in a microwave. After completion of reaction (by LC-MS), the reaction mixture was concentrated in vacuo and the remaining residue was purified by flash chromatography on silica gel using forced flow of indicated solvent system on Biotage KP-Sil pre-packed cartridges and using the Biotage SP-1 automated chromatography system to afford the coupled product 6.2.

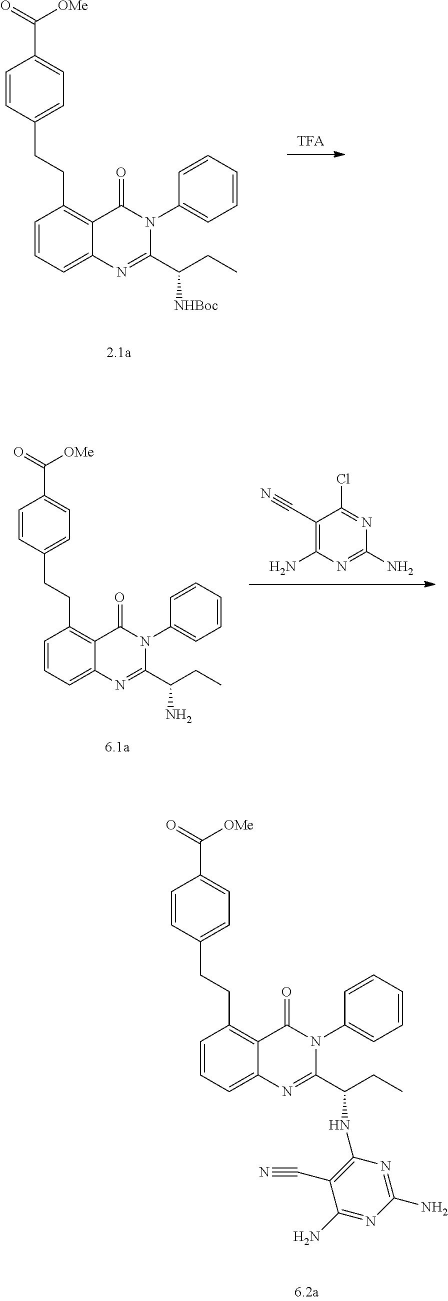 [SMILES: CC[C@H](NC(=O)OC(C)(C)C)c1nc2cccc(CCc3ccc(C(=O)OC)cc3)c2c(=O)n1-c1ccccc1 ; CC[C@H](N)c1nc2cccc(CCc3ccc(C(=O)OC)cc3)c2c(=O)n1-c1ccccc1 ; N#Cc1c(N)nc(N)nc1Cl ; CC[C@H](Nc1nc(N)nc(N)c1C#N)c1nc2cccc(CCc3ccc(C(=O)OC)cc3)c2c(=O)n1-c1ccccc1]

The procedure mentioned in Scheme 6 was used with compound 2.1a (79.0 mg, 0.15 mmol)) and trifluoroacetic acid (333.0 mg, 2.92 mmol, 0.22 ml) in DCM (1.5 ml). The resulting mixture was stirred at room temperature for 3 hours and worked-up (Method B) to afford methyl (S)-4-(2-(2-(1-aminopropyl)-4-oxo-3-phenyl-3,4-dihydroquinazolin-5-yl)ethyl)benzoate 6.1a. This free amine 6.1a was used with 2,4-diamino-6-chloropyrimidine-5-carbonitrile (50.0 mg, 0.29 mmol, Patel, L. et al. J. Med. Chem. 2016, 59, 3532) and DIPEA (75.0 mg, 0.58 mmol, 0.1 ml) in n-butanol (0.4 ml) and heated at 130° C. for 20 hours. The remaining residue was purified by flash chromatography on silica gel using 0-100% EtOAc/Hexanes to afford the product methyl (S)-4-(2-(2-(1-((2,6-diamino-5-cyanopyrimidin-4-yl)amino)propyl)-4-oxo-3-phenyl-3,4-dihydroquinazolin-5-yl)ethyl)benzoate 6.2a (64.0 mg, 0.11 mmol) in 76% yield. LC-MS (method 1): t_{R}=3.23 min, m/z (M+H)⁺=575.3.

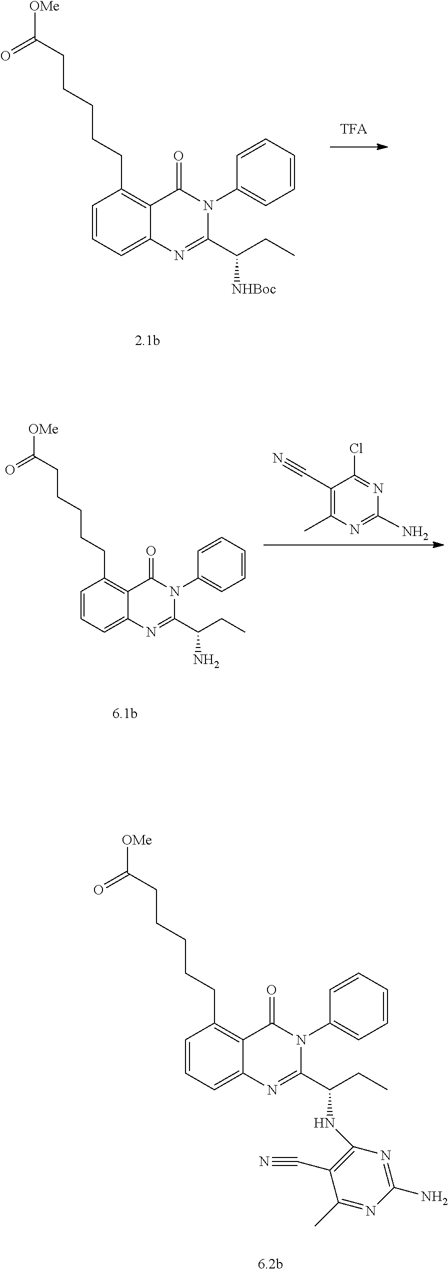 [SMILES: CC[C@H](NC(=O)OC(C)(C)C)c1nc2cccc(CCCCCC(=O)OC)c2c(=O)n1-c1ccccc1 ; CC[C@H](N)c1nc2cccc(CCCCCC(=O)OC)c2c(=O)n1-c1ccccc1 ; Cc1nc(N)nc(Cl)c1C#N ; CC[C@H](Nc1nc(N)nc(C)c1C#N)c1nc2cccc(CCCCCC(=O)OC)c2c(=O)n1-c1ccccc1]

The procedure mentioned in Scheme 6 was used with compound 2.1b (143.0 mg, 0.28 mmol)) and trifluoroacetic acid (643.0 mg, 5.64 mmol, 0.43 ml) in DCM (2.8 ml). The resulting mixture was stirred at room temperature for 3 hours and worked-up (Method A) to afford methyl (S)-6-(2-(1-aminopropyl)-4-oxo-3-phenyl-3,4-dihydroquinazolin-5-yl)hexanoate 6.1b. This free amine 6.1b was used with 2-amino-4-chloro-6-methylpyrimidine-5-carbonitrile (71.0 mg, 0.42 mmol) and DIPEA (109.0 mg, 0.85 mmol, 0.15 ml) in n-butanol (0.7 ml) and heated at 130° C. for 1 hour. The remaining residue was purified by flash chromatography on silica gel using 0-70% EtOAc/Hexanes to afford the product methyl (S)-6-(2-(1-((2-amino-5-cyano-6-methylpyrimidin-4-yl)amino)propyl)-4-oxo-3-phenyl-3,4-dihydroquinazolin-5-yl)hexanoate 6.2b (76.0 mg, 0.14 mmol) in 50% yield. LC-MS (method 1): t_{R}=3.19 min, m/z (M+H)⁺=540.3.

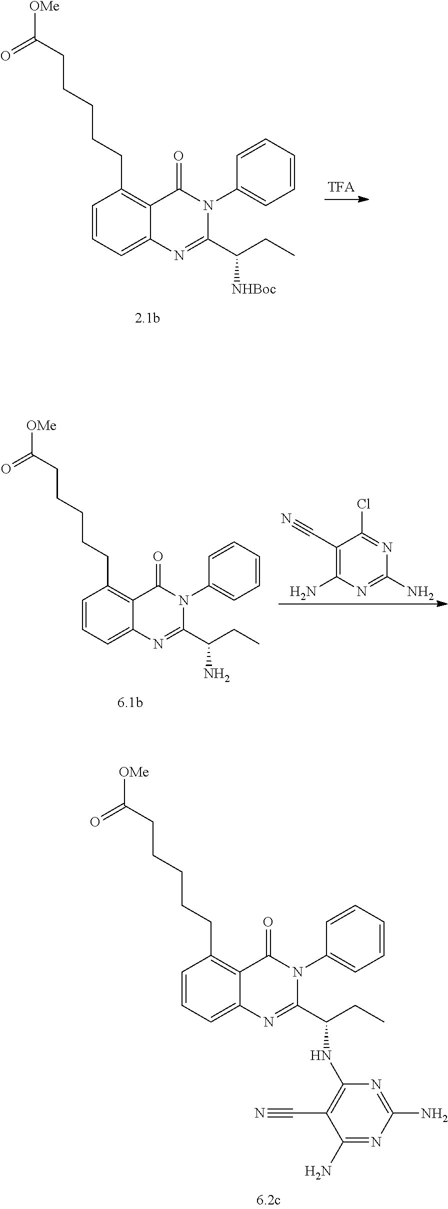

The procedure mentioned in Scheme 6 was used with compound 2.1b (98.0 mg, 0.19 mmol)) and trifluoroacetic acid (440.0 mg, 3.86 mmol, 0.30 ml) in DCM (2.0 ml). The resulting mixture was stirred at room temperature for 3 hours and worked-up (Method B) to afford methyl (S)-6-(2-(1-aminopropyl)-4-oxo-3-phenyl-3,4-dihydroquinazolin-5-yl)hexanoate 6.1b. This free amine 6.1b was used with 2,4-diamino-6-chloropyrimidine-5-carbonitrile (49.0 mg, 0.29 mmol, Patel, L. J. Med. Chem. 2016, 59, 3532) and DIPEA (75.0 mg, 0.58 mmol, 0.10 ml) in n-butanol (0.5 ml) and heated at 130° C. for 16 hours. The remaining residue was purified by flash chromatography on silica gel using 0-70% EtOAc/Hexanes to afford the product methyl (S)-6-(2-(1-((2,6-diamino-5-cyanopyrimidin-4-yl)amino)propyl)-4-oxo-3-phenyl-3,4-dihydroquinazolin-5-yl)hexanoate 6.2c (66.0 mg, 0.12 mmol) in 63% yield. LC-MS (method 1): t_{R}=3.13 min, m/z (M+H)⁺=541.3.

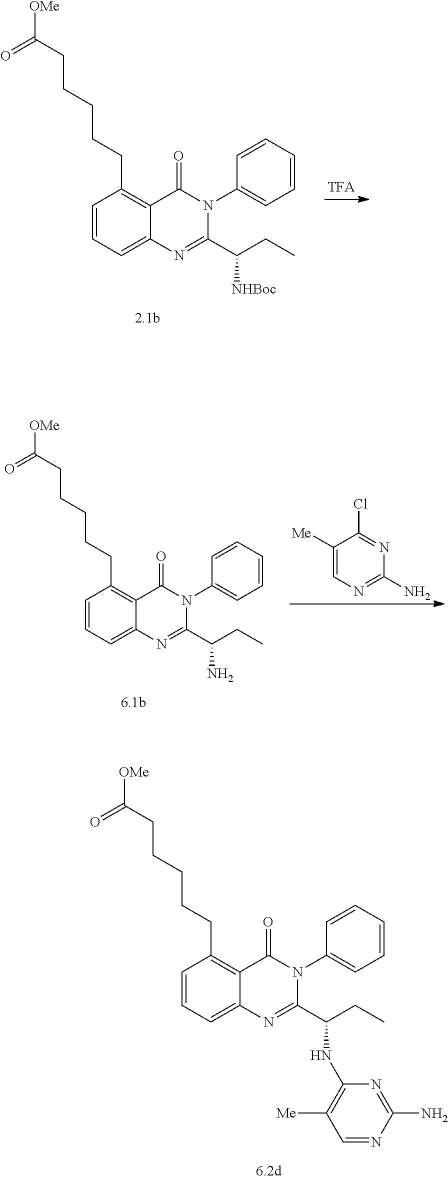 [SMILES: CC[C@H](NC(=O)OC(C)(C)C)c1nc2cccc(CCCCCC(=O)OC)c2c(=O)n1-c1ccccc1 ; CC[C@H](N)c1nc2cccc(CCCCCC(=O)OC)c2c(=O)n1-c1ccccc1 ; Cc1cnc(N)nc1Cl ; CC[C@H](Nc1nc(N)ncc1C)c1nc2cccc(CCCCCC(=O)OC)c2c(=O)n1-c1ccccc1]

The procedure mentioned in Scheme 6 was used with compound 2.1b (98.0 mg, 0.19 mmol)) and trifluoroacetic acid (440.0 mg, 3.86 mmol, 0.30 ml) in DCM (2.0 ml). The resulting mixture was stirred at room temperature for 3 hours and worked-up (Method B) to afford methyl (S)-6-(2-(1-aminopropyl)-4-oxo-3-phenyl-3,4-dihydroquinazolin-5-yl)hexanoate 6.1b. This free amine 6.1b was used with 4-chloro-5-methylpyrimidin-2-amine (42.0 mg, 0.29 mmol) and DIPEA (75.0 mg, 0.58 mmol, 0.10 ml) in n-butanol (0.5 ml) and heated at 130° C. for 24 hours. The remaining residue was purified by flash chromatography on silica gel using 0-10% MeOH/DCM to afford the product methyl (S)-6-(2-(1-((2-amino-5-methylpyrimidin-4-yl)amino)propyl)-4-oxo-3-phenyl-3,4-dihydroquinazolin-5-yl)hexanoate 6.2d (38.0 mg, 0.07 mmol) in 38% yield. LC-MS (method 1): t_{R}=3.2 min, m/z (M+H)⁺=515.3.

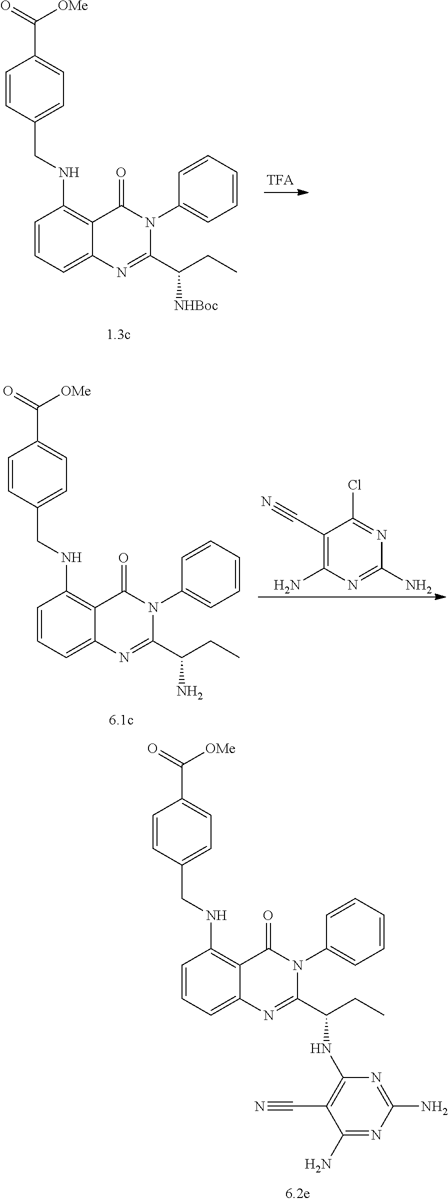 [SMILES: CC[C@H](NC(=O)OC(C)(C)C)c1nc2cccc(NCc3ccc(C(=O)OC)cc3)c2c(=O)n1-c1ccccc1 ; CC[C@H](N)c1nc2cccc(NCc3ccc(C(=O)OC)cc3)c2c(=O)n1-c1ccccc1 ; N#Cc1c(N)nc(N)nc1Cl ; CC[C@H](Nc1nc(N)nc(N)c1C#N)c1nc2cccc(NCc3ccc(C(=O)OC)cc3)c2c(=O)n1-c1ccccc1]

The procedure mentioned in Scheme 6 was used with compound 1.3c (80.0 mg, 0.15 mmol) and trifluoroacetic acid (336.0 mg, 2.95 mmol, 0.23 ml) in DCM (1.7 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 2,4-diamino-6-chloropyrimidine-5-carbonitrile (50.0 mg, 0.29 mmol) and DIPEA (76.0 mg, 0.59 mmol, 102.0 μl) in isopropanol (0.7 ml) and heated at 130° C. for 1 hour. The remaining residue was purified by flash chromatography on silica gel using 0-100% EtOAc/Hexanes to afford the product methyl (S)-4-(((2-(1-((2,6-diamino-5-cyanopyrimidin-4-yl)amino)propyl)-4-oxo-3-phenyl-3,4-dihydroquinazolin-5-yl)amino)methyl)benzoate 6.2e (70.0 mg, 0.10 mmol) in 69% yield. LC-MS (method 1): t_{R}=3.18 min, m/z (M+H)⁺=576.3.

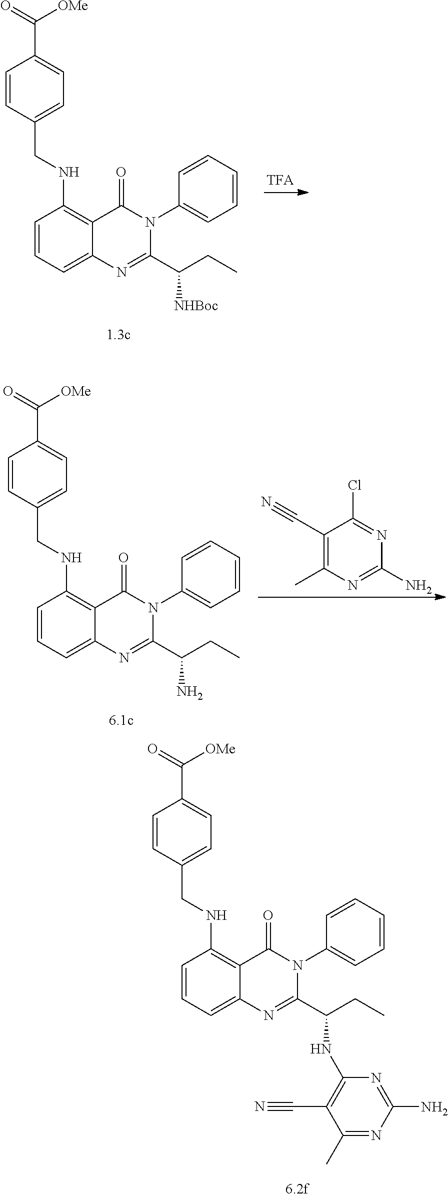 [SMILES: CC[C@H](NC(=O)OC(C)(C)C)c1nc2cccc(NCc3ccc(C(=O)OC)cc3)c2c(=O)n1-c1ccccc1 ; CC[C@H](N)c1nc2cccc(NCc3ccc(C(=O)OC)cc3)c2c(=O)n1-c1ccccc1 ; Cc1nc(N)nc(Cl)c1C#N ; CC[C@H](Nc1nc(N)nc(C)c1C#N)c1nc2cccc(NCc3ccc(C(=O)OC)cc3)c2c(=O)n1-c1ccccc1]

The procedure mentioned in Scheme 6 was used with compound 1.3c (68.0 mg, 0.125 mmol) and trifluoroacetic acid (286.0 mg, 2.51 mmol, 0.19 ml) in DCM (1.2 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 2-amino-4-chloro-6-methylpyrimidine-5-carbonitrile (32.0 mg, 0.19 mmol) and DIPEA (48.0 mg, 0.38 mmol, 65.0 μl) in n-butanol (0.3 ml) and heated at 130° C. for 1 hour. The remaining residue was purified by flash chromatography on silica gel using 0-70% EtOAc/Hexanes to afford the product methyl (S)-4-(((2-(1-((2-amino-5-cyano-6-methylpyrimidin-4-yl)amino)propyl)-4-oxo-3-phenyl-3,4-dihydroquinazolin-5-yl)amino)methyl)benzoate 6.2f (60.0 mg, 0.10 mmol) in 84% yield. LC-MS (method 1): t_{R}=3.24 min, m/z (M+H)⁺=575.3.

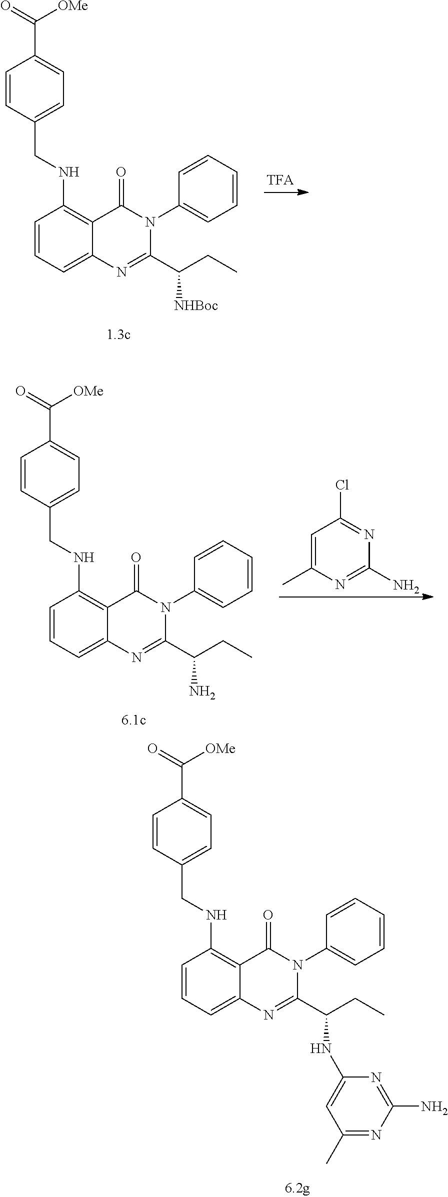

The procedure mentioned in Scheme 6 was used with compound 1.3c (52.0 mg, 0.096 mmol) and trifluoroacetic acid (219.0 mg, 1.92 mmol, 0.15 ml) in DCM (1.0 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 4-chloro-6-methylpyrimidin-2-amine (21.0 mg, 0.144 mmol) and DIPEA (37.0 mg, 0.29 mmol, 50.0 μl) in n-butanol (0.3 ml) and heated at 130° C. for 5 hours. The remaining residue was purified by flash chromatography on silica gel using 0-10% MeOH/DCM to afford the product methyl (S)-4-(((2-(1-((2-amino-6-methylpyrimidin-4-yl)amino)propyl)-4-oxo-3-phenyl-3,4-dihydroquinazolin-5-yl)amino)methyl)benzoate 6.2g (29.0 mg, 0.053 mmol) in 55% yield. LC-MS (method 1): t_{R}=2.97 min, m/z (M+H)⁺=550.3.

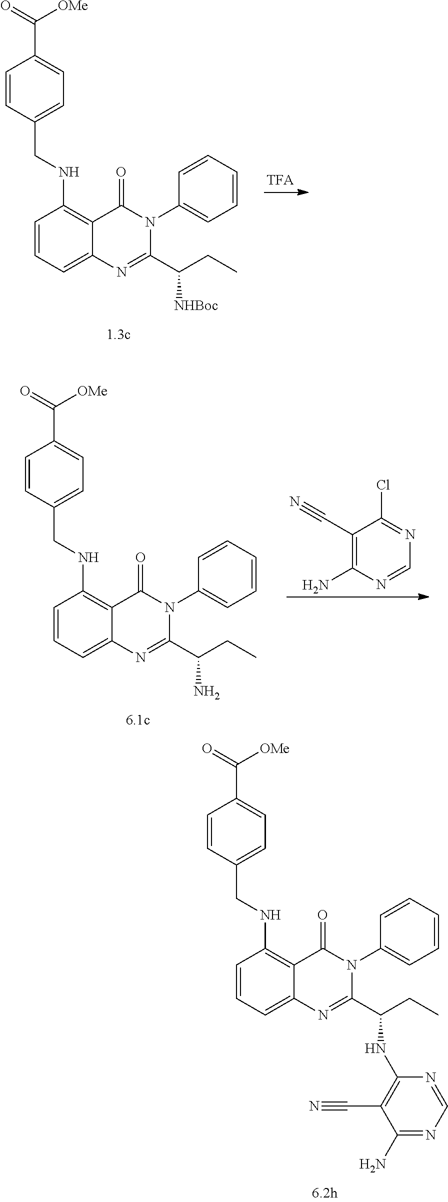 [SMILES: CC[C@H](NC(=O)OC(C)(C)C)c1nc2cccc(NCc3ccc(C(=O)OC)cc3)c2c(=O)n1-c1ccccc1 ; CC[C@H](N)c1nc2cccc(NCc3ccc(C(=O)OC)cc3)c2c(=O)n1-c1ccccc1 ; N#Cc1c(N)ncnc1Cl ; CC[C@H](Nc1ncnc(N)c1C#N)c1nc2cccc(NCc3ccc(C(=O)OC)cc3)c2c(=O)n1-c1ccccc1]

The procedure mentioned in Scheme 6 was used with compound 1.3c (48.3 mg, 0.09 mmol) and trifluoroacetic acid (203.0 mg, 1.78 mmol, 0.14 ml) in DCM (1.0 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 4-amino-6-chloropyrimidine-5-carbonitrile (27.5 mg, 0.18 mmol) and DIPEA (46.0 mg, 0.36 mmol, 62.0 μl) in n-butanol (0.3 ml) and heated at 130° C. for 1 hour. The remaining residue was purified by flash chromatography on silica gel using 0-100% EtOAc/hexanes to afford the product methyl (S)-4-(((2-(1-((6-amino-5-cyanopyrimidin-4-yl)amino)propyl)-4-oxo-3-phenyl-3,4-dihydroquinazolin-5-yl)amino)methyl)benzoate 6.2h (25.0 mg, 0.045 mmol) in 50% yield. LC-MS (method 1): t_{R}=3.39 min, m/z (M+H)⁺=561.3.

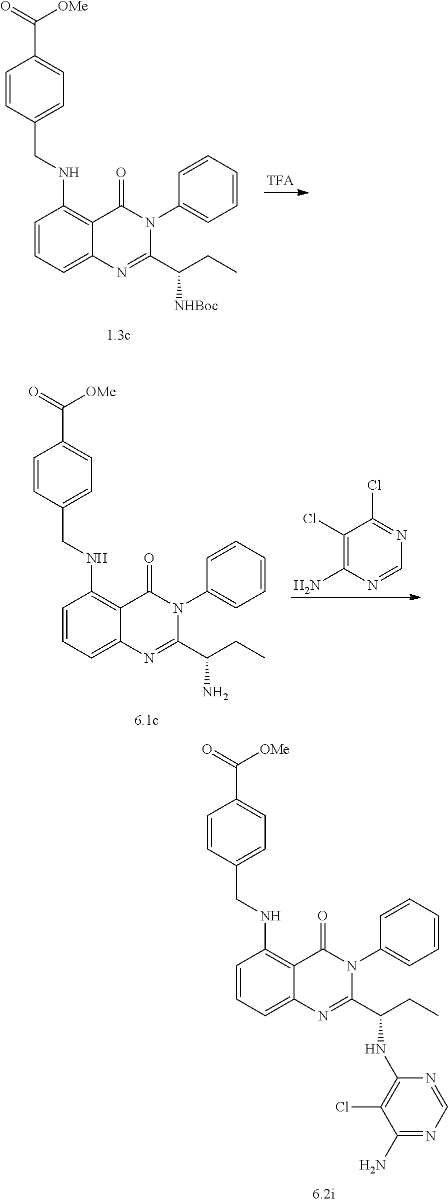 [SMILES: CC[C@H](NC(=O)OC(C)(C)C)c1nc2cccc(NCc3ccc(C(=O)OC)cc3)c2c(=O)n1-c1ccccc1 ; CC[C@H](N)c1nc2cccc(NCc3ccc(C(=O)OC)cc3)c2c(=O)n1-c1ccccc1 ; Nc1ncnc(Cl)c1Cl ; CC[C@H](Nc1ncnc(N)c1Cl)c1nc2cccc(NCc3ccc(C(=O)OC)cc3)c2c(=O)n1-c1ccccc1]

The procedure mentioned in Scheme 6 was used with compound 1.3c (61.8 mg, 0.114 mmol) and trifluoroacetic acid (260.0 mg, 2.28 mmol, 0.17 ml) in DCM (1.2 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 5,6-dichloropyrimidin-4-amine (37.0 mg, 0.23 mmol) and DIPEA (59.0 mg, 0.46 mmol, 79.0 μl) in n-butanol (0.3 ml) and heated at 130° C. for 20 hours. The remaining residue was purified by flash chromatography on silica gel using 0-100% EtOAc/hexanes to afford the product methyl (S)-4-(((2-(1-((6-amino-5-chloropyrimidin-4-yl)amino)propyl)-4-oxo-3-phenyl-3,4-dihydroquinazolin-5-yl)amino)methyl)benzoate 6.2i (55.0 mg, 0.096 mmol) in 85% yield. LC-MS (method 1): t_{R}=3.25 min, m/z (M)⁺=570.2.

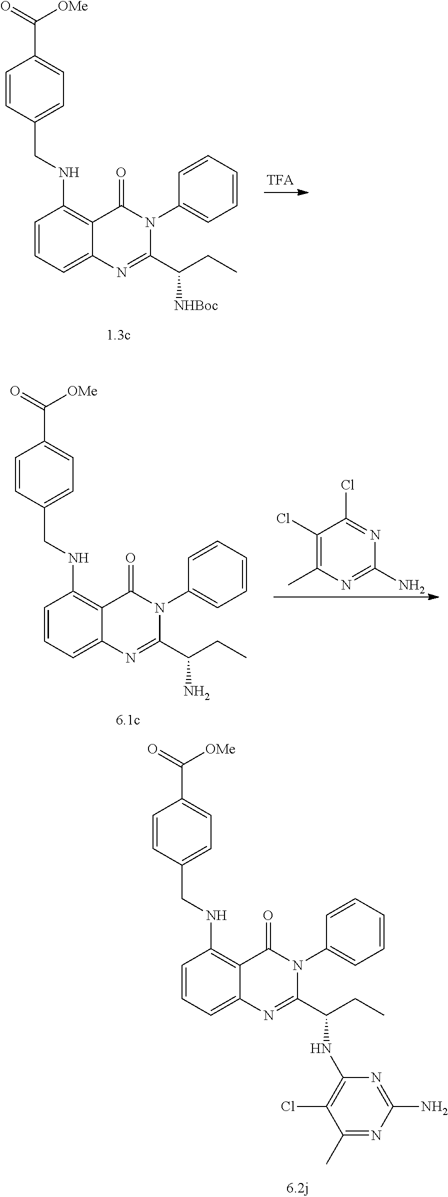

The procedure mentioned in Scheme 6 was used with compound 1.3c (72.0 mg, 0.133 mmol) and trifluoroacetic acid (303.0 mg, 2.65 mmol, 0.20 ml) in DCM (1.3 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 4,5-dichloro-6-methylpyrimidin-2-amine (36.0 mg, 0.20 mmol) and DIPEA (52.0 mg, 0.40 mmol, 70.0 μl) in n-butanol (0.3 ml) and heated at 130° C. for 5 hours in a MW reactor. The remaining residue was purified by flash chromatography on silica gel using 0-100% EtOAc/hexanes to afford the product methyl (S)-4-(((2-(1-((2-amino-5-chloro-6-methylpyrimidin-4-yl)amino)propyl)-4-oxo-3-phenyl-3,4-dihydroquinazolin-5-yl)amino)methyl)benzoate 6.2j (68.0 mg, 0.116 mmol) in 88% yield. LC-MS (method 1): t_{R}=3.15 min, m/z (M)⁺=584.2.

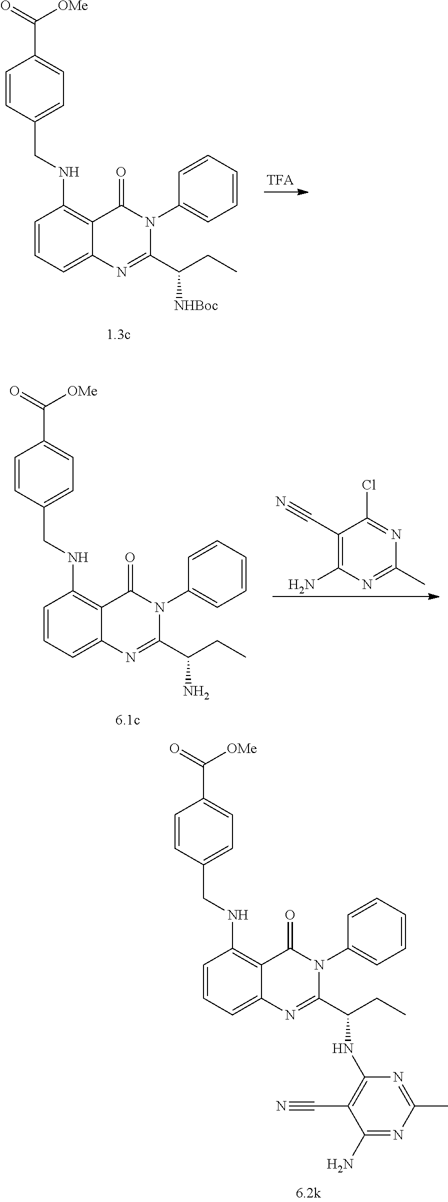 [SMILES: CC[C@H](NC(=O)OC(C)(C)C)c1nc2cccc(NCc3ccc(C(=O)OC)cc3)c2c(=O)n1-c1ccccc1 ; CC[C@H](N)c1nc2cccc(NCc3ccc(C(=O)OC)cc3)c2c(=O)n1-c1ccccc1 ; Cc1nc(N)c(C#N)c(Cl)n1 ; CC[C@H](Nc1nc(C)nc(N)c1C#N)c1nc2cccc(NCc3ccc(C(=O)OC)cc3)c2c(=O)n1-c1ccccc1]

The procedure mentioned in Scheme 6 was used with compound 1.3c (86.0 mg, 0.158 mmol) and trifluoroacetic acid (361.0 mg, 3.17 mmol, 0.24 ml) in DCM (1.6 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)amino)methyl)benzoate 6.1c. The free amine 6.1c was used with 4-amino-6-chloro-2-methylpyrimidine-5-carbonitrile (40.0 mg, 0.24 mmol) and DIPEA (61.0 mg, 0.47 mmol, 84.0 μl) in n-butanol (0.3 ml) and heated at 130° C. for 2 hours in a MW reactor. The remaining residue was purified by flash chromatography on silica gel using 0-100% EtOAc/hexanes to afford the product methyl (S)-4-(((2-(1-((6-amino-5-cyano-2-methylpyrimidin-4-yl)amino)propyl)-4-oxo-3-phenyl-3,4-dihydroquinazolin-5-yl)amino)methyl)benzoate 6.2k (75.0 mg, 0.131 mmol) in 83% yield. LC-MS (method 1): t_{R}=3.33 min, m/z (M+H)⁺=575.3.

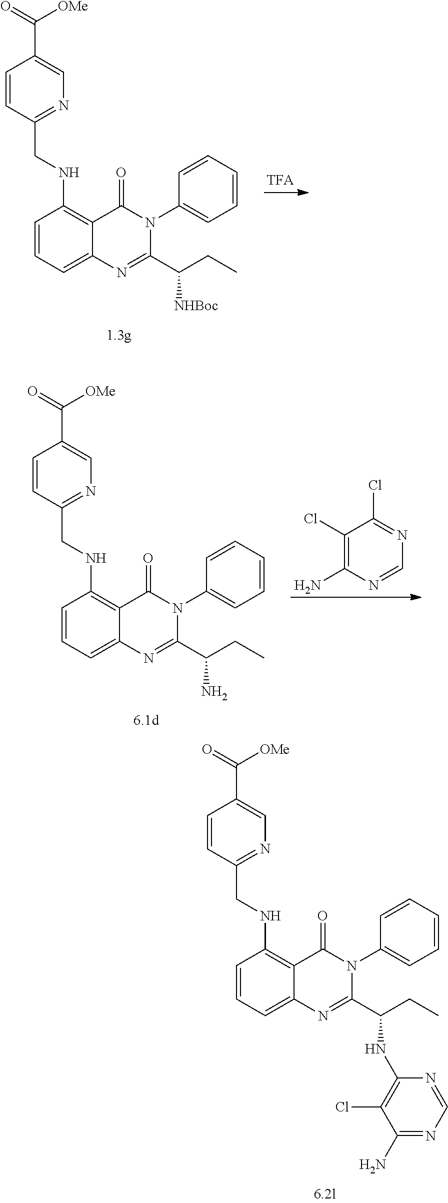 [SMILES: CC[C@H](NC(=O)OC(C)(C)C)c1nc2cccc(NCc3ccc(C(=O)OC)cn3)c2c(=O)n1-c1ccccc1 ; CC[C@H](N)c1nc2cccc(NCc3ccc(C(=O)OC)cn3)c2c(=O)n1-c1ccccc1 ; Nc1ncnc(Cl)c1Cl ; CC[C@H](Nc1ncnc(N)c1Cl)c1nc2cccc(NCc3ccc(C(=O)OC)cn3)c2c(=O)n1-c1ccccc1]

The procedure mentioned in Scheme 6 was used with compound 1.3g (66.0 mg, 0.12 mmol) and trifluoroacetic acid (277.0 mg, 2.45 mmol, 0.19 ml) in DCM (1.2 ml). The resulting mixture was stirred at room temperature for 3 hours and worked-up (Method B) to afford methyl (S)-6-(((2-(1-aminopropyl)-4-oxo-3-phenyl-3,4-dihydroquinazolin-5-yl)amino)methyl)nicotinate 6.1d. The free amine 6.1d was used with 5,6-dichloropyrimidin-4-amine (40.0 mg, 0.24 mmol) and DIPEA (63.0 mg, 0.48 mmol, 83.0 μl) in n-butanol (0.3 ml) and heated at 130° C. for 2 hours. The remaining residue was purified by flash chromatography on silica gel using 0-100% EtOAc/hexanes to afford the product methyl (S)-6-(((2-(1-((6-amino-5-chloropyrimidin-4-yl)amino)propyl)-4-oxo-3-phenyl-3,4-dihydroquinazolin-5-yl)amino)methyl)nicotinate 6.21 (30.0 mg, 0.053 mmol) in 43% yield. LC-MS (method 1): t_{R}=3.06 min, m/z (M)⁺=571.2.

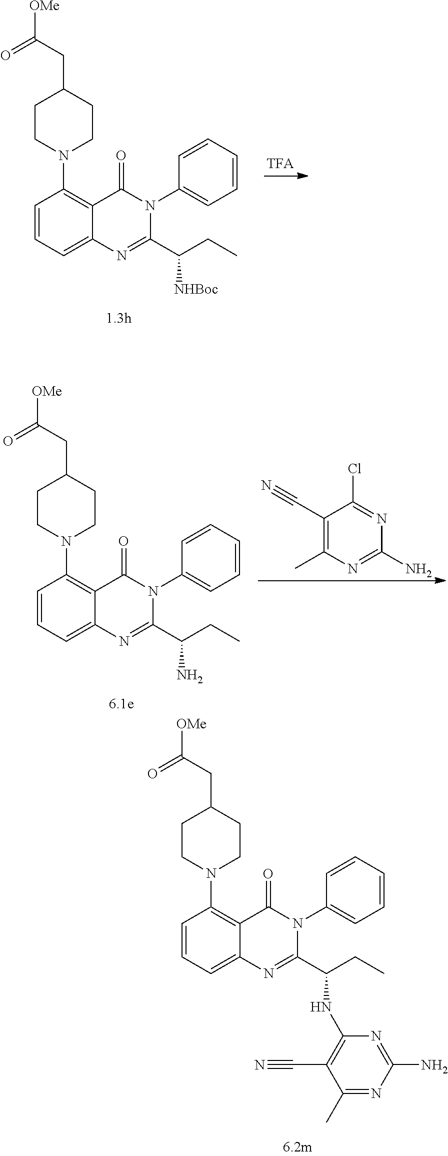 [SMILES: CC[C@H](NC(=O)OC(C)(C)C)c1nc2cccc(N3CCC(CC(=O)OC)CC3)c2c(=O)n1-c1ccccc1 ; CC[C@H](N)c1nc2cccc(N3CCC(CC(=O)OC)CC3)c2c(=O)n1-c1ccccc1 ; Cc1nc(N)nc(Cl)c1C#N ; CC[C@H](Nc1nc(N)nc(C)c1C#N)c1nc2cccc(N3CCC(CC(=O)OC)CC3)c2c(=O)n1-c1ccccc1]

The procedure mentioned in Scheme 6 was used with compound 1.3h (45.0 mg, 0.084 mmol) and trifluoroacetic acid (192.0 mg, 1.68 mmol, 0.13 ml) in DCM (0.8 ml). The resulting mixture was stirred at room temperature for 3 hours and worked-up (Method B) to afford methyl (S)-2-(1-(2-(1-aminopropyl)-4-oxo-3-phenyl-3,4-dihydroquinazolin-5-yl)piperidin-4-yl)acetate 6.1e. The free amine 6.1e was used with 2-amino-4-chloro-6-methylpyrimidine-5-carbonitrile (21.0 mg, 0.13 mmol) and DIPEA (33.0 mg, 0.25 mmol, 44.0 μl) in n-butanol (0.3 ml) and heated at 130° C. for 2 hours. The remaining residue was purified by flash chromatography on silica gel using 0-100% EtOAc/hexanes to afford the product methyl (S)-2-(1-(2-(1-((2-amino-5-cyano-6-methylpyrimidin-4-yl)amino)propyl)-4-oxo-3-phenyl-3,4-dihydroquinazolin-5-yl)piperidin-4-yl)acetate 6.2m (34.0 mg, 0.06 mmol) in 71% yield. LC-MS (method 1): t_{R}=2.57 min, m/z (M+H)⁺=567.3.

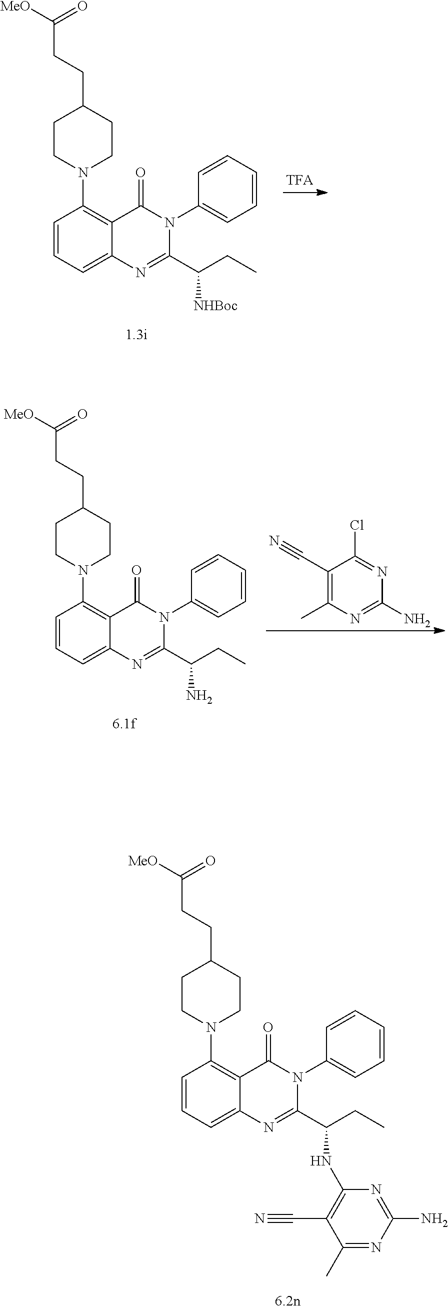

The procedure mentioned in Scheme 6 was used with compound 1.3i (32.0 mg, 0.058 mmol) and trifluoroacetic acid (133.0 mg, 1.17 mmol, 89.0 μl) in DCM (0.6 ml). The resulting mixture was stirred at room temperature for 3 hours and worked-up (Method B) to afford methyl (S)-3-(1-(2-(1-aminopropyl)-4-oxo-3-phenyl-3,4-dihydroquinazolin-5-yl)piperidin-4-yl)propanoate 6.1f. The free amine 6.1f was used with 2-amino-4-chloro-6-methylpyrimidine-5-carbonitrile (15.0 mg, 0.09 mmol) and DIPEA (22.0 mg, 0.17 mmol, 30.0 μl) in n-butanol (0.3 ml) and heated at 130° C. for 2 hours. The remaining residue was purified by flash chromatography on silica gel using 0-100% EtOAc/hexanes to afford the product methyl (S)-3-(1-(2-(1-((2-amino-5-cyano-6-methylpyrimidin-4-yl)amino)propyl)-4-oxo-3-phenyl-3,4-dihydroquinazolin-5-yl)piperidin-4-yl)propanoate 6.2n (21.0 mg, 0.04 mmol) in 63% yield. LC-MS (method 1): t_{R}=2.61 min, m/z (M+H)⁺=581.3.

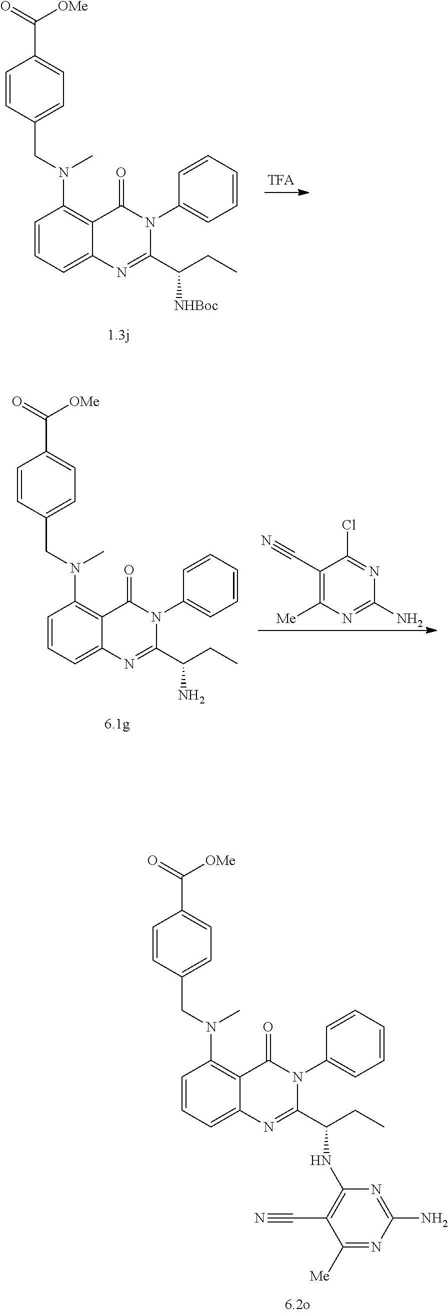

The procedure mentioned in Scheme 6 was used with compound 1.3j (58.0 mg, 0.104 mmol) and trifluoroacetic acid (238.0 mg, 2.08 mmol, 160.0 μl) in DCM (1.0 ml). The resulting mixture was stirred at room temperature for 3 hours and worked-up (Method B) to afford methyl (S)-4-(((2-(1-aminopropyl)-4-oxo-3-phenyl-3,4-dihydroquinazolin-5-yl)(methyl)amino)methyl)benzoate 6.1g. The free amine 6.1g was used with 2-amino-4-chloro-6-methylpyrimidine-5-carbonitrile (26.3 mg, 0.16 mmol) and DIPEA (40.3 mg, 0.31 mmol, 53.0 μl) in n-butanol (0.3 ml) and heated at 130° C. for 2 hours. The remaining residue was purified by flash chromatography on silica gel using 0-100% EtOAc/hexanes to afford the product methyl (S)-4-(((2-(1-((2-amino-5-cyano-6-methylpyrimidin-4-yl)amino)propyl)-4-oxo-3-phenyl-3,4-dihydroquinazolin-5-yl)(methyl)amino)methyl)benzoate 6.2o (40.0 mg, 0.07 mmol) in 65% yield. LC-MS (method 1): t_{R}=2.68 min, m/z (M+H)⁺=589.3.

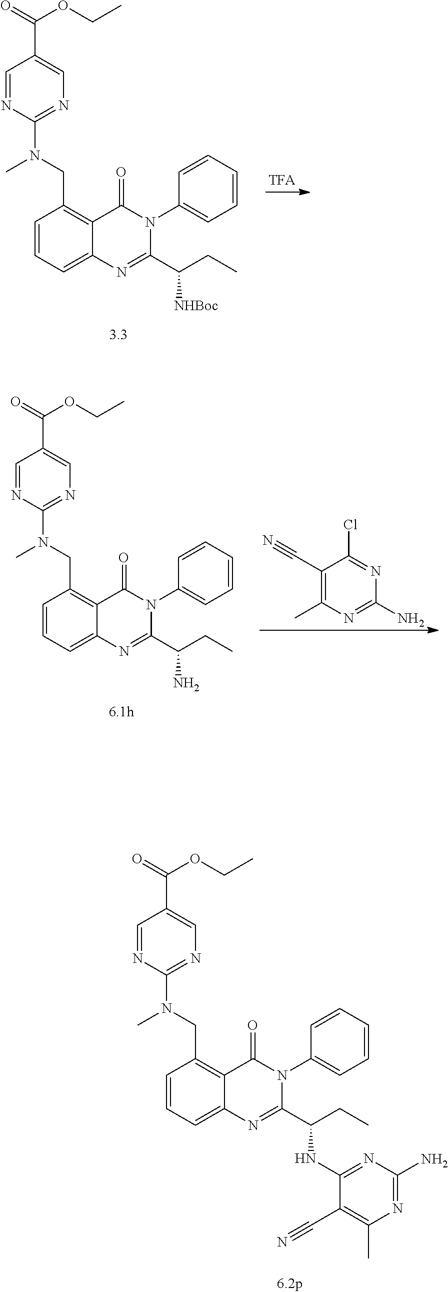

The procedure mentioned in Scheme 6 was used with compound 3.3 (22.0 mg, 0.04 mmol) and trifluoroacetic acid (88.0 mg, 0.77 mmol, 59.0 μl) in DCM (0.5 ml). The resulting mixture was stirred at room temperature for 3 hours and worked-up (Method B) to afford ethyl (S)-2-(((2-(1-aminopropyl)-4-oxo-3-phenyl-3,4-dihydroquinazolin-5-yl)methyl)(methyl)amino)pyrimidine-5-carboxylate 6.1h. The free amine 6.1h was used with 2-amino-4-chloro-6-methylpyrimidine-5-carbonitrile (9.6 mg, 0.06 mmol) and DIPEA (15.0 mg, 0.11 mmol, 20.0 μl) in n-butanol (0.3 ml) and heated at 130° C. for 2 hours. The remaining residue was purified by flash chromatography on silica gel using 0-100% EtOAc/hexanes to afford the product ethyl (S)-2-(((2-(1-((2-amino-5-cyano-6-methylpyrimidin-4-yl)amino)propyl)-4-oxo-3-phenyl-3,4-dihydroquinazolin-5-yl)methyl)(methyl)amino)pyrimidine-5-carboxylate 6.2p (15.0 mg, 0.025 mmol) in 65% yield. LC-MS (method 1): t_{R}=3.30 min, m/z (M+H)⁺=605.3.

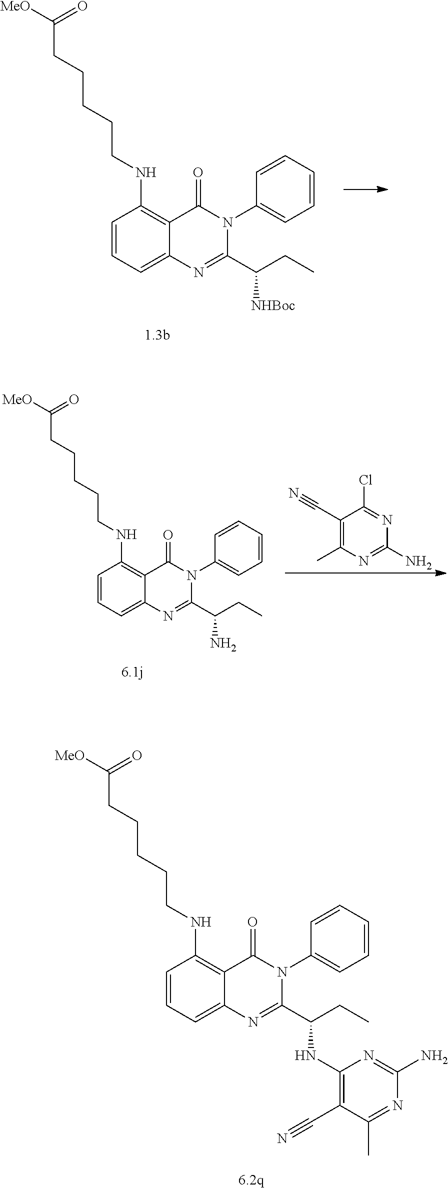 [SMILES: CC[C@H](NC(=O)OC(C)(C)C)c1nc2cccc(NCCCCCC(=O)OC)c2c(=O)n1-c1ccccc1 ; CC[C@H](N)c1nc2cccc(NCCCCCC(=O)OC)c2c(=O)n1-c1ccccc1 ; Cc1nc(N)nc(Cl)c1C#N ; CC[C@H](Nc1nc(N)nc(C)c1C#N)c1nc2cccc(NCCCCCC(=O)OC)c2c(=O)n1-c1ccccc1]

The procedure mentioned in Scheme 6 was used with (S)-methyl 6-((2-(1-((tert-butoxycarbonyl)amino)propyl)-4-oxo-3-phenyl-3,4-dihydroquinazolin-5-yl)amino)hexanoate 1.3b (110.0 mg, 0.21 mmol) and trifluoroacetic acid (480.0 mg, 4.21 mmol, 0.32 ml) in dichloromethane (2.1 ml). The resulting mixture was stirred at room temperature for 3 hours and worked-up (Method A) to form (S)-methyl 6-((2-(1-aminopropyl)-4-oxo-3-phenyl-3,4-dihydroquinazolin-5-yl)amino)hexanoate 6.1i. This free amine 6.1i was used with 2-amino-4-chloro-6-methylpyrimidine-5-carbonitrile (53.0 mg, 0.32 mmol) and diisopropylethylamine (81.0 mg, 0.63 mmol, 110.0 μl) in n-butanol (0.5 ml) and heated at 130° C. for 2 hours. The remaining residue was purified by flash chromatography on silica gel using 0-100% EtOAc/Hexanes to afford the product methyl (S)-6-((2-(1-((2-amino-5-cyano-6-methylpyrimidin-4-yl)amino)propyl)-4-oxo-3-phenyl-3,4-dihydroquinazolin-5-yl)amino)hexanoate 6.2q (97.0 mg, 0.174 mmol) in 83% yield. LC-MS (method 1): tR=3.25 min, m/z (M+H)+=555.3.

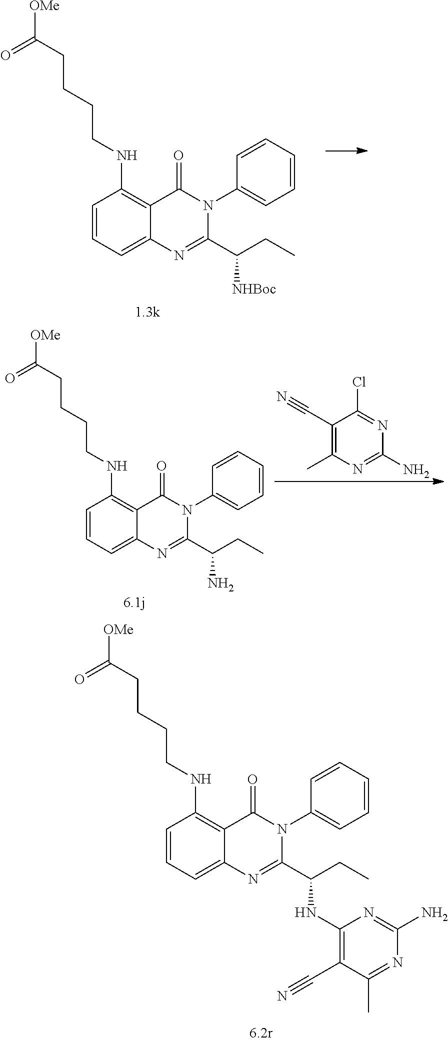

The procedure mentioned in Scheme 6 was used with compound 1.3k (98.0 mg, 0.193 mmol) and trifluoroacetic acid (439.0 mg, 3.85 mmol, 295.0 μl) in DCM (2.0 ml). The resulting mixture was stirred at room temperature for 3 hours and worked-up (Method B) to afford methyl (S)-5-((2-(1-aminopropyl)-4-oxo-3-phenyl-3,4-dihydroquinazolin-5-yl)amino)pentanoate 6.1j. The free amine 6.1j was used with 2-amino-4-chloro-6-methylpyrimidine-5-carbonitrile (49.0 mg, 0.29 mmol) and DIPEA (75.0 mg, 0.56 mmol, 101.0 μl) in n-butanol (0.5 ml) and heated at 130° C. for 2 hours. The remaining residue was purified by flash chromatography on silica gel using 0-100% EtOAc/hexanes to afford the product methyl (S)-5-((2-(1-((2-amino-5-cyano-6-methylpyrimidin-4-yl)amino)propyl)-4-oxo-3-phenyl-3,4-dihydroquinazolin-5-yl)amino)pentanoate 6.2r (89.0 mg, 0.164 mmol) in 85% yield. LC-MS (method 1): tR=3.18 min, m/z (M+H)+=541.3.

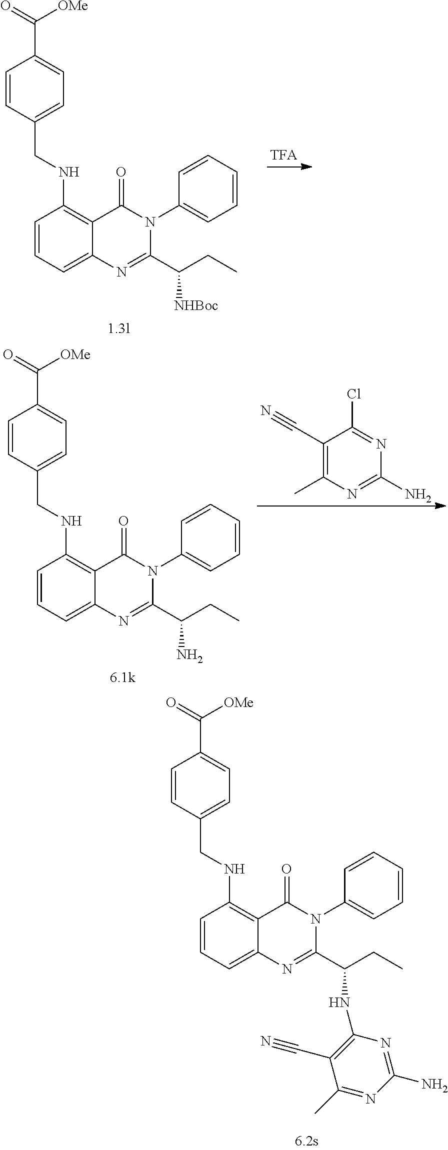 [SMILES: CC[C@H](NC(=O)OC(C)(C)C)c1nc2cccc(NCc3ccc(C(=O)OC)cc3)c2c(=O)n1-c1ccccc1 ; CC[C@H](N)c1nc2cccc(NCc3ccc(C(=O)OC)cc3)c2c(=O)n1-c1ccccc1 ; Cc1nc(N)nc(Cl)c1C#N ; CC[C@H](Nc1nc(N)nc(C)c1C#N)c1nc2cccc(NCc3ccc(C(=O)OC)cc3)c2c(=O)n1-c1ccccc1]

The procedure mentioned in Scheme 6 was used with compound 1.31 (109.0 mg, 0.254 mmol) and trifluoroacetic acid (580.0 mg, 5.09 mmol, 0.39 ml) in DCM (2.5 ml). The resulting mixture was stirred at room temperature for 3 hours and worked-up (Method B) to afford methyl (S)-4-(((2-(1-aminoethyl)-4-oxo-3-phenyl-3,4-dihydroquinazolin-5-yl)amino)methyl)benzoate 6.1k. The free amine 6.1k was used with 2-amino-4-chloro-6-methylpyrimidine-5-carbonitrile (64.2 mg, 0.38 mmol) and DIPEA (98.0 mg, 0.76 mmol, 133.0 μl) in n-butanol (0.5 ml) and heated at 130° C. for 2 hours. The remaining residue was purified by flash chromatography on silica gel using 0-100% EtOAc/Hexanes to afford the product methyl (S)-4-(((2-(1-((2-amino-5-cyano-6-methylpyrimidin-4-yl)amino)ethyl)-4-oxo-3-phenyl-3,4-dihydroquinazolin-5-yl)amino)methyl)benzoate 6.2s (90.0 mg, 0.16 mmol) in 63% yield. LC-MS (method 1): t_{R}=3.12 min, m/z (M+H)⁺=561.2.

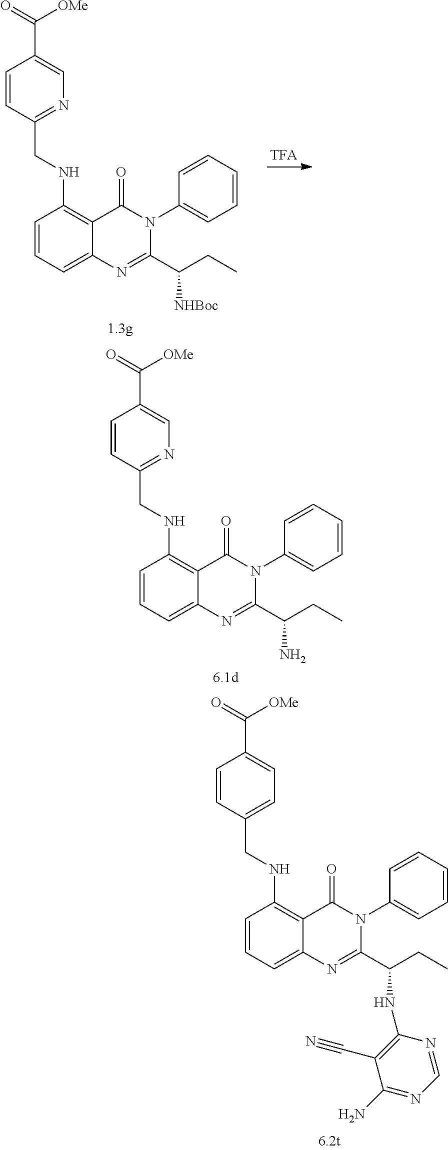 [SMILES: CC[C@H](NC(=O)OC(C)(C)C)c1nc2cccc(NCc3ccc(C(=O)OC)cn3)c2c(=O)n1-c1ccccc1 ; CC[C@H](N)c1nc2cccc(NCc3ccc(C(=O)OC)cn3)c2c(=O)n1-c1ccccc1 ; CC[C@H](Nc1ncnc(N)c1C#N)c1nc2cccc(NCc3ccc(C(=O)OC)cc3)c2c(=O)n1-c1ccccc1]

The procedure mentioned in Scheme 6 was used with compound 1.3g (66.0 mg, 0.12 mmol) and trifluoroacetic acid (277.0 mg, 2.45 mmol, 0.19 ml) in DCM (1.2 ml). The resulting mixture was stirred at room temperature for 3 hours and worked-up (Method B) to afford methyl (S)-6-(((2-(1-aminopropyl)-4-oxo-3-phenyl-3,4-dihydroquinazolin-5-yl)amino)methyl)nicotinate 6.1d. The free amine 6.1d was used with 4-amino-6-chloropyrimidine-5-carbonitrile (37.0 mg, 0.24 mmol) and DIPEA (63.0 mg, 0.48 mmol, 84.0 μl) in n-butanol (0.3 ml) and heated at 130° C. for 1 hour. The remaining residue was purified by flash chromatography on silica gel using 0-100% EtOAc/hexanes to afford the product methyl (S)-6-(((2-(1-((6-amino-5-cyanopyrimidin-4-yl)amino)propyl)-4-oxo-3-phenyl-3,4-dihydroquinazolin-5-yl)amino)methyl)nicotinate 6.2t (30.0 mg, 0.053 mmol) in 44% yield. LC-MS (method 1): t_{R}=3.15 min, m/z (M+H)⁺=562.3.

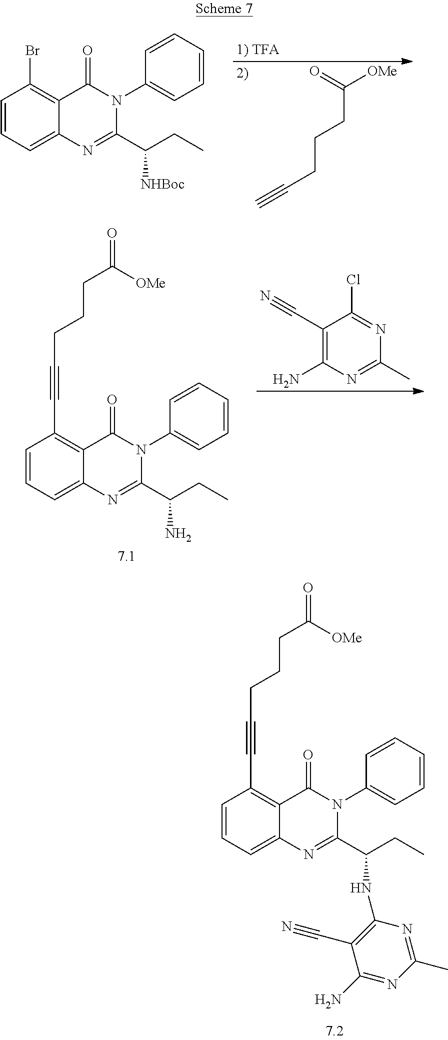

### Scheme 7

Tert-butyl (S)-(1-(5-bromo-4-oxo-3-phenyl-3,4-dihydroquinazolin-2-yl)propyl)carbamate (144.4 mg, 0.32 mmol) was dissolved in DCM (3.0 ml) in a vial and trifluoroacetic acid (718.0 mg, 6.30 mmol, 482.0 μl) was added dropwise to it. The resulting mixture was stirred at room temperature for 3 hours. After completion of reaction (by LC-MS) the reaction mixture was quenched with aqueous saturated NaHCO₃ solution and extracted three times with DCM. The combined organic layers were dried over anhydrous MgSO₄, filtered and concentrated in vacuo to afford the free amine, (S)-2-(1-aminopropyl)-5-bromo-3-phenylquinazolin-4(3H)-one. This free amine was added to a vial containing allylpalladium(II) chloride dimer (5.8 mg, 0.02 mmol) and tri-tert-butylphosphonium tetrafluoroborate (18.0 mg, 0.06 mmol). The vial was covered with a rubber septum and placed under nitrogen atmosphere. In a separate scintillation vial, DABCO (71.0 mg, 6.3 mmol) and methyl hex-5-ynoate (48.0 mg, 0.38 mmol) were dissolved in dry 1,4-dioxane (1.5 ml) and added to the MW vial via syringe. The resulting mixture is bubbled with nitrogen for 5 min followed by stirring for 16 hours at room temperature under nitrogen atmosphere. After 16 hours, the crude reaction mixture is filtered through a short pad of celite, concentrated in vacuo and purified by flash chromatography on silica gel using forced flow of 0-5% MeOH(0.1% TEA)/DCM to afford the coupled product 7.1 (70.0 mg, 0.17 mmol) in 55% yield. Compound 7.1 was dissolved in n-butanol (0.5 ml) in a microwave vial equipped with a stir bar followed by the addition of 2-amino-4-chloro-6-methylpyrimidine-5-carbonitrile (44.0 mg, 0.26 mmol) and diisopropylethylamine (67.0 mg, 0.52 mmol, 91.0 μl) to it. The vial was sealed and heated for 3 hours at 130° C. in a microwave. After completion of reaction (by LC-MS), the reaction mixture was concentrated in vacuo and the remaining residue was purified by flash chromatography on silica gel using forced flow of 0-5% MeOH/DCM to afford the coupled product 7.2 (35.0 mg, 0.07 mmol) in 38% yield.

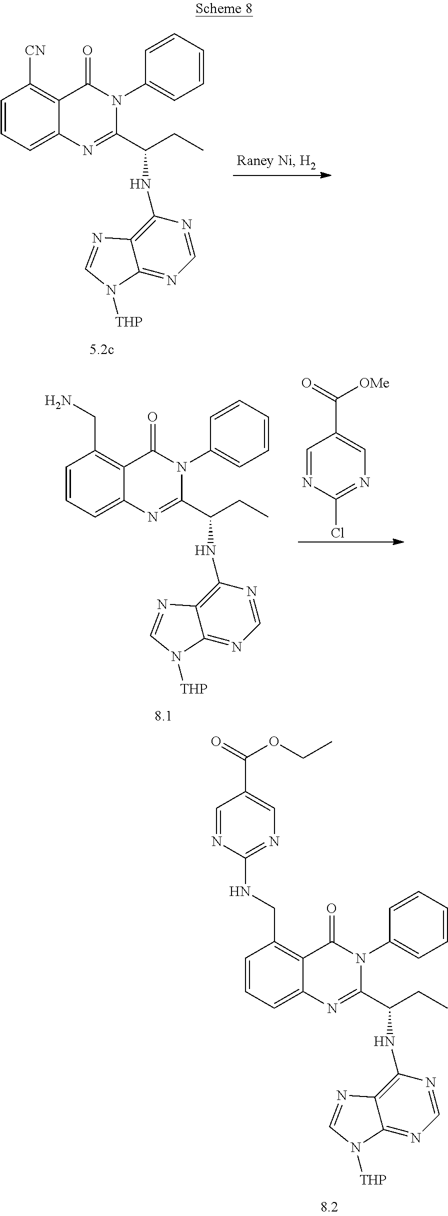

### Scheme 8

Dissolved 4-oxo-3-phenyl-2-((1S)-1-((9-(tetrahydro-2H-pyran-2-yl)-9H-purin-6-yl)amino)propyl)-3,4-dihydroquinazoline-5-carbonitrile (540.0 mg, 1.07 mmol) in ammonia (5.3 mL, 7N in MeOH) in a 20 ml scintillation vial and added Raney Ni (60.0 mg (approx.)) to it. The reaction vial is evacuated and then kept under hydrogen atmosphere using a balloon. The resulting suspension was stirred at room temperature for 20 hours. After completion of reaction (by LC-MS), the crude reaction mixture is carefully filtered under nitrogen and concentrated in vacuo to afford the product 5-(aminomethyl)-3-phenyl-2-((1S)-1-((9-(tetrahydro-2H-pyran-2-yl)-9H-purin-6-yl)amino)propyl)quinazolin-4(3H)-one 8.1 (523.0 mg, 1.023 mmol) in 96% yield. LC-MS (method 1): t_{R}=2.67 min, m/z (M+H)⁺=511.3.

5-(aminomethyl)-3-phenyl-2-((1S)-1-((9-(tetrahydro-2H-pyran-2-yl)-9H-purin-6-yl)amino)propyl)quinazolin-4(3H)-one 8.1 (160.0 mg, 0.31 mmol) was dissolved in ethanol (0.7 ml) in a microwave vial equipped with a sir bar and ethyl 2-chloropyrimidine-5-carboxylate (117.0 mg, 0.63 mmol) and triethylamine (127.0 mg, 1.25 mmol, 0.18 ml) were added to it. The microwave vial was sealed and the resulting mixture was heated at 90° C. for 3 hours in a microwave. After completion of the reaction, the reaction mixture is concentrated in vacuo and the remaining residue was purified using 0-5% MeOH/DCM to afford the product ethyl 2-(((4-oxo-3-phenyl-2-((1S)-1-((9-(tetrahydro-2H-pyran-2-yl)-9H-purin-6-yl)amino)propyl)-3,4-dihydroquinazolin-5-yl)methyl)amino)pyrimidine-5-carboxylate 8.2 (170.0 mg, 0.26 mmol) in 82% yield. LC-MS (method 1): t_{R}=3.41 min, m/z (M+H)⁺=661.3.

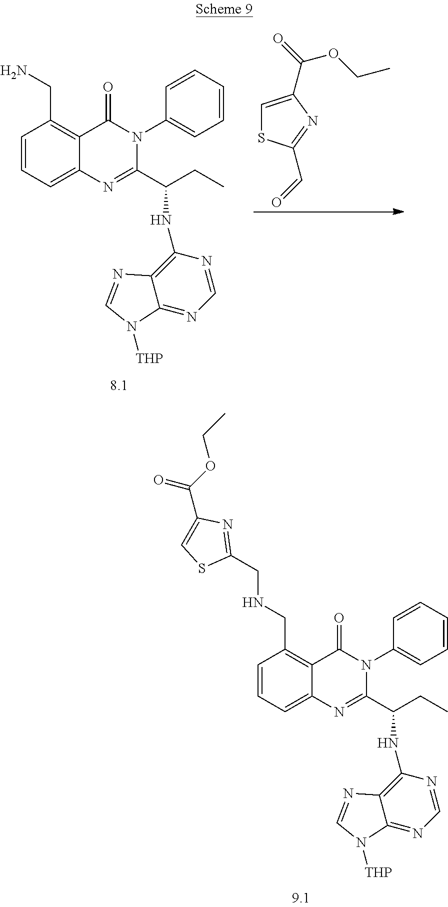

### Scheme 9

5-(aminomethyl)-3-phenyl-2-((1S)-1-((9-(tetrahydro-2H-pyran-2-yl)-9H-purin-6-yl)amino)propyl)quinazolin-4(3H)-one 8.1 (20.0 mg, 0.04 mmol) was dissolved in DCE (1 mL) and ethyl 2-formylthiazole-4-carboxylate (7.3 mg, 0.04 mmol) and a drop of acetic acid were added. The resulting mixture was stirred at room temperature for 2 hours. After 2 hours of stirring, Sodium triacetoxy borohydride (24.9 mg, 0.12 mmol) was added and the reaction mixture was stirred at room temperature for 2 hours. After completion of the reaction (by LC-MS), a saturated aqueous solution of sodium bicarbonate was added. The product was extracted three times with DCM. The combined organic layers were washed, dried over MgSO₄, filtered and concentrated in vacuo. The remaining residue was purified by flash chromatography on silica gel using 0-5% MeOH (with 1% triethylamine)/DCM to afford the product ethyl 2-((((4-oxo-3-phenyl-2-((1S)-1-((9-(tetrahydro-2H-pyran-2-yl)-9H-purin-6-yl)amino)propyl)-3,4-dihydroquinazolin-5-yl)methyl)amino)methyl)thiazole-4-carboxylate 9.1 (17.4 mg, 0.026 mmol) in 65% yield. LC-MS (method 1): t_{R}=2.96 min, m/z (M+H)⁺=680.3.

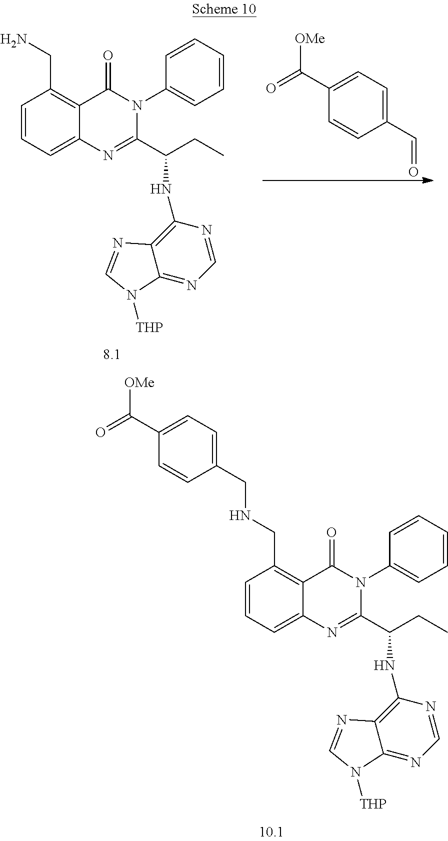

### Scheme 10

5-(aminomethyl)-3-phenyl-2-((1S)-1-((9-(tetrahydro-2H-pyran-2-yl)-9H-purin-6-yl)amino)propyl)quinazolin-4(3H)-one 8.1 (44.1 mg, 0.09 mmol) was dissolved in DCE (2 mL) and ethyl 2-formylthiazole-4-carboxylate (14.1 mg, 0.09 mmol) and a drop of acetic acid were added. The resulting mixture was stirred at room temperature for 2 hours. After 2 hours of stirring, Sodium triacetoxy borohydride (54.9 mg, 0.26 mmol) was added and the reaction mixture was stirred at room temperature for 2 hours. After completion of the reaction (by LC-MS), a saturated aqueous solution of sodium bicarbonate was added. The product was extracted three times with DCM. The combined organic layers were washed, dried over MgSO₄, filtered and concentrated in vacuo. The remaining residue was purified by flash chromatography on silica gel using 0-5% MeOH (with 1% triethylamine)/DCM to afford the product methyl 4-((((4-oxo-3-phenyl-2-((1S)-1-((9-(tetrahydro-2H-pyran-2-yl)-9H-purin-6-yl)amino)propyl)-3,4-dihydroquinazolin-5-yl)methyl)amino)methyl)benzoate 10.1 (53.3 mg, 0.08 mmol) in 94% yield. LC-MS (method 1): t_{R}=2.98 min, m/z (M+H)⁺=659.3.

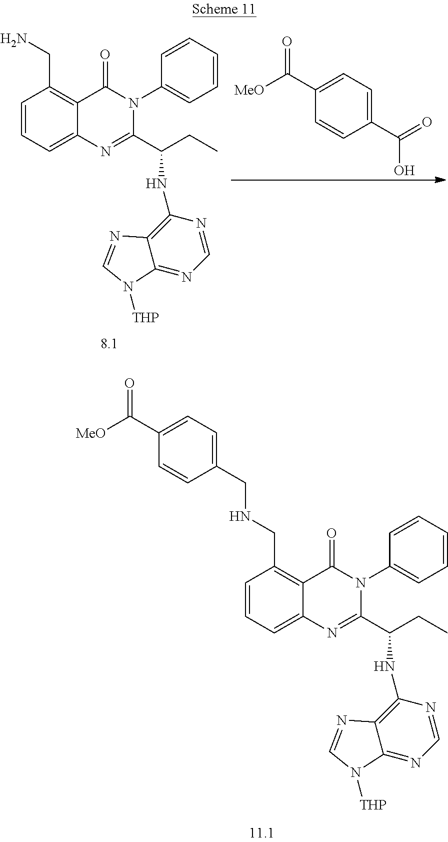

### Scheme 11

5-(aminomethyl)-3-phenyl-2-((1S)-1-((9-(tetrahydro-2H-pyran-2-yl)-9H-purin-6-yl)amino)propyl)quinazolin-4(3H)-one 8.1 (140.0 mg, 0.27 mmol) was dissolved in DMF (1.3 ml) in a 20 ml scintillation vial equipped with a stir bar and 4-acetylbenzoic acid (68.0 mg, 0.41 mmol), DIPEA (106.0 mg, 0.82, 0.14 ml) and 2-(3H-[1,2,3]triazolo[4,5-b]pyridin-3-yl)-1,1,3,3-tetramethylisouronium hexafluorophosphate(V) [HATU] (125.0 mg, 0.33 mmol) were added to it. The resulting mixture was stirred at room temperature for 16 hours and concentrated in vacuo. The remaining residue was purified using 0-10% MeOH/DCM to afford the product methyl 4-(((4-oxo-3-phenyl-2-((1S)-1-((9-(tetrahydro-2H-pyran-2-yl)-9H-purin-6-yl)amino)propyl)-3,4-dihydroquinazolin-5-yl)methyl)carbamoyl)benzoate 11.1 (160.0 mg, 0.24 mmol) in 87% yield. LC-MS (method 1): t_{R}=3.32 min, m/z (M+H)⁺=673.3.

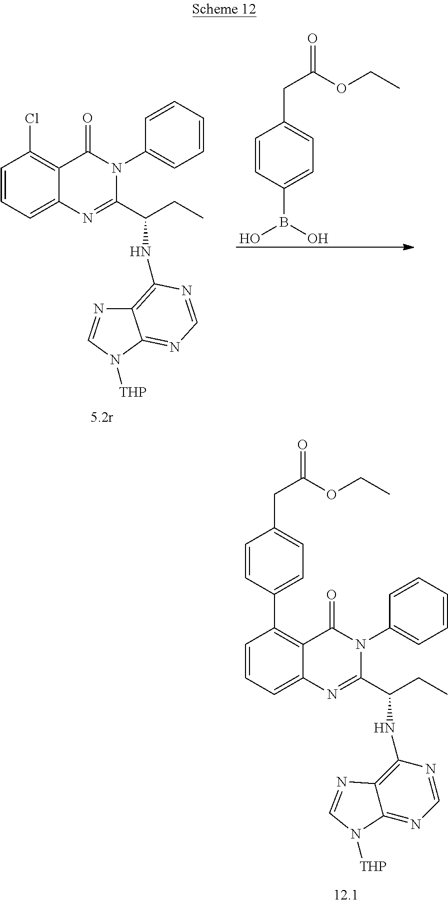

### Scheme 12

5-chloro-3-phenyl-2-((1S)-1-((9-(tetrahydro-2H-pyran-2-yl)-9H-purin-6-yl)amino)propyl)quinazolin-4(3H)-one 5.2r (70.0 mg, 0.14 mmol), chloro(crotyl)(2-dicyclohexylphosphino-2′,4′,6′-triisopropylbiphenyl)palladium(II) [Pd-170] (4.6 mg, 0.07 mmol) (4-(2-ethoxy-2-oxoethyl)phenyl)boronic acid (42.0 mg, 0.20 mmol) were suspended in dioxane/water (0.7 ml, 4:1) in a MW vial equipped with a stir bar under N₂ atmosphere and potassium phosphate (86.0 mg, 0.41 mmol) was added to it. The MW vial was sealed and heated at 100° C. for 2 hours in a MW reactor. The reaction mixture was allowed to cool to RT, quenched with water, and then extracted 3 times with ethyl acetate. The combined organic fractions were dried over MgSO₄ and then concentrated in vacuo. The remaining residue was purified using 0-100% EtOAc/Hexanes to afford ethyl 2-(4-(4-oxo-3-phenyl-2-((1S)-1-((9-(tetrahydro-2H-pyran-2-yl)-9H-purin-6-yl)amino)propyl)-3,4-dihydroquinazolin-5-yl)phenyl)acetate 12.1 (44.0 mg, 0.07 mmol) in 50% yield. LC-MS (method 1): t_{R}=3.51 min, m/z (M+H)⁺=644.4.

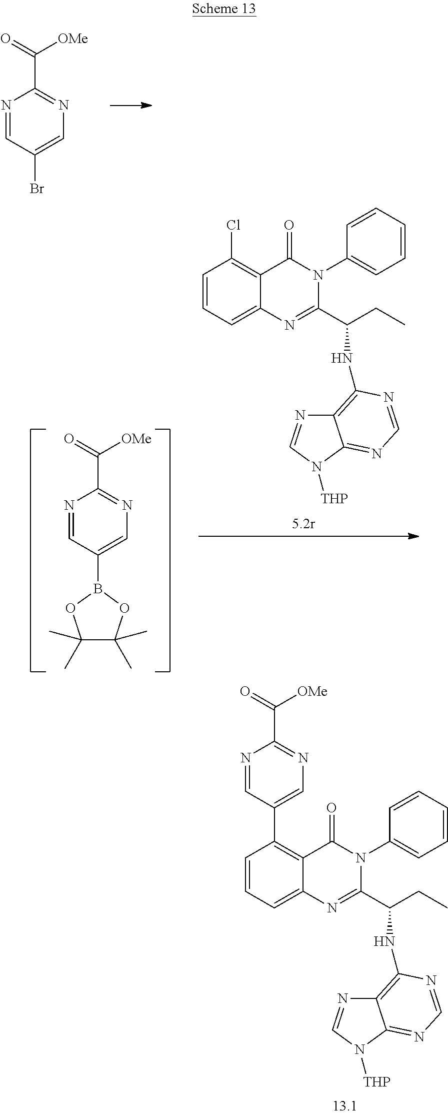

### Scheme 13

To a mixture of methyl 5-bromopyrimidine-2-carboxylate (150.0 mg, 0.69 mmol) and 4,4,4′,4′,5,5,5′,5′-octamethyl-2,2′-bi(1,3,2-dioxaborolane) [BPin]₂ (211.0 mg, 0.83 mmol) in 1,4-dioxane (1.8 ml) in a MW tube equipped with a stirring bar, Pd(dppf)Cl₂ (25.0 mg, 0.04 mmol) and potassium acetate (204.0 mg, 2.07 mmol) were added under N₂ bubbling through the solvent. The resulting mixture was stirred at 100° C. for 2 hours. After completion of the reaction, the crude reaction mixture is filtered into a MW vial equipped with a stir bar and 5-chloro-3-phenyl-2-((1S)-1-((9-(tetrahydro-2H-pyran-2-yl)-9H-purin-6-yl)amino)propyl)quinazolin-4(3H)-one 5.2r (120.0 mg, 0.23 mmol) and 0.2 ml of water were added to it. Added [Pd-170] (7.8 mg, 0.012 μmol) and potassium phosphate (148.0 mg, 0.70 mmol) to this mixture under nitrogen atmosphere. The MW vial was sealed and heated at 100° C. for 2 hours. The reaction mixture was allowed to cool to room temperature, quenched with water, and then extracted 3 times with ethyl acetate. The combined organic fractions were dried over MgSO₄ and then concentrated in vacuo. The remaining residue was purified by flash chromatography on silica using 0-5% MeOH/DCM to afford the coupled product methyl 5-(4-oxo-3-phenyl-2-((1S)-1-((9-(tetrahydro-2H-pyran-2-yl)-9H-purin-6-yl)amino)propyl)-3,4-dihydroquinazolin-5-yl)pyrimidine-2-carboxylate (135.0 mg, 0.22 mmol) 1.4b in 94% yield. LC-MS (method 1): t_{R}=3.12 min, m/z (M+H)⁺=618.3.

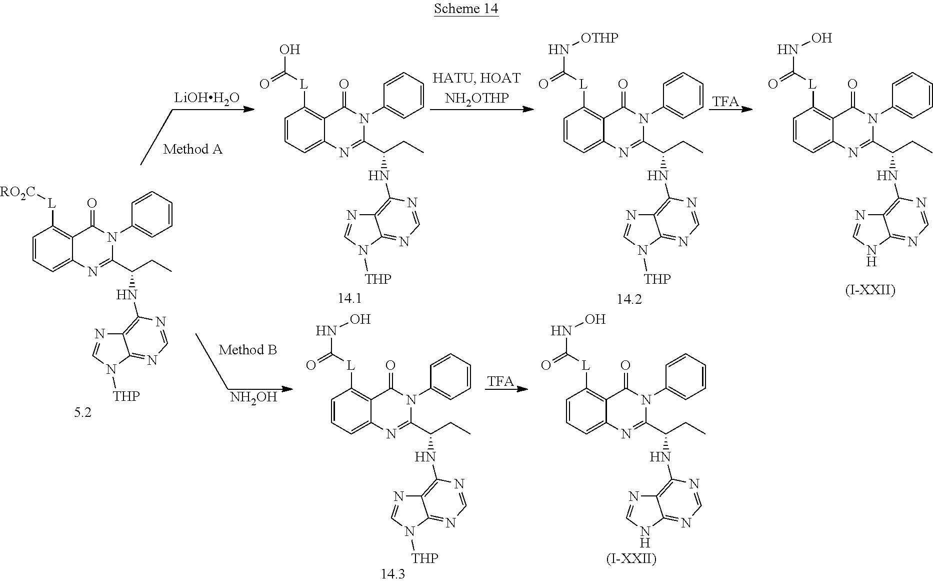

### Scheme 14

Method A: Suspended compound 5.2 in MeOH/water (0.1 M, 1:1) in a vial equipped with a stir bar and added LiOH.H₂O (2.0 equiv) to it. The resulting mixture was stirred at room temperature for 10 hours and concentrated in vacuo to afford crude 14.1. Suspended crude compound 14.1 in DMF (0.1 M) and added O-(tetrahydro-2H-pyran-2-yl)hydroxylamine [NH₂OTHP] (3.1 equiv), N-methyl morpholine (3.0 equiv), 3-(((ethylimino)methylene)amino)-N,N-dimethylpropan-1-amine hydrochloride [EDC.HCl] (1.4 equiv) and 1H-[1,2,3]triazolo[4,5-b]pyridin-1-ol [HOAT] (1.2 equiv) to it. Stirred the resulting suspension at room temperature for 16 hours and concentrated in vacuo. Purified the remaining residue by flash chromatography on silica using forced flow of 0-10% MeOH/DCM system on Biotage KP-Sil pre-packed cartridges and using the Biotage SP-1 automated chromatography system to afford the product 14.2. Dissolved compound 14.2 in DCM (0.1M) and added TFA (20.0 equiv) to it. Stirred the resulting mixture for 20 hours. After completion of reaction (by LC-MS), concentrated the reaction mixture in vacuo and purified by C-18 reverse phase chromatography to afford the final compound (I-XXII).

Method B: Dissolved compound 5.2 in MeOH (0.1M) in a MW vial equipped with a stir bar and added 50% hydroxylamine in water solution (30.0 equiv) and lithium hydroxide (1.2 equiv) at 0° C. to it. The MW vial was sealed and the resulting solution was stirred at 0° C. for 2 hours, then allowed to warmup to room temperature overnight. After completion of reaction by LC-MS, the reaction mixture was concentrated in vacuo to afford the crude product 14.3. Dissolved compound 14.3 in DCM/MeOH (0.1M, 1:1 by vol) and added TFA (20.0 equiv) to it. Stirred the resulting mixture for 20 hours. After completion of reaction (by LC-MS), concentrated the reaction mixture in vacuo and purified by C-18 reverse phase chromatography to afford the final compound (I-XXII).

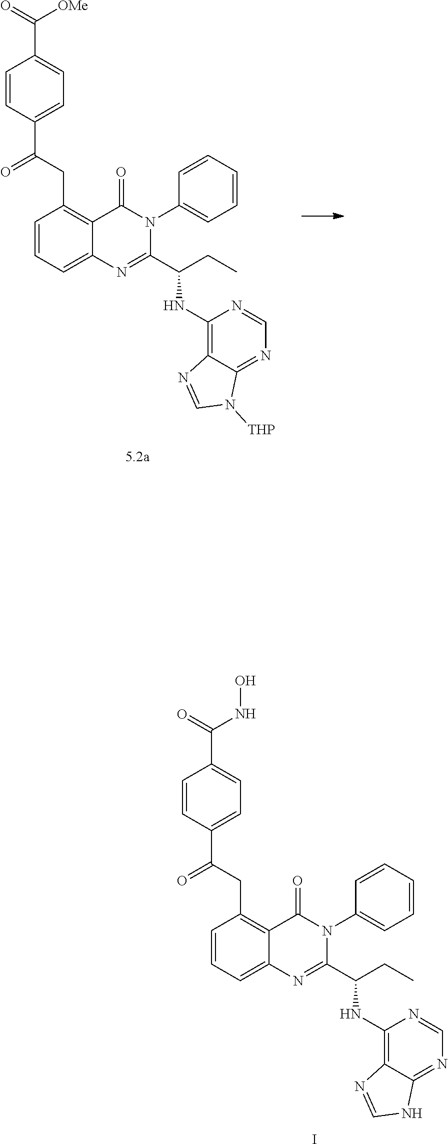

The procedure mentioned in Scheme 14 (Method A) was used with compound 5.2a (69.0 mg, 0.105 mmol) to afford product (S)-4-(2-(2-(1-((9H-purin-6-yl)amino)propyl)-4-oxo-3-phenyl-3,4-dihydroquinazolin-5-yl)acetyl)-N-hydroxybenzamide, TFA I (32.0 mg, mmol) in 44% yield. LC-MS (method 2): t_{R}=3.79 min, m/z (M+H)⁺=575.1. ¹H NMR (400 MHz, DMSO-d6) δ 11.34 (s, 1H), 8.70 (s, 2H), 8.42 (s, 2H), 8.03 (d, J=7.9 Hz, 2H), 7.81 (dd, J=25.9, 7.9 Hz, 3H), 7.62 (d, J=8.2 Hz, 1H), 7.56-7.35 (m, 6H), 4.98-4.81 (m, 3H), 4.75 (s, 1H), 2.02 (s, 1H), 1.91-1.81 (m, 1H), 0.77 (t, J=7.3 Hz, 3H).

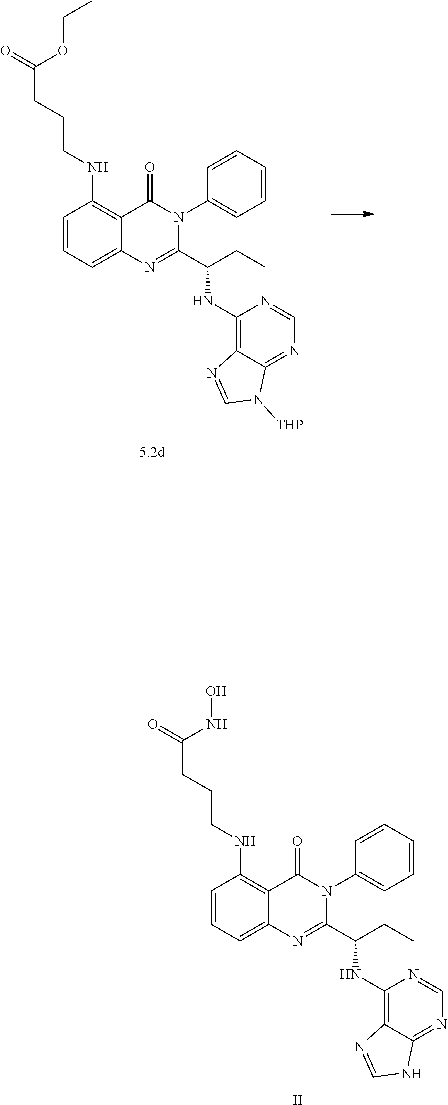

The procedure mentioned in Scheme 14 (Method B) was used with compound 5.2d (65.0 mg, 0.106 mmol) to afford product (S)-4-((2-(1-((9H-purin-6-yl)amino)propyl)-4-oxo-3-phenyl-3,4-dihydroquinazolin-5-yl)amino)-N-hydroxybutanamide, TFA 11 (11.5 mg, 0.022 mmol) in 21% yield. LC-MS (method 2): t_{R}=3.50 min, m/z (M+H)⁺=514.2. 1H NMR (400 MHz, DMSO-d6) δ 10.39 (s, 1H), 8.49 (s, 1H), 8.38 (s, 2H), 7.59-7.47 (m, 5H), 6.70 (d, J=7.9 Hz, 1H), 6.57 (d, J=8.5 Hz, 1H), 5.75 (s, 1H), 4.74 (s, 1H), 3.16 (d, J=4.9 Hz, 3H), 2.08-1.92 (m, 3H), 1.79 (h, J=7.3, 6.9 Hz, 3H), 0.76 (t, J=7.3 Hz, 3H). [Note: In addition to compound II, a side product originating from the hydrolysis of ethyl ester in 5.2d to carboxylic acid was also isolated after TFA deprotection step, in 7% yield].

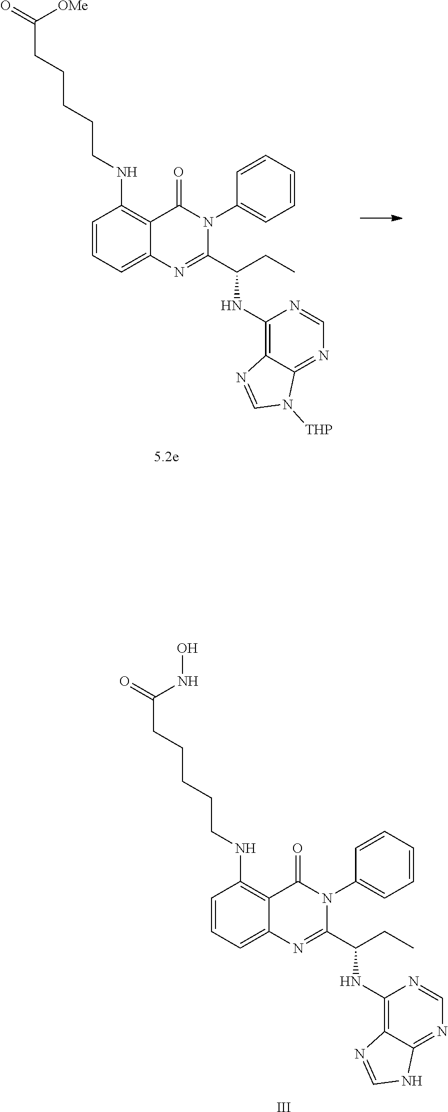

The procedure mentioned in Scheme 14 (Method B) was used with compound 5.2e (135.0 mg, 0.216 mmol) to afford product N-hydroxy-6-((4-oxo-3-phenyl-2-((1S)-1-((9-(tetrahydro-2H-pyran-2-yl)-9H-purin-6-yl)amino)propyl)-3,4-dihydroquinazolin-5-yl)amino)hexanamide, TFA III (30.2 mg, 0.056 mmol) in 32% yield. LC-MS (method 2): t_{R}=4.02 min, m/z (M+H)⁺=542.3. ¹H NMR (400 MHz, DMSO-d6) δ 10.33-10.28 (m, 1H), 8.89 (s, 1H), 8.48 (s, 2H), 7.53 (dq, J=13.3, 8.0, 4.8 Hz, 5H), 6.70 (d, J=7.9 Hz, 1H), 6.56 (d, J=8.5 Hz, 1H), 4.77 (s, 1H), 3.13 (t, J=6.9 Hz, 2H), 2.01 (ddd, J=14.2, 7.5, 4.5 Hz, 1H), 1.93 (t, J=7.4 Hz, 2H), 1.83 (h, J=7.4 Hz, 1H), 1.53 (dp, J=22.8, 7.3 Hz, 4H), 1.37-1.25 (m, 2H), 0.77 (t, J=7.3 Hz, 3H). [Note: In addition to compound III, a side product originating from the hydrolysis of methyl ester in 5.2e to carboxylic acid was also isolated after TFA deprotection step, in 14% yield].

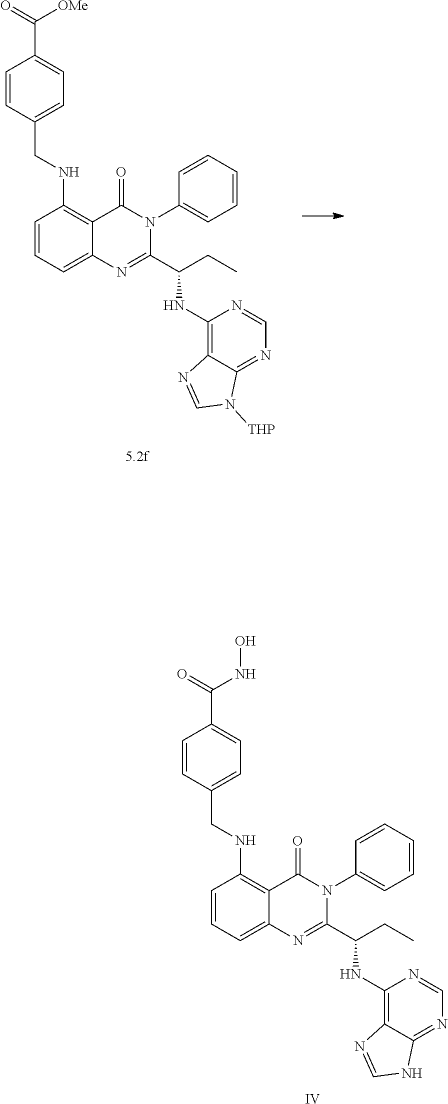

The procedure mentioned in Scheme 14 (Method A) was used with compound 5.2f (78.1 mg, 0.121 mmol) to afford product (S)-4-(((2-(1-((9H-purin-6-yl)amino)propyl)-4-oxo-3-phenyl-3,4-dihydroquinazolin-5-yl)amino)methyl)-N-hydroxybenzamide, TFA IV (35.5 mg, 0.106 mmol) in 50% yield. LC-MS (method 2): t_{R}=3.88 min, m/z (M+H)⁺=562.2. ¹H NMR (400 MHz, DMSO-d6) δ 11.15 (s, 1H), 8.97 (s, 1H), 8.77 (s, 1H), 8.45 (s, 2H), 7.73-7.66 (m, 2H), 7.58 (s, 2H), 7.55 (t, J=1.6 Hz, 1H), 7.52-7.36 (m, 5H), 6.73 (d, J=7.9 Hz, 1H), 6.48 (d, J=8.4 Hz, 1H), 4.77 (s, 1H), 4.52-4.46 (m, 2H), 2.01 (ddd, J=14.2, 7.6, 4.6 Hz, 1H), 1.83 (dt, J=14.4, 7.5 Hz, 1H), 0.77 (t, J=7.3 Hz, 3H).

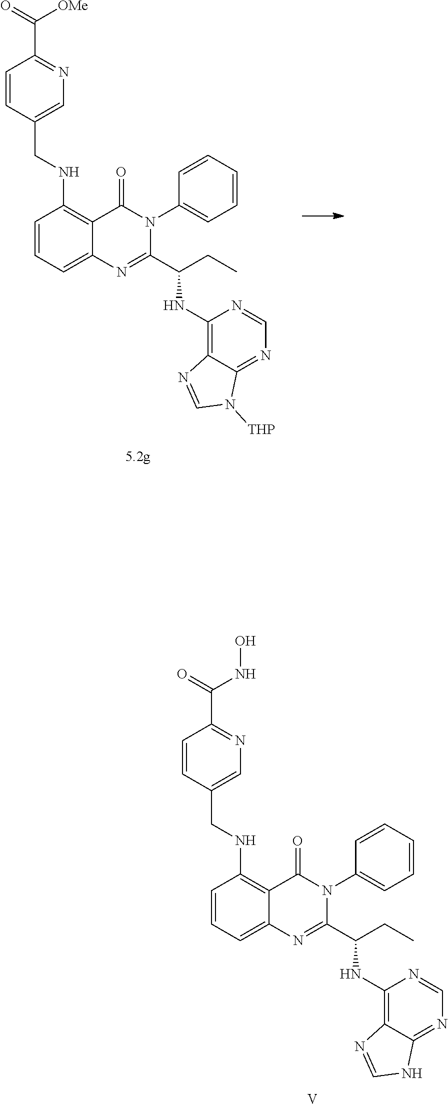

The procedure mentioned in Scheme 14 (Method A) was used with compound 5.2g (60.5 mg, 0.094 mmol) to afford product(S)-5-(((2-(1-((9H-purin-6-yl)amino)propyl)-4-oxo-3-phenyl-3,4-dihydroquinazolin-5-yl)amino)methyl)-N-hydroxypicolinamide, TFA V (20.0 mg, 0.03 mmol) in 32% yield. LC-MS (method 2): t_{R}=3.77 min, m/z (M+H)⁺=563.2. ¹H NMR (400 MHz, DMSO-d6) δ 11.35 (s, 1H), 9.02 (s, 1H), 8.85 (s, 1H), 8.58 (d, J=2.0 Hz, 1H), 8.47 (s, 2H), 7.96-7.83 (m, 2H), 7.66-7.40 (m, 6H), 6.75 (d, J=7.9 Hz, 1H), 6.53 (d, J=8.4 Hz, 1H), 4.78 (s, 1H), 4.58 (d, J=3.4 Hz, 2H), 2.02 (ddd, J=14.4, 7.5, 4.5 Hz, 1H), 1.82 (dt, J=14.7, 7.7 Hz, 1H), 0.77 (t, J=7.3 Hz, 3H).

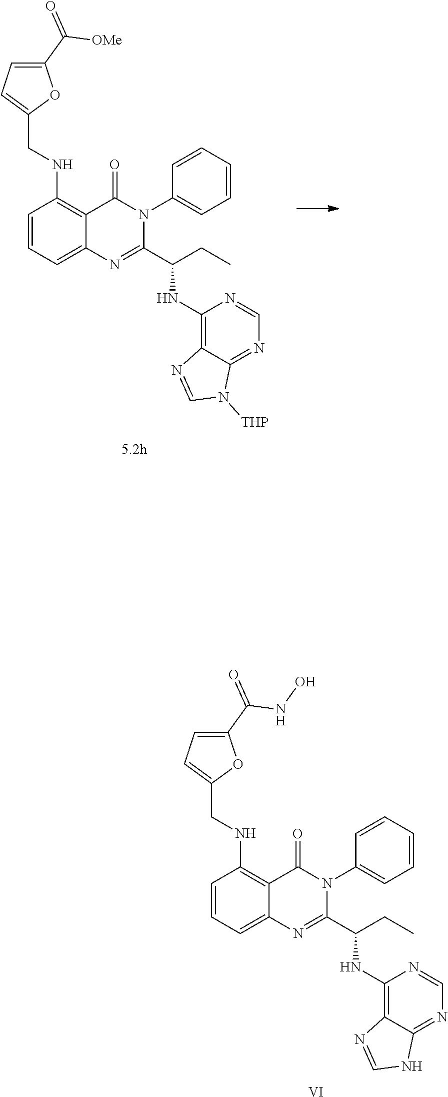

The procedure mentioned in Scheme 14 (Method A) was used with compound 5.2h (61.5 mg, 0.097 mmol) to afford product (S)-5-(((2-(1-((9H-purin-6-yl)amino)propyl)-4-oxo-3-phenyl-3,4-dihydroquinazolin-5-yl)amino)methyl)-N-hydroxyfuran-2-carboxamide, TFA VI (41.0 mg, 0.076 mmol) in 78% yield. LC-MS (method 2): t_{R}=3.73 min, m/z (M+H)=552.2. ¹H NMR (400 MHz, DMSO-d6) δ 11.09 (s, 2H), 8.83 (s, 3H), 8.46 (d, J=2.9 Hz, 2H), 7.65-7.42 (m, 6H), 6.95 (d, J=3.4 Hz, 1H), 6.77 (d, J=7.9 Hz, 1H), 6.69 (d, J=8.4 Hz, 1H), 6.45 (d, J=3.4 Hz, 1H), 4.76 (dd, J=10.5, 4.9 Hz, 1H), 4.48 (d, J=3.7 Hz, 2H), 2.00 (ddd, J=14.3, 7.4, 4.3 Hz, 1H), 1.82 (dt, J=14.5, 7.6 Hz, 1H), 0.76 (t, J=7.3 Hz, 3H).

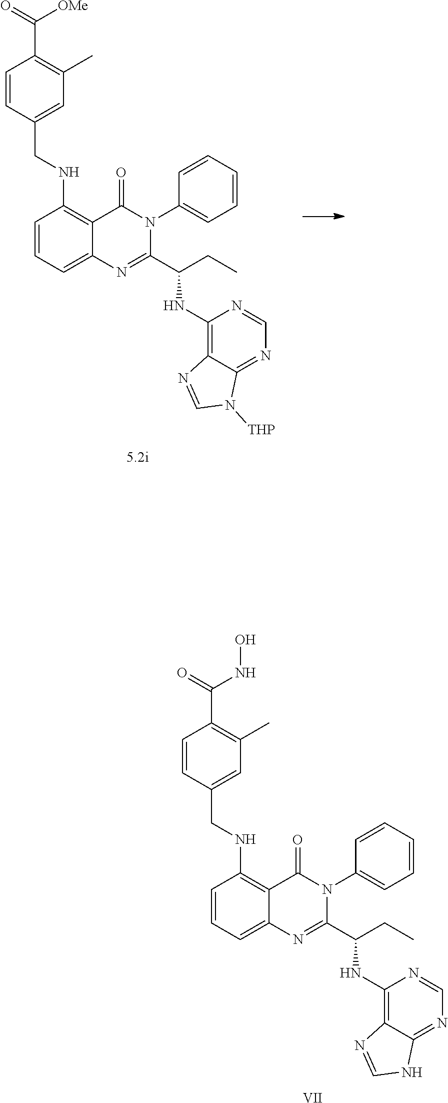

The procedure mentioned in Scheme 14 (Method B) was used with compound 5.2i (105.0 mg, 0.159 mmol) to afford product (S)-4-(((2-(1-((9H-purin-6-yl)amino)propyl)-4-oxo-3-phenyl-3,4-dihydroquinazolin-5-yl)amino)methyl)-N-hydroxy-2-methylbenzamide, TFA VII (60.0 mg, 0.087 mmol) in 55% yield. LC-MS (method 2): t_{R}=4.18 min, m/z (M+H)⁺=576.2. 1H NMR (400 MHz, DMSO-d6) δ 10.76 (s, 1H), 8.88 (s, 1H), 8.74 (s, 1H), 8.44 (s, 2H), 7.61-7.52 (m, 3H), 7.56-7.43 (m, 4H), 7.26-7.13 (m, 3H), 6.73 (d, J=7.9 Hz, 1H), 6.51 (dd, J=8.5, 1.0 Hz, 1H), 4.78 (s, 1H), 4.41 (s, 2H), 2.30 (s, 3H), 2.01 (ddd, J=14.0, 7.4, 4.4 Hz, 1H), 1.83 (dt, J=14.8, 8.0 Hz, 1H), 0.77 (t, J=7.3 Hz, 3H). [Note: In addition to compound VII, a side product originating from the hydrolysis of methyl ester in 5.2i to carboxylic acid was also isolated after TFA deprotection step, in 13% yield].

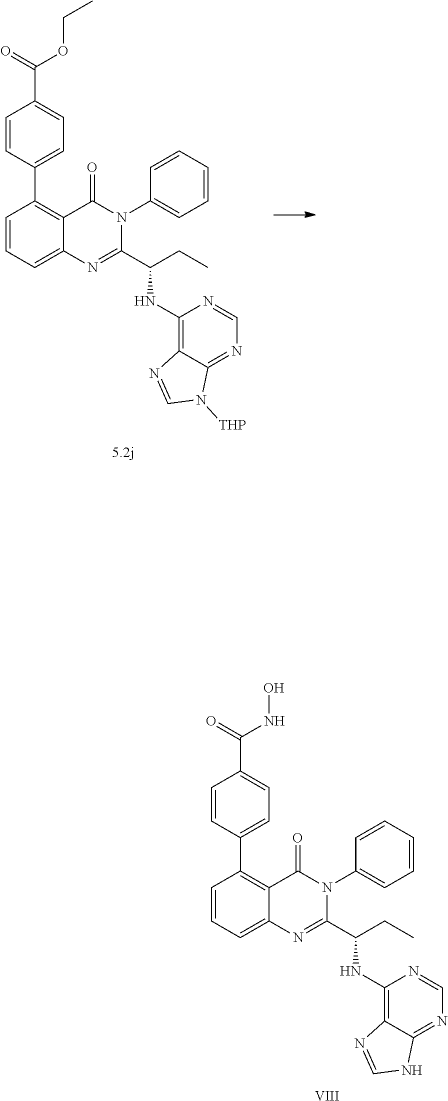

The procedure mentioned in Scheme 14 (Method A) was used with compound 5.2j (73.2 mg, 0.116 mmol) to afford product (S)-4-(2-(1-((9H-purin-6-yl)amino)propyl)-4-oxo-3-phenyl-3,4-dihydroquinazolin-5-yl)-N-hydroxybenzamide, TFA VIII (53.0 mg, 0.082 mmol) in 71% yield. LC-MS (method 2): t_{R}=3.56 min, m/z (M+H)⁺=533.2. ¹H NMR (400 MHz, DMSO-d6) δ 11.18 (s, 1H), 8.69 (s, 1H), 8.42 (s, 2H), 7.84 (dd, J=8.2, 7.4 Hz, 1H), 7.74-7.64 (m, 3H), 7.57-7.37 (m, 5H), 7.34-7.24 (m, 3H), 5.75 (s, 1H), 4.80 (s, 1H), 2.03 (ddd, J=11.6, 7.4, 3.6 Hz, 1H), 1.87 (dt, J=14.7, 7.6 Hz, 1H), 0.79 (t, J=7.3 Hz, 3H).

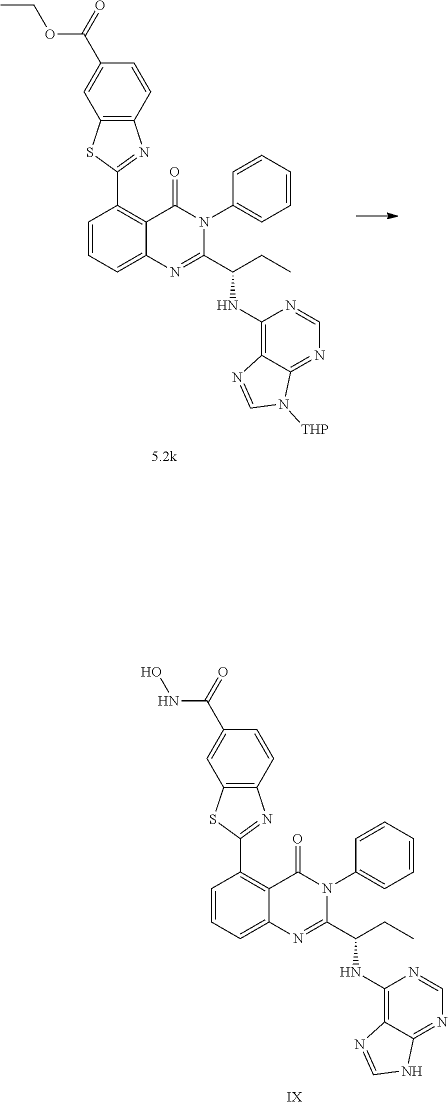

The procedure mentioned in Scheme 14 (Method B) was used with compound 5.2k (16.4 mg, 0.024 mmol) to afford product (S)-2-(2-(1-((9H-purin-6-yl)amino)propyl)-4-oxo-3-phenyl-3,4-dihydroquinazolin-5-yl)-N-hydroxybenzo[d]thiazole-6-carboxamide, TFA IX (5.0 mg, 0.007 mmol) in 32% yield. LC-MS (method 2): t_{R}=3.64 min, m/z (M+H)⁺=590.2. ¹H NMR (400 MHz, DMSO-d6) δ 11.33 (s, 1H), 8.47 (d, J=1.7 Hz, 1H), 8.27 (s, 2H), 8.02 (d, J=8.5 Hz, 1H), 7.97-7.83 (m, 4H), 7.67-7.60 (m, 2H), 7.58-7.47 (m, 4H), 7.42 (s, 3H), 4.73 (s, 1H), 2.01 (s, 1H), 1.89 (s, 1H), 0.78 (t, J=7.3 Hz, 3H).

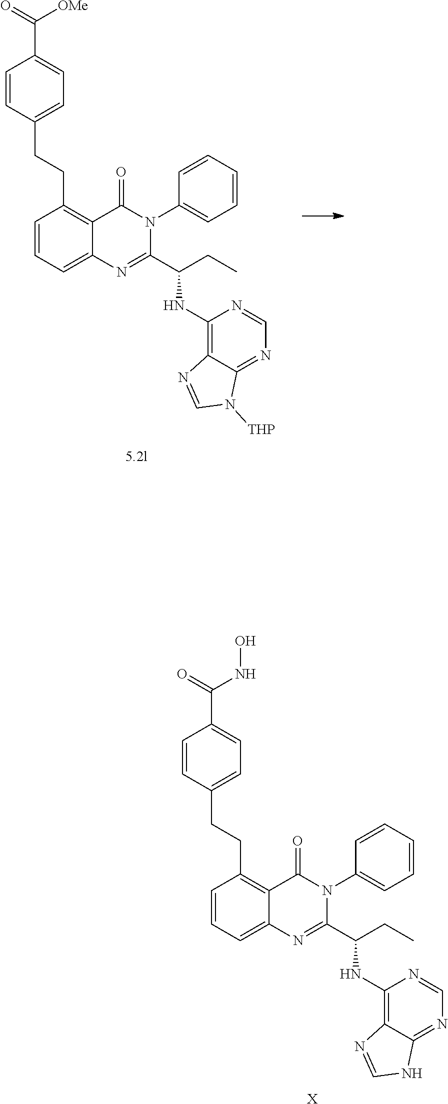

The procedure mentioned in Scheme 14 (Method A) was used with compound 5.21 (12.0 mg, 0.019 mmol) to afford product (S)-4-(2-(2-(1-((9H-purin-6-yl)amino)propyl)-4-oxo-3-phenyl-3,4-dihydroquinazolin-5-yl)ethyl)-N-hydroxybenzamide, TFA X (3.0 mg, 0.005 mmol) in 25% yield. LC-MS (method 2): t_{R}=3.94 min, m/z (M+H)⁺=561.3.

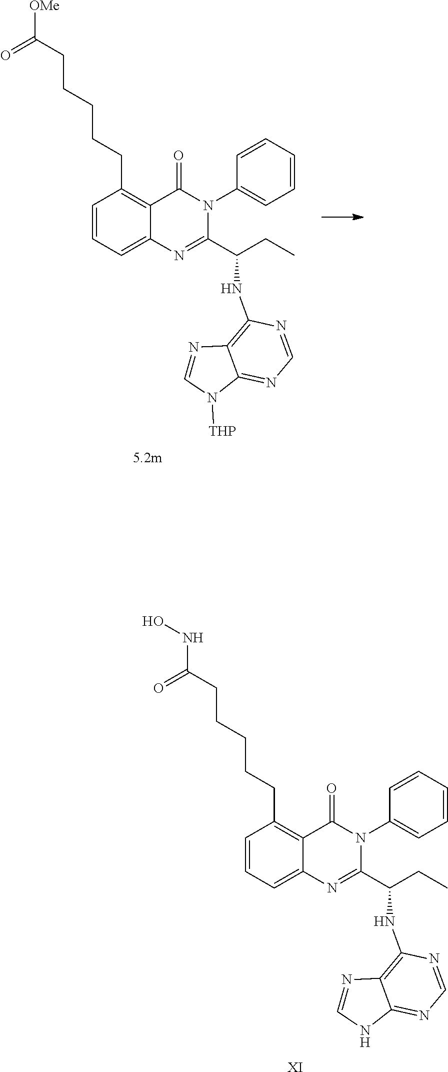

The procedure mentioned in Scheme 14 (Method B) was used with compound 5.2m (60.0 mg, 0.098 mmol) to afford product (S)-6-(2-(1-((9H-purin-6-yl)amino)propyl)-4-oxo-3-phenyl-3,4-dihydroquinazolin-5-yl)-N-hydroxyhexanamide, TFA XI (15.0 mg, 0.023 mmol) in 24% yield. LC-MS (method 2): t_{R}=3.75 min, m/z (M+H)⁺=527.3. 1H NMR (400 MHz, DMSO-d6) δ 10.27 (s, 1H), 8.32 (s, 2H), 7.73-7.64 (m, 1H), 7.59-7.46 (m, 5H), 7.29 (dd, J=7.5, 1.3 Hz, 1H), 4.76 (s, 1H), 3.13 (t, J=7.7 Hz, 2H), 2.04-1.95 (m, 1H), 1.94-1.78 (m, 3H), 1.47 (p, J=7.6 Hz, 4H), 1.27 (dt, J=14.4, 7.6 Hz, 2H), 0.77 (t, J=7.3 Hz, 3H).

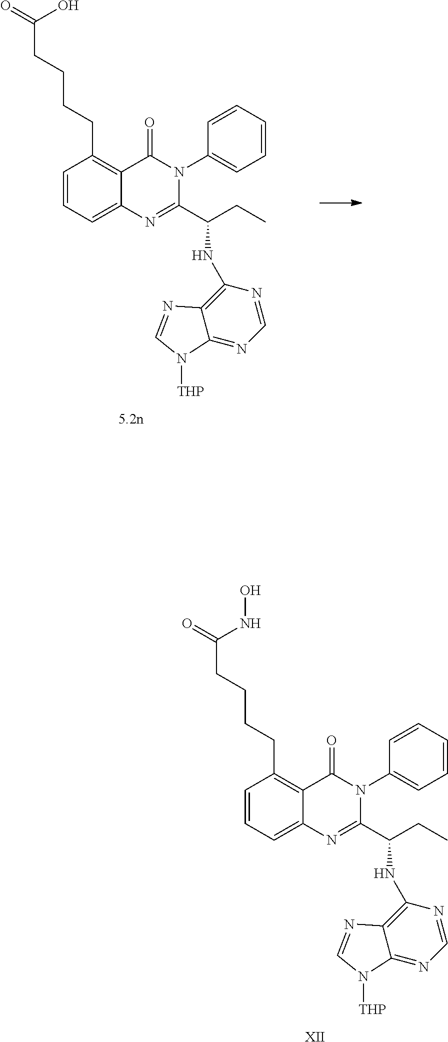

The procedure mentioned in Scheme 14 (Method A) was used with compound 5.2n (17.0 mg, 0.03 mmol) to afford product (S)-5-(2-(1-((9H-purin-6-yl)amino)propyl)-4-oxo-3-phenyl-3,4-dihydroquinazolin-5-yl)-N-hydroxypentanamide, TFA XII (8.0 mg, 0.013 mmol) in 43% yield. LC-MS (method 2): t_{R}=3.74 min, m/z (M+H)⁺=513.2. ¹H NMR (400 MHz, DMSO-d6) δ 10.28 (s, 1H), 8.26 (s, 1H), 7.68 (t, J=7.7 Hz, 1H), 7.56 (d, J=7.3 Hz, 3H), 7.50 (d, J=1.2 Hz, 1H), 7.48 (s, 1H), 7.28 (dd, J=7.6, 1.2 Hz, 1H), 7.20 (s, 1H), 7.07 (s, 1H), 6.95 (s, 1H), 4.74 (s, 1H), 3.81 (s, 1H), 3.14 (s, 2H), 1.97-1.90 (m, 2H), 1.90-1.79 (m, 1H), 1.53-1.48 (m, 3H), 0.76 (t, J=7.3 Hz, 3H).

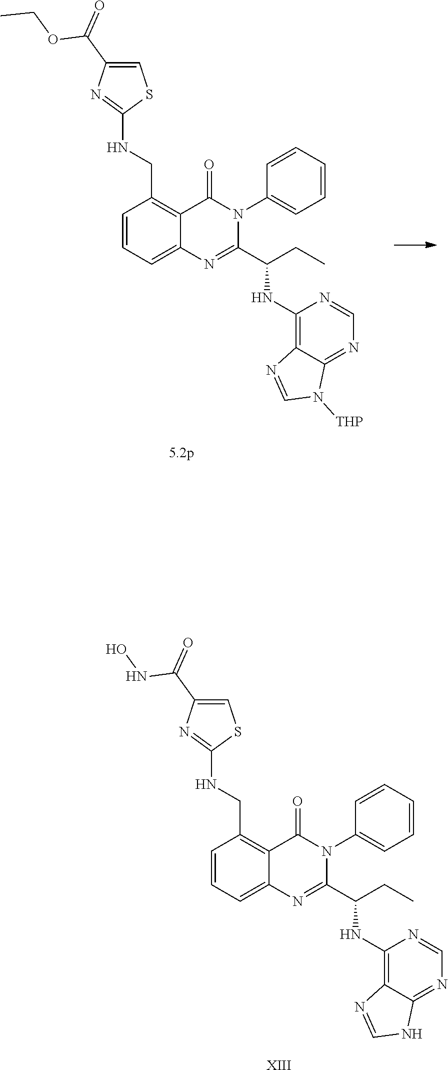

The procedure mentioned in Scheme 14 (Method A) was used with compound 5.2p (52.4 mg, 0.09 mmol) to afford product(S)-2-(((2-(1-((9H-purin-6-yl)amino)propyl)-4-oxo-3-phenyl-3,4-dihydroquinazolin-5-yl)methyl)amino)-N-hydroxythiazole-4-carboxamide, TFA XIII (20.5 mg, 0.03 mmol) in 33% yield. LC-MS (method 2): t_{R}=3.71 min, m/z (M+H)⁺=569.2. 1H NMR (400 MHz, DMSO-d6) δ 10.62 (s, 1H), 8.50 (s, 1H), 8.37 (s, 2H), 8.05 (t, J=6.2 Hz, 1H), 7.76 (t, J=7.8 Hz, 1H), 7.65-7.55 (m, 5H), 7.52 (s, 2H), 7.17 (s, 1H), 5.03 (d, J=6.0 Hz, 2H), 4.79 (s, 1H), 2.00 (d, J=11.7 Hz, 1H), 1.90-1.82 (m, 1H), 0.78 (t, J=7.3 Hz, 3H).

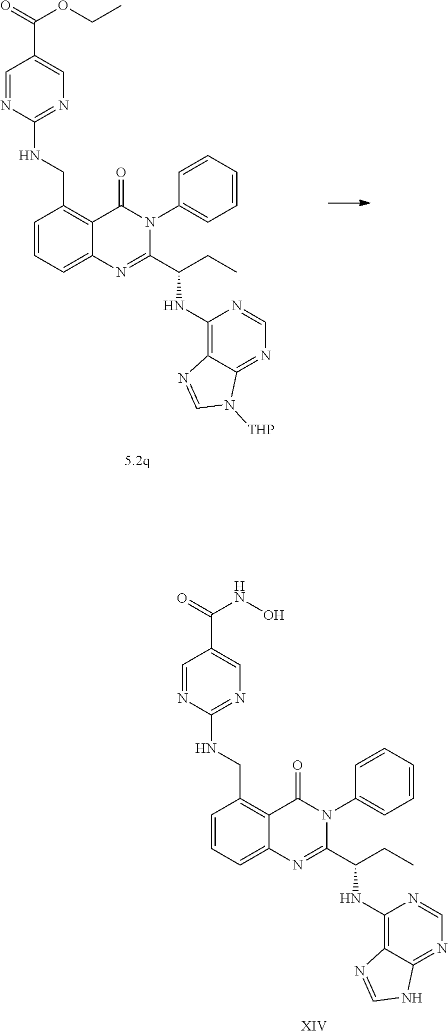

The procedure mentioned in Scheme 14 (Method A) was used with compound 5.2q (20.6 mg, 0.031 mmol) to afford product (S)-2-(1-((9H-purin-6-yl)amino)propyl)-N-(5-(hydroxycarbamoyl)pyrimidin-2-yl)-4-oxo-3-phenyl-3,4-dihydroquinazoline-5-carboxamide, TFA XIV (10.2 mg, 0.015 mmol) in 48% yield. LC-MS (method 2): t_{R}=3.09 min, m/z (M+H)⁺=578.2. ¹H NMR (400 MHz, DMSO-d6) δ 11.32 (s, 1H), 11.16 (s, 1H), 8.79 (s, 1H), 8.30 (s, 2H), 7.83 (dd, J=8.2, 7.3 Hz, 2H), 7.70 (dd, J=8.2, 1.2 Hz, 1H), 7.60-7.53 (m, 2H), 7.48 (s, 2H), 7.42 (dd, J=7.3, 1.2 Hz, 1H), 7.21 (s, 1H), 7.09 (s, 1H), 6.96 (s, 1H), 4.75 (s, 1H), 1.97 (s, 1H), 1.90 (d, J=7.8 Hz, 1H), 0.78 (t, J=7.3 Hz, 3H).

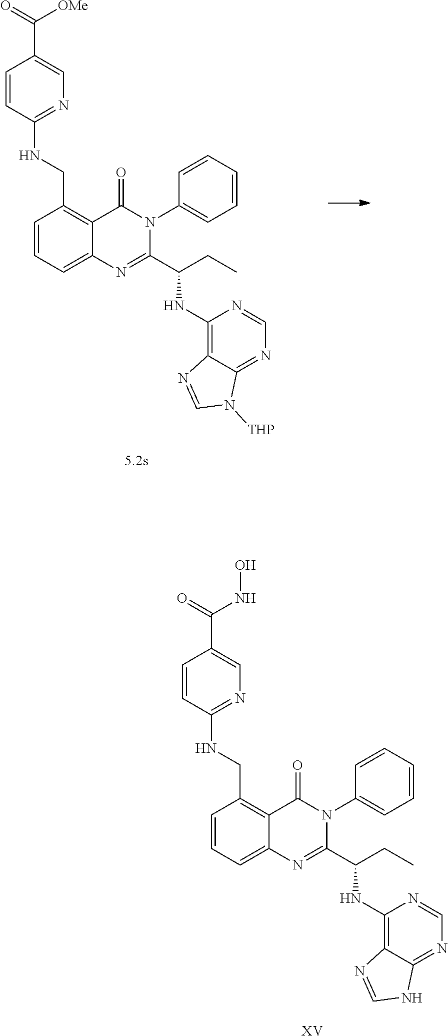

The procedure mentioned in Scheme 14 (Method B) was used with compound 5.2s (79.1 mg, 0.122 mmol) to afford product (S)-6-(((2-(1-((9H-purin-6-yl)amino)propyl)-4-oxo-3-phenyl-3,4-dihydroquinazolin-5-yl)amino)methyl)-N-hydroxynicotinamide, TFA XV (30.0 mg, 0.044 mmol) in 36% yield. LC-MS (method 2): t_{R}=3.54 min, m/z (M+H)⁺=563.3. ¹H NMR (400 MHz, DMSO-d6) δ 11.32 (s, 1H), 9.22 (s, 1H), 8.96 (s, 1H), 8.85 (dd, J=2.2, 0.8 Hz, 1H), 8.51 (s, 2H), 8.06 (dd, J=8.1, 2.3 Hz, 1H), 7.63-7.42 (m, 6H), 6.76 (d, J=7.9 Hz, 1H), 6.54-6.49 (m, 1H), 4.81 (s, 1H), 4.59 (s, 2H), 2.08-1.98 (m, 1H), 1.83 (dt, J=14.7, 7.8 Hz, 1H), 0.79 (t, J=7.3 Hz, 3H).

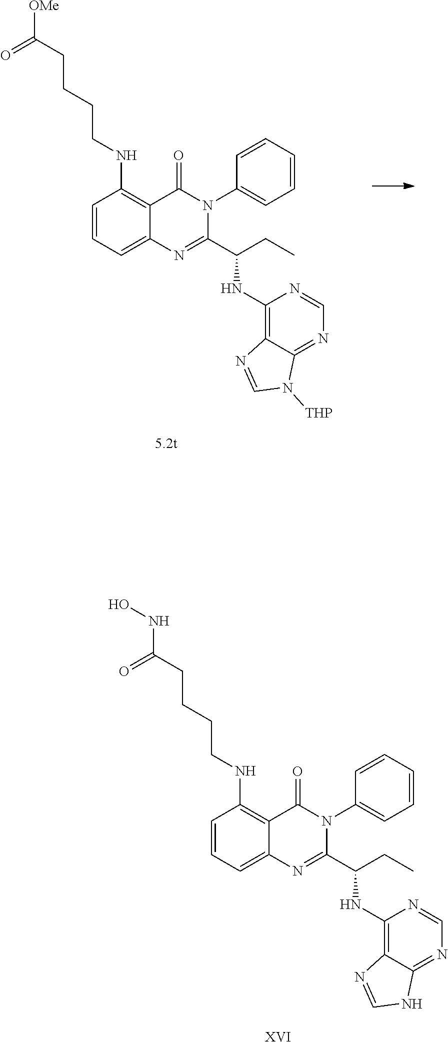

The procedure mentioned in Scheme 14 (Method B) was used with compound 5.2t (68.5 mg, 0.112 mmol) to afford product (S)-5-((2-(1-((9H-purin-6-yl)amino)propyl)-4-oxo-3-phenyl-3,4-dihydroquinazolin-5-yl)amino)-N-hydroxypentanamide, TFA XVI (42.0 mg, 0.065 mmol) in 58% yield. LC-MS (method 2): tR=3.69 min, m/z (M+H)+=528.3. 1H NMR (400 MHz, DMSO-d6) δ 10.33 (s, 1H), 8.71 (s, 1H), 8.44 (s, 2H), 7.52 (td, J=13.5, 5.2 Hz, 5H), 6.70 (d, J=7.9 Hz, 1H), 6.56 (d, J=8.4 Hz, 1H), 4.76 (s, 1H), 3.14 (d, J=5.7 Hz, 2H), 2.10-1.93 (m, 3H), 1.82 (dt, J=15.1, 7.6 Hz, 1H), 1.56 (dt, J=7.0, 3.5 Hz, 4H), 0.77 (t, J=7.3 Hz, 3H).

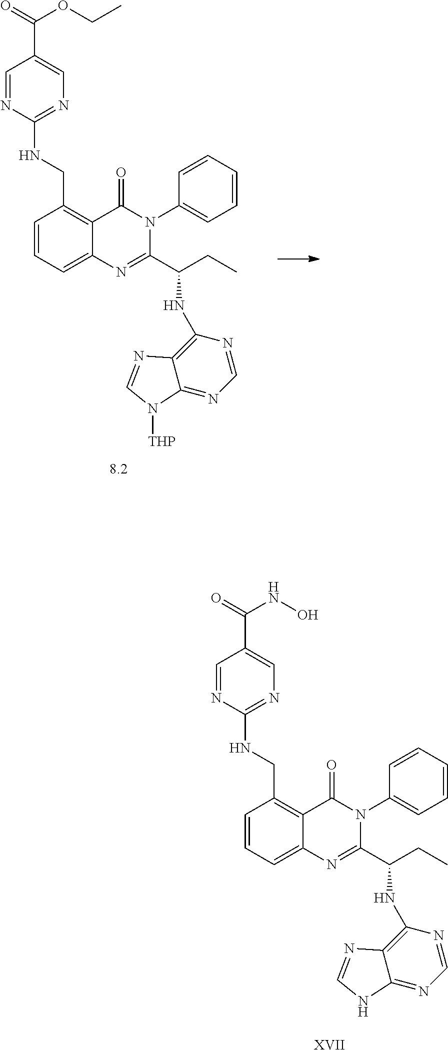

The procedure mentioned in Scheme 14 (Method A) was used with compound 8.2 (12.0 mg, 0.018 mmol) to afford product (S)-2-(((2-(1-((9H-purin-6-yl)amino)propyl)-4-oxo-3-phenyl-3,4-dihydroquinazolin-5-yl)methyl)amino)-N-hydroxypyrimidine-5-carboxamide, TFA XVII (7.0 mg, 0.010 mmol) in 57% yield. LC-MS (method 2): t_{R}=3.84 min, m/z (M+H)⁺=564.3.

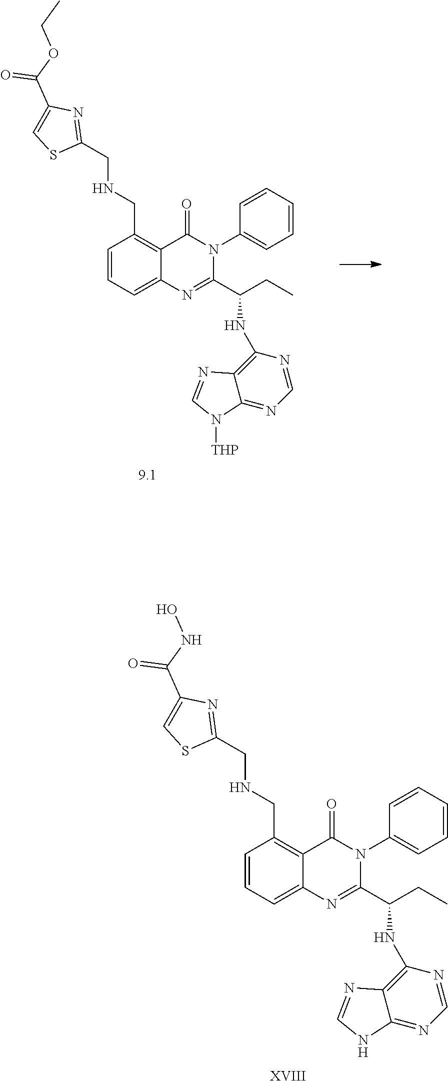

The procedure mentioned in Scheme 14 (Method B) was used with compound 9.1 (11.0 mg, 0.018 mmol) to afford product (S)-2-((((2-(1-((9H-purin-6-yl)amino)propyl)-4-oxo-3-phenyl-3,4-dihydroquinazolin-5-yl)methyl)amino)methyl)-N-hydroxythiazole-4-carboxamide, TFA XVIII (5.0 mg, 0.007 mmol) in 39% yield. LC-MS (method 2): t_{R}=3.14 min, m/z (M+H)⁺=583.2.

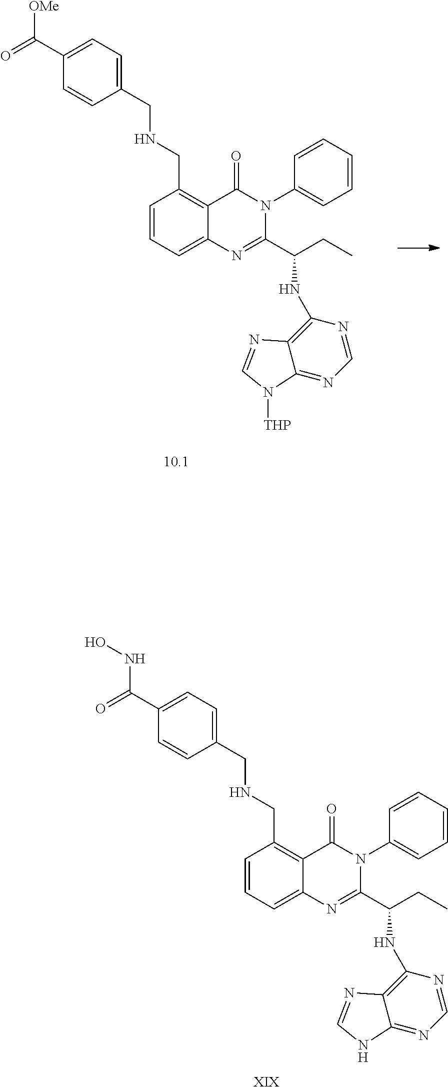

The procedure mentioned in Scheme 14 (Method A) was used with compound 10.1 (53.3 mg, 0.081 mmol) to afford product (S)-4-((((2-(1-((9H-purin-6-yl)amino)propyl)-4-oxo-3-phenyl-3,4-dihydroquinazolin-5-yl)methyl)amino)methyl)-N-hydroxybenzamide, TFA XIX (33.5 mg, 0.049 mmol) in 41% yield. LC-MS (method 2): t_{R}=3.09 min, m/z (M+H)⁺=576.3. 1H NMR (400 MHz, DMSO-d6) δ 11.27 (s, 1H), 8.99 (s, 2H), 8.26 (d, J=5.8 Hz, 2H), 8.18 (s, 1H), 7.85 (dd, J=8.3, 7.3 Hz, 1H), 7.81-7.71 (m, 3H), 7.67 (qt, J=6.2, 3.5 Hz, 2H), 7.59-7.50 (m, 5H), 4.78 (s, 1H), 4.58 (dd, J=11.5, 5.8 Hz, 2H), 4.29 (t, J=5.8 Hz, 2H), 1.97-1.85 (m, 2H), 0.77 (t, J=7.3 Hz, 3H).

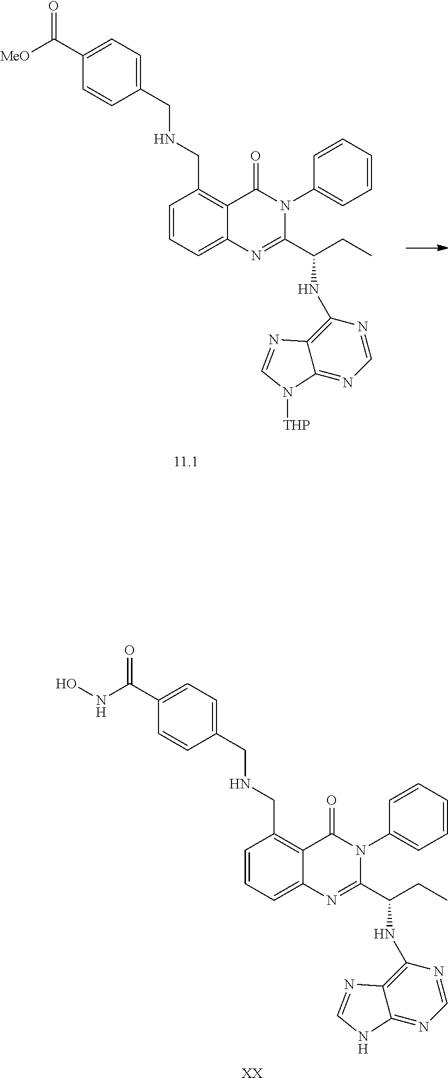

The procedure mentioned in Scheme 14 (Method A) was used with compound 11.1 (80.0 mg, 0.119 mmol) to afford product (S)—N1-((2-(1-((9H-purin-6-yl)amino)propyl)-4-oxo-3-phenyl-3,4-dihydroquinazolin-5-yl)methyl)-N4-hydroxyterephthalamide, TFA XX (26.0 mg, 0.037 mmol) in 31% yield. LC-MS (method 2): t_{R}=3.54 min, m/z (M+H)⁺=590.3. ¹H NMR (400 MHz, DMSO-d6) δ 11.35 (s, 1H), 9.04 (t, J=6.0 Hz, 1H), 8.56 (s, 1H), 8.39 (s, 2H), 7.98-7.94 (m, 2H), 7.86-7.82 (m, 2H), 7.76 (t, J=7.9 Hz, 1H), 7.65-7.48 (m, 6H), 7.41 (d, J=7.6 Hz, 1H), 5.02 (d, J=5.8 Hz, 2H), 4.79 (s, 1H), 2.03 (ddd, J=14.3, 7.5, 4.5 Hz, 1H), 1.89-1.82 (m, 1H), 0.78 (t, J=7.3 Hz, 3H).

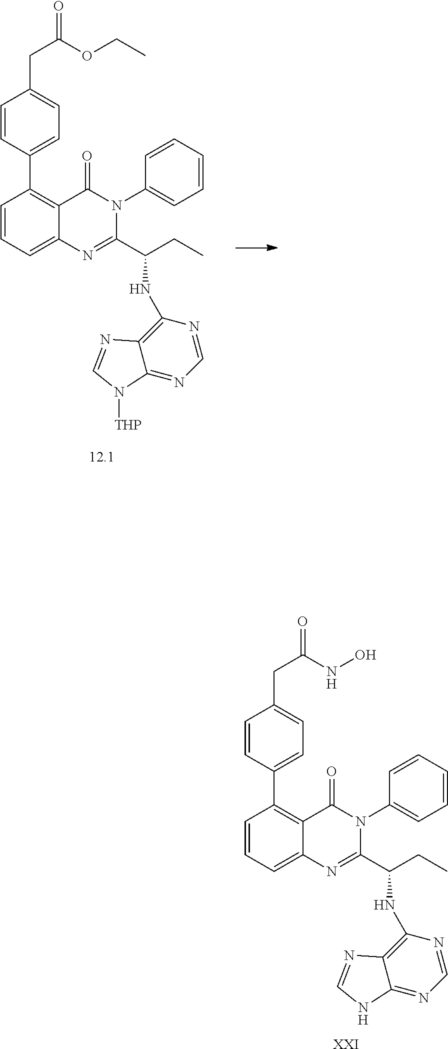

The procedure mentioned in Scheme 14 (Method A) was used with compound 12.1 (33.4 mg, 0.052 mmol) to afford product (S)-2-(4-(2-(1-((9H-purin-6-yl)amino)propyl)-4-oxo-3-phenyl-3,4-dihydroquinazolin-5-yl)phenyl)-N-hydroxyacetamide, TFA XXI (12.7 mg, 0.019 mmol) in 36% yield. LC-MS (method 2): t_{R}=3.66 min, m/z (M+H)⁺=546.2. ¹H NMR (400 MHz, DMSO-d6) δ 10.64 (s, 1H), 8.67 (s, 1H), 8.42 (s, 2H), 7.81 (t, J=7.8 Hz, 1H), 7.67 (dd, J=8.1, 1.3 Hz, 1H), 7.57-7.39 (m, 5H), 7.25 (dd, J=7.4, 1.3 Hz, 1H), 7.21-7.14 (m, 4H), 5.75 (s, 2H), 4.81 (s, 1H), 2.03 (dd, J=12.1, 6.8 Hz, 1H), 1.86 (dt, J=14.7, 7.7 Hz, 1H), 0.79 (t, J=7.3 Hz, 3H).

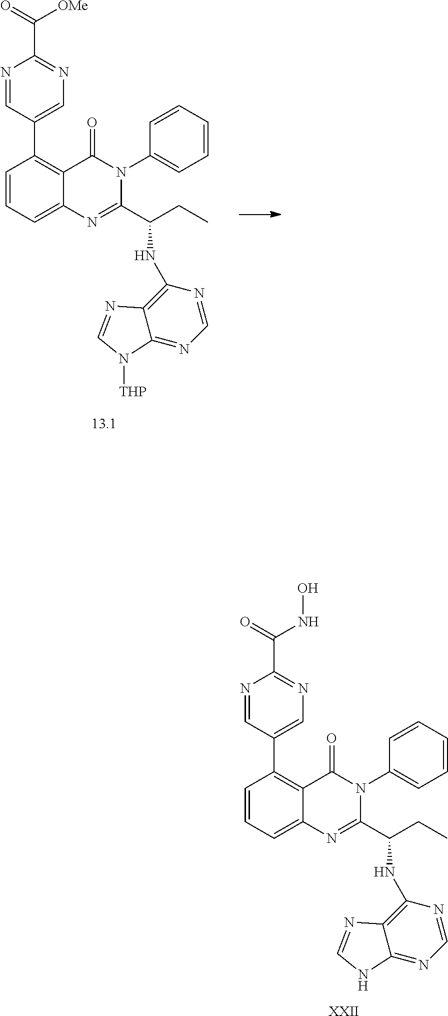

The procedure mentioned in Scheme 14 (Method A) was used with compound 13.1 (40.0 mg, 0.065 mmol) to afford product (S)-5-(2-(1-((9H-purin-6-yl)amino)propyl)-4-oxo-3-phenyl-3,4-dihydroquinazolin-5-yl)-N-hydroxypyrimidine-2-carboxamide, TFA XXII (14.7 mg, 0.023 mmol) in 35% yield. LC-MS (method 2): t_{R}=3.31 min, m/z (M+H)⁺=535.3. ¹H NMR (400 MHz, DMSO-d6) δ 11.53 (s, 1H), 8.85 (d, J=5.8 Hz, 2H), 8.50 (s, 1H), 8.37 (s, 2H), 7.93 (t, J=7.8 Hz, 1H), 7.80 (d, J=8.1 Hz, 1H), 7.61-7.40 (m, 6H), 4.81 (s, 1H), 2.06-1.97 (m, 1H), 1.94-1.84 (m, 1H), 0.78 (t, J=7.2 Hz, 3H).

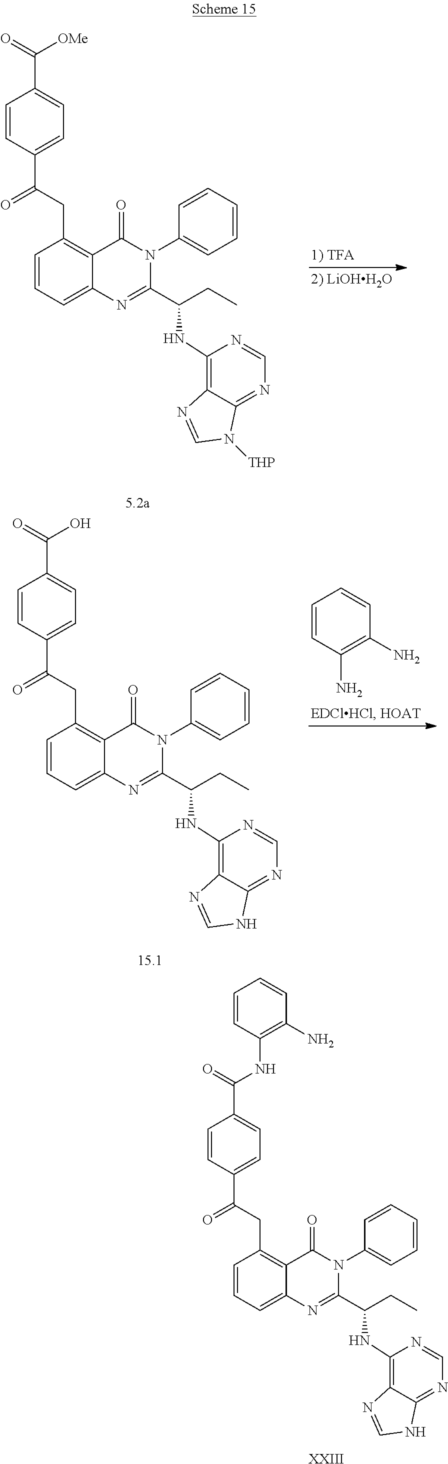

### Scheme 15

Compound 5.2a (69.0 mg, 0.105 mmol) was dissolved in DCM (1.0 ml) in a vial equipped with a stirring bar and TFA (239.4 mg, 2.1 mmol, 0.16 ml) was added dropwise to it. The resulting mixture was stirred at room temperature for 10 hours. After completion of reaction (by LC-MS), the crude reaction mixture is concentrated in vacuo, re-dissolved in 2 ml of DCM and passed through pre-conditioned PL-HCO₃ MP SPE device and washed with 2 ml of DCM. The filtrate was concentrated in vacuo and suspended in MeOH/water (1.0 ml, 1:1) in a vial equipped with a stir bar and LiOH.H₂O (8.8 mg, 0.21 mmol) was added to it. The resulting mixture was stirred at room temperature for 10 hours and concentrated in vacuo to afford crude 15.1. The crude compound 15.1 was suspended in DMF (1.0 ml) and benzene-1,2-diamine (15.4 mg, 0.14 mmol), N-methyl morpholine (28.7 mg, 0.28 mmol), 3-(((ethylimino)methylene)amino)-N,N-dimethylpropan-1-amine hydrochloride [EDC.HCl] (20.0 mg, 0.104 mmol) and 1H-[1,2,3]triazolo[4,5-b]pyridin-1-ol [HOAT] (13.5 mg, 0.10 mmol) were added to it. The resulting suspension was stirred at room temperature for 16 hours and concentrated in vacuo. Purified by C-18 reverse phase chromatography to afford the final compound (S)-4-(2-(2-(1-((9H-purin-6-yl)amino)propyl)-4-oxo-3-phenyl-3,4-dihydroquinazolin-5-yl)acetyl)-N-(2-aminophenyl)benzamide, TFA XXIII (26.5 mg, 0.035 mmol) in 33% yield. LC-MS (method 2): t_{R}=3.76 min, m/z (M+H)⁺=650.2.

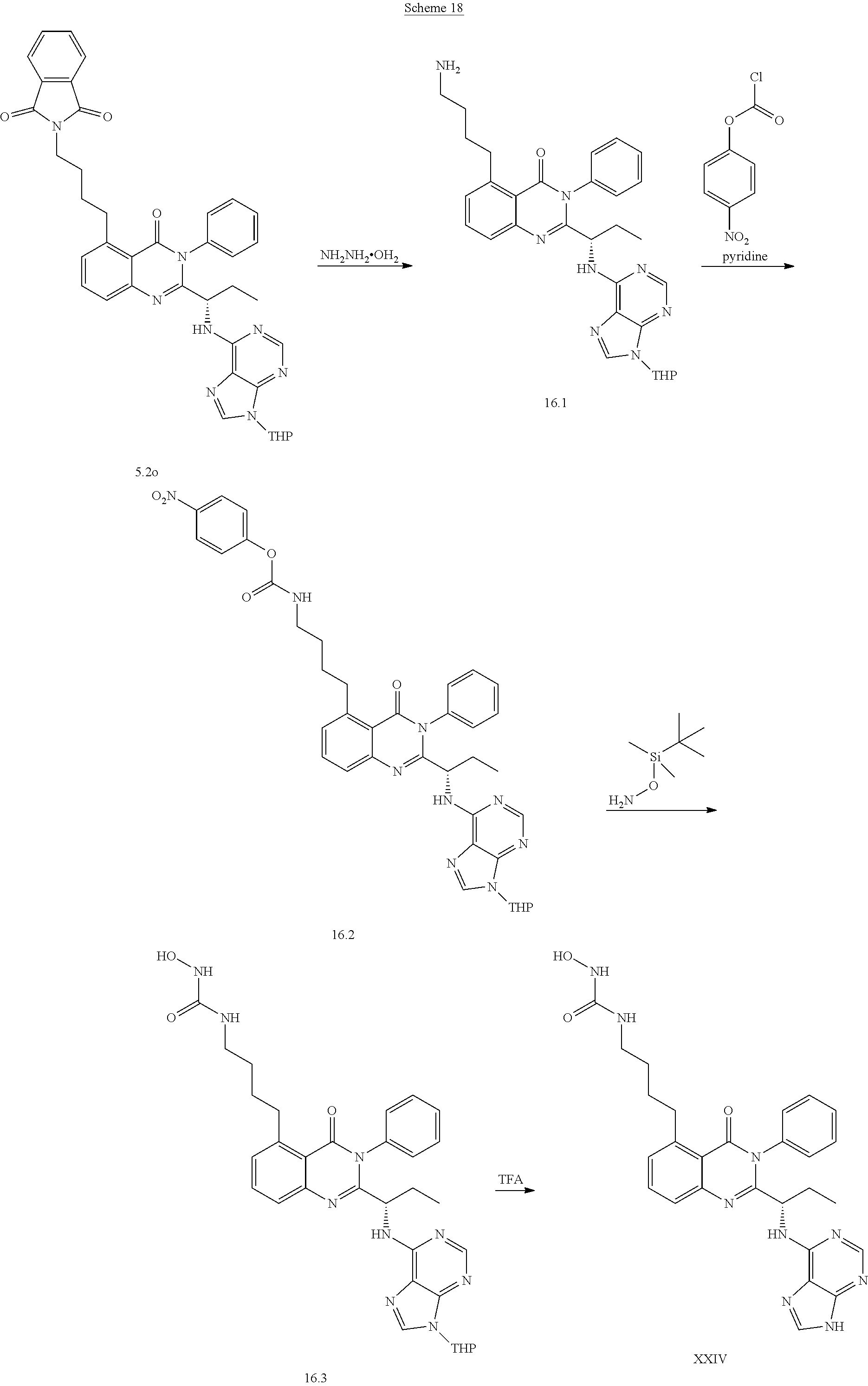

### Scheme 16

Compound 5.2o (171.0 mg, 0.25 mmol) was dissolved in THF (1.0 ml) in a MW vial equipped with a stir bar and hydrazine monohydrate (50.0 mg, 1.0 mmol) was added to it. The MW vial was sealed, heated at reflux for 2 hours and cooled down to room temperature. The crude material was filtered and washed with THF (2.0 ml). The filtrate was concentrated in vacuo to afford crude 16.1 that was dissolved in DCM (2.0 ml) and dry pyridine (79.0 mg, 1.0 mmol) and 4-nitrophenyl carbonochloridate (50.0 mg, 0.25 mmol) were added to it. The resulting mixture was stirred at room temperature for 2 hours and concentrated in vacuo to afford crude 16.2. Compound 16.2 was dissolved in acetonitrile (ml) and O-(tert-butyldimethylsilyl)hydroxylamine (55.2 mg, 0.38 mmol) was added to it. The resulting mixture was refluxed for 4 hours, concentrated in vacuo to provide crude 16.3 that was dissolved in DCM followed by dropwise addition of TFA (570.0 mg, 5.0 mmol). The resulting mixture was stirred at room temperature for 10 hours. After completion of reaction (by LC-MS), the crude reaction mixture is concentrated in vacuo and purified by C-18 reverse phase chromatography to afford product, TFA XXIV (19.5 mg, 0.03 mmol) in 12% yield. LC-MS (method 2): t_{R}=3.61 min, m/z (M+H)⁺=528.3.

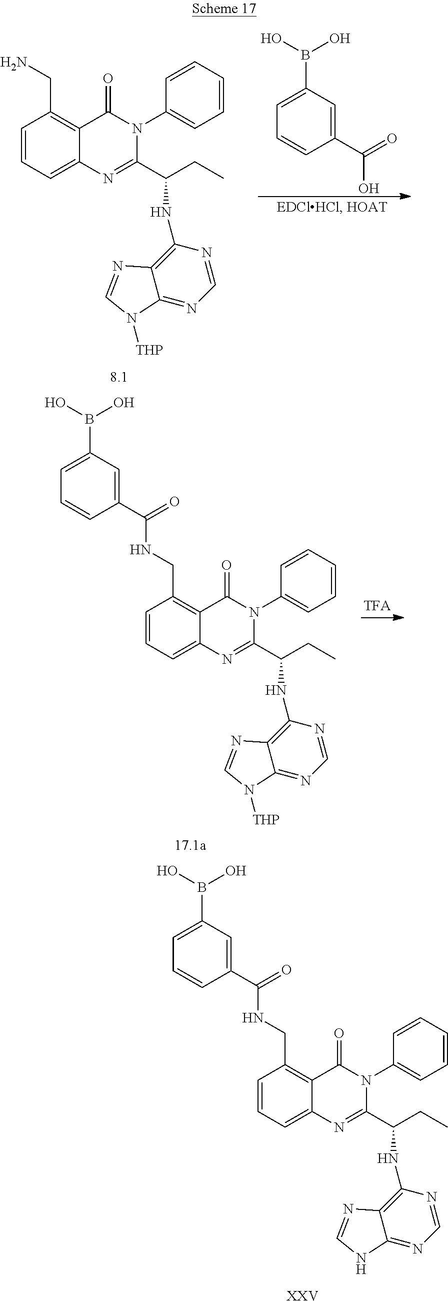

### Scheme 17

Compound 8.1 (66.1 mg, 0.13 mmol) was suspended in DMF (1.3 ml) and 3-boronobenzoic acid (32.2 mg, 0.194 mmol), N-methyl morpholine (39.3 mg, 0.39 mmol), 3-(((ethylimino)methylene)amino)-N,N-dimethylpropan-1-amine hydrochloride [EDC.HCl] (34.7 mg, 0.18 mmol) and 1H-[1,2,3]triazolo[4,5-b]pyridin-1-ol [HOAT] (21.1 mg, 0.16 mmol) were added to it. The resulting suspension was stirred at room temperature for 16 hours and concentrated in vacuo to provide crude 17.1a that was dissolved in DCM (1.3 ml) in a vial, followed by dropwise addition of TFA (294.0 mg, 2.58 mmol, 0.2 ml) into it. The resulting mixture was stirred for 10 hours, concentrated in vacuo and purified by C-18 reverse phase chromatography to afford the final compound (S)-(3-(((2-(1-((9H-purin-6-yl)amino)propyl)-4-oxo-3-phenyl-3,4-dihydroquinazolin-5-yl)methyl)carbamoyl)phenyl)boronic acid, TFA XXV (63.0 mg, 0.092 mmol) in 71% yield. LC-MS (method 2): t_{R}=3.83 min, m/z (M+H)⁺=575.3. 1H NMR (400 MHz, DMSO-d6) δ 8.87 (t, J=6.0 Hz, 1H), 8.46 (s, 2H), 8.30 (t, J=1.6 Hz, 1H), 7.97-7.88 (m, 2H), 7.77 (t, J=7.9 Hz, 2H), 7.59 (dt, J=14.3, 5.2 Hz, 4H), 7.52 (s, 1H), 7.49-7.39 (m, 3H), 5.75 (s, 1H), 5.00 (d, J=5.9 Hz, 2H), 4.81 (s, 1H), 2.05 (ddd, J=14.3, 7.4, 4.4 Hz, 1H), 1.86 (dt, J=14.6, 7.7 Hz, 1H), 0.79 (t, J=7.3 Hz, 3H).

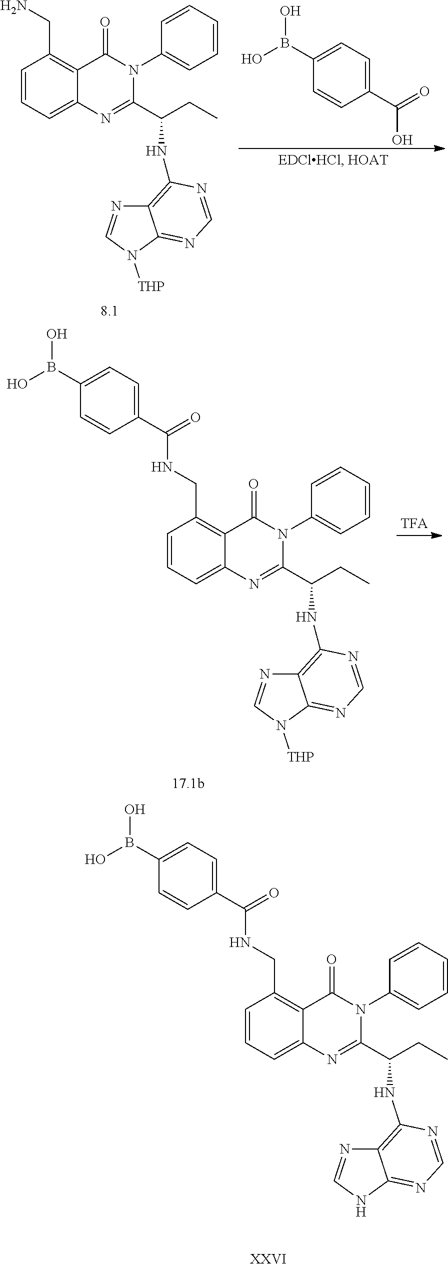

Compound 8.1 (84.7 mg, 0.17 mmol) was suspended in DMF (1.7 ml) and 4-boronobenzoic acid (41.3 mg, 0.25 mmol), N-methyl morpholine (50.3 mg, 0.50 mmol), 3-(((ethylimino)methylene)amino)-N,N-dimethylpropan-1-amine hydrochloride [EDC.HCl] (44.5 mg, 0.23 mmol) and 1H-[1,2,3]triazolo[4,5-b]pyridin-1-ol [HOAT] (27.1 mg, 0.20 mmol) were added to it. The resulting suspension was stirred at room temperature for 16 hours and concentrated in vacuo to provide crude 17.1b that was dissolved in DCM (1.7 ml) in a vial, followed by dropwise addition of TFA (387.7 mg, 3.4 mmol, 0.26 ml) into it. The resulting mixture was stirred for 10 hours, concentrated in vacuo and purified by C-18 reverse phase chromatography to afford the final compound (S)-(3-(((2-(1-((9H-purin-6-yl)amino)propyl)-4-oxo-3-phenyl-3,4-dihydroquinazolin-5-yl)methyl)carbamoyl)phenyl)boronic acid, TFA XXVI (75.5 mg, 0.11 mmol) in 66% yield. LC-MS (method 2): t_{R}=3.78 min, m/z (M+H)⁺=575.3. ¹H NMR (400 MHz, DMSO-d6) δ 8.93 (t, J=6.0 Hz, 1H), 8.84 (s, 1H), 8.47 (s, 1H), 7.92-7.80 (m, 4H), 7.77 (t, J=7.9 Hz, 1H), 7.64-7.48 (m, 6H), 7.42 (dd, J=7.6, 1.2 Hz, 1H), 5.01 (d, J=5.9 Hz, 2H), 4.81 (s, 1H), 2.05 (ddd, J=14.2, 7.2, 4.3 Hz, 1H), 1.86 (dt, J=14.6, 7.7 Hz, 1H), 0.79 (t, J=7.3 Hz, 3H).

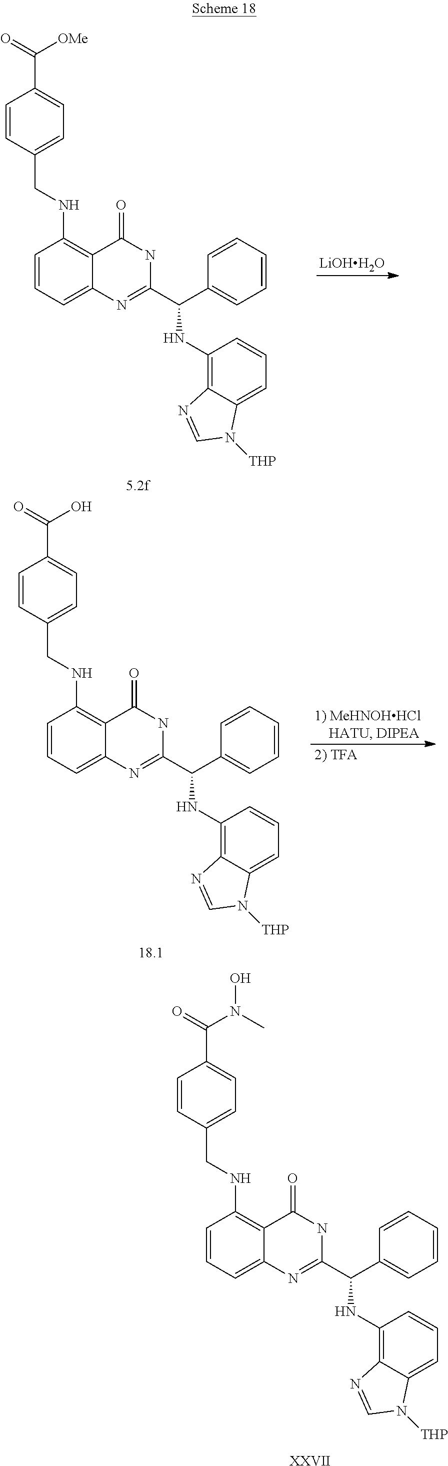

### Scheme 18

Compound 5.2f (40.6 mg, 0.063) was suspended in MeOH/water (1.2 ml, 1:1) in a vial equipped with a stir bar and LiOH.H₂O (5.3 mg, 0.13 mmol) was added to it. The resulting mixture was stirred at room temperature for 10 hours and concentrated in vacuo to afford crude 18.1. This crude compound 18.1 was dissolved in DMF (1.2 ml) in a vial equipped with a stir bar and N-methylhydroxylamine hydrochloride (8.0 mg, 0.95 mmol), DIPEA (18.0 mg, 0.16 mmol) and 2-(3H-[1,2,3]triazolo[4,5-b]pyridin-3-yl)-1,1,3,3-tetramethylisouronium hexafluorophosphate(V) [HATU] (36.0 mg, 0.095 mmol) were added to it. The resulting mixture was stirred at room temperature for 16 hours, concentrated in vacuo and re-dissolved in DCM (1.2 ml) in a vial followed by dropwise addition of TFA (144.0 mg, 1.26 mmol, 0.10 ml) into it. The resulting mixture was stirred for 10 hours, concentrated in vacuo and purified by C-18 reverse phase chromatography to afford the final compound (S)-4-(((2-(1-((9H-purin-6-yl)amino)propyl)-4-oxo-3-phenyl-3,4-dihydroquinazolin-5-yl)amino)methyl)-N-hydroxy-N-methylbenzamide, TFA XXVII (13.0 mg, 0.019 mmol) in 30% yield. LC-MS (method 2): t_{R}=4.15 min, m/z (M+H)⁺=576.3. ¹H NMR (400 MHz, DMSO-d6) δ 9.95 (s, 1H), 8.96 (s, 1H), 8.54 (s, 1H), 8.39 (s, 1H), 7.57 (dt, J=6.6, 1.9 Hz, 4H), 7.47 (dd, J=10.6, 5.7 Hz, 3H), 7.40-7.32 (m, 2H), 6.73 (d, J=7.8 Hz, 1H), 6.50 (dd, J=8.5, 1.0 Hz, 1H), 4.77 (s, 1H), 4.48 (s, 2H), 3.22 (s, 3H), 1.99 (ddd, J=11.7, 7.4, 3.7 Hz, 1H), 1.87-1.78 (m, 1H), 0.77 (t, J=7.3 Hz, 3H).

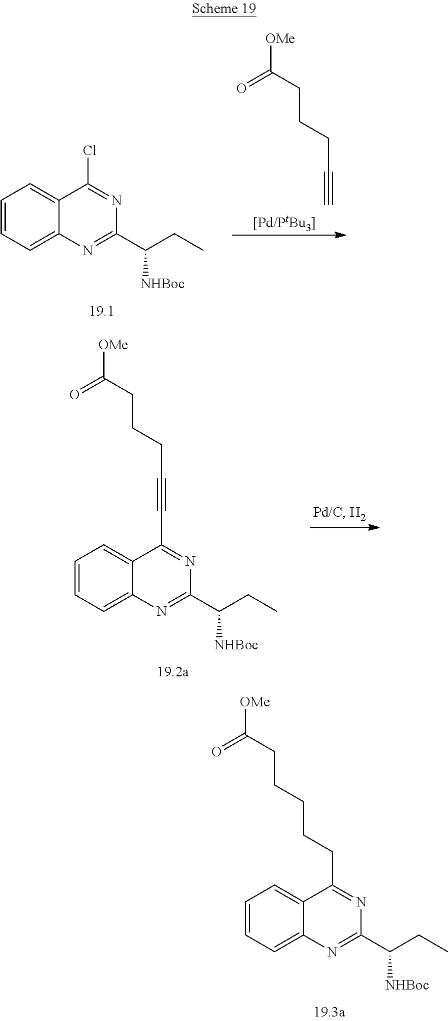

### Scheme 19

(S)-tert-butyl (1-(4-chloroquinazolin-2-yl)propyl)carbamate 19.1 (225.0 mg, 0.70 mmol, Cakici, M. et al. Tetrahedron: Asymmetry 2011, 22, 300), Allylpalladium(II) chloride dimer (13.0 mg, 0.035) and Tri-tert-butylphosphonium tetrafluoroborate (20.0 mg, 0.07 mmol) were added to a MW vial equipped with a stir bar. The vial was covered with a rubber septum and placed under nitrogen atmosphere. In a separate scintillation vial, DABCO (157.0 mg, 1.4 mmol) was dissolved in dry 1,4-dioxane (3.5 ml). This DABCO solution and methyl hex-5-ynoate (115.0 mg, 0.91 mmol) were added to the microwave vial via syringe and the resulting mixture is bubbled with nitrogen for 5 min followed by stirring for 16 hours at room temperature under nitrogen atmosphere. After 16 hours, the crude reaction mixture is filtered through a short pad of celite and concentrated in vacuo. The remaining residue was purified by flash chromatography on silica using 0-30% ethyl acetate/hexanes system to afford the product methyl (S)-6-(2-(1-((tert-butoxycarbonyl)amino)propyl)quinazolin-4-yl)hex-5-ynoate 19.2a (163.6 mg, 0.398 mmol) as a yellow oil in 57% yield. LC-MS (method 1): t_{R}=3.51 min, m/z (M+H)⁺=412.3. 1H NMR (400 MHz, Chloroform-d) δ 8.26 (ddd, J=8.3, 1.5, 0.7 Hz, 1H), 8.03-7.97 (m, 1H), 7.90 (ddd, J=8.4, 6.9, 1.4 Hz, 1H), 7.65 (ddd, J=8.2, 6.8, 1.2 Hz, 1H), 5.91 (d, J=8.2 Hz, 1H), 5.03 (d, J=6.9 Hz, 1H), 3.72 (s, 3H), 2.74 (t, J=7.1 Hz, 2H), 2.60 (t, J=7.3 Hz, 2H), 2.10 (p, J=7.2 Hz, 2H), 1.90 (dt, J=13.6, 7.2 Hz, 2H), 1.46 (s, 9H), 0.91 (dd, J=8.2, 7.0 Hz, 3H).

Compound 19.2a (120.0 mg, 0.292 mmol) and 10 wt % Pd/C (12.0 mg) were added to a round-bottomed flask fitted with a rubber septum. The reaction flask is evacuated followed by the addition of dry EtOAc (2.9 ml). The vacuum is removed and the reaction flask is kept under an atmosphere of hydrogen using a balloon and was stirred for 20 h. After completion of reaction (by LC-MS), the crude reaction mixture is filtered using celite, concentrated in vacuo to afford the product methyl (S)-6-(2-(1-((tert-butoxycarbonyl)amino)propyl)quinazolin-4-yl)hexanoate 19.3a (119.0 mg, 0.286 mmol) in 98% yield. LC-MS (method 1): t_{R}=3.66 min, m/z (M+H)⁺=415.3.

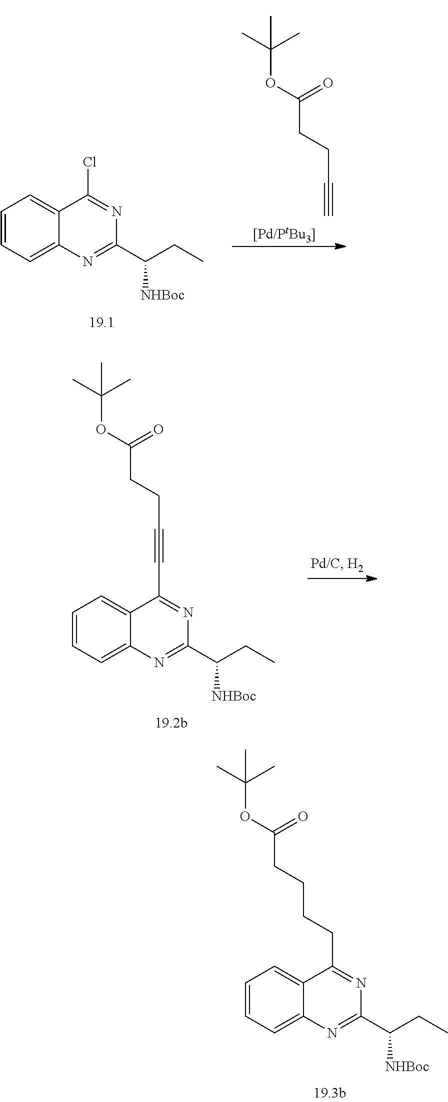 [SMILES: CC[C@H](NC(=O)OC(C)(C)C)c1nc(Cl)c2ccccc2n1 ; C#CCCC(=O)OC(C)(C)C ; CC[C@H](NC(=O)OC(C)(C)C)c1nc(C#CCCC(=O)OC(C)(C)C)c2ccccc2n1 ; CC[C@H](NC(=O)OC(C)(C)C)c1nc(CCCCC(=O)OC(C)(C)C)c2ccccc2n1]

The procedure mentioned in Scheme 19 was used with (S)-tert-butyl (1-(4-chloroquinazolin-2-yl)propyl)carbamate 19.1 (180.0 mg, 0.56 mmol), Allylpalladium(II) chloride dimer (10.1 mg, 0.028), Tri-tert-butylphosphonium tetrafluoroborate (16.0 mg, 0.06 mmol), DABCO (125.0 mg, 1.12 mmol) and tert-butyl pent-4-ynoate (104.0 mg, 0.67 mmol) in dry 1,4-dioxane (2.8 ml). The resulting mixture is stirred at room temperature for 16 hours under nitrogen atmosphere. After 16 hours, the crude reaction mixture is filtered through a short pad of celite and concentrated in vacuo. The remaining residue was purified by flash chromatography on silica using 0-25% ethyl acetate/hexanes system to afford the product (S)-tert-butyl 5-(2-(1-((tert-butoxycarbonyl)amino)propyl)quinazolin-4-yl)pent-4-ynoate 19.2b (150.0 mg, 0.34 mmol) as a yellow oil in 61% yield. LC-MS (method 1): t_{R}=3.85 min, m/z (M+H)⁺=440.3. 1H NMR (400 MHz, Chloroform-d) δ 8.29-8.24 (m, 1H), 7.99 (d, J=8.5 Hz, 1H), 7.88 (ddd, J=8.5, 6.9, 1.4 Hz, 1H), 7.62 (ddd, J=8.3, 6.9, 1.2 Hz, 1H), 5.90 (d, J=8.2 Hz, 1H), 5.02 (d, J=7.3 Hz, 1H), 2.91 (t, J=7.4 Hz, 2H), 2.69 (t, J=7.1 Hz, 2H), 2.15-2.02 (m, 1H), 1.89 (dt, J=14.0, 7.3 Hz, 1H), 1.49 (s, 9H), 1.47 (s, 9H) 0.90 (t, J=7.4 Hz, 3H).

Compound 19.2a (102.0 mg, 0.192 mmol) and 10 wt % Pd/C (10.0 mg) were added to a round-bottomed flask fitted with a rubber septum. The reaction flask is evacuated followed by the addition of dry EtOAc (1.9 ml). The vacuum is removed and the reaction flask is kept under an atmosphere of hydrogen using a balloon and was stirred for 20 h. After completion of reaction (by LC-MS), the crude reaction mixture is filtered using celite, concentrated in vacuo to afford the product tert-butyl (S)-5-(2-(1-((tert-butoxycarbonyl)amino)propyl)-4-oxo-3-phenyl-3,4-dihydroquinazolin-5-yl)pentanoate 19.3b (101.0 mg, 0.19 mmol) in 98% yield. LC-MS (method 1): t_{R}=4.04 min, m/z (M+H)⁺=536.3.

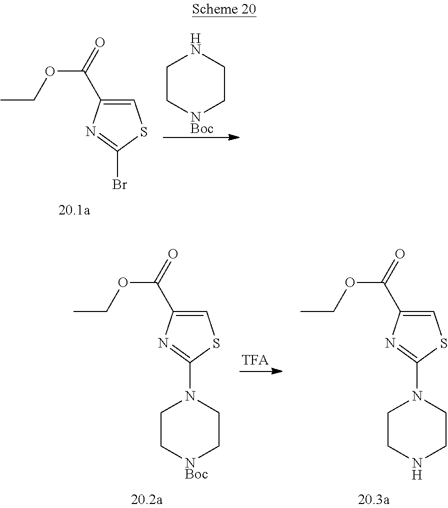

### Scheme 20

Ethyl 2-bromothiazole-4-carboxylate 20.1a (100.0 mg, 0.42 mmol) was dissolved in n-butanol (1.0 ml) in a microwave vial equipped with a stir bar and tert-butyl piperazine-1-carboxylate (83.0 mg, 0.44 mmol) was added to it. The vial was sealed and heated at 150° C. for 20 min in a microwave. After completion of reaction (by LC-MS), the reaction mixture was concentrated in vacuo and the remaining residue was purified by flash chromatography on silica gel using 0-30% ethyl acetate/hexanes to afford the product ethyl 2-(4-(tert-butoxycarbonyl)piperazin-1-yl)thiazole-4-carboxylate 20.2a (92.2 mg, 0.27 mmol) in 64% yield). LC-MS (method 1): t_{R}=3.36 min, m/z (M+H)⁺=342.2.

Compound 20.2a (92.2 mg, 0.27 mmol) was dissolved in DCM (2.7 ml) and TFA (616.0 mg, 5.4 mmol, 0.41 ml) was added dropwise to it. The resulting mixture was stirred at room temperature for 3 hours. After the completion of reaction (by LC-MS), the reaction mixture was concentrated in vacuo to afford the product ethyl 2-(piperazin-1-yl)thiazole-4-carboxylate, TFA 20.3a. LC-MS (method 1): t_{R}=2.18 min, m/z (M+H)⁺=242.1.

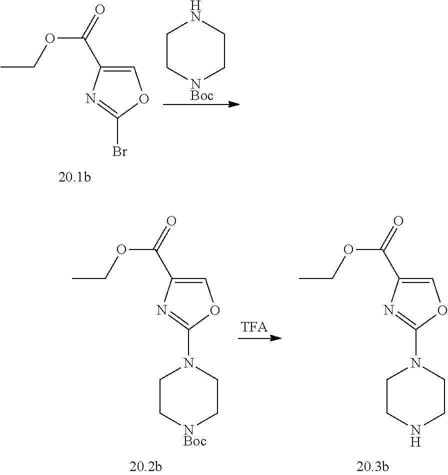

Ethyl 2-bromooxazole-4-carboxylate 20.1b (92.2 mg, 0.42 mmol) was dissolved in 1,4-dioxane (1.0 ml) in a microwave vial equipped with a stir bar and tert-butyl piperazine-1-carboxylate (94.0 mg, 0.5 mmol) and triethylamine (127.0 mg, 1.26 mmol) were added to it. The vial was sealed and heated at 120° C. for 1 hour in a microwave. After completion of reaction (by LC-MS), the reaction mixture was concentrated in vacuo and the remaining residue was purified by flash chromatography on silica gel using 0-5% MeOH/DCM to afford the product ethyl 2-(4-(tert-butoxycarbonyl)piperazin-1-yl)oxazole-4-carboxylate 20.2b (125.0 mg, 0.384 mmol) in 92% yield). LC-MS (method 1): t_{R}=3.25 min, m/z (M+H)⁺=326.2.

Compound 20.2b (125.0 mg, 0.384 mmol) was dissolved in DCM (3.8 ml) and TFA (876.0 mg, 7.68 mmol, 0.56 ml) was added dropwise to it. The resulting mixture was stirred at room temperature for 3 hours. After the completion of reaction (by LC-MS), the reaction mixture was concentrated in vacuo to afford the product(125.0 mg, 0.384 mmol), TFA 20.3a. LC-MS (method 1): t_{R}=2.07 min, m/z (M+H)⁺=226.1.

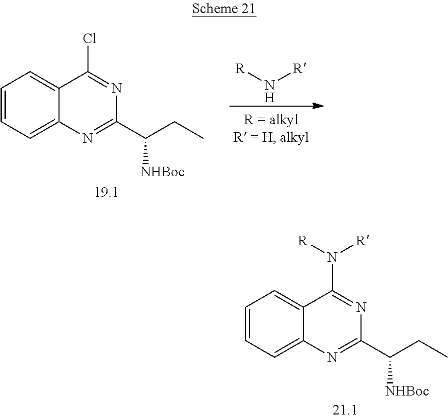

### Scheme 21

Compound 19.1a (1 equiv) was dissolved in ethanol (0.4 M) in a microwave vial equipped with a stir bar and alkyl amine [HNRR′] (1.5-2.0 equiv) and triethylamine (3.0-4.0 equiv) was added to it. The vial was sealed and heated at 100° C. for 1 hour in a microwave. After completion of reaction (by LC-MS), the reaction mixture was concentrated in vacuo and the remaining residue was purified by flash chromatography on silica using forced flow of indicated solvent system on Biotage KP-Sil pre-packed cartridges and using the Biotage SP-1 automated chromatography system to afford the coupled product 21.1.

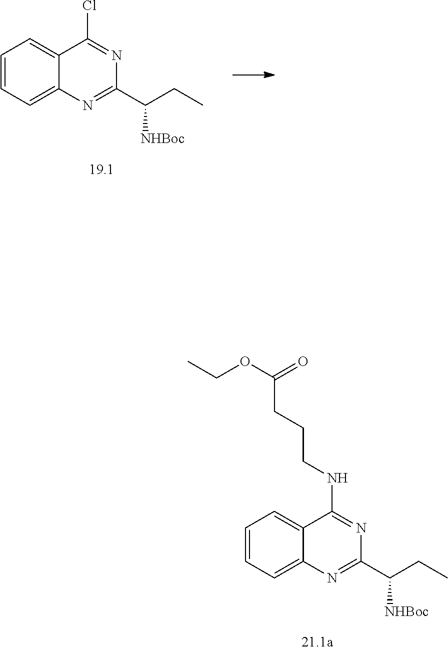 [SMILES: CC[C@H](NC(=O)OC(C)(C)C)c1nc(Cl)c2ccccc2n1 ; CCOC(=O)CCCNc1nc([C@H](CC)NC(=O)OC(C)(C)C)nc2ccccc12]

The procedure mentioned in Scheme 20 was used with (S)-tert-butyl (1-(4-chloroquinazolin-2-yl)propyl)carbamate 19.1 (103.0 mg, 0.32 mmol), ethyl 4-aminobutyrate hydrochloride (107.0 mg, 0.64 mmol) and triethylamine (130.0 mg, 1.28 mmol, 0.18 ml) in ethanol (0.7 ml). The remaining residue was purified on silica using 0-30% EtOAc/hexanes to afford (S)-ethyl 4-((2-(1-((tert-butoxycarbonyl)amino)propyl)quinazolin-4-yl)amino)butanoate 21.1a (125.0 mg, 0.30 mmol) in 94% yield. LC-MS (method 1): t_{R}=2.85 min, m/z (M+H)⁺=417.3. 1H NMR (400 MHz, Chloroform-d) δ 7.78 (d, J=8.4 Hz, 1H), 7.75-7.63 (m, 2H), 7.40 (t, J=7.6 Hz, 1H), 6.51 (s, 1H), 6.04 (d, J=7.9 Hz, 1H), 4.75 (q, J=6.6 Hz, 1H), 4.12 (q, J=7.2 Hz, 2H), 3.71 (q, J=6.2 Hz, 2H), 2.50 (t, J=6.7 Hz, 2H), 2.07 (p, J=6.8 Hz, 2H), 2.03-1.97 (m, 1H), 1.88 (dt, J=13.8, 7.0 Hz, 1H), 1.47 (s, 9H), 1.22 (t, J=7.1 Hz, 3H), 0.89 (t, J=7.5 Hz, 3H).

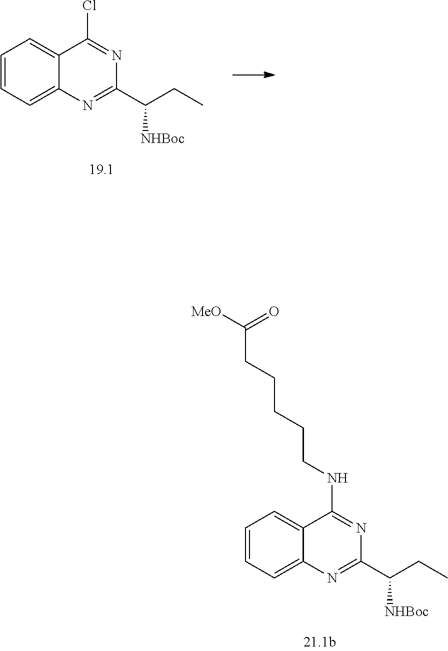 [SMILES: CC[C@H](NC(=O)OC(C)(C)C)c1nc(Cl)c2ccccc2n1 ; CC[C@H](NC(=O)OC(C)(C)C)c1nc(NCCCCCC(=O)OC)c2ccccc2n1]

The procedure mentioned in Scheme 20 was used with (S)-tert-butyl (1-(4-chloroquinazolin-2-yl)propyl)carbamate 19.1 (200.0 mg, 0.62 mmol), 6-methoxy-6-oxohexan-1-aminium chloride (169.0 mg, 0.932 mmol) and triethylamine (189.0 mg, 1.86 mmol, 0.26 ml) in ethanol (1.5 ml). The remaining residue was purified on silica using 0-5% MeOH/DCM to afford methyl (S)-6-((2-(1-((tert-butoxycarbonyl)amino)propyl)quinazolin-4-yl)amino)hexanoate 21.1b (252.0 mg, 0.585 mmol) in 94% yield. LC-MS (method 1): t_{R}=2.92 min, m/z (M+H)⁺=431.3. 1H NMR (400 MHz, Chloroform-d) δ 7.82-7.75 (m, 1H), 7.69 (q, J=8.5, 8.0 Hz, 2H), 7.39 (t, J=7.6 Hz, 1H), 6.03 (d, J=10.3 Hz, 1H), 4.75 (q, J=6.6 Hz, 1H), 3.67 (m, J=1.3 Hz, 4H), 2.35 (t, J=7.3 Hz, 2H), 2.02 (dq, J=14.0, 6.8 Hz, 1H), 1.88 (dt, J=14.1, 6.7 Hz, 1H), 1.79-1.64 (m, 4H), 1.47 (s, 9H), 1.44 (t, J=4.4 Hz, 2H), 0.90 (t, J=7.4 Hz, 3H).

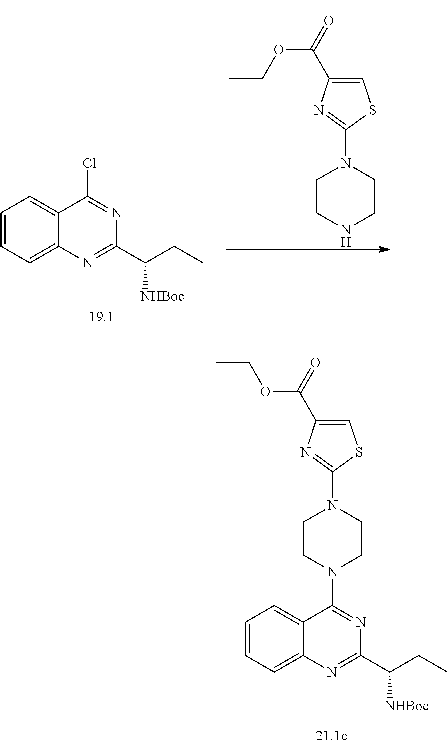 [SMILES: CC[C@H](NC(=O)OC(C)(C)C)c1nc(Cl)c2ccccc2n1 ; CCOC(=O)c1csc(N2CCNCC2)n1 ; CCOC(=O)c1csc(N2CCN(c3nc([C@H](CC)NC(=O)OC(C)(C)C)nc4ccccc34)CC2)n1]

The procedure mentioned in Scheme 20 was used with (S)-tert-butyl (1-(4-chloroquinazolin-2-yl)propyl)carbamate 19.1 (62.0 mg, 0.193 mmol), ethyl 2-(piperazin-1-yl)thiazole-4-carboxylate, TFA (65.0 mg, 0.27 mmol) and triethylamine (58.0 mg, 0.58 mmol, 81.0 μl) in ethanol (0.4 ml). The remaining residue was purified on silica using 0-5% MeOH/DCM to afford ethyl (S)-2-(4-(2-(1-((tert-butoxycarbonyl)amino)propyl)quinazolin-4-yl)piperazin-1-yl)thiazole-4-carboxylate 21.1c (92.0 mg, 0.175 mmol) in 91% yield. LC-MS (method 1): t_{R}=2.93 min, m/z (M+H)⁺=527.3.

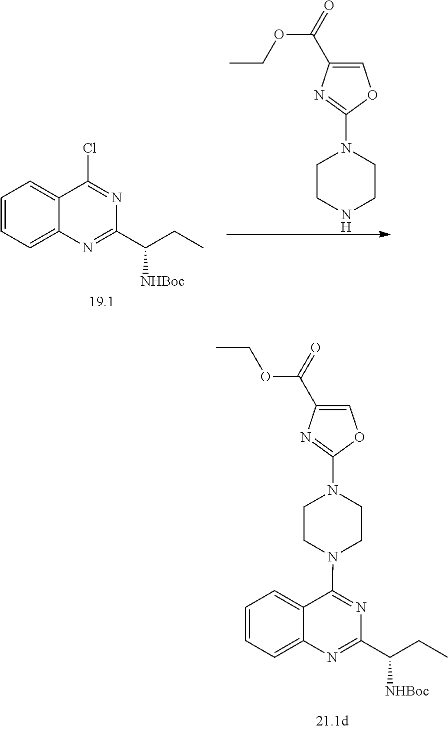 [SMILES: CC[C@H](NC(=O)OC(C)(C)C)c1nc(Cl)c2ccccc2n1 ; CCOC(=O)c1coc(N2CCNCC2)n1 ; CCOC(=O)c1coc(N2CCN(c3nc([C@H](CC)NC(=O)OC(C)(C)C)nc4ccccc34)CC2)n1]

The procedure mentioned in Scheme 20 was used with (S)-tert-butyl (1-(4-chloroquinazolin-2-yl)propyl)carbamate 19.1 (124.0 mg, 0.384 mmol), ethyl 2-(piperazin-1-yl)oxaazole-4-carboxylate, TFA (130.0 mg, 0.58 mmol) and triethylamine (117.0 mg, 1.15 mmol, 0.16 ml) in ethanol (1.0 ml). The remaining residue was purified on silica using 0-5% MeOH/DCM to afford ethyl (S)-2-(4-(2-(1-((tert-butoxycarbonyl)amino)propyl)quinazolin-4-yl)piperazin-1-yl)oxazole-4-carboxylate 21.1d (140.0 mg, 0.27 mmol) in 71% yield. LC-MS (method 1): t_{R}=2.92 min, m/z (M+H)⁺=511.3.

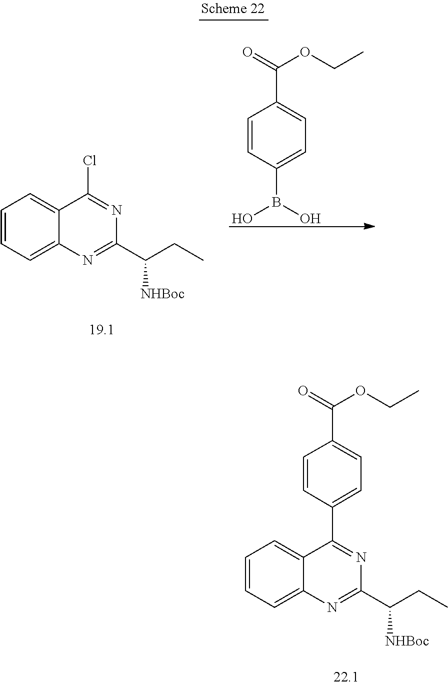 [SMILES: CC[C@H](NC(=O)OC(C)(C)C)c1nc(Cl)c2ccccc2n1 ; CCOC(=O)c1ccc(B(O)O)cc1 ; CCOC(=O)c1ccc(-c2nc([C@H](CC)NC(=O)OC(C)(C)C)nc3ccccc23)cc1]

### Scheme 22

Compound 19.1 (133.0 mg, 0.41 mmol), chloro(crotyl)(2-dicyclohexylphosphino-2′,4′,6′-triisopropybiphenyl)palladium(II) [Pd-170] (14.0 mg, 0.02 mmol) and (4-(ethoxycarbonyl)phenyl)boronic acid (96.0 mg, 0.50 mmol) were suspended in 1,4-dioxane (2.0 ml) in a MW vial equipped with a stir bar under N₂ atmosphere and potassium phosphate (263.0 mg, 1.24 mmol) was added to it. The MW vial was sealed and heated at 100° C. for 2 hours in a MW reactor. The reaction mixture was allowed to cool to room temperature, quenched with water, and then extracted three times with ethyl acetate. The combined organic fractions were dried over MgSO₄ and then concentrated in vacuo. The remaining residue was purified by flash chromatography on silica using 0-25% ethyl acetate/hexanes to afford the product ethyl (S)-4-(2-(1-((tert-butoxycarbonyl)amino)propyl)quinazolin-4-yl)benzoate 22.1 (100.0 mg, 0.23 mmol) in 56% yield. LC-MS (method 1): t_{R}=3.86 min, m/z (M+H)⁺=436.2.

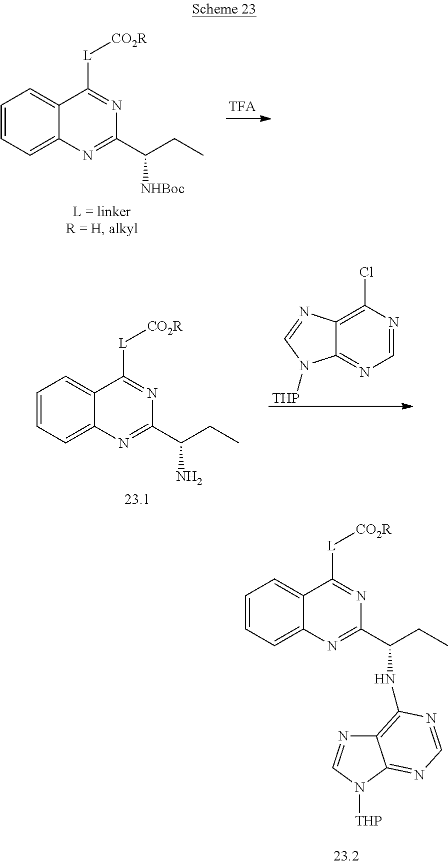

### Scheme 23

The Boc-protected amine (1 equiv) was dissolved in DCM (0.1 M) in a vial and trifluoroacetic acid (20 equiv) was added dropwise to it. The resulting mixture was stirred at room temperature for 3 hours. After completion of reaction (by LC-MS) the reaction mixture is worked-up by either of the following two methods:

Method A: The crude reaction is quenched with aqueous saturated NaHCO₃ solution and extracted three times with DCM. The combined organic layers were dried over anhydrous MgSO₄, filtered and concentrated in vacuo to afford the free amine 23.1.

Method B: The crude reaction mixture is concentrated in vacuo, re-dissolved in 1-2 ml of DCM and passed through pre-conditioned PL-HCO₃ MP SPE device and washed with 2 ml of DCM. The filtrate was concentrated in vacuo to afford the free amine 23.1.

The free amine 23.1 was dissolved in ethanol (0.4 M) in a microwave vial equipped with a stir bar followed by the addition of 6-chloro-9-(tetrahydro-2H-pyran-2-yl)-9H-purine (1.5-2.0 equiv) and triethylamine (3.0-4.0 equiv) to it. The vial was sealed and heated for 4 hours at 100° C. in a microwave. After completion of reaction (by LC-MS), the reaction mixture was concentrated in vacuo and the remaining residue was purified by flash chromatography on silica gel using forced flow of indicated solvent system on Biotage KP-Sil pre-packed cartridges and using the Biotage SP-1 automated chromatography system to afford the coupled product 23.2.

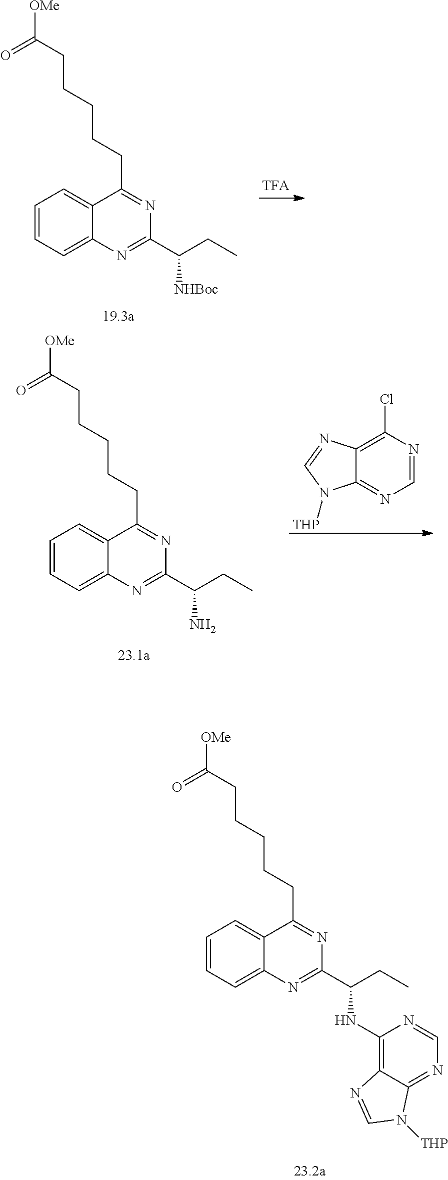

The procedure mentioned in Scheme 23 was used with compound 19.3a (141.0 mg, 0.34 mmol) and trifluoroacetic acid (775.0 mg, 6.28 mmol, 0.52 ml) in dichloromethane (3.4 ml). The resulting mixture was stirred at room temperature for 3 hours and worked-up (Method A) to afford (S)-methyl 6-(2-(1-((tert-butoxycarbonyl)amino)propyl)quinazolin-4-yl)hexanoate 23.1a. This free amine 23.1a was used with 6-chloro-9-(tetrahydro-2H-pyran-2-yl)-9H-purine (162.0 mg, 0.68 mmol) and triethylamine (138.0 mg, 0.36 mmol, 190 μl) in ethanol (0.8 ml). The remaining residue was purified by flash chromatography on silica gel using 0-5% MeOH/DCM to afford the product methyl 6-(2-((1S)-1-((9-(tetrahydro-2H-pyran-2-yl)-9H-purin-6-yl)amino)propyl)quinazolin-4-yl)hexanoate 23.2a (130.0 mg, 0.25 mmol) in 74% yield. LC-MS (method 1): t_{R}=3.27 min, m/z (M+H)⁺=518.4.

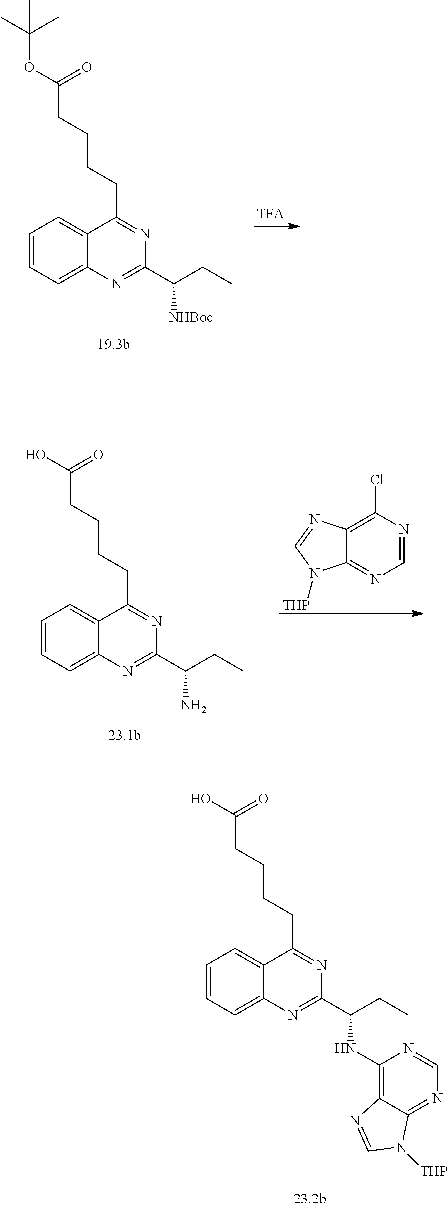

The procedure mentioned in Scheme 23 was used with compound 19.3b (45.0 mg, 0.102 mmol) and trifluoroacetic acid (233.0 mg, 2.04 mmol, 0.16 ml) in dichloromethane (1.0 ml). The resulting mixture was stirred at room temperature for 3 hours and worked-up (Method B) to afford (S)-5-(2-(1-aminopropyl)quinazolin-4-yl)pentanoic acid 23.1b. This free amine 23.1b was used with 6-chloro-9-(tetrahydro-2H-pyran-2-yl)-9H-purine (48.7 mg, 0.20 mmol) and triethylamine (41.3 mg, 0.41 mmol, 56.9 μl) in ethanol (0.25 ml). The remaining residue was purified by flash chromatography on silica gel using 0-5% MeOH (0.5% AcOH)/DCM to afford the product 5-(2-((1S)-1-((9-(tetrahydro-2H-pyran-2-yl)-9H-purin-6-yl)amino)propyl)quinazolin-4-yl)pentanoic acid 23.2b (27.7 mg, 0.057 mmol) in 56% yield. LC-MS (method 1): t_{R}=2.92 min, m/z (M+H)⁺=490.3.

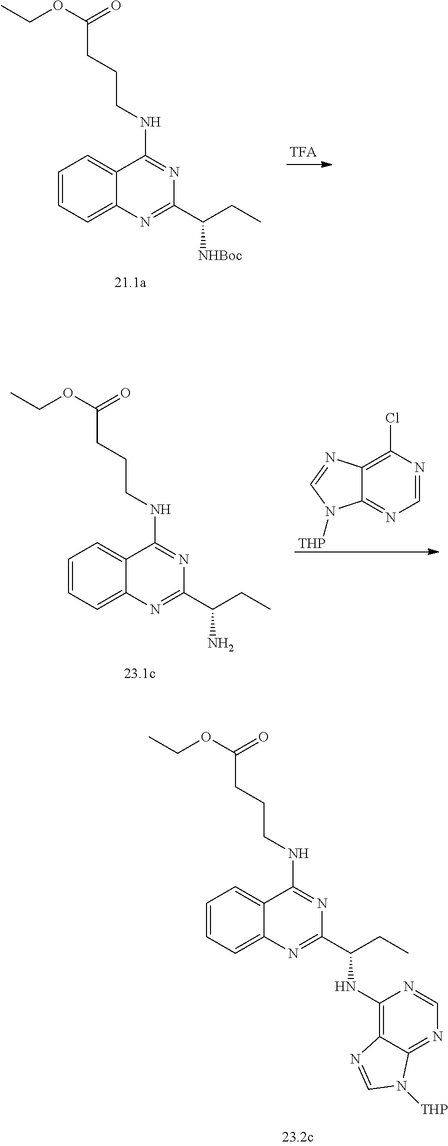

The procedure mentioned in Scheme 23 was used with compound 21.1a (133.0 mg, 0.32 mmol) and trifluoroacetic acid (730.0 mg, 6.40 mmol, 0.49 ml) in dichloromethane (3.2 ml). The resulting mixture was stirred at room temperature for 3 hours and worked-up (Method A) to afford (S)-ethyl 4-((2-(1-aminopropyl)quinazolin-4-yl)amino)butanoate 23.1c. This free amine 23.1c was used with 6-chloro-9-(tetrahydro-2H-pyran-2-yl)-9H-purine (153.0 mg, 0.64 mmol) and triethylamine (130.0 mg, 1.28 mmol, 179 μl) in ethanol (0.8 ml). The remaining residue was purified by flash chromatography on silica gel using 0-5% MeOH/DCM to afford the product ethyl 4-((2-((1S)-1-((9-(tetrahydro-2H-pyran-2-yl)-9H-purin-6-yl)amino)propyl)quinazolin-4-yl)amino)butanoate 23.2c (128.0 mg, 0.25 mmol) in 77% yield. LC-MS (method 1): t_{R}=2.78 min, m/z (M+H)⁺=519.3.

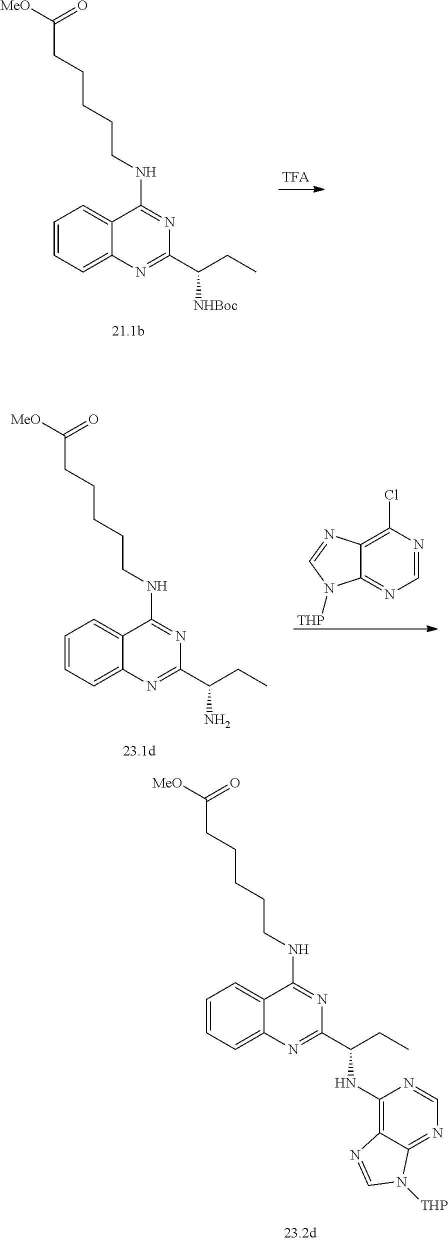

The procedure mentioned in Scheme 23 was used with compound 21.1b (252.0 mg, 0.585 mmol) and trifluoroacetic acid (1.35 g, 11.71 mmol, 0.90 ml) in dichloromethane (5.9 ml). The resulting mixture was stirred at room temperature for 3 hours and worked-up (Method A) to methyl (S)-6-((2-(1-aminopropyl)quinazolin-4-yl)amino)hexanoate 23.1d. This free amine 23.1d was used with 6-chloro-9-(tetrahydro-2H-pyran-2-yl)-9H-purine (209.0 mg, 0.88 mmol) and triethylamine (178.0 mg, 1.76 mmol, 0.25 ml) in ethanol (1.5 ml). The remaining residue was purified by flash chromatography on silica gel using 0-5% MeOH/DCM to afford the product methyl 6-((2-((1S)-1-((9-(tetrahydro-2H-pyran-2-yl)-9H-purin-6-yl)amino)propyl)quinazolin-4-yl)amino)hexanoate 23.2d (295.9 mg, 0.554 mmol) in 95% yield. LC-MS (method 1): t_{R}=2.87 min, m/z (M+H)⁺=533.3.

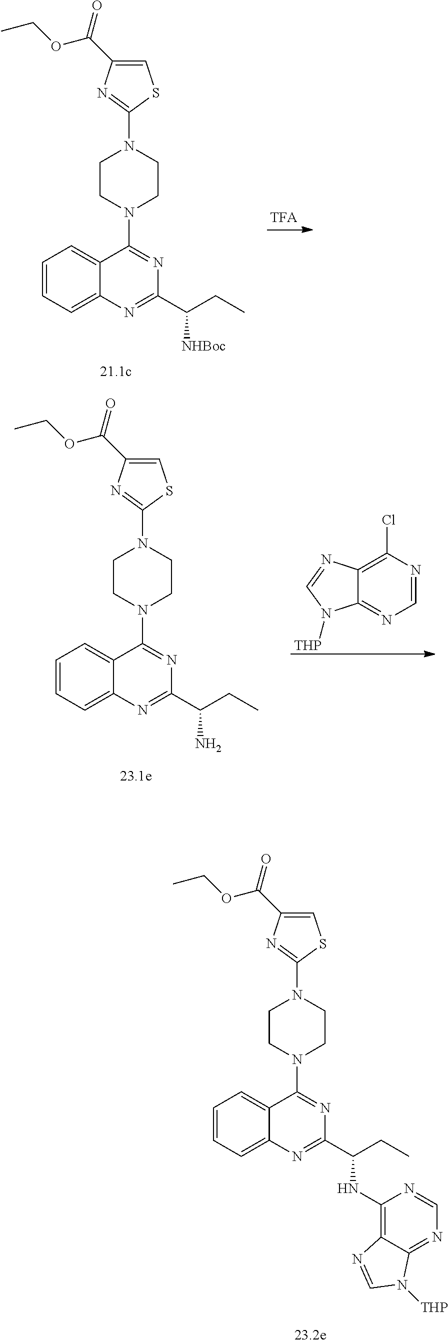

The procedure mentioned in Scheme 23 was used with compound 21.1c (92.0 mg, 0.175 mmol) and trifluoroacetic acid (398.0 mg, 3.49 mmol, 0.27 ml) in dichloromethane (1.8 ml). The resulting mixture was stirred at room temperature for 3 hours and worked-up (Method A) to afford ethyl (S)-2-(4-(2-(1-aminopropyl)quinazolin-4-yl)piperazin-1-yl)thiazole-4-carboxylate 23.1e. This free amine 23.1e was used with 6-chloro-9-(tetrahydro-2H-pyran-2-yl)-9H-purine (63.0 mg, 0.26 mmol) and triethylamine (53.0 mg, 0.53 mmol, 73.0 μl) in ethanol (0.40 ml). The remaining residue was purified by flash chromatography on silica gel using 0-5% MeOH/DCM to afford the product ethyl 2-(4-(2-((1S)-1-((9-(tetrahydro-2H-pyran-2-yl)-9H-purin-6-yl)amino)propyl)quinazolin-4-yl)piperazin-1-yl)thiazole-4-carboxylate 23.2e (106.0 mg, 0.69 mmol) in 96% yield. LC-MS (method 1): t_{R}=2.89 min, m/z (M+H)⁺=629.3.

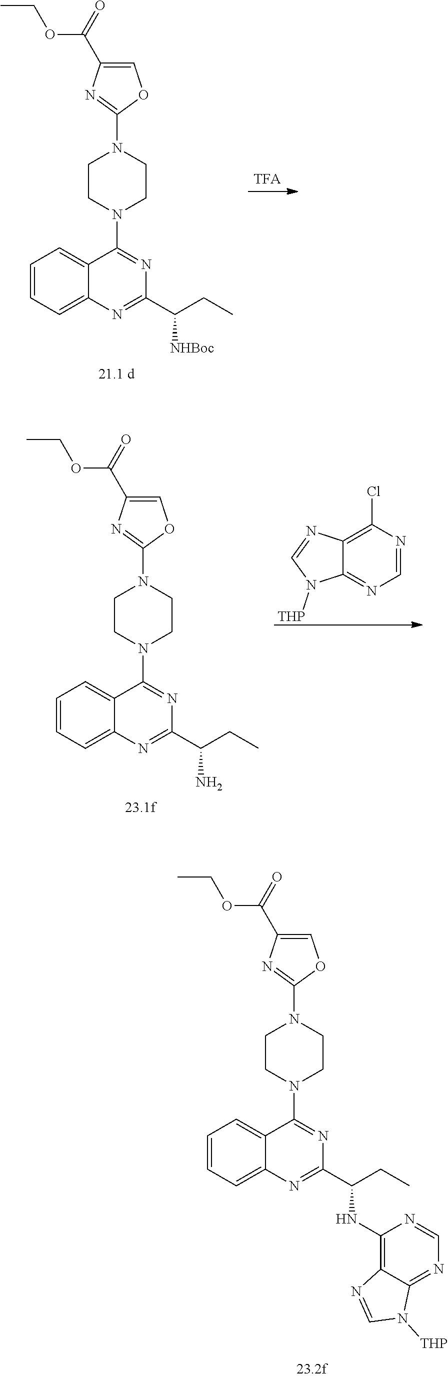

The procedure mentioned in Scheme 23 was used with compound 21.1d (140.0 mg, 0.274 mmol) and trifluoroacetic acid (625.0 mg, 5.48 mmol, 0.42 ml) in dichloromethane (2.7 ml). The resulting mixture was stirred at room temperature for 3 hours and worked-up (Method A) to afford ethyl (S)-2-(4-(2-(1-aminopropyl)quinazolin-4-yl)piperazin-1-yl)oxazole-4-carboxylate 23.1f. This free amine 23.1f was used with 6-chloro-9-(tetrahydro-2H-pyran-2-yl)-9H-purine (98.0 mg, 0.41 mmol) and triethylamine (83.0 mg, 0.82 mmol, 115.0 μl) in ethanol (0.7 ml). The remaining residue was purified by flash chromatography on silica gel using 0-10% MeOH/DCM to afford the product ethyl 2-(4-(2-((1S)-1-((9-(tetrahydro-2H-pyran-2-yl)-9H-purin-6-yl)amino)propyl)quinazolin-4-yl)piperazin-1-yl)oxazole-4-carboxylate 23.2f (150.0 mg, 0.245 mmol) in 89% yield. LC-MS (method 1): t_{R}=2.63 min, m/z (M+H)⁺=616.3.

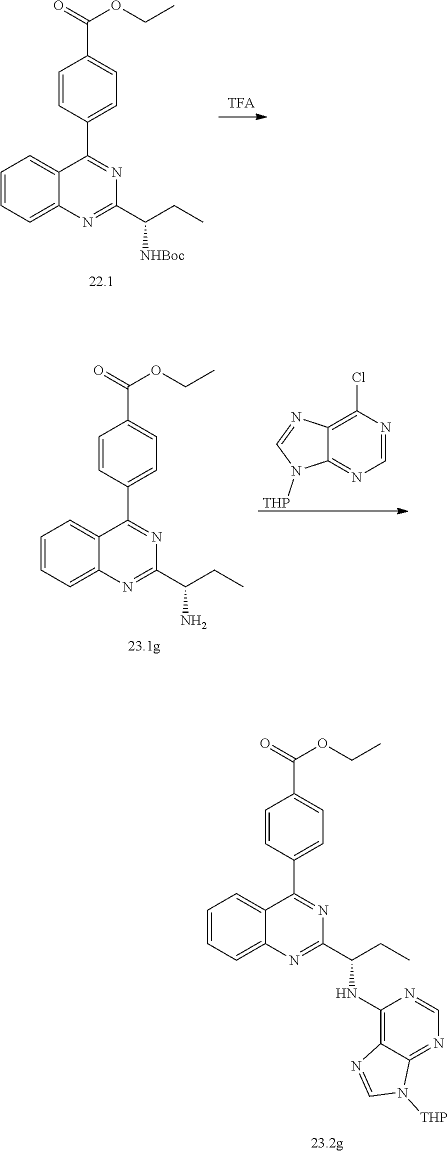

The procedure mentioned in Scheme 23 was used with compound 22.1 (131.0 mg, 0.30 mmol) and trifluoroacetic acid (686.0 mg, 6.02 mmol, 0.46 ml) in dichloromethane (3.0 ml). The resulting mixture was stirred at room temperature for 3 hours and worked-up (Method A) to afford ethyl (S)-4-(2-(1-aminopropyl)quinazolin-4-yl)benzoate 23.1g. This free amine 23.1g was used with 6-chloro-9-(tetrahydro-2H-pyran-2-yl)-9H-purine (108.0 mg, 0.45 mmol) and triethylamine (91.0 mg, 0.90 mmol, 0.13 ml) in ethanol (0.7 ml). The remaining residue was purified by flash chromatography on silica gel using 0-5% MeOH/DCM to afford the product ethyl 4-(2-((1S)-1-((9-(tetrahydro-2H-pyran-2-yl)-9H-purin-6-yl)amino)propyl)quinazolin-4-yl)benzoate 23.2g (115.5 mg, 0.215 mmol) in 71% yield. LC-MS (method 1): t_{R}=3.44 min, m/z (M+H)⁺=538.3.

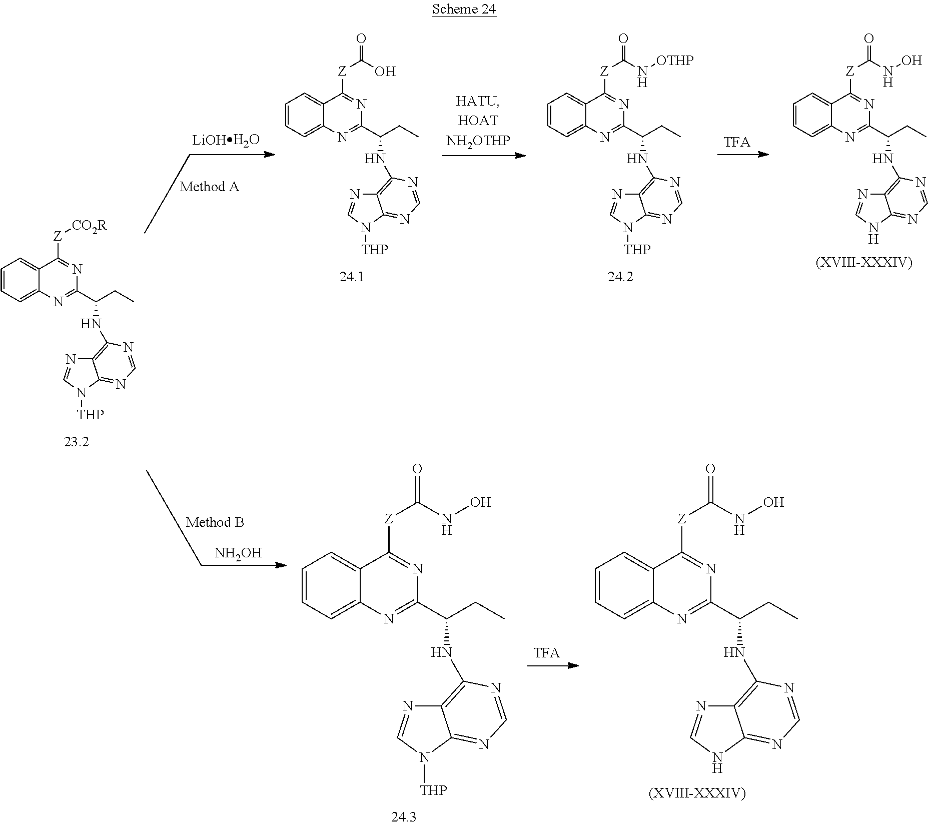

### Scheme 24

Method A: Suspended compound 23.2 in MeOH/water (0.1 M, 1:1) in a vial equipped with a stir bar and added LiOH.H₂O (2.0 equiv) to it. The resulting mixture was stirred at room temperature for 10 hours and concentrated in vacuo to afford crude 24.1. Suspended crude compound 24.1 in DMF (0.1 M) and added O-(tetrahydro-2H-pyran-2-yl)hydroxylamine [NH₂OTHP] (3.1 equiv), N-methyl morpholine (3.0 equiv), 3-(((ethylimino)methylene)amino)-N,N-dimethylpropan-1-amine hydrochloride [EDC.HCl] (1.4 equiv) and 1H-[1,2,3]triazolo[4,5-b]pyridin-1-ol [HOAT] (1.2 equiv) to it. Stirred the resulting suspension at room temperature for 16 hours and concentrated in vacuo. Purified the remaining residue by flash chromatography on silica using forced flow of 0-10% MeOH/DCM system on Biotage KP-Sil pre-packed cartridges and using the Biotage SP-1 automated chromatography system to afford the product 24.2. Dissolved compound 24.2 in DCM (0.1 M) and added TFA (20.0 equiv) to it. Stirred the resulting mixture for 20 hours. After completion of reaction (by LC-MS), concentrated the reaction mixture in vacuo and purified by C-18 reverse phase chromatography to afford the final compound (XXVIII-XXXIV).

Method B: Dissolved compound 23.2 in MeOH (0.1 M) in a MW vial equipped with a stir bar and added 50% hydroxylamine in water solution (10.0 equiv) and lithium hydroxide (1.2 equiv) at 0° C. to it. The MW vial was sealed and the resulting solution was stirred at 0° C. for 2 hours, then allowed to warmup to room temperature overnight. After completion of reaction by LC-MS, the reaction mixture was concentrated in vacuo to afford the crude product 24.3. Dissolved compound 24.3 in DCM/MeOH (0.1M, 1:1 by vol) and added TFA (20.0 equiv) to it. Stirred the resulting mixture for 20 hours. After completion of reaction (by LC-MS), concentrated the reaction mixture in vacuo and purified by C-18 reverse phase chromatography to afford the final compound (XXVIII-XXXIV).

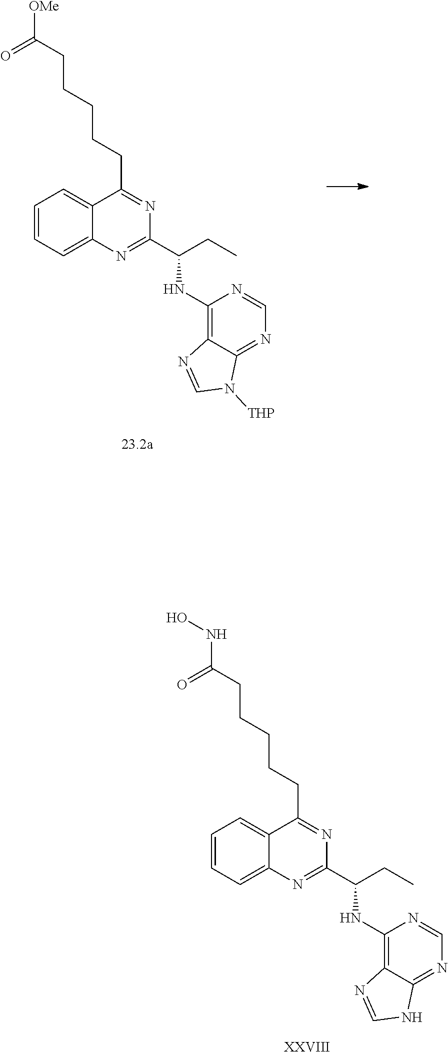

The procedure mentioned in Scheme 24 (Method A) was used with compound 23.2a (70.4 mg, 0.136 mmol) to afford product (S)-6-(2-(1-((9H-purin-6-yl)amino)propyl)quinazolin-4-yl)-N-hydroxyhexanamide, TFA XXVIII (45.0 mg, 0.082 mmol) in 60% yield. LC-MS (method 2): t_{R}=3.53 min, m/z (M+H)⁺=435.3. ¹H NMR (400 MHz, DMSO-d6) δ 10.35 (s, 1H), 8.39 (d, J=8.8 Hz, 2H), 8.32 (dt, J=8.4, 1.1 Hz, 1H), 7.99-7.95 (m, 2H), 7.76-7.70 (m, 1H), 5.59 (s, 1H), 3.29 (h, J=7.1 Hz, 2H), 2.20 (dd, J=14.6, 7.7 Hz, 1H), 2.08 (dt, J=13.6, 7.4 Hz, 1H), 1.93 (t, J=7.3 Hz, 2H), 1.79 (p, J=7.5 Hz, 2H), 1.53 (p, J=7.3 Hz, 2H), 1.34 (p, J=7.7 Hz, 2H), 0.94 (t, J=7.3 Hz, 3H).

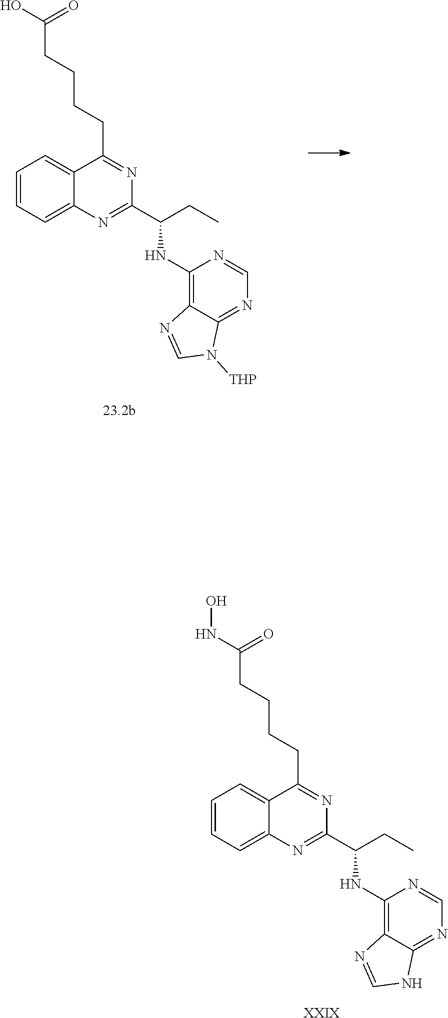

The procedure mentioned in Scheme 24 (Method A) was used with compound 23.2b (16.3 mg, 0.033 mmol) to afford product (S)-5-(2-(1-((9H-purin-6-yl)amino)propyl)quinazolin-4-yl)-N-hydroxypentanamide, TFA XXIX (7.5 mg, 0.014 mmol) in 55% yield. LC-MS (method 2): t_{R}=3.40 min, m/z (M+H)⁺=421.2. 1H NMR (400 MHz, DMSO-d6) δ 10.50 (s, 1H), 8.40-8.28 (m, 3H), 8.03-7.94 (m, 2H), 7.76-7.67 (m, 1H), 3.32 (h, J=7.9 Hz, 2H), 2.23-2.14 (m, 1H), 2.07 (dt, J=19.8, 6.9 Hz, 3H), 1.85-1.76 (m, 2H), 1.76-1.66 (m, 2H), 1.62-1.57 (m, 1H), 0.93 (t, J=7.3 Hz, 3H).

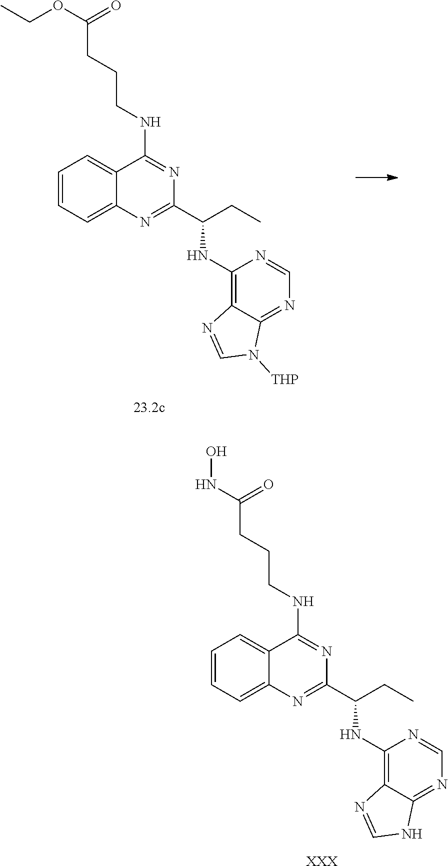

The procedure mentioned in Scheme 24 (Method A) was used with compound 23.2c (100.0 mg, 0.193 mmol) to afford product (S)-4-((2-(1-((9H-purin-6-yl)amino)propyl)quinazolin-4-yl)amino)-N-hydroxybutanamide, TFA XXX (39.0 mg, 0.073 mmol) in 38% yield. LC-MS (method 2): t_{R}=2.70 min, m/z (M+H)⁺=422.1. 1H NMR (400 MHz, DMSO-d6) δ 10.56 (s, 1H), 8.38 (d, J=8.1 Hz, 1H), 8.28 (d, J=16.0 Hz, 2H), 8.05-7.96 (m, 1H), 7.88-7.81 (m, 1H), 7.74 (t, J=7.7 Hz, 1H), 3.63 (ddd, J=35.6, 13.3, 6.7 Hz, 3H), 2.20-2.02 (m, 4H), 2.02-1.88 (m, 1H), 1.78 (dq, J=13.4, 6.7 Hz, 1H), 1.03 (t, J=7.4 Hz, 3H).

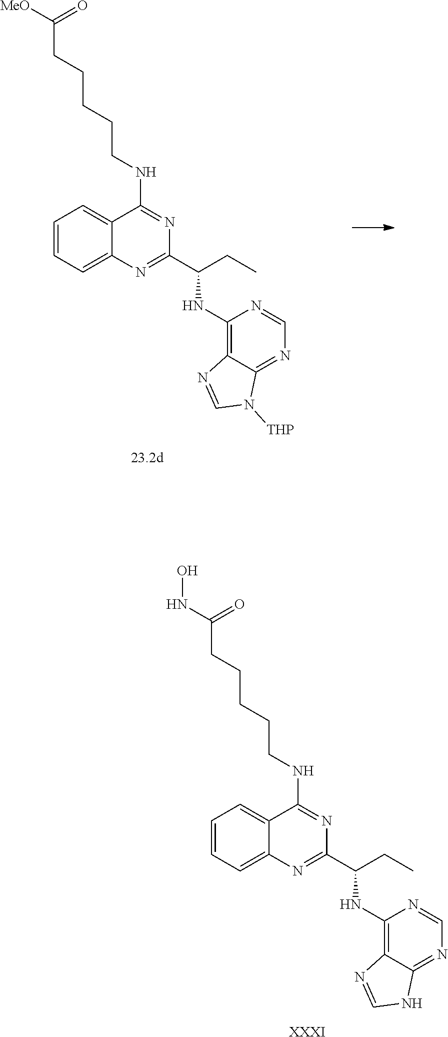

The procedure mentioned in Scheme 24 (Method A) was used with compound 23.2d (280.0 mg, 0.526 mmol) to afford product (S)-6-((2-(1-((9H-purin-6-yl)amino)propyl)quinazolin-4-yl)amino)-N-hydroxyhexanamide, TFA XXXI (112.5 mg, 0.200 mmol) in 38% yield. LC-MS (method 2): t_{R}=2.77 min, m/z (M+H)⁺=450.3. 1H NMR (400 MHz, DMSO-d6) δ 10.09 (s, 1H), 8.38 (dd, J=8.5, 1.2 Hz, 2H), 8.31 (s, 1H), 8.22 (s, 1H), 8.01 (ddd, J=8.3, 7.1, 1.2 Hz, 1H), 7.85 (dd, J=8.5, 1.1 Hz, 1H), 7.74 (ddd, J=8.3, 7.2, 1.2 Hz, 1H), 5.24 (s, 1H), 3.69 (dq, J=13.2, 6.6 Hz, 1H), 3.55 (dt, J=13.1, 6.6 Hz, 1H), 2.14 (q, J=7.2 Hz, 2H), 1.88 (t, J=7.3 Hz, 2H), 1.56-1.41 (m, 2H), 1.41-1.34 (m, 2H), 1.17 (q, J=7.7 Hz, 2H), 1.06 (t, J=7.4 Hz, 3H).

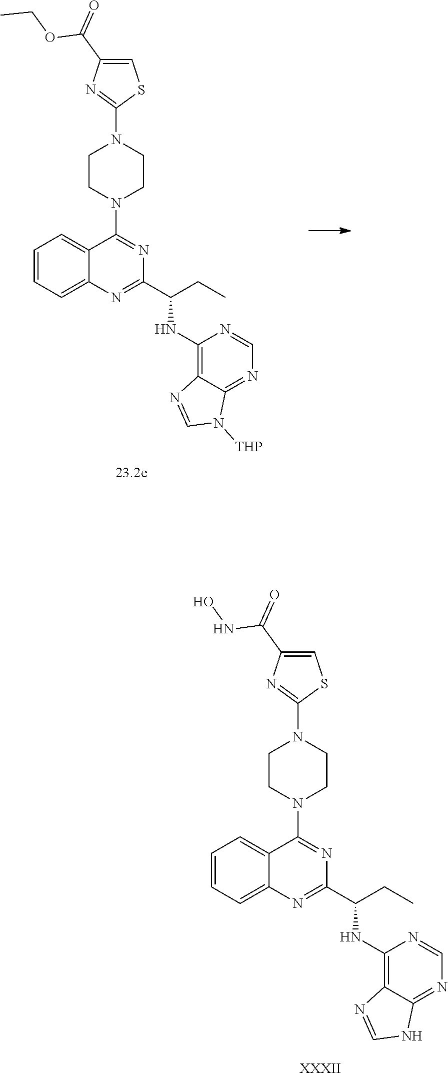

The procedure mentioned in Scheme 24 (Method B) was used with compound 23.2e (98.5 mg, 0.157 mmol) to afford product (S)-2-(4-(2-(1-((9H-purin-6-yl)amino)propyl)quinazolin-4-yl)piperazin-1-yl)-N-hydroxythiazole-4-carboxamide, TFA XXXII (46.5 mg, 0.072 mmol) in 46% yield. LC-MS (method 2): t_{R}=3.05 min, m/z (M+H)⁺=532.2. ¹H NMR (400 MHz, DMSO-d6) δ 10.84 (s, 1H), 8.71 (s, 1H), 8.41 (s, 1H), 8.33 (s, 1H), 8.23-8.16 (m, 1H), 7.99 (ddd, J=8.3, 7.0, 1.2 Hz, 1H), 7.88 (dd, J=8.5, 1.2 Hz, 1H), 7.68 (ddd, J=8.4, 7.0, 1.2 Hz, 1H), 7.45 (s, 1H), 5.36 (s, 1H), 4.25 (s, 4H), 3.74-3.61 (m, 4H), 2.15 (qq, J=14.1, 7.3 Hz, 3H), 1.04 (t, J=7.4 Hz, 3H).

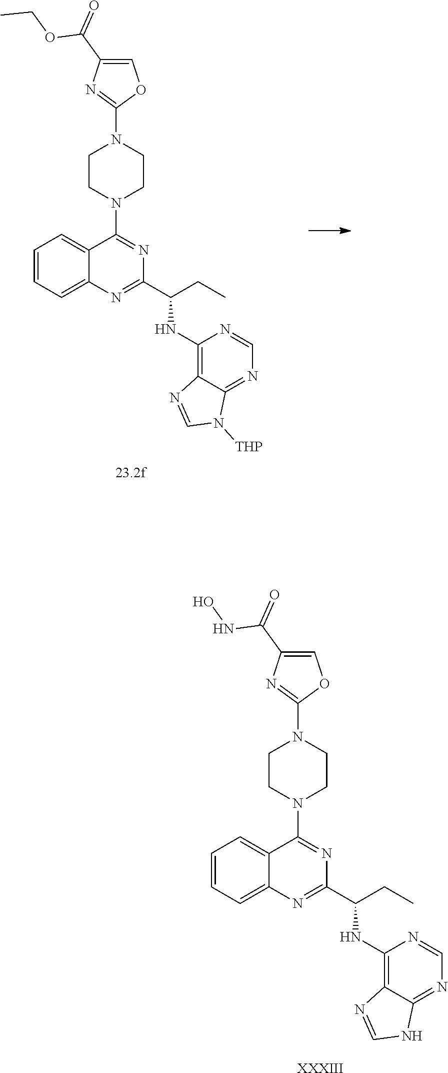

The procedure mentioned in Scheme 24 (Method A) was used with compound 23.2f (130.0 mg, 0.212 mmol) to afford product (S)-2-(4-(2-(1-((9H-purin-6-yl)amino)propyl)quinazolin-4-yl)piperazin-1-yl)-N-hydroxyoxazole-4-carboxamide XXXIII (15.0 mg, 0.03 mmol) in 14% yield. LC-MS (method 2): t_{R}=2.94 min, m/z (M+H)⁺=516.1.

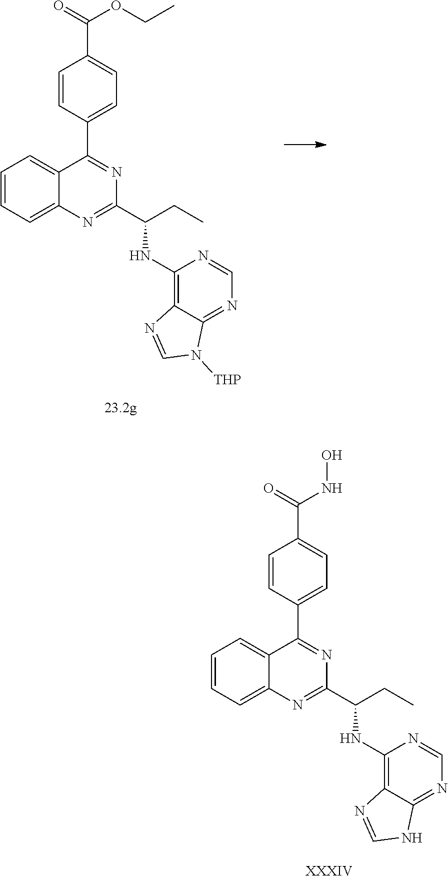

The procedure mentioned in Scheme 24 (Method A) was used with compound 23.2a (115.5 mg, 0.215 mmol) to afford product (S)-4-(2-(1-((9H-purin-6-yl)amino)propyl)quinazolin-4-yl)-N-hydroxybenzamide, TFA XXXIV (43.5 mg, 0.078 mmol) in 36% yield. LC-MS (method 2): t_{R}=3.31 min, m/z (M+H)⁺=441.2. 1H NMR (400 MHz, DMSO-d6) δ 11.43 (s, 1H), 8.81 (s, 1H), 8.45 (d, J=12.3 Hz, 2H), 8.11-8.03 (m, 3H), 8.02-7.96 (m, 2H), 7.91-7.85 (m, 2H), 7.75 (ddd, J=8.4, 6.6, 1.6 Hz, 1H), 5.70 (s, 1H), 2.30-2.22 (m, 1H), 2.13 (dt, J=13.6, 7.5 Hz, 1H), 1.00 (t, J=7.4 Hz, 3H).

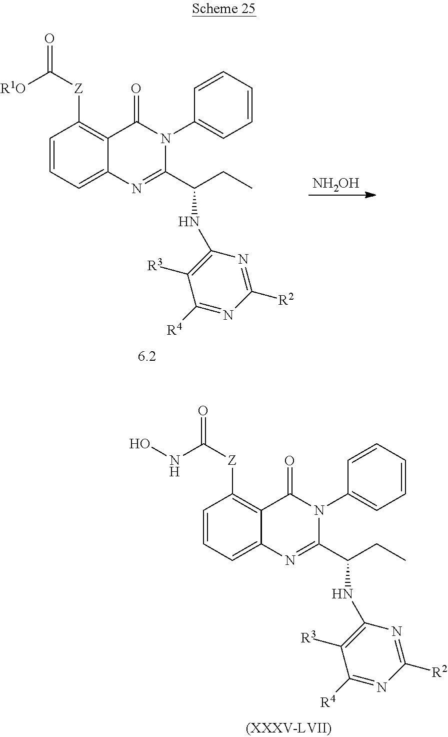

### Scheme 25

Dissolved compound 6.2 (1.0 equiv) in MeOH (0.1M) in a vial equipped with a stir bar and added 50% hydroxylamine in water solution (30.0 equiv) and lithium hydroxide (1.2-2.0 equiv) at 0° C. to it. The resulting solution was stirred at 0° C. for 2 hours and then allowed to warmup to room temperature overnight. After completion of reaction by LC-MS, the reaction mixture was concentrated in vacuo and purified by C-18 reverse phase chromatography to afford the final compound (XXXV-LVII).

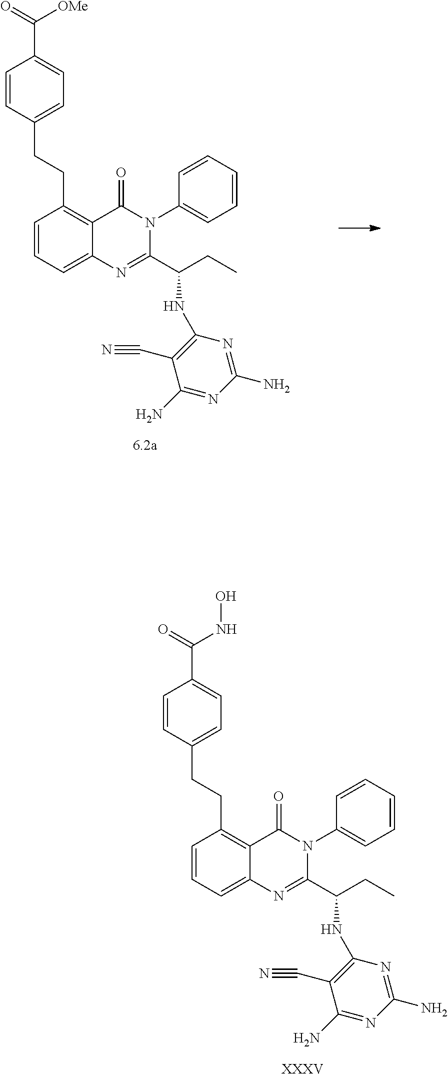

The procedure mentioned in Scheme 25 was used with compound 6.2a (56.0 mg, 0.097 mmol) to afford the product (S)-4-(2-(2-(1-((2,6-diamino-5-cyanopyrimidin-4-yl)amino)propyl)-4-oxo-3-phenyl-3,4-dihydroquinazolin-5-yl)ethyl)-N-hydroxybenzamide, TFA XXXV (27.2 mg, 0.039 mmol) in 40% yield. LC-MS (method 2): t_{R}=4.34 min, m/z (M+H)⁺=576.2. ¹H NMR (400 MHz, DMSO-d6) δ 11.10 (s, 1H), 7.77-7.71 (m, 1H), 7.67-7.61 (m, 3H), 7.60-7.44 (m, 6H), 7.36-7.25 (m, 4H), 4.76 (q, J=7.3 Hz, 1H), 2.91-2.82 (m, 2H), 1.94-1.85 (m, 1H), 1.75 (dt, J=14.5, 7.4 Hz, 1H), 0.74-0.65 (m, 3H). [Note: In addition to compound XXXV, a side product originating from the hydrolysis of methyl ester in 6.2a to carboxylic acid was also isolated in 7% yield].

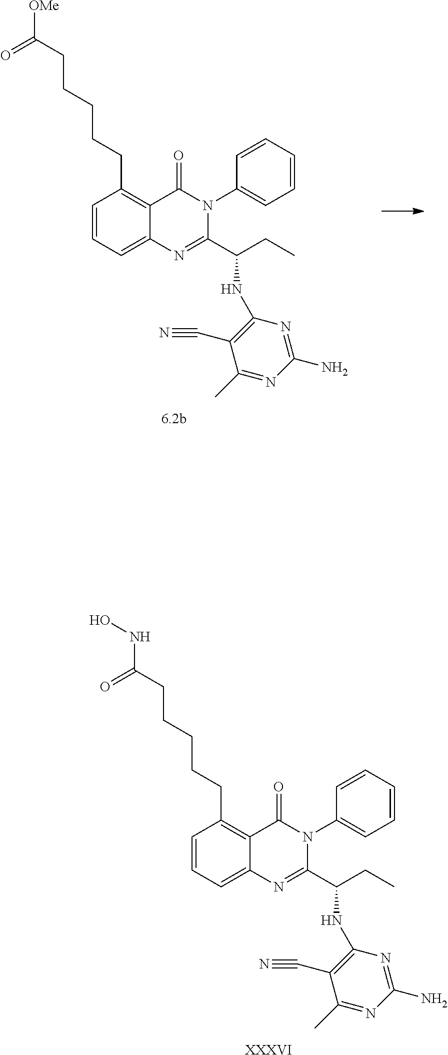

The procedure mentioned in Scheme 25 was used with compound 6.2b (54.0 mg, 0.10 mmol) to afford the product (S)-6-(2-(1-((2-amino-5-cyano-6-methylpyrimidin-4-yl)amino)propyl)-4-oxo-3-phenyl-3,4-dihydroquinazolin-5-yl)-N-hydroxyhexanamide, TFA XXXVI (45.0 mg, 0.069 mmol) in 69% yield. LC-MS (method 2): t_{R}=4.04 min, m/z (M+H)⁺=541.3. 1H NMR (400 MHz, DMSO-d6) δ 10.28 (s, 1H), 8.09 (s, 1H), 7.73 (t, J=7.8 Hz, 1H), 7.59-7.45 (m, 6H), 7.45-7.36 (m, 2H), 7.32 (dd, J=7.5, 1.3 Hz, 1H), 4.77 (td, J=7.6, 4.8 Hz, 1H), 3.14 (t, J=7.7 Hz, 2H), 2.54 (s, 1H), 2.34 (s, 3H), 2.00-1.86 (m, 3H), 1.79 (dp, J=14.7, 7.4 Hz, 1H), 1.49 (dq, J=15.1, 7.6 Hz, 4H), 1.28 (p, J=7.7 Hz, 2H), 0.72 (t, J=7.3 Hz, 3H).

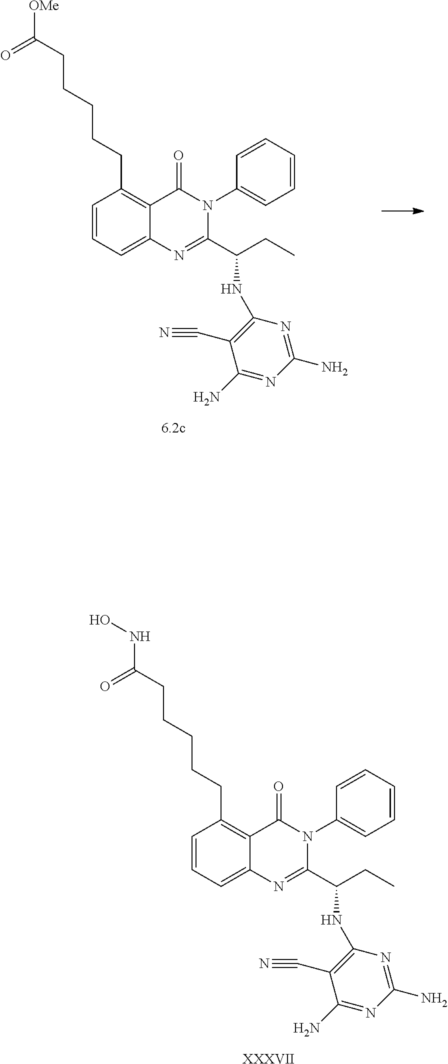

The procedure mentioned in Scheme 25 was used with compound 6.2c (54.0 mg, 0.10 mmol) to afford the product (S)-6-(2-(1-((2,6-diamino-5-cyanopyrimidin-4-yl)amino)propyl)-4-oxo-3-phenyl-3,4-dihydroquinazolin-5-yl)-N-hydroxyhexanamide, TFA XXXVII (40.0 mg, 0.061 mmol) in 61% yield. LC-MS (method 2): t_{R}=3.91 min, m/z (M+H)⁺=542.3. ¹H NMR (400 MHz, DMSO-d6) δ 10.29 (s, 1H), 8.05 (s, 3H), 7.74 (t, J=7.8 Hz, 2H), 7.57-7.42 (m, 7H), 7.33 (dd, J=7.6, 1.3 Hz, 1H), 4.77 (td, J=7.6, 4.9 Hz, 1H), 3.14 (ddq, J=12.4, 8.0, 5.5, 4.2 Hz, 2H), 1.90 (t, J=7.2 Hz, 3H), 1.82-1.71 (m, 1H), 1.49 (dp, J=15.0, 7.4 Hz, 4H), 1.28 (q, J=8.1 Hz, 2H), 0.71 (t, J=7.3 Hz, 3H).

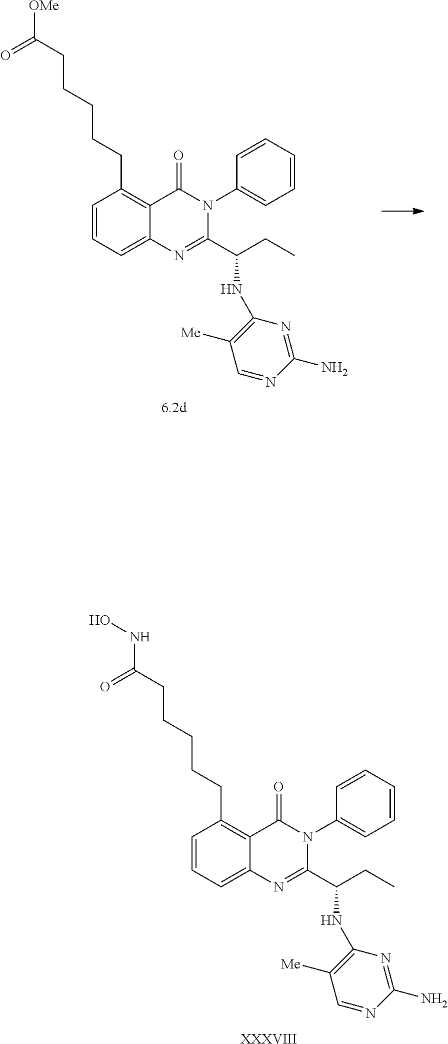

The procedure mentioned in Scheme 25 was used with compound 6.2d (35.0 mg, 0.068 mmol) to afford the product (S)-6-(2-(1-((2-amino-5-methylpyrimidin-4-yl)amino)propyl)-4-oxo-3-phenyl-3,4-dihydroquinazolin-5-yl)-N-hydroxyhexanamide, TFA XXXVIII (30.0 mg, 0.048 mmol) in 70% yield. LC-MS (method 2): t_{R}=4.06 min, m/z (M+H)⁺=516.3. ¹H NMR (400 MHz, DMSO-d₆) δ 11.72 (s, 1H), 10.29 (s, 1H), 8.09 (d, J=7.7 Hz, 1H), 7.73 (t, J=7.8 Hz, 1H), 7.57-7.50 (m, 4H), 7.42-7.31 (m, 5H), 4.85 (td, J=7.9, 5.2 Hz, 1H), 3.14 (t, J=7.7 Hz, 2H), 2.08-1.78 (m, 8H), 1.47 (dt, J=14.9, 7.0 Hz, 4H), 1.27 (p, J=7.5, 7.1 Hz, 2H), 0.76 (t, J=7.3 Hz, 3H).

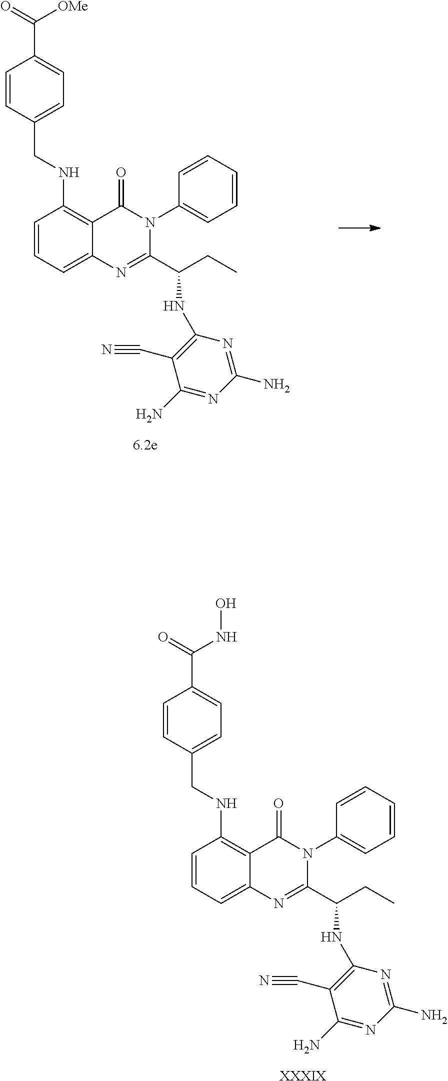

The procedure mentioned in Scheme 25 was used with compound 6.2e (41.7 mg, 0.072 mmol) to afford product (S)-4-(((2-(1-((2,6-diamino-5-cyanopyrimidin-4-yl)amino)propyl)-4-oxo-3-phenyl-3,4-dihydroquinazolin-5-yl)amino)methyl)-N-hydroxybenzamide, TFA XXXIX (21.2 mg, 0.031 mmol) in 42% yield. LC-MS (method 2): t_{R}=3.98 min, m/z (M+H)⁺=577.3. 1H NMR (400 MHz, DMSO-d6) δ 11.12 (s, 1H), 8.96 (s, 1H), 7.70 (dd, J=8.2, 1.7 Hz, 2H), 7.55 (t, J=7.3 Hz, 2H), 7.52-7.43 (m, 5H), 7.40 (dd, J=8.2, 1.7 Hz, 2H), 6.76 (d, J=7.8 Hz, 1H), 6.51 (d, J=8.4 Hz, 1H), 4.72 (q, J=7.1 Hz, 1H), 4.50 (d, J=4.4 Hz, 2H), 1.85 (q, J=6.4, 5.8 Hz, 1H), 1.71 (dt, J=14.5, 7.4 Hz, 1H), 0.72-0.64 (m, 3H).

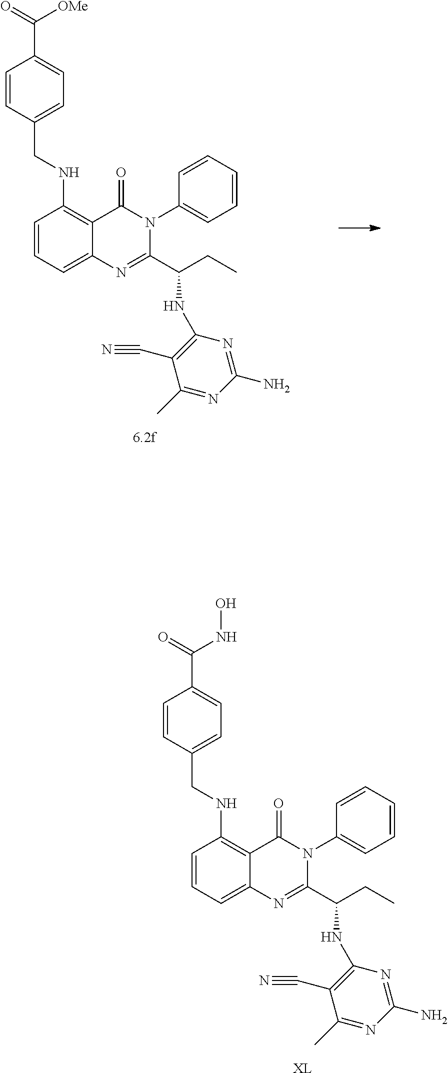

The procedure mentioned in Scheme 25 was used with compound 6.2f (54.0 mg, 0.094 mmol) to afford product (S)-4-(((2-(1-((2-amino-5-cyano-6-methylpyrimidin-4-yl)amino)propyl)-4-oxo-3-phenyl-3,4-dihydroquinazolin-5-yl)amino)methyl)-N-hydroxybenzamide, TFA XL (34.0 mg, 0.049 mmol) in 52% yield. LC-MS (method 2): t_{R}=4.18 min, m/z (M+H)⁺=576.3. ¹H NMR (400 MHz, DMSO-d6) δ 11.13 (s, 1H), 8.97 (s, 1H), 8.00 (s, 1H), 7.73-7.66 (m, 2H), 7.59-7.45 (m, 5H), 7.45-7.36 (m, 4H), 6.76 (dd, J=7.9, 0.8 Hz, 1H), 6.51 (dd, J=8.5, 0.9 Hz, 1H), 4.74 (td, J=7.7, 4.7 Hz, 1H), 4.50 (s, 2H), 2.33 (s, 3H), 1.90 (dtd, J=14.2, 7.2, 4.7 Hz, 1H), 1.82-1.71 (m, 1H), 0.70 (t, J=7.3 Hz, 3H).

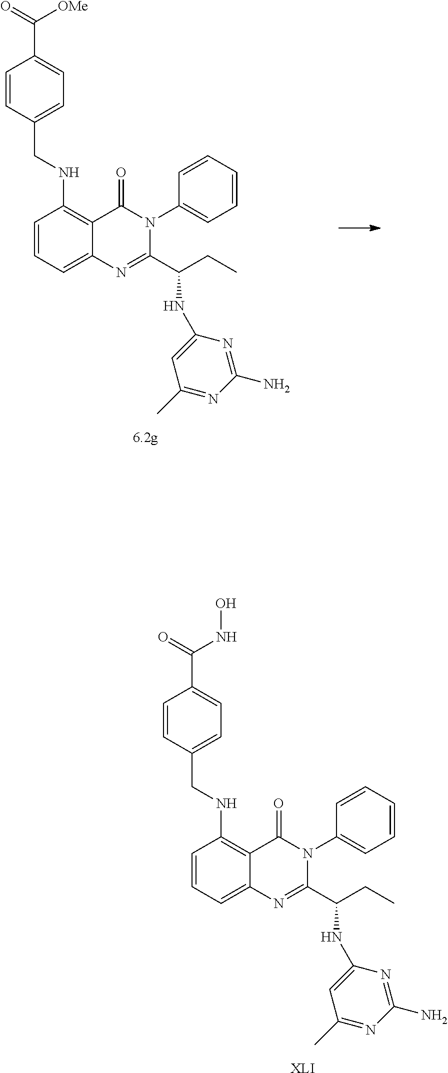

The procedure mentioned in Scheme 25 was used with compound 6.2g (35.0 mg, 0.064 mmol) to afford product (S)-4-(((2-(1-((2-amino-6-methylpyrimidin-4-yl)amino)propyl)-4-oxo-3-phenyl-3,4-dihydroquinazolin-5-yl)amino)methyl)-N-hydroxybenzamide, TFA XLI (18.0 mg, 0.027 mmol) in 42% yield. LC-MS (method 2): t_{R}=4.23 min, m/z (M+H)⁺=551.2. 1H NMR (400 MHz, DMSO-d6) δ 12.08 (s, 1H), 11.14 (s, 1H), 8.96 (d, J=7.2 Hz, 2H), 7.72-7.68 (m, 2H), 7.60-7.45 (m, 6H), 7.42-7.37 (m, 3H), 6.73 (d, J=7.8 Hz, 1H), 6.51 (d, J=8.4 Hz, 1H), 5.99 (s, 1H), 4.51 (ddd, J=11.5, 5.5, 2.7 Hz, 3H), 2.20 (s, 3H), 1.88 (ddd, J=14.2, 7.4, 4.1 Hz, 1H), 1.64 (ddd, J=13.9, 9.0, 7.0 Hz, 1H), 0.67 (t, J=7.3 Hz, 3H). [Note: In addition to compound XLI, a side product originating from the hydrolysis of methyl ester in 6.2g to carboxylic acid was also isolated in 7% yield].

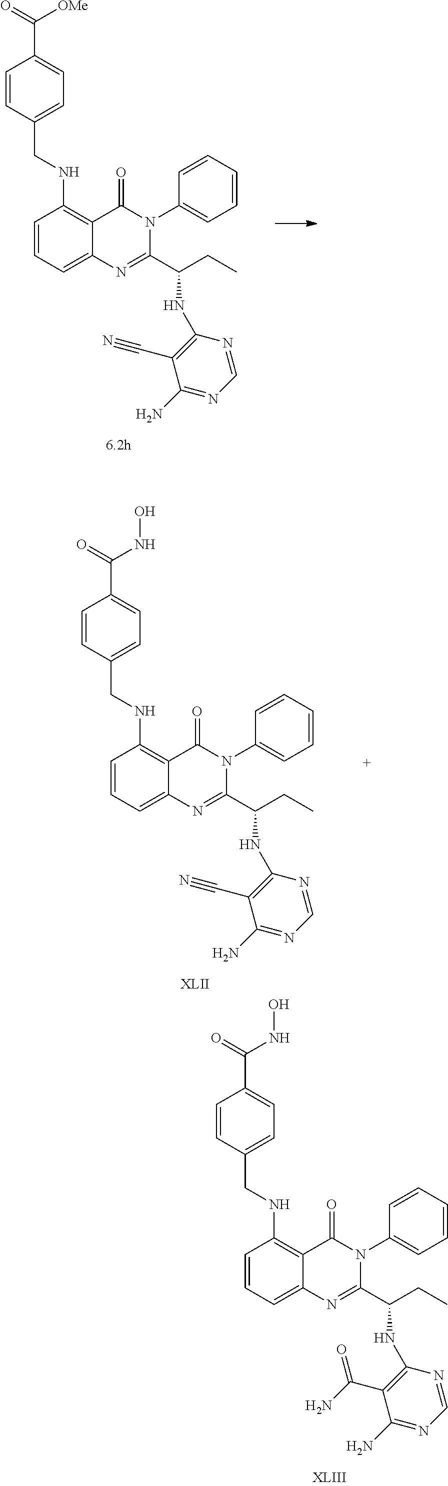 [SMILES: CC[C@H](Nc1ncnc(N)c1C#N)c1nc2cccc(NCc3ccc(C(=O)OC)cc3)c2c(=O)n1-c1ccccc1 ; CC[C@H](Nc1ncnc(N)c1C#N)c1nc2cccc(NCc3ccc(C(=O)NO)cc3)c2c(=O)n1-c1ccccc1 ; CC[C@H](Nc1ncnc(N)c1C(N)=O)c1nc2cccc(NCc3ccc(C(=O)NO)cc3)c2c(=O)n1-c1ccccc1]

The procedure mentioned in Scheme 25 was used with compound 6.2h (72.0 mg, 0.13 mmol) to afford products, (S)-4-(((2-(1-((6-amino-5-cyanopyrimidin-4-yl)amino)propyl)-4-oxo-3-phenyl-3,4-dihydroquinazolin-5-yl)amino)methyl)-N-hydroxybenzamide, TFA XLII (34.0 mg, 0.05 mmol) in 39% yield, and (S)-4-amino-6-((1-(5-((4-(hydroxycarbamoyl)benzyl)amino)-4-oxo-3-phenyl-3,4-dihydroquinazolin-2-yl)propyl)amino)pyrimidine-5-carboxamide, TFA XLIII (7.3 mg, 10.52 mol) in 8% yield.

XLII: LC-MS (method 2): t_{R}=4.36 min, m/z (M+H)⁺=562.2. ¹H NMR (400 MHz, DMSO-d6) δ 11.13 (s, 1H), 8.96 (s, 1H), 7.93 (s, 1H), 7.73-7.66 (m, 2H), 7.60-7.45 (m, 6H), 7.42-7.32 (m, 3H), 6.78-6.70 (m, 1H), 6.49 (d, J=8.4 Hz, 1H), 4.65 (td, J=7.9, 4.8 Hz, 1H), 4.49 (s, 2H), 2.54 (s, 1H), 1.86 (tq, J=12.2, 7.4, 6.2 Hz, 1H), 1.80-1.72 (m, 1H), 0.70 (t, J=7.3 Hz, 3H).

XLIII: LC-MS (method 2): t_{R}=3.76 min, m/z (M+H)⁺=580.3. [Note: In addition to compounds XLII and XLIII, a side product originating from the hydrolysis of methyl ester in 6.2h to carboxylic acid was also isolated in 2% yield].

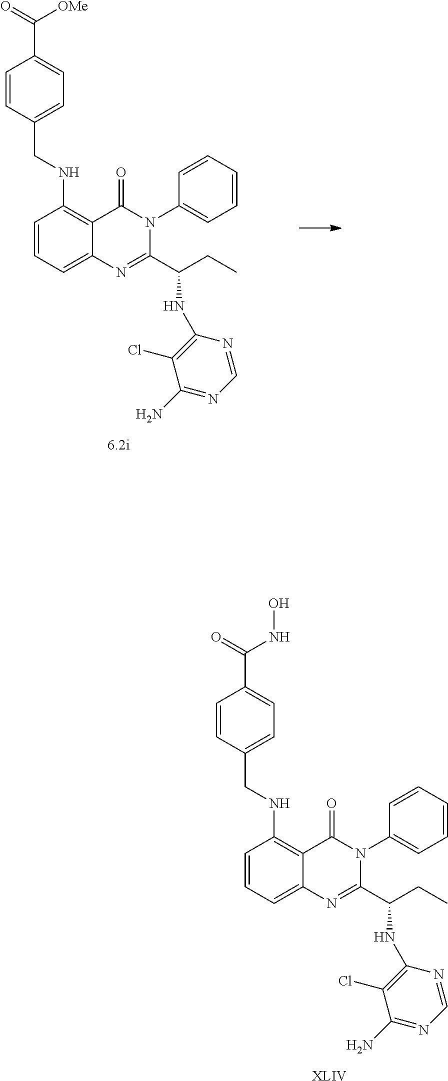 [SMILES: CC[C@H](Nc1ncnc(N)c1Cl)c1nc2cccc(NCc3ccc(C(=O)OC)cc3)c2c(=O)n1-c1ccccc1 ; CC[C@H](Nc1ncnc(N)c1Cl)c1nc2cccc(NCc3ccc(C(=O)NO)cc3)c2c(=O)n1-c1ccccc1 ; "XLIV"]

The procedure mentioned in Scheme 25 was used with compound 6.2i (55.0 mg, 0.096 mmol) to afford product (S)-4-(((2-(1-((6-amino-5-chloropyrimidin-4-yl)amino)propyl)-4-oxo-3-phenyl-3,4-dihydroquinazolin-5-yl)amino)methyl)-N-hydroxybenzamide, TFA XLIV (53.0 mg, 0.077 mmol) in 80% yield. LC-MS (method 2): t_{R}=4.50 min, m/z (M)⁺=571.1. ¹H NMR (400 MHz, DMSO-d6) δ 11.13 (s, 1H), 8.95 (s, 1H), 8.03 (s, 1H), 7.73-7.66 (m, 2H), 7.60-7.51 (m, 2H), 7.51-7.44 (m, 5H), 7.40 (d, J=8.1 Hz, 3H), 6.75 (d, J=7.8 Hz, 1H), 6.49 (d, J=8.4 Hz, 1H), 4.63 (td, J=8.1, 4.2 Hz, 1H), 4.49 (s, 2H), 1.93-1.75 (m, 2H), 0.70 (t, J=7.3 Hz, 3H). [Note: In addition to compound XLIV, a side product originating from the hydrolysis of methyl ester in 6.2i to carboxylic acid was also isolated in 9% yield].

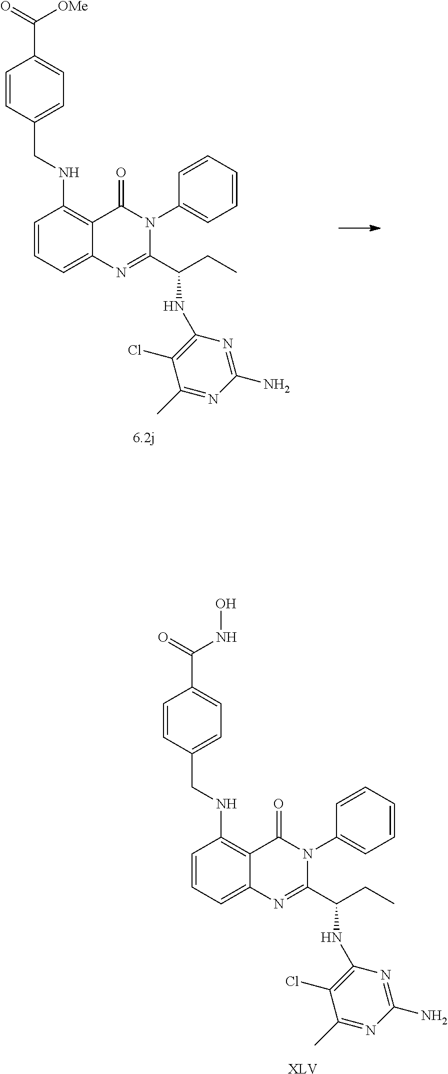

The procedure mentioned in Scheme 25 was used with compound 6.2j (68.0 mg, 0.116 mmol) to afford product (S)-4-(((2-(1-((2-amino-5-chloro-6-methylpyrimidin-4-yl)amino)propyl)-4-oxo-3-phenyl-3,4-dihydroquinazolin-5-yl)amino)methyl)-N-hydroxybenzamide, TFA XLV (62.0 mg, 0.089 mmol) in 76% yield. LC-MS (method 2): t_{R}=4.19 min, m/z (M)⁺=585.2. 1H NMR (400 MHz, DMSO-d6) δ 11.13 (s, 1H), 8.97 (t, J=5.8 Hz, 1H), 8.39 (d, J=7.4 Hz, 1H), 7.70 (d, J=8.0 Hz, 2H), 7.61-7.46 (m, 4H), 7.46-7.36 (m, 5H), 6.78 (d, J=7.8 Hz, 1H), 6.53 (d, J=8.4 Hz, 1H), 4.78 (td, J=7.7, 4.8 Hz, 1H), 4.51 (d, J=4.8 Hz, 2H), 2.33 (s, 3H), 2.03-1.91 (m, 1H), 1.83 (dq, J=14.4, 7.4 Hz, 1H), 0.72 (t, J=7.3 Hz, 3H). [Note: In addition to compound XLV, a side product originating from the hydrolysis of methyl ester in 6.2j to carboxylic acid was also isolated in 4% yield].

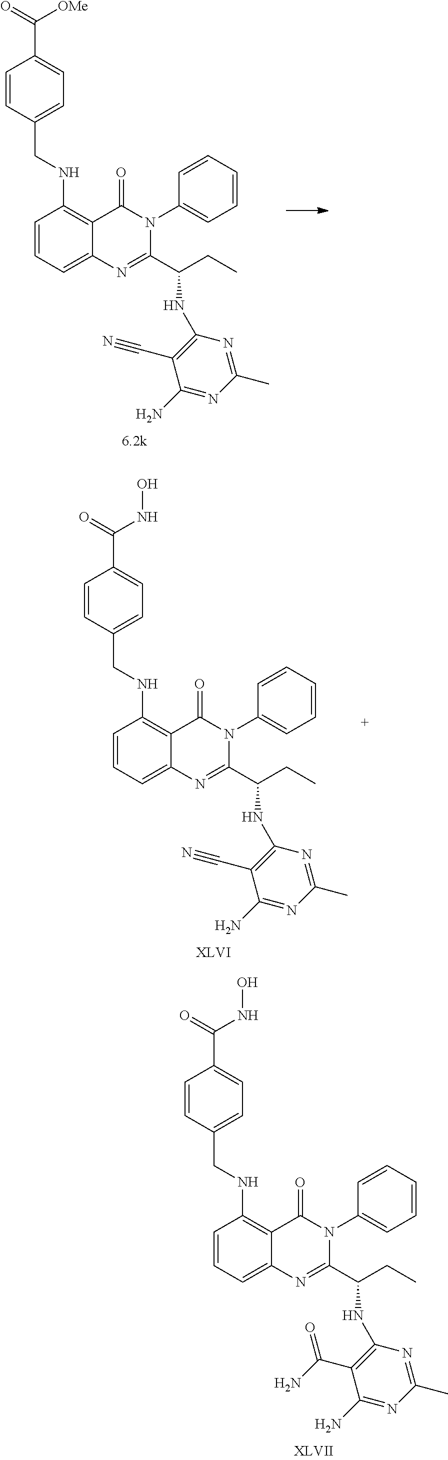 [SMILES: CC[C@H](Nc1nc(C)nc(N)c1C#N)c1nc2cccc(NCc3ccc(C(=O)OC)cc3)c2c(=O)n1-c1ccccc1 ; CC[C@H](Nc1nc(C)nc(N)c1C#N)c1nc2cccc(NCc3ccc(C(=O)NO)cc3)c2c(=O)n1-c1ccccc1 ; CC[C@H](Nc1nc(C)nc(N)c1C(N)=O)c1nc2cccc(NCc3ccc(C(=O)NO)cc3)c2c(=O)n1-c1ccccc1]

The procedure mentioned in Scheme 25 was used with compound 6.2k (73.0 mg, 0.13 mmol) to afford products, (S)-4-(((2-(1-((6-amino-5-cyano-2-methylpyrimidin-4-yl)amino)propyl)-4-oxo-3-phenyl-3,4-dihydroquinazolin-5-yl)amino)methyl)-N-hydroxybenzamide, TFA XLVI (56.0 mg, 0.08 mmol) in 64% yield, and (S)-4-amino-6-((1-(5-((4-(hydroxycarbamoyl)benzyl)amino)-4-oxo-3-phenyl-3,4-dihydroquinazolin-2-yl)propyl)amino)-2-methylpyrimidine-5-carboxamide, TFA XLVII (13.5 mg, 19.0 μmol) in 15% yield.

XLVI: LC-MS (method 2): t_{R}=4.17 min, m/z (M+H)⁺=576.3. ¹H NMR (400 MHz, DMSO-d6) δ 11.13 (s, 1H), 8.96 (s, 1H), 7.69 (d, J=8.1 Hz, 2H), 7.59-7.42 (m, 8H), 7.40 (d, J=8.0 Hz, 2H), 6.76 (d, J=7.9 Hz, 1H), 6.49 (d, J=8.4 Hz, 1H), 4.78 (td, J=8.3, 4.3 Hz, 1H), 4.50 (s, 2H), 2.17 (s, 3H), 1.92-1.75 (m, 2H), 0.68 (t, J=7.3 Hz, 3H).

XLVII: LC-MS (method 2): t_{R}=3.98 min, m/z (M+H)⁺=594.3. ¹H NMR (400 MHz, DMSO-d6) δ 11.13 (s, 1H), 8.94 (s, 1H), 8.69 (s, 1H), 7.91 (s, 2H), 7.69 (d, J=8.2 Hz, 2H), 7.64-7.52 (m, 5H), 7.49 (t, J=8.1 Hz, 1H), 7.41 (dd, J=15.4, 7.5 Hz, 3H), 6.72 (d, J=7.9 Hz, 1H), 6.51 (d, J=8.4 Hz, 1H), 4.92-4.82 (m, 1H), 4.50 (d, J=4.7 Hz, 2H), 3.17 (s, 1H), 2.30 (s, 3H), 1.93-1.83 (m, 1H), 1.66 (dt, J=14.5, 7.7 Hz, 1H), 0.70 (t, J=7.3 Hz, 3H). [Note: In addition to compounds XLVI and XLVII, a side product originating from the hydrolysis of methyl ester in 6.2k to carboxylic acid was also isolated in 3% yield].

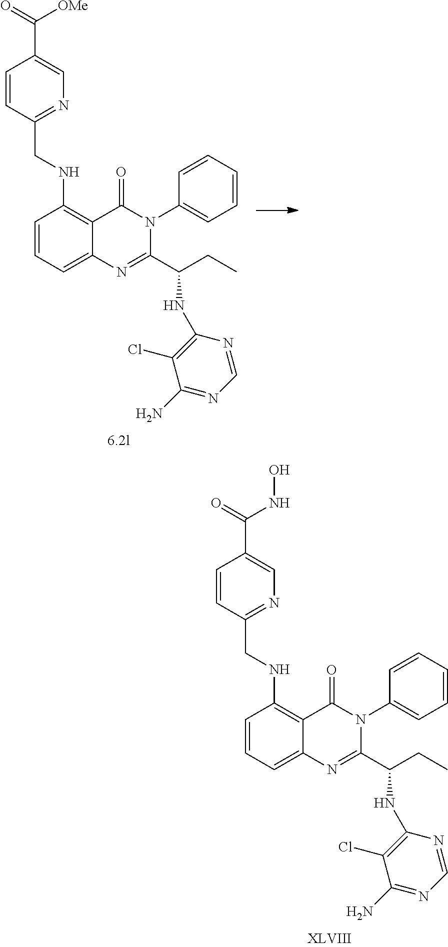 [SMILES: CC[C@H](Nc1ncnc(N)c1Cl)c1nc2cccc(NCc3ccc(C(=O)OC)cn3)c2c(=O)n1-c1ccccc1 ; CC[C@H](Nc1ncnc(N)c1Cl)c1nc2cccc(NCc3ccc(C(=O)NO)cn3)c2c(=O)n1-c1ccccc1]

The procedure mentioned in Scheme 25 was used with compound 6.21 (30.0 mg, 0.053 mmol) to afford product (S)-6-(((2-(1-((6-amino-5-chloropyrimidin-4-yl)amino)propyl)-4-oxo-3-phenyl-3,4-dihydroquinazolin-5-yl)amino)methyl)-N-hydroxynicotinamide, TFA XLVIII (13.0 mg, 0.019 mmol) in 36% yield. LC-MS (method 2): t_{R}=3.89 min, m/z (M)⁺=572.3.

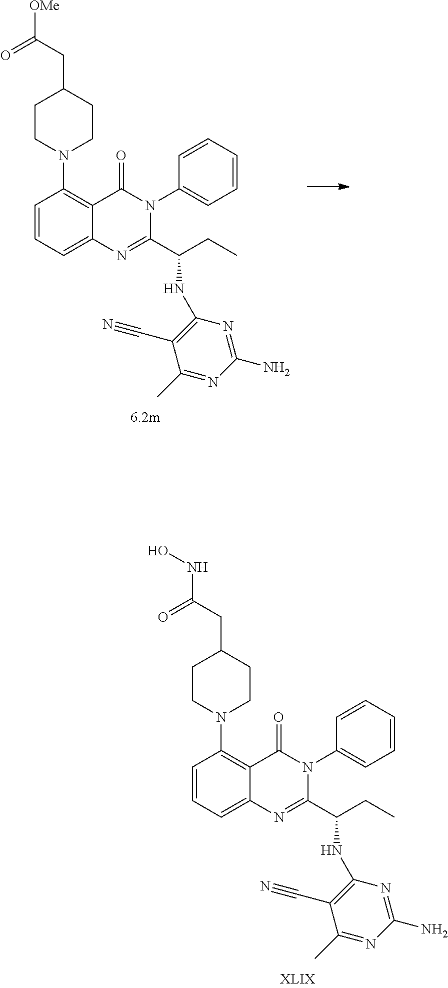 [SMILES: CC[C@H](Nc1nc(N)nc(C)c1C#N)c1nc2cccc(N3CCC(CC(=O)OC)CC3)c2c(=O)n1-c1ccccc1 ; CC[C@H](Nc1nc(N)nc(C)c1C#N)c1nc2cccc(N3CCC(CC(=O)NO)CC3)c2c(=O)n1-c1ccccc1]

The procedure mentioned in Scheme 25 was used with compound 6.2m (32.0 mg, 0.056 mmol) to afford product (S)-2-(1-(2-(1-((2-amino-5-cyano-6-methylpyrimidin-4-yl)amino)propyl)-4-oxo-3-phenyl-3,4-dihydroquinazolin-5-yl)piperidin-4-yl)-N-hydroxyacetamide, TFA XLV (14.5 mg, 0.021 mmol) in 38% yield. LC-MS (method 2): t_{R}=3.22 min, m/z (M+H)⁺=568.3. ¹H NMR (400 MHz, DMSO-d6) δ 10.38 (s, 1H), 7.91 (s, 1H), 7.75 (s, 1H), 7.55 (d, J=23.8 Hz, 4H), 7.48 (q, J=7.5 Hz, 2H), 7.26 (s, 1H), 4.75 (d, J=6.0 Hz, 1H), 3.51 (bs, 2H), 2.54 (d, J=1.4 Hz, 2H), 2.30 (s, 3H), 2.01-1.75 (m, 7H), 1.50 (s, 2H), 0.70 (t, J=7.3 Hz, 3H).

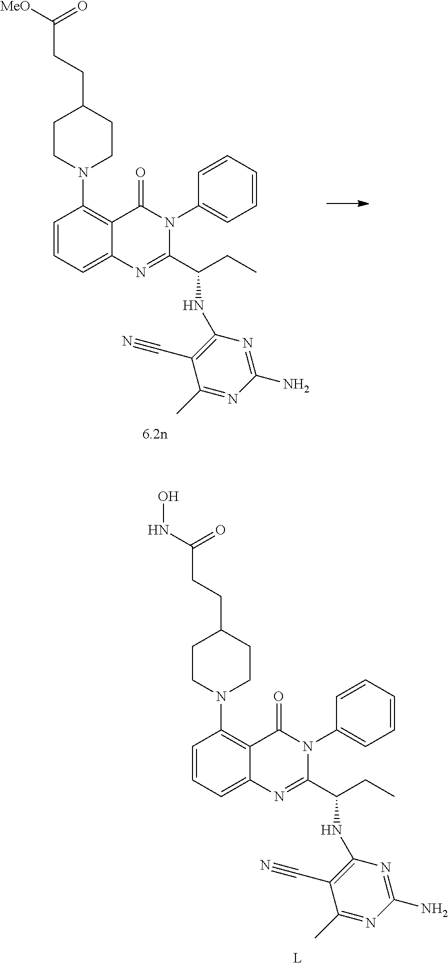 [SMILES: CC[C@H](Nc1nc(N)nc(C)c1C#N)c1nc2cccc(N3CCC(CCC(=O)OC)CC3)c2c(=O)n1-c1ccccc1 ; CC[C@H](Nc1nc(N)nc(C)c1C#N)c1nc2cccc(N3CCC(CCC(=O)NO)CC3)c2c(=O)n1-c1ccccc1]

The procedure mentioned in Scheme 25 was used with compound 6.2n (20.0 mg, 0.034 mmol) to afford product (S)-3-(1-(2-(1-((2-amino-5-cyano-6-methylpyrimidin-4-yl)amino)propyl)-4-oxo-3-phenyl-3,4-dihydroquinazolin-5-yl)piperidin-4-yl)-N-hydroxypropanamide, TFA L (5.0 mg, 7.19 mol) in 21% yield. LC-MS (method 2): t_{R}=3.28 min, m/z (M+H)⁺=582.3. ¹H NMR (400 MHz, DMSO-d6) δ 10.34 (s, 1H), 7.89 (s, 1H), 7.53 (td, J=19.3, 17.5, 8.1 Hz, 6H), 7.21 (s, 2H), 4.76-4.70 (m, 1H), 3.51 (bs, 2H), 2.54 (d, J=1.5 Hz, 3H), 2.29 (s, 4H), 1.99 (t, J=7.1 Hz, 2H), 1.89-1.73 (m, 4H), 1.48 (s, 4H), 0.69 (t, J=7.3 Hz, 3H).

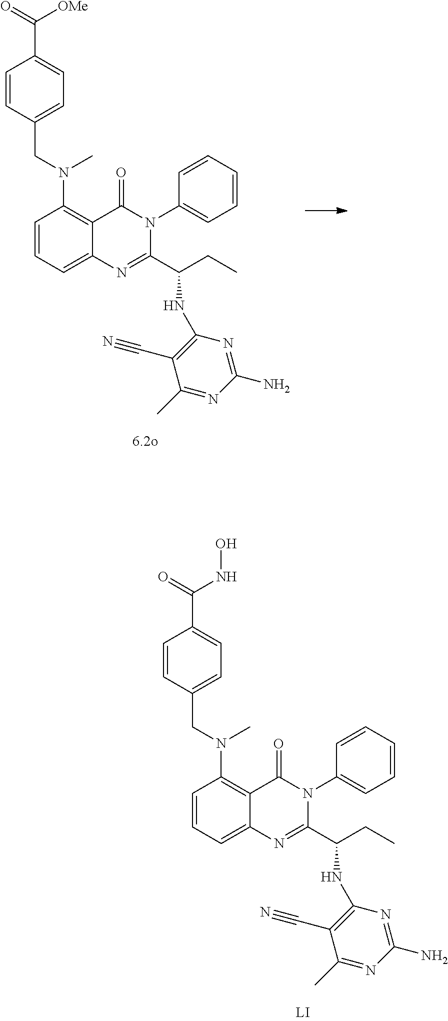 [SMILES: CC[C@H](Nc1nc(N)nc(C)c1C#N)c1nc2cccc(N(C)Cc3ccc(C(=O)OC)cc3)c2c(=O)n1-c1ccccc1 ; CC[C@H](Nc1nc(N)nc(C)c1C#N)c1nc2cccc(N(C)Cc3ccc(C(=O)NO)cc3)c2c(=O)n1-c1ccccc1]

The procedure mentioned in Scheme 25 was used with compound 6.2o (11.0 mg, 0.019 mmol) to afford product (S)-4-(((2-(1-((2-amino-5-cyano-6-methylpyrimidin-4-yl)amino)propyl)-4-oxo-3-phenyl-3,4-dihydroquinazolin-5-yl)(methyl)amino)methyl)-N-hydroxybenzamide, TFA LI (3.5 mg, 5.0 μmol) in 27% yield. LC-MS (method 2): t_{R}=3.25 min, m/z (M+H)⁺=590.3. 1H NMR (400 MHz, DMSO-d6) δ 11.11 (s, 1H), 7.64 (d, J=8.0 Hz, 3H), 7.52 (dd, J=15.9, 5.8 Hz, 4H), 7.46-7.40 (m, 1H), 7.35 (d, J=8.0 Hz, 2H), 7.17 (d, J=8.5 Hz, 2H), 7.07 (s, 1H), 4.72 (m, 1H), 4.41 (s, 2H), 3.17 (s, 1H), 2.72 (d, J=18.3 Hz, 3H), 2.30 (s, 3H), 1.86 (d, J=13.6 Hz, 1H), 1.75 (dt, J=14.5, 7.4 Hz, 1H), 0.69 (t, J=7.3 Hz, 3H).

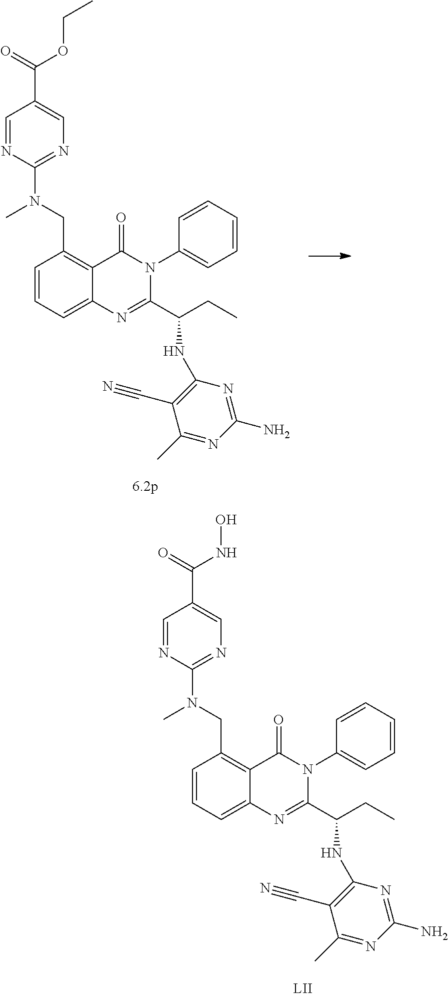 [SMILES: CCOC(=O)c1cnc(N(C)Cc2cccc3nc([C@H](CC)Nc4nc(N)nc(C)c4C#N)n(-c4ccccc4)c(=O)c23)nc1 ; CC[C@H](Nc1nc(N)nc(C)c1C#N)c1nc2cccc(CN(C)c3ncc(C(=O)NO)cn3)c2c(=O)n1-c1ccccc1]

The procedure mentioned in Scheme 25 was used with compound 6.2p (15.0 mg, 0.025 mmol) to afford product (S)-2-(((2-(1-((2-amino-5-cyano-6-methylpyrimidin-4-yl)amino)propyl)-4-oxo-3-phenyl-3,4-dihydroquinazolin-5-yl)methyl)(methyl)amino)-N-hydroxypyrimidine-5-carboxamide, TFA LII (11.0 mg, 0.016 mmol) in 63% yield. LC-MS (method 2): t_{R}=3.95 min, m/z (M+H)⁺=592.3. 1H NMR (400 MHz, DMSO-d6) δ 11.05 (s, 1H), 8.75 (s, 1H), 8.55 (s, 1H), 7.97 (s, 1H), 7.74 (t, J=7.9 Hz, 1H), 7.61-7.47 (m, 5H), 7.45 (dd, J=6.8, 1.9 Hz, 1H), 7.42 (d, J=2.1 Hz, 1H), 7.37 (s, 1H), 7.00 (d, J=7.7 Hz, 1H), 5.43 (s, 2H), 4.75 (td, J=7.7, 4.8 Hz, 1H), 3.26 (s, 3H), 2.33 (s, 3H), 1.94 (dt, J=13.2, 6.7 Hz, 1H), 1.80 (dq, J=15.7, 8.4, 7.9 Hz, 1H), 0.72 (t, J=7.3 Hz, 3H).

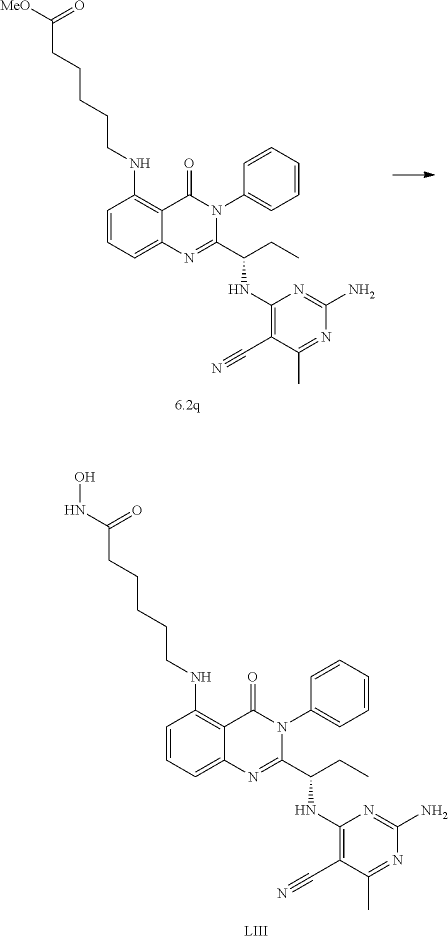 [SMILES: CC[C@H](Nc1nc(N)nc(C)c1C#N)c1nc2cccc(NCCCCCC(=O)OC)c2c(=O)n1-c1ccccc1 ; CC[C@H](Nc1nc(N)nc(C)c1C#N)c1nc2cccc(NCCCCCC(=O)NO)c2c(=O)n1-c1ccccc1]

The procedure mentioned in Scheme 25 was used with compound 6.2q (76.0 mg, 0.14 mmol) to afford product (S)-6-((2-(1-((2-amino-5-cyano-6-methylpyrimidin-4-yl)amino)propyl)-4-oxo-3-phenyl-3,4-dihydroquinazolin-5-yl)amino)-N-hydroxyhexanamide, TFA LIII (67.1 mg, 0.10 mmol) in 73% yield. LC-MS (method 2): t_{R}=4.11 min, m/z (M+H)⁺=556.3. ¹H NMR (400 MHz, DMSO-d6) δ 10.27 (s, 1H), 8.03 (s, 1H), 7.60-7.48 (m, 4H), 7.48-7.37 (m, 4H), 6.74 (dd, J=7.9, 1.0 Hz, 1H), 6.59 (d, J=8.4 Hz, 1H), 4.74 (td, J=7.6, 4.8 Hz, 1H), 3.15 (t, J=6.9 Hz, 2H), 2.54 (s, 1H), 2.35 (s, 3H), 1.98-1.84 (m, 3H), 1.76 (dp, J=14.5, 7.3 Hz, 1H), 1.58 (dt, J=17.6, 8.5 Hz, 3H), 1.51 (d, J=7.5 Hz, 2H), 1.39-1.27 (m, 2H), 0.71 (t, J=7.3 Hz, 3H). [Note: In addition to compound LIII, a side product originating from the hydrolysis of methyl ester in 6.2q to carboxylic acid was also isolated in 8% yield].

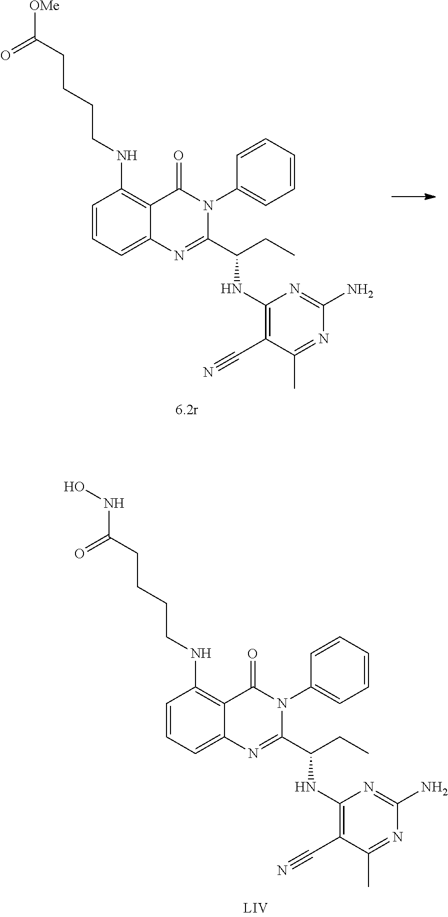 [SMILES: CC[C@H](Nc1nc(N)nc(C)c1C#N)c1nc2cccc(NCCCCC(=O)OC)c2c(=O)n1-c1ccccc1 ; CC[C@H](Nc1nc(N)nc(C)c1C#N)c1nc2cccc(NCCCCC(=O)NO)c2c(=O)n1-c1ccccc1]

The procedure mentioned in Scheme 25 was used with compound 6.2r (74.0 mg, 0.14 mmol) to afford product (S)-5-((2-(1-((2-amino-5-cyano-6-methylpyrimidin-4-yl)amino)propyl)-4-oxo-3-phenyl-3,4-dihydroquinazolin-5-yl)amino)-N-hydroxypentanamide, TFA LIIV (73.5 mg, 0.112 mmol) in 82% yield. LC-MS (method 2): t_{R}=4.02 min, m/z (M+H)⁺=542.3. 1H NMR (400 MHz, DMSO-d6) δ 10.30 (s, 1H), 8.03 (s, 1H), 7.63-7.51 (m, 3H), 7.51-7.46 (m, 3H), 7.46-7.37 (m, 2H), 6.74 (dd, J=7.9, 1.0 Hz, 1H), 6.59 (d, J=8.5 Hz, 1H), 4.74 (td, J=7.6, 4.8 Hz, 1H), 3.18 (d, J=6.1 Hz, 2H), 2.54 (s, 1H), 2.35 (s, 3H), 2.02-1.85 (m, 3H), 1.76 (dp, J=14.6, 7.4 Hz, 1H), 1.57 (q, J=4.1 Hz, 4H), 0.70 (t, J=7.3 Hz, 3H). [Note: In addition to compound LIV, a side product originating from the hydrolysis of methyl ester in 6.2r to carboxylic acid was also isolated in 7% yield].

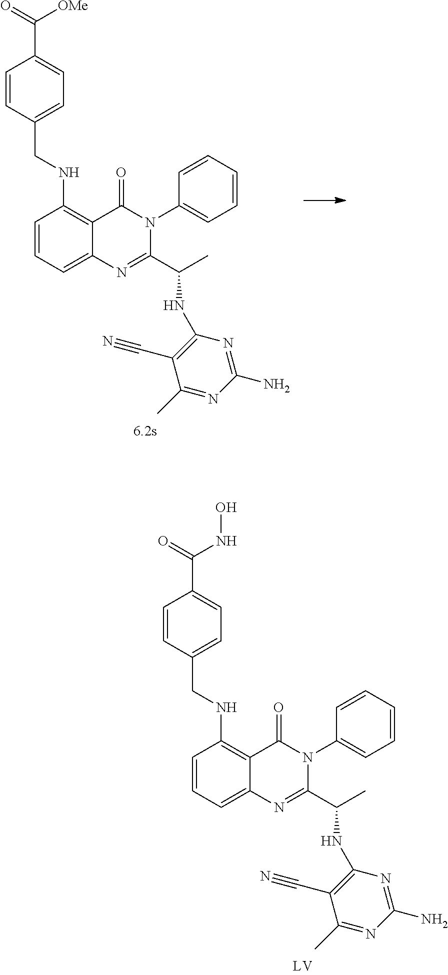 [SMILES: COC(=O)c1ccc(CNc2cccc3nc([C@H](C)Nc4nc(N)nc(C)c4C#N)n(-c4ccccc4)c(=O)c23)cc1 ; Cc1nc(N)nc(N[C@@H](C)c2nc3cccc(NCc4ccc(C(=O)NO)cc4)c3c(=O)n2-c2ccccc2)c1C#N]

The procedure mentioned in Scheme 25 was used with compound 6.2s (84.0 mg, 0.15 mmol) to afford product (S)-4-(((2-(1-((2-amino-5-cyano-6-methylpyrimidin-4-yl)amino)ethyl)-4-oxo-3-phenyl-3,4-dihydroquinazolin-5-yl)amino)methyl)-N-hydroxybenzamide, TFA LV (53.0 mg, 0.08 mmol) in 52% yield. LC-MS (method 2): t_{R}=3.93 min, m/z (M+H)⁺=562.3. 1H NMR (400 MHz, DMSO-d6) δ 11.14 (s, 1H), 8.98 (s, 1H), 7.95 (dq, J=16.2, 8.1 Hz, 1H), 7.70 (d, J=8.0 Hz, 2H), 7.55-7.39 (m, 7H), 7.37 (dd, J=5.0, 2.2 Hz, 1H), 7.33 (s, 1H), 6.81-6.67 (m, 1H), 6.51 (d, J=8.4 Hz, 1H), 4.86 (p, J=6.7 Hz, 1H), 4.54-4.48 (m, 2H), 2.54 (s, 1H), 2.30 (s, 3H), 1.34 (d, J=6.6 Hz, 3H). [Note: In addition to compound LV, a side product originating from the hydrolysis of methyl ester in 6.2s to carboxylic acid was also isolated in 3% yield].

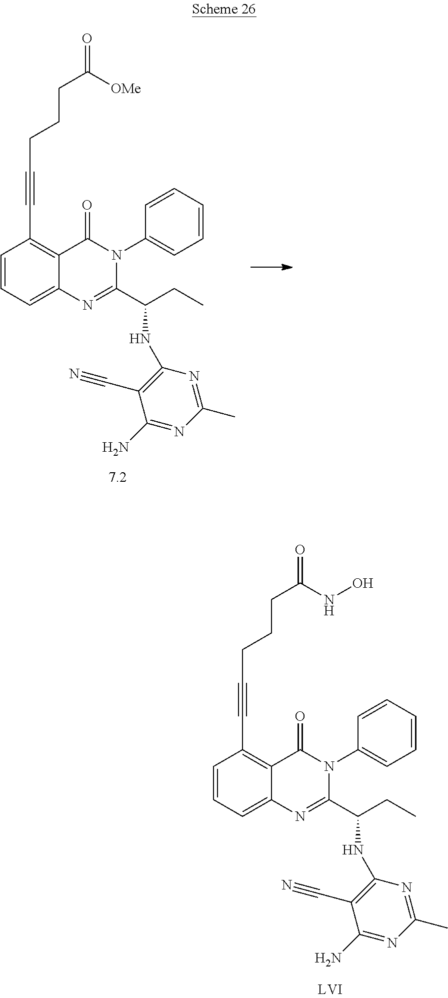 [SMILES: CC[C@H](Nc1nc(C)nc(N)c1C#N)c1nc2cccc(C#CCCCC(=O)OC)c2c(=O)n1-c1ccccc1 ; CC[C@H](Nc1nc(C)nc(N)c1C#N)c1nc2cccc(C#CCCCC(=O)NO)c2c(=O)n1-c1ccccc1]

### Scheme 26

Dissolved compound 7.2 (28.0 mg, 0.052 mmol) in MeOH (1.0 ml) in a vial equipped with a stir bar and added 50% hydroxylamine in water solution (30.0 equiv) and lithium hydroxide (2.6 mg, 0.063 mmol)) at 0° C. to it. The resulting solution was stirred at 0° C. for 2 hours and then allowed to warmup to room temperature overnight. After completion of reaction by LC-MS, the reaction mixture was concentrated in vacuo and purified by C-18 reverse phase chromatography under neutral H₂O/CH₃CN system to afford the final compound (S)-6-(2-(1-((2-amino-5-cyano-6-methylpyrimidin-4-yl)amino)propyl)-4-oxo-3-phenyl-3,4-dihydroquinazolin-5-yl)-N-hydroxyhex-5-ynamide LVI (5.0 mg, 9.32 mol) in 18% yield. LC-MS (method 2): t_{R}=3.97 min, m/z (M+H)⁺=537.3. ¹H NMR (400 MHz, DMSO-d6) δ 10.31 (s, 1H), 7.75 (t, J=7.9 Hz, 1H), 7.63-7.41 (m, 7H), 7.39 (s, 1H), 5.76 (s, 1H), 4.66 (td, J=7.7, 4.5 Hz, 1H), 2.54 (d, J=1.2 Hz, 1H), 2.41 (t, J=7.1 Hz, 2H), 2.25 (s, 3H), 2.09 (t, J=7.5 Hz, 2H), 1.92-1.75 (m, 3H), 1.75-1.67 (m, 2H), 0.67 (t, J=7.3 Hz, 3H).

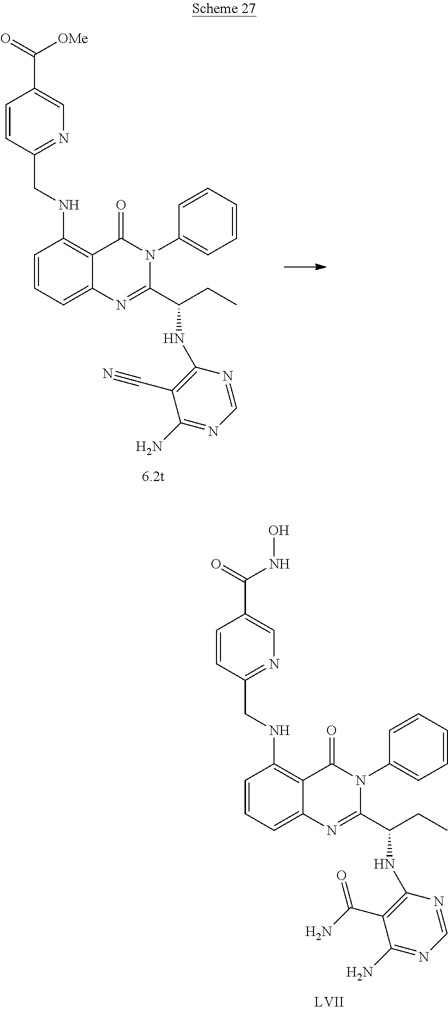 [SMILES: CC[C@H](Nc1ncnc(N)c1C#N)c1nc2cccc(NCc3ccc(C(=O)OC)cn3)c2c(=O)n1-c1ccccc1 ; CC[C@H](Nc1ncnc(N)c1C(N)=O)c1nc2cccc(NCc3ccc(C(=O)NO)cn3)c2c(=O)n1-c1ccccc1]

### Scheme 27

Dissolved compound 6.2t (30.0 mg, 0.053 mmol) in MeOH (1.0 ml) in a vial equipped with a stir bar and added freshly prepared methanolic hydroxylamine solution (30.0 equiv, Cai, X. et al. WO 2012/13571 A1) at 0° C. to it. The resulting solution was stirred at 0° C. for 2 hours and then allowed to warmup to room temperature overnight. After completion of reaction by LC-MS, the reaction mixture was concentrated in vacuo and purified by C-18 reverse phase chromatography to afford the final compound (S)-4-amino-6-((1-(5-(((5-(hydroxycarbamoyl)pyridin-2-yl)methyl)amino)-4-oxo-3-phenyl-3,4-dihydroquinazolin-2-yl)propyl)amino)pyrimidine-5-carboxamide, TFA LVII (15.0 mg, 0.022 mmol) in 40% yield. LC-MS (method 2): t_{R}=3.44 min, m/z (M+H)⁺=581.3. 1H NMR (400 MHz, DMSO-d6) δ 11.32 (s, 1H), 9.20 (s, 1H), 8.85 (d, J=2.2 Hz, 1H), 8.78 (d, J=7.6 Hz, 1H), 8.20 (s, 1H), 8.06 (dd, J=8.1, 2.3 Hz, 1H), 7.91 (s, 2H), 7.73 (s, 1H), 7.66-7.60 (m, 1H), 7.60-7.48 (m, 5H), 7.45 (d, J=8.2 Hz, 1H), 6.75 (d, J=7.8 Hz, 1H), 6.52 (d, J=8.4 Hz, 1H), 4.79 (td, J=7.8, 4.2 Hz, 1H), 4.60 (s, 2H), 1.87 (ddd, J=14.2, 7.4, 4.5 Hz, 1H), 1.67 (dq, J=14.5, 7.3 Hz, 1H), 0.71 (t, J=7.3 Hz, 3H).

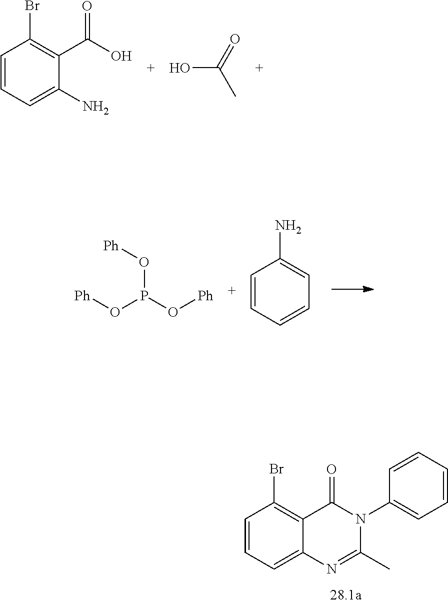

### Scheme 28

Glacial acetic acid (180.0 mg, 2.99 mmol, 171.0 μl) was added to a stirred solution of 2-amino-6-bromobenzoic acid (646.0 mg, 2.99 mmol) and triphenyl phosphite (928.0 mg, 2.99 mmol, 786.0 μl) in pyridine (8 mL) in a 20 ml MW vial under nitrogen atmosphere. After addition, the MW vial was sealed and the reaction mixture was heated to reflux for 4 h. Aniline (278.0 mg, 2.99 mmol, 272.0 μl) was added to the aforementioned reaction mixture, and heating was continued for another 12 h. After completion of the reaction (monitored by LC-MS), the reaction mixture was cooled to RT, concentrated in vacuo and purified by 0-35% EtOAc/Hexanes to afford the product 5-bromo-2-methyl-3-phenylquinazolin-4(3H)-one 28.1a (664.0 mg, 2.11 mmol) in 70% yield. LC-MS (method 1): t_{R}=2.97 min, m/z (M+2)⁺=317.1.

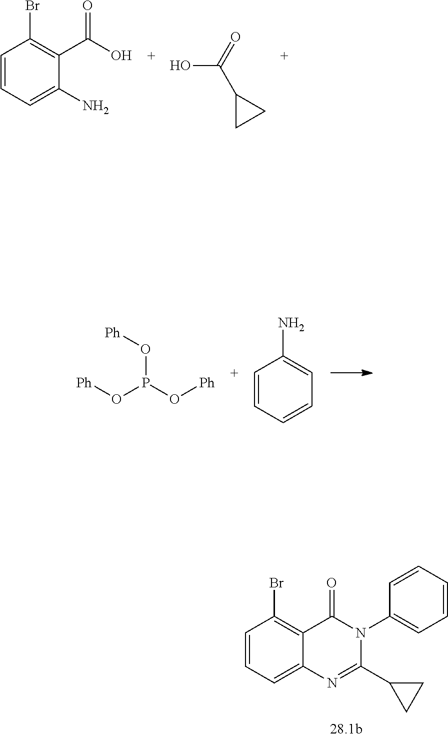 [SMILES: Nc1cccc(Br)c1C(=O)O ; O=C(O)C1CC1 ; c1ccc(OP(Oc2ccccc2)Oc2ccccc2)cc1 ; Nc1ccccc1 ; O=c1c2c(Br)cccc2nc(C2CC2)n1-c1ccccc1]

Cyclopropanecarboxylic acid (246.0 mg, 2.86 mmol, 227.0 μl) was added to a stirred solution of 2-amino-6-bromobenzoic acid (617.2 mg, 2.86 mmol) and triphenyl phosphite (886.0 mg, 2.86 mmol, 751.0 μl) in pyridine (8 mL) in a 20 ml MW vial under nitrogen atmosphere. After addition, the MW vial was sealed and the reaction mixture was heated to reflux for 4 h. Aniline (266.0 mg, 2.86 mmol, 260.0 μl) was added to the aforementioned reaction mixture, and heating was continued for another 12 h. After completion of the reaction (monitored by LC-MS), the reaction mixture was cooled to RT, concentrated in vacuo and purified by 0-20% EtOAc/Hexanes to afford the product 5-bromo-2-cyclopropyl-3-phenylquinazolin-4(3H)-one 28.1b (496.0 mg, 1.45 mmol) in 51% yield. LC-MS (method 1): t_{R}=3.34 min, m/z (M)⁺=341.1.

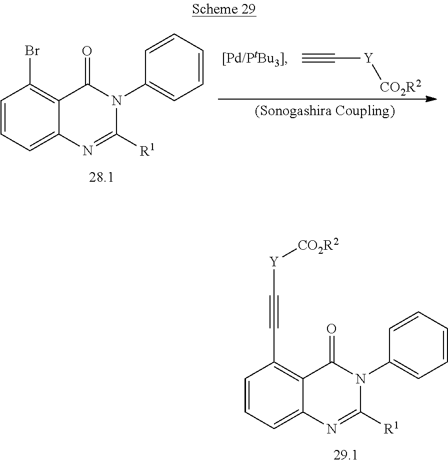

### Scheme 29

The substituted aryl bromide 28.1 (1 equiv), Allylpalladium(II) chloride dimer (0.05 equiv), Tri-tert-butylphosphonium tetrafluoroborate (0.20 equiv) and alkyne (1.2 equiv) [if solid at room temperature] were weighed and added to a MW vial equipped with a stir bar. The vial was covered with a rubber septum and placed under nitrogen atmosphere. In a separate scintillation vial, DABCO was weighed and dissolved in dry 1,4-dioxane (5 ml/mmol of aryl bromide). This DABCO solution and alkyne [if oil at room temperature] were added to the MW vial via syringe and the resulting mixture is bubbled with nitrogen for 5 min followed by stirring for 16 hours at room temperature under nitrogen atmosphere. After 16 hours, the crude reaction mixture is filtered through a short pad of celite and concentrated in vacuo. The remaining residue was purified by flash chromatography on silica using forced flow of ethyl acetate/hexanes system on Biotage KP-Sil pre-packed cartridges and using the Biotage SP-1 automated chromatography system to afford the coupled product 29.1.

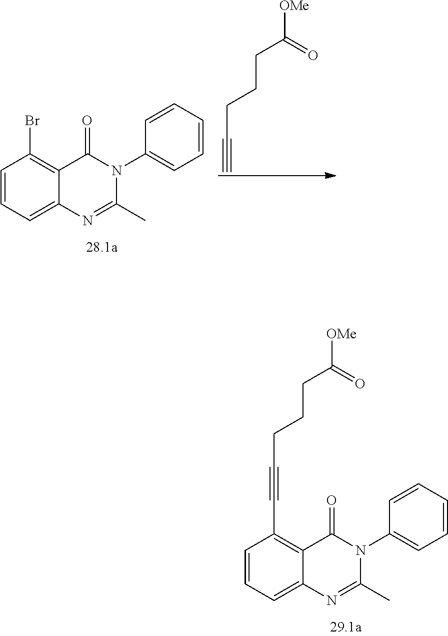

The procedure mentioned in Scheme 29 was used with compound 28.1a (162.9 mg, 0.52 mmol), Allylpalladium(II) chloride dimer (9.5 mg, 0.03 mmol), Tri-tert-butylphosphonium tetrafluoroborate (30.0 mg, 0.10 mmol), methyl hex-5-ynoate (78.0 mg, 0.62 mmol) and DABCO (116.0 mg, 1.03 mmol) in 2.5 ml of dry 1,4-dioxane. The resulting mixture was stirred at room temperature for 16 hours and concentrated in vacuo. The remaining residue was purified by flash chromatography on silica using 0-30% ethyl acetate/hexanes to afford the product methyl 6-(2-methyl-4-oxo-3-phenyl-3,4-dihydroquinazolin-5-yl)hex-5-ynoate 29.1a (133.0 mg, 0.37 mmol) as yellow oil in 71% yield. LC-MS (method 1): t_{R}=3.20 min, m/z (M+H)⁺=361.1. ¹H NMR (400 MHz, Chloroform-d) δ 7.65 (d, J=6.1 Hz, 2H), 7.59-7.48 (m, 4H), 7.29-7.27 (m, 2H), 3.66 (d, J=1.0 Hz, 3H), 2.54 (dt, J=16.3, 7.2 Hz, 4H), 2.24 (s, 3H), 1.95 (p, J=7.2 Hz, 2H).

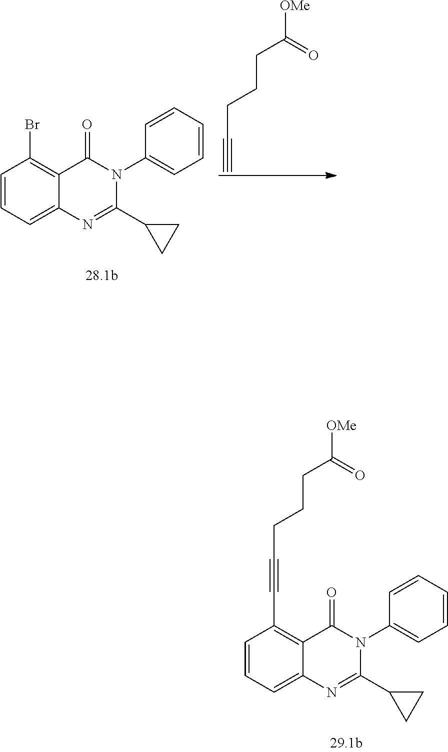

The procedure mentioned in Scheme 29 was used with compound 28.1b (163.0 mg, 0.48 mmol), Allylpalladium(II) chloride dimer (8.7 mg, 0.02 mmol), Tri-tert-butylphosphonium tetrafluoroborate (27.7 mg, 0.10 mmol), methyl hex-5-ynoate (72.3 mg, 0.57 mmol) and DABCO (107.0 mg, 0.96 mmol) in 2.4 ml of dry 1,4-dioxane. The resulting mixture was stirred at room temperature for 16 hours and concentrated in vacuo. The remaining residue was purified by flash chromatography on silica using 0-45% ethyl acetate/hexanes to afford the product methyl 6-(2-methyl-4-oxo-3-phenyl-3,4-dihydroquinazolin-5-yl)hex-5-ynoate 29.1a (160.0 mg, 0.41 mmol) as yellow oil in 87% yield. LC-MS (method 1): t_{R}=3.59 min, m/z (M+H)⁺=387.2. ¹H NMR (400 MHz, Chloroform-d) δ 7.62-7.47 (m, 6H), 7.38-7.30 (m, 2H), 3.66 (s, 3H), 2.54 (dt, J=15.1, 7.3 Hz, 4H), 1.98-1.91 (m, 2H), 1.42-1.30 (m, 3H), 0.86 (dq, J=7.1, 3.9 Hz, 2H).

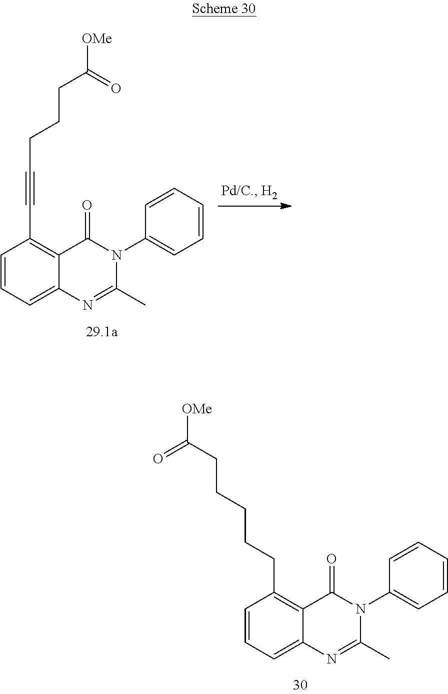 [SMILES: COC(=O)CCCC#Cc1cccc2nc(C)n(-c3ccccc3)c(=O)c12 ; COC(=O)CCCCCc1cccc2nc(C)n(-c3ccccc3)c(=O)c12]

### Scheme 30

The internal alkyne 29.1 (60.7 mg, 0.17 mmol) and 10 wt % Pd/C were added to a round-bottomed flask fitted with a rubber septum. The reaction flask is evacuated followed by the addition of dry EtOAc (0.1 M). The vacuum is removed and the reaction flask is kept under an atmosphere of hydrogen using a balloon and was stirred for 20 h. After completion of reaction (by LC MS), the crude reaction mixture is filtered using celite, concentrated in vacuo to afford the product methyl 6-(2-methyl-4-oxo-3-phenyl-3,4-dihydroquinazolin-5-yl)hexanoate 30. LC-MS (method 1): t_{R}=3.16 min, m/z (M+H)⁺=365.3.

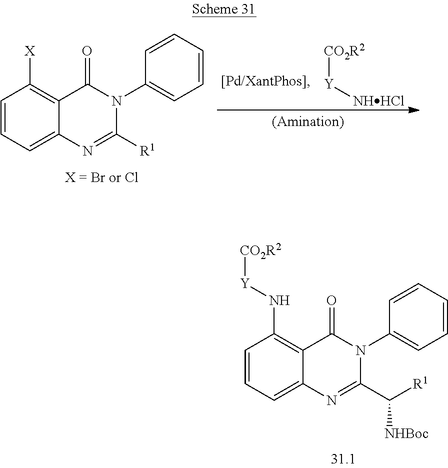

### Scheme 31

The substituted aryl halide (1 equiv), Methanesulfonato[9,9-dimethyl-4,5-bis(diphenylphosphino)xanthene] (2′-methylamino-1,1′-biphenyl-2-yl)palladium(II) XantPhos Palladacycle (Methanesulfonato [9,9-dimethyl-4,5-bis(diphenylphosphino)xanthene](2′-methylamino-1,1′-biphenyl-2-yl)palladium(II), Strem Chemicals Inc) (0.03 equiv) and amine (1.3 equiv) were weighed and added to a microwave vial equipped with a stir bar. The vial was covered with a rubber septum, evacuated and then filled with nitrogen. Dry 1,4-dioxane (0.2 M) was added to the vial followed by the addition of Cs₂CO₃ (3.0 equiv) under nitrogen bubbling through the solvent. The microwave vial is sealed and heated at 110° C. for 20 hours. After 20 hours, the crude reaction mixture is filtered through a short pad of celite and concentrated in vacuo. The remaining residue was purified by flash chromatography on silica using forced flow of ethyl acetate/hexanes system on Biotage KP-Sil pre-packed cartridges and using the Biotage SP-1 automated chromatography system to afford the coupled product 31.1.

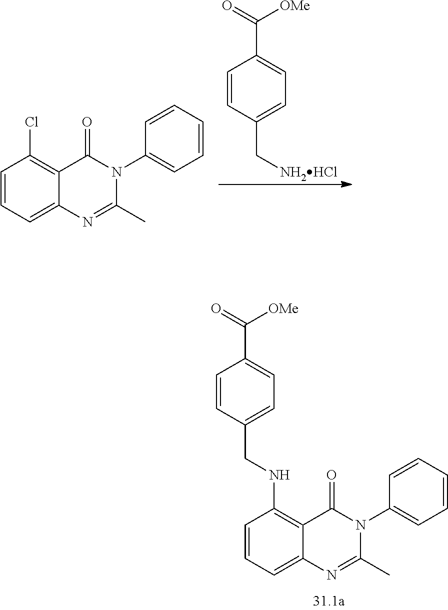

The procedure mentioned in Scheme 31 was used with 5-chloro-2-methyl-3-phenylquinazolin-4(3H)-one (46.6 mg, 0.17 mmol), [XantPhos Palladacycle] (8.2 mg, 8.6 μmol), Cs₂CO₃ (168.0 mg, 0.52 mmol) and methyl 4-(aminomethyl)benzoate hydrochloride (41.6 mg, 0.21 mmol) were combined in dry dioxane (0.8 ml). The resulting mixture was heated at 110° C. for 20 hours and concentrated in vacuo. The remaining residue was purified by flash chromatography on silica using 0-50% ethyl acetate/hexanes to afford the product methyl 4-(((2-methyl-4-oxo-3-phenyl-3,4-dihydroquinazolin-5-yl)amino)methyl)benzoate 31.1a (54.3 mg, 0.14 mmol) in 79% yield. LC-MS (method 1): t_{R}=3.07 min, m/z (M+H)⁺=400.2. ¹H NMR (400 MHz, Chloroform-d) δ 9.00 (s, 1H), 8.02-7.91 (m, 2H), 7.64-7.49 (m, 3H), 7.45 (dd, J=22.2, 8.1 Hz, 3H), 7.29 (dt, J=8.1, 1.1 Hz, 2H), 6.91 (s, 1H), 6.40 (d, J=8.4 Hz, 1H), 4.49 (d, J=5.8 Hz, 2H), 3.93-3.87 (m, 3H), 2.24 (s, 3H).

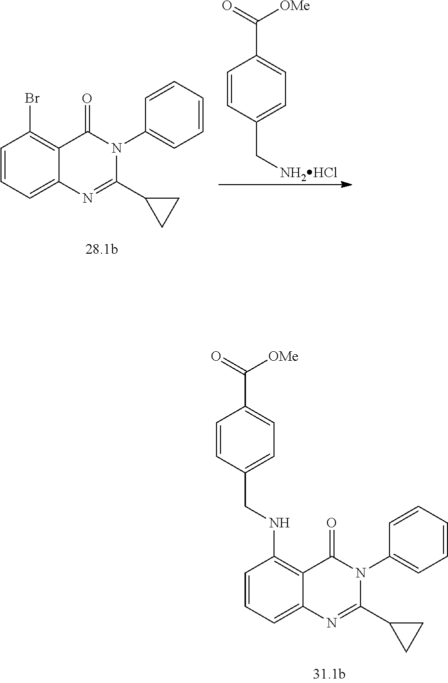

The procedure mentioned in Scheme 31 was used with compound 28.1b (64.1 mg, 0.19 mmol), [XantPhos Palladacycle] (5.4 mg, 5.64 μmol), Cs₂CO₃ (184.0 mg, 0.56 mmol) and methyl 4-(aminomethyl)benzoate hydrochloride (45.5 mg, 0.23 mmol) were combined in dry dioxane (0.9 ml). The resulting mixture was heated at 110° C. for 20 hours and concentrated in vacuo. The remaining residue was purified by flash chromatography on silica using 0-50% ethyl acetate/hexanes to afford the product methyl 4-(((2-cyclopropyl-4-oxo-3-phenyl-3,4-dihydroquinazolin-5-yl)amino)methyl)benzoate 31.1b (75.0 mg, 0.18 mmol) in 94% yield. LC-MS (method 1): t_{R}=3.47 min, m/z (M+H)⁺=426.2. ¹H NMR (400 MHz, Chloroform-d) δ 9.04 (t, J=5.9 Hz, 1H), 8.02-7.94 (m, 2H), 7.63-7.55 (m, 2H), 7.55-7.46 (m, 1H), 7.46-7.32 (m, 5H), 6.80 (d, J=7.9 Hz, 1H), 6.33 (dd, J=8.4, 1.0 Hz, 1H), 4.48 (d, J=5.8 Hz, 2H), 3.90 (d, J=1.0 Hz, 3H), 1.37 (ddt, J=12.9, 8.0, 4.3 Hz, 1H), 1.29-1.25 (m, 2H), 0.82 (dq, J=7.0, 3.8 Hz, 2H).

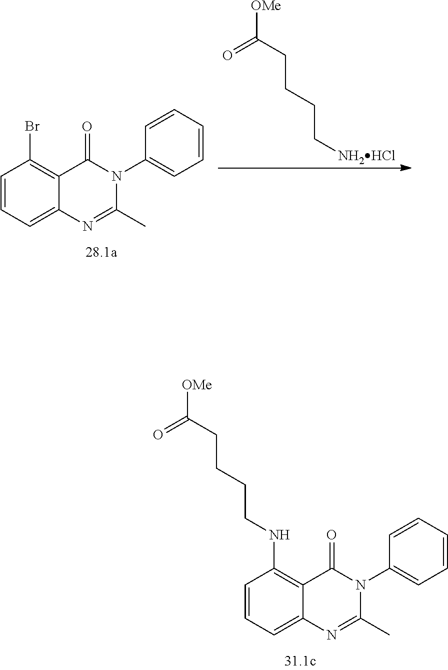

The procedure mentioned in Scheme 31 was used with compound 28.1a (95.2 mg, 0.30 mmol), [XantPhos Palladacycle] (8.7 mg, 9.1 mol), Cs₂CO₃ (295.0 mg, 0.91 mmol) and 5-methoxy-5-oxopentan-1-aminium chloride (60.8 mg, 0.31 mmol) were combined in dry dioxane (0.9 ml). The resulting mixture was heated at 110° C. for 20 hours and concentrated in vacuo. The remaining residue was purified by flash chromatography on silica using 0-70% ethyl acetate/hexanes to afford the product methyl 5-((2-methyl-4-oxo-3-phenyl-3,4-dihydroquinazolin-5-yl)amino)pentanoatee 31.1c (59.0 mg, 0.16 mmol) in 54% yield. LC-MS (method 1): t_{R}=3.04 min, m/z (M+H)⁺=366.2. ¹H NMR (400 MHz, Chloroform-d) δ 8.46 (t, J=5.1 Hz, 1H), 7.62-7.46 (m, 4H), 7.27-7.24 (m, 2H), 6.85-6.78 (m, 1H), 6.52-6.45 (m, 1H), 3.65 (s, 3H), 3.24-3.14 (m, 2H), 2.33 (t, J=7.0 Hz, 2H), 2.18 (s, 3H), 1.82-1.63 (m, 4H).

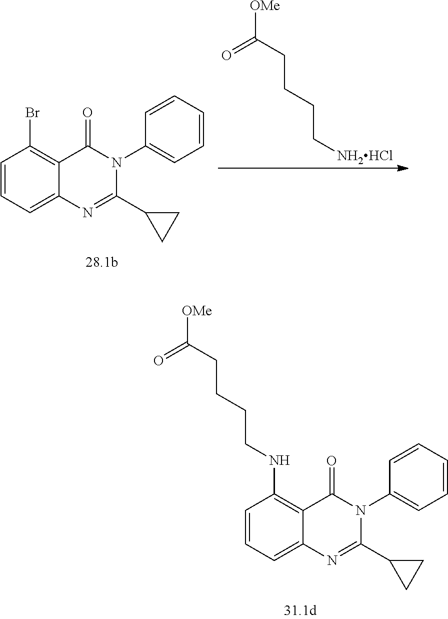

The procedure mentioned in Scheme 31 was used with compound 28.1b (82.2 mg, 0.24 mmol), [XantPhos Palladacycle] (7.0 mg, 7.23 μmol), Cs₂CO₃ (235.0 mg, 0.72 mmol) and 5-methoxy-5-oxopentan-1-aminium chloride (48.5 mg, 0.29 mmol) were combined in dry dioxane (1.2 ml). The resulting mixture was heated at 110° C. for 20 hours and concentrated in vacuo. The remaining residue was purified by flash chromatography on silica using 0-60% ethyl acetate/hexanes to afford the product methyl 5-((2-cyclopropyl-4-oxo-3-phenyl-3,4-dihydroquinazolin-5-yl)amino)pentanoate 31.1d (40.0 mg, 0.10 mmol) in 40% yield. LC-MS (method 1): t_{R}=3.52 min, m/z (M+H)⁺=392.2. ¹H NMR (400 MHz, Chloroform-d) δ 8.47 (t, J=5.0 Hz, 1H), 7.63-7.45 (m, 4H), 7.35-7.31 (m, 2H), 6.76 (d, J=7.8 Hz, 1H), 6.47-6.40 (m, 1H), 3.66 (s, 3H), 3.23-3.14 (m, 2H), 2.34 (t, J=7.0 Hz, 2H), 1.79-1.65 (m, 4H), 1.38-1.25 (m, 3H), 0.80 (dq, J=7.1, 3.8 Hz, 2H).

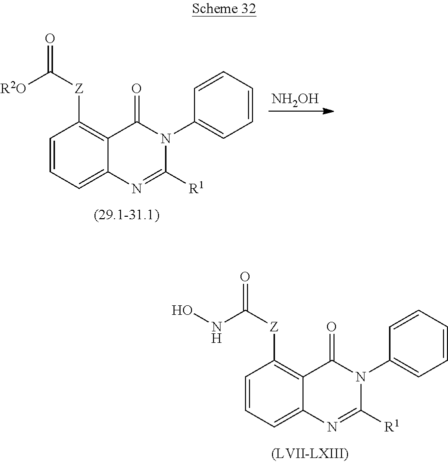

### Scheme 32

The methyl ester bearing compound (1 equiv) [29.1-31.1] was dissolved in MeOH (0.1M) in a MW vial equipped with a stir bar and 50% hydroxylamine in water solution (30.0 equiv) and lithium hydroxide (1.2-2.0 equiv) were added at 0° C. to it. The MW vial was sealed and the resulting solution was stirred at 0° C. for 2 hours and then allowed to warmup to room temperature overnight. After completion of reaction by LC-MS, the reaction mixture was concentrated in vacuo and purified by C-18 reverse phase chromatography to afford the final compound (LVII-LXIII).

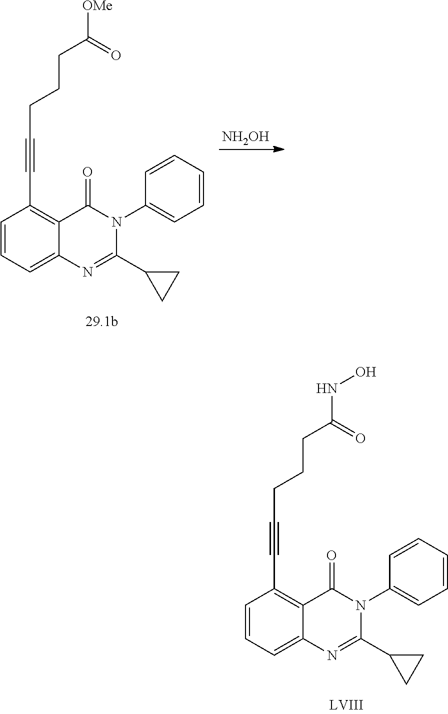

The procedure mentioned in Scheme 32 was used with compound 29.1b (104.6 mg, 0.27 mmol) to afford product 6-(2-cyclopropyl-4-oxo-3-phenyl-3,4-dihydroquinazolin-5-yl)-N-hydroxyhex-5-ynamide, TFA LVIII (25.0 mg, 0.05 mmol) in 18% yield. LC-MS (method 2): t_{R}=4.75 min, m/z (M+H)⁺=388.2. ¹H NMR (400 MHz, DMSO-d6) δ 10.30 (s, 1H), 7.68 (dd, J=8.4, 7.3 Hz, 1H), 7.64-7.56 (m, 2H), 7.56-7.39 (m, 6H), 3.17 (s, 1H), 2.41 (t, J=7.1 Hz, 2H), 2.10 (t, J=7.4 Hz, 2H), 1.73 (p, J=7.3 Hz, 2H), 1.30 (tt, J=8.0, 4.6 Hz, 1H), 1.12 (dt, J=4.6, 3.1 Hz, 2H), 0.81 (dt, J=8.2, 3.4 Hz, 2H). [Note: In addition to compound LVIII, a side product originating from the hydrolysis of methyl ester in 29.1b to carboxylic acid was also isolated in 2% yield].

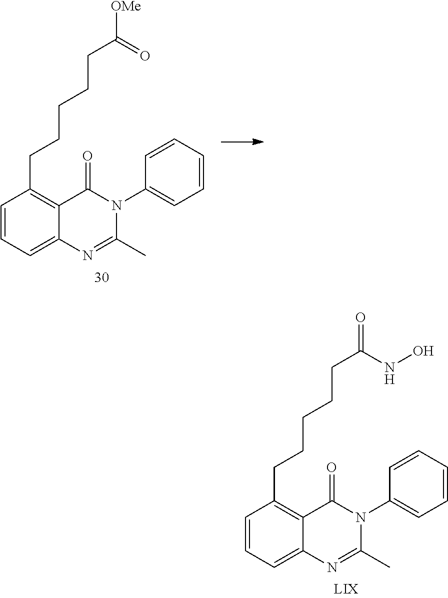 [SMILES: COC(=O)CCCCCc1cccc2nc(C)n(-c3ccccc3)c(=O)c12 ; Cc1nc2cccc(CCCCCC(=O)NO)c2c(=O)n1-c1ccccc1]

The procedure mentioned in Scheme 32 was used with compound 30 (60.0 mg, 0.165 mmol) to afford product N-hydroxy-6-(2-methyl-4-oxo-3-phenyl-3,4-dihydroquinazolin-5-yl)hexanamide, TFA LIX (50.0 mg, 0.10 mmol) in 63% yield. LC-MS (method 2): t_{R}=3.54 min, m/z (M+H)⁺=366.2. ¹H NMR (400 MHz, DMSO-d6) δ 10.28 (s, 1H), 7.74 (t, J=7.8 Hz, 1H), 7.63-7.37 (m, 7H), 7.35-7.28 (m, 1H), 3.13 (t, J=7.7 Hz, 2H), 2.14 (s, 3H), 1.90 (t, J=7.3 Hz, 2H), 1.49 (dt, J=15.5, 7.7 Hz, 4H), 1.28 (q, J=7.6, 7.1 Hz, 2H).

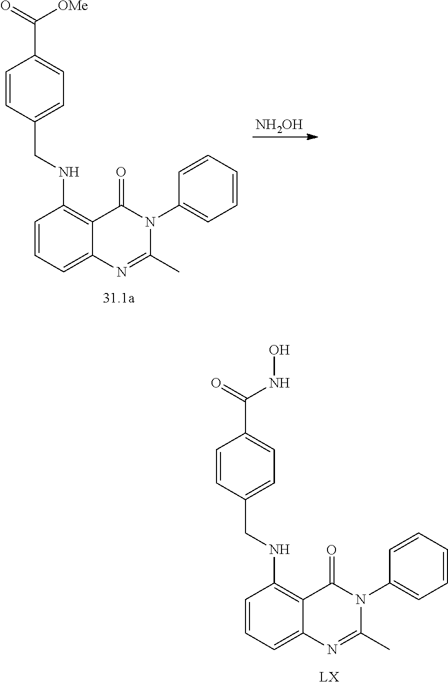 [SMILES: COC(=O)c1ccc(CNc2cccc3nc(C)n(-c4ccccc4)c(=O)c23)cc1 ; "NH2OH" ; Cc1nc2cccc(NCc3ccc(C(=O)NO)cc3)c2c(=O)n1-c1ccccc1]

The procedure mentioned in Scheme 32 was used with compound 31.1a (52.3 mg, 0.131 mmol) to afford product N-hydroxy-4-(((2-methyl-4-oxo-3-phenyl-3,4-dihydroquinazolin-5-yl)amino)methyl)benzamide, TFA LX (42.0 mg, 0.082 mmol) in 62% yield. LC-MS (method 2): t_{R}=3.59 min, m/z (M+H)⁺=401.1. 1H NMR (400 MHz, DMSO-d6) δ 11.16 (s, 1H), 8.91 (s, 1H), 7.74-7.66 (m, 2H), 7.63-7.37 (m, 9H), 6.75 (d, J=7.9 Hz, 1H), 6.53 (d, J=8.4 Hz, 1H), 4.51 (s, 2H), 2.13 (s, 3H).

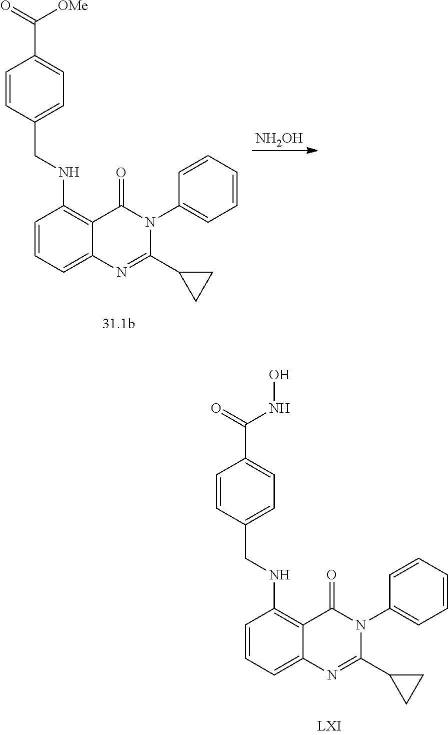

The procedure mentioned in Scheme 32 was used with compound 31.1b (37.0 mg, 0.09 mmol) to afford the product 4-(((2-cyclopropyl-4-oxo-3-phenyl-3,4-dihydroquinazolin-5-yl)amino)methyl)-N-hydroxybenzamide, TFA LXI (21.0 mg, 0.04 mmol) in 45% yield. LC-MS (method 2): t_{R}=4.53 min, m/z (M+H)⁺=427.2. 1H NMR (400 MHz, DMSO-d6) δ 11.14 (s, 1H), 8.95 (s, 1H), 7.73-7.66 (m, 2H), 7.66-7.54 (m, 2H), 7.54-7.47 (m, 2H), 7.47-7.32 (m, 5H), 6.67-6.60 (m, 1H), 6.41 (d, J=8.3 Hz, 1H), 4.48 (s, 2H), 1.29 (tt, J=8.1, 4.7 Hz, 1H), 1.08 (dq, J=6.3, 3.8 Hz, 2H), 0.77 (dt, J=10.2, 3.3 Hz, 2H). [Note: In addition to compound LXI, a side product originating from the hydrolysis of methyl ester in 31.1b to carboxylic acid was also isolated in 9% yield].

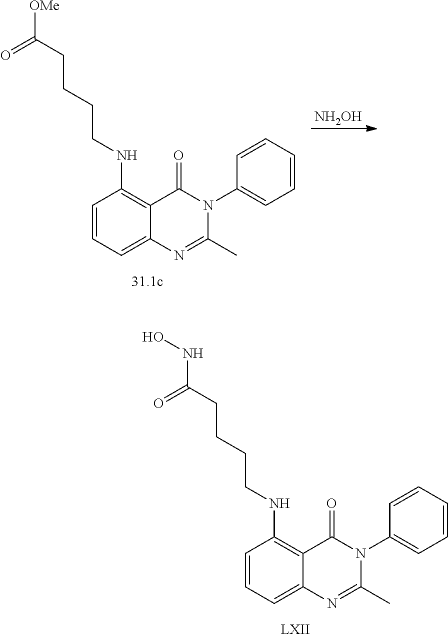

The procedure mentioned in Scheme 32 was used with compound 31.1c (55.0 mg, 0.15 mmol) to afford the product N-hydroxy-5-((2-methyl-4-oxo-3-phenyl-3,4-dihydroquinazolin-5-yl)amino)pentanamide, TFA LXII (19.0 mg, 0.04 mmol) in 26% yield. LC-MS (method 2): t_{R}=3.36 min, m/z (M+H)⁺=367.2. 1H NMR (400 MHz, DMSO-d6) δ 10.33 (s, 1H), 8.42 (s, 1H), 7.62-7.47 (m, 4H), 7.47-7.39 (m, 2H), 6.70 (d, J=7.9 Hz, 1H), 6.57 (d, J=8.4 Hz, 1H), 3.16 (d, J=3.6 Hz, 2H), 2.08 (s, 3H), 1.96 (d, J=6.7 Hz, 2H), 1.61-1.53 (m, 4H). [Note: In addition to compound LXII, a side product originating from the hydrolysis of methyl ester in 31.1c to carboxylic acid was also isolated in 4% yield].

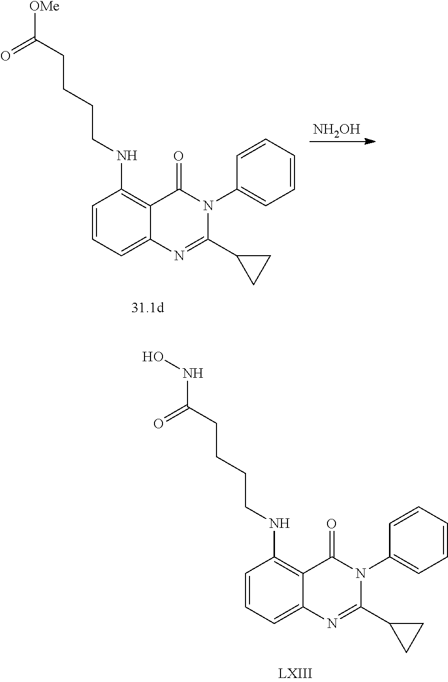

The procedure mentioned in Scheme 32 was used with compound 31.1d (36.0 mg, 0.092 mmol) to afford the product 5-((2-cyclopropyl-4-oxo-3-phenyl-3,4-dihydroquinazolin-5-yl)amino)-N-hydroxypentanamide, TFA LXIII (15.0 mg, 0.03 mmol) in 32% yield. LC-MS (method 2): t_{R}=4.35 min, m/z (M+H)⁺=393.2. 1H NMR (400 MHz, DMSO-d6) δ 10.34 (s, 1H), 8.44 (s, 1H), 7.58 (dd, J=8.2, 6.7 Hz, 2H), 7.54-7.41 (m, 4H), 6.61 (d, J=7.9 Hz, 1H), 6.48 (d, J=8.4 Hz, 1H), 3.15 (q, J=6.1, 5.7 Hz, 2H), 1.97 (d, J=6.8 Hz, 2H), 1.64-1.50 (m, 4H), 1.27 (tt, J=8.2, 4.6 Hz, 1H), 1.08 (dq, J=6.4, 3.8 Hz, 2H), 0.77 (dt, J=8.2, 3.4 Hz, 2H).

### Biological Assays

### PI3Kα, PI3Kβ, PI3Kγ, and PI3Kδ Kinase Assay Protocol

### HTRF Assay Platform

### 1: Assay Description

Assay Principle:

The PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed by PI 3-Kinase(h) activity displaces biotin-PIP3 from the complex resulting in a loss of energy transfer, and thus, a decrease in signal.

This is a 3-Step Reaction:

First, the kinase reaction with PIP2 substrate is carried out in the presence of ATP, and the reaction is quenched with stop Solution, and then, finally detect by adding Detection Mixture followed by incubation.

### 2: Reaction Conditions:

Assay Buffer:

HEPES 50 mM (pH7.0), NaN3 0.02%, BSA 0.01%, Orthovanadate 0.1 mM, 1% DMSO.

Detection buffer: HEPES 10 mM (pH7.0), BSA 0.02%, KF 0.16 M, EDTA 4 mM.

Substrate:

10 μM PIP2 substrate (PI(4,5)P2)

ATP:

10 μM ATP under standard conditions

Control Inhibitor:

PI-103

### 3: Assay Procedure:

1. Prepare substrate in freshly prepared Reaction Buffer.

2. Deliver kinase into the substrate solution and gently mix.

3. Deliver compounds in 100% DMSO into the kinase reaction mixture by Acoustic technology (Echo550; nanoliter range), incubate for 10 min at room temperature.

4. Deliver ATP into the reaction mixture to initiate the reaction.

5. Incubate for 30 min at 30° C.

6. Quench the reaction with Stop Solution.

7. Add Detection Mixture, and incubate for overnight.

8. Measure HTRF: Ex=320 nm, ratio of Em=615 nm and Em=665 nm.

### 4: Data Analysis:

The emission ratio is converted into μM PIP3 production based on PIP3 standard curves.

The nonlinear regression to obtain the standard curve and IC₅₀ values are performed using Graphpad Prism software.

### HDAC Fluorescent Activity Assay:

This protocol is to determine the IC₅₀s or percentage of inhibition values of the test compound against HDACs.

### Assay Description:

The HDAC Fluorescent Activity Assay is based on the unique Fluorogenic Substrate and Developer combination. This assay is a highly sensitive and validated. The assay procedure has two steps (FIG. 1, Howitz, 2015 Drug Discovery Today: Technologies). First, the Fluorogenic Substrate, which comprises an acetylated lysine side chain, is incubated with a purified HDAC enzyme. Deacetylation of the substrate sensitizes the substrate so that, in the second step, treatment with the Developer produces a fluorophore.

Compound Handling:

Testing compounds were dissolved in 100% DMSO to specific concentration. The serial dilution was conducted by epMotion 5070 in DMSO.

Materials and Reagents:

HDAC reaction buffer: 50 mM Tris-HCl, pH8.0, 137 mM NaCl, 2.7 mM KCl, and 1 mM MgCl2, Add fresh: 1 mg/ml BSA, 1% DMSO

Substrate: HDAC1,2,3,6,10: Fluorogenic peptide from p53 residues 379-382 (RHKK(Ac)AMC). HDAC4,5,7, 9 and 11: Fluorogenic HDAC Class2a Substrate (Trifluoroacetyl Lysine). HDAC 8: Fluorogenic peptide from p53 residues 379-382 (RHK(Ac)K(Ac)AMC)

### General Reaction Procedure: (Standard IC₅₀ Determination)

### Deacetylation Step:

1. Deliver 2× enzyme in wells of reaction plate except No Enzyme control wells. Add buffer in No En wells.

2. Deliver compounds in 100% DMSO into the enzyme mixture by Acoustic technology (Echo550; nanoliter range). Spin down and pre-incubation.

3. Deliver 2× Substrate Mixture (Fluorogenic HDAC Substrate and co-factor if applicable) in all reaction wells to initiate the reaction. Spin and shake.

4. Incubate for 1-2 hr at 30° C. with seal.

### Development Step:

5. Add Developer with Trichostatin A (or TMP269) to stop the reaction and to generate fluorescent color.

6. Fluorescence was read (excitatory, 360; emission, 460) using the EnVision Multilabel

### Plate Reader (Perkin Elmer).

7. Take endpoint reading for analysis after the development reaches plateau.

Data Analysis:

The percentages of enzyme activity (relative to DMSO controls) and IC50 values were calculated using the GraphPad Prism 4 program based on a sigmoidal dose-response equation.

TABLE 11
PI3Kδ Inhibition

| Compound Number | Activity |
|---|---|
| I | ** |
| II | ** |
| III | ** |
| IV | ++ |
| V | ++ |
| VI | * |
| VII | ++ |
| VIII | ++ |
| IX | ** |
| X | ++ |
| XI | ++ |
| XII | ++ |
| XIII | ** |
| XIV | ++ |
| XV | ++ |
| XVII | ++ |
| XVIII | * |
| XIX | + |
| XX | ++ |
| XXI | ** |
| XXII | ** |
| XXIII | ** |
| XXIV | ++ |
| XXV | ** |
| XXVI | ** |
| XXVII | ++ |
| XXVIII | ++ |
| XXIX | ++ |
| XXX | + |
| XXXI | + |
| XXXII | * |
| XXXIII | * |
| XXXIV | * |
| XXXV | ++ |
| XXXVI | ++ |
| XXXVII | ** |
| XXXVIII | ++ |
| XXXIX | ++ |
| XL | ++ |
| XLII | ++ |
| XLIII | + |
| XLIV | + |
| XLVIII | + |
| XLIX | ** |
| L | ++ |
| LI | ++ |
| LII | ++ |
| LVI | ++ |
| LVII | + |

Abbreviations:
“++” - IC₅₀ < 100 nM
“+” - IC₅₀ > 100 nM
“**” - % Inhibition @ 1 μM > 90%
“*” - % Inhibition @ 1 μM < 90%

PI3K8 Selectivity Over PI3Kα

Compounds XI, XV, XX, XXXV, XXXVI, XXXVIII, XLIII, XLIV, XLVIII, LII and LVII showed >10-fold selectivity.

Compounds IV, XVII, XXXIX, XL and XLII showed <10-fold selectivity.

PI3Kδ Selectivity Over PI3Kβ

Compounds IV, XI, XV, XVII, XX, XXXV, XXXVI, XXXVIII, XXXIX, XL, XLII, XLIII, XLIV, XLVIII, XLIX, LII, LVII, LX, LXI, LXII and LXIII showed >10-fold selectivity.

PI3Kδ Selectivity Over PI3Kγ

Compounds XI, XVII, XX, XXXVIII, XLII, XLIII, XLIV, XLVIII, LII and LVII showed >10-fold selectivity.

Compounds IV, XV, XXXV, XXXVI, XXXIX and XL showed <10-fold selectivity.

PI3Kα, PI3Kβ, PI3Kγ Inhibition

Compounds I, II, III, IV, V, VI, VIII, IX, XIII, XIV, XVII, XVIII, XIX, XX, XXI, XXII, XXIII, XXIV, XXV, XXVI, XXXII, XXXIII, and XXXIV showed % inhibition @ 1 uM<85%, except Compound IX (PI3Kγ % inhibition @ 1 uM=88) and Compound XXIV (PI3Kγ % inhibition @ 1 uM=91).

TABLE 2
HDAC6 Inhibition

| Compound Number | Activity |
|---|---|
| I | ** |
| II | * |
| III | * |
| IV | ++ |
| V | + |
| VI | * |
| VII | + |
| VIII | ** |
| IX | ** |
| X | + |
| XI | ++ |
| XII | ++ |
| XIII | * |
| XIV | + |
| XV | ++ |
| XVII | ++ |
| XVIII | * |
| XIX | ** |
| XX | ++ |
| XXI | * |
| XXII | * |
| XXIII | * |
| XXIV | + |
| XXV | * |
| XXVI | * |
| XXVII | + |
| XXVIII | ++ |
| XXIX | + |
| XXX | ++ |
| XXXI | ++ |
| XXXII | * |
| XXXIII | ** |
| XXXIV | ** |
| XXXV | ++ |
| XXXVI | ++ |
| XXXVII | ** |
| XXXVIII | ++ |
| XXXIX | ++ |
| XL | ++ |
| XLI | ++ |
| XLII | ++ |
| XLIII | ++ |
| XLIV | ++ |
| XLVIII | ++ |
| XLIX | * |
| L | ++ |
| LI | ++ |
| LII | ++ |
| LVI | ** |
| LVII | ++ |
| LX | ++ |
| LXI | ++ |
| LXII | + |
| LXIII | + |

Abbreviations:
“++” - IC₅₀ < 100 nM
“+” - IC₅₀ > 100 nM
“**” - % Inhibition @ 1 μM > 90%
“*” - % Inhibition @ 1 μM < 90%

HDAC6 Selectivity Over HDAC1

Compounds IV, XI, XII, XV, XVII, XX, XXVIII, XXXI, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

Compounds XIV and XXIX showed <10-fold selectivity.

HDAC6 Selectivity Over HDAC2

Compounds IV, XI, XII, XV, XVII, XX, XXVIII, XXXI, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

Compound XXIX showed <10-fold selectivity.

HDAC6 Selectivity Over HDAC3

Compounds IV, XI, XII, XV, XVII, XX, XXVIII, XXXI, XV, XXXV, XXXVI, XXXVIII, XXXIX, XL, XLI, XLII, XLIII, XLIV, XLVIII, XLIX, LII, LVII LX, LXI, LXII and LXIII showed >10-fold selectivity.

Compound XXIX showed <10-fold selectivity.

HDAC6 Selectivity Over HDAC4

Compounds IV, XI, XV, XVII, XX, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

HDAC6 Selectivity Over HDAC5

Compounds IV, XI, XV, XVII, XX, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

HDAC6 Selectivity Over HDAC7

Compounds IV, XI, XV, XVII, XX, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

HDAC6 Selectivity Over HDAC8

Compounds IV, XI, XVI, XV, XVII, XX, XXX, XXXI, XXXVI, XXXVIII, XXXIX, XL, XLI, XLII, XLIII, XLVIII, XLIX, LII, LVII LX and LXI showed >10-fold selectivity.

Compounds V, X, XIV, XXIV, XXVIII, XXIX, XXXV, XLIV, LXII and LXIII showed <10-fold selectivity.

HDAC6 Selectivity Over HDAC9

Compounds IV, XI, XV, XVII, XX, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

HDAC6 Selectivity Over HDAC10

Compounds IV, XI, XII, XV, XVII, XX, XXVIII, XXIX, XXXI XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

HDAC6 Selectivity Over HDAC11

Compounds IV, XI, XV, XVII, XX, XXXV, XXXVI, XXXVIII, XXXIX, XL, XLI, XLII, XLIII, XLIV, XLVIII, XLIX, LII, LVII, LX, LXI, LXII and LXIII showed >10-fold selectivity.

### Evaluation Against NCI-60 Cancer Cell Lines:

Assay Protocol:

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of experimental drugs.

After 24 h, two plates of each cell line are fixed in situ with TCA, to represent a measurement of the cell population for each cell line at the time of drug addiction (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC₅₀ (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

Interpretation of One-Dose Data:

The data is reported as a mean graph of the percent growth of treated cells. The number reported for the One-dose assay is growth relative to the no-drug control, and relative to the time zero number of cells. This allows detection of both growth inhibition (values between 0 and 100) and lethality (values less than 0). For example, a value of 100 means no growth inhibition. A value of 40 would mean 60% growth inhibition. A value of 0 means no net growth over the course of the experiment. A value of −40 would mean 40% lethality. A value of −100 means all cells are dead.

Using the above protocol, single dose data for selected compounds have been obtained as follows. Inhibition of proliferation in NCI60 cancer cell line panel was measured at 10 uM compound concentration.

Activity Against Leukemia Cell Line CCRF-CEM

Compounds IV, V, XXXV, XXXVI, XXXVII, XXXVIII, XXXIX, XL, XLI, XLII, XLIII, XLIV, XLVIII, LII and LX showed >50% inhibition.

Compounds XII, XXIV and XLIX showed 25%-50% inhibition.

Activity Against Leukemia Cell Line HL-60(TB)

Compounds IV, V, XXIV, XXXV, XXXVI, XXXVIII, XXXIX, XL, XLI, XLII, XLIII, XLIV, XLVIII, LII and LX showed >50% inhibition.

Compounds XXX, XXXVII and XLIX showed 25%-50% inhibition.

Activity Against Leukemia Cell Line K-562

Compounds IV, V, XXXVI, XXXVIII, XLVIII, LII AND LX showed >50% inhibition.

Compounds XXXVII and XLIII showed 25%-50% inhibition.

Activity Against Leukemia Cell Line MOLT-4

Compounds IV, V, XXIV, XXXV, XXXVI, XXXVIII, XXXIX, XL, XLI, XLII, XLIV, XLVIII, LII and LX showed >50% inhibition.

Compounds I, XII, XXIV, XXXVII and XLIII showed 25%-50% inhibition.

Activity Against Leukemia Cell Line RPMI-8226

Compounds I, IV, V, XII, XXXV, XXXVI, XXXVIII, XXXIX, XL, XLI, XLII, XLIV, XLVIII, LII and LX showed >50% inhibition.

Compounds XI, XXXVII and XLIII showed 25%-50% inhibition.

Activity Against Leukemia Cell Line SR

Compounds IV, V, XII, XXXVI, XXXVII, XXXVIII, XLIII, XLVIII, LII and LX showed >50% inhibition.

Compounds I and XXIV showed 25%-50% inhibition.

Activity Against Non-Small Cell Lung Cancer Cell Line A549/ATCC

Compounds XXXV, XXXVI, XL, XLII, XLIV, LII and LX showed >50% inhibition.

Compounds V, XXXI, XXXVIII and XLI showed 25%-50% inhibition.

Activity Against Non-Small Cell Lung Cancer Cell Line EKVX

Compounds XXXV, XXXVI, XL, XLIV and LII showed >50% inhibition.

Compounds XXXVIII, XXXIX, XLI, XLII and LX showed 25%-50% inhibition.

Activity Against Non-Small Cell Lung Cancer Cell Line HOP-62

Compounds IV, V, XXXV, XXXVI, XXXIX, XL, XLI, XLII, XLIV LII and LX showed >50% inhibition.

Compounds XXXVIII, XLIII and XLVIII showed 25%-50% inhibition.

Activity Against Non-Small Cell Lung Cancer Cell Line HOP-92

Compounds XXXV, XXXVI, XXXVII, XXXVIII, XXXIX, XL, XLI, XLII, XLIII, XLIV, XLVIII, LII and LX showed >50% inhibition.

Compounds I, V, XXIV and XLIX showed 25%-50% inhibition.

Activity Against Non-Small Cell Lung Cancer Cell Line NCI-H226

Compounds V, XXXV, XL and LII showed >50% inhibition.

Compounds IV, XXXVI, XXXIX, XLI, XLII, XLIV and LX showed 25%-50% inhibition.

Activity Against Non-Small Cell Lung Cancer Cell Line NCI-H23

Compounds XXXV, XXXVI, XXXVIII, XXXIX, XL, XLI, XLII, XLIV, XLVIII, LII, and LX showed >50% inhibition.

Compound XXXVIII showed 25%-50% inhibition.

Activity Against Non-Small Cell Lung Cancer Cell Line NCI-H322M

Compounds XXXV, XXXVI, XXXVIII, XXXIX, XL, XLI, XLII, XLIV and LII showed >50% inhibition.

Compounds XXXVIII, XLVIII and LX showed 25%-50% inhibition.

Activity Against Non-Small Cell Lung Cancer Cell Line NCI-H460

Compounds IV, V, XXXV, XXXVI, XXXIX, XL, XLI, XLII, XLIV, LII and LX showed >50% inhibition.

Compounds XX, XXXVIII and XLVIII showed 25%-50% inhibition.

Activity Against Non-Small Cell Lung Cancer Cell Line NCI-H522

Compounds XII, XXXV, XXXVI, XXXVII, XXXVIII, XXXIX, XL, XLI, XLII, XLIII, XLIV, XLVIII, LII and LX showed >50% inhibition.

Compounds I, IV, V, XI and XLIX showed 25%-50% inhibition.

Activity Against Colon Cancer Cell Line COLO-205

Compounds IV, V, XXXV, XXXVI, XXXVIII, XXXIX, XL, XLI, XLII, XLIV, XLVIII, LII, and LX showed >50% inhibition.

Activity Against Colon Cancer Cell Line HCC-2998

Compounds XXXV, XXXVI, XXXIX, XL, XLI, XLII, XLIV and LII showed >50% inhibition.

Compounds XXXVIII and XLVIII showed 25%-50% inhibition.

Activity Against Colon Cancer Cell Line HCT-116

Compounds XXXV, XXXVI, XXXVIII, XXXIX, XL, XLI, XLII, XLIV, LII and LX >50% inhibition.

Compounds XXXVII and XLIII showed 25%-50% inhibition

Activity Against Colon Cancer Cell Line HCT-15

Compounds XXXVI, XL, XLIV, LII and LX showed >50% inhibition.

Compounds IV and V showed 25%-50% inhibition.

Activity Against Colon Cancer Cell Line HT-29

Compounds IV, V, XXXV, XXXVI, XXXVIII, XXXIX, XL, XLI, XLII, XLIV, and LII showed >50% inhibition.

Compound XLVIII showed 25%-50% inhibition.

Activity Against Colon Cancer Cell Line KM12

Compounds IV, V, IV, V, XXXV, XXXVI, XXXVIII, XXXIX, XL, XLI, XLII, XLIV, and LII showed >50% inhibition.

Compound XLVIII showed 25%-50% inhibition.

Activity Against Colon Cancer Cell Line SW-620

Compounds XXXV, XXXVI, XXXVIII, XXXIX, XL, XLI, XLII, XLIV, and LII showed >50% inhibition.

Compounds XXXVII and XLIII showed 25%-50% inhibition.

Activity Against CNS Cancer Cell Line SF-268

Compounds IV, V, XXXV, XXXVI, XXXIX, XL, XLI, XLII, XLIV, LII and LX showed >50% inhibition.

Compounds XXIV, XXXVIII and XLVIII showed 25%-50% inhibition.

Activity Against CNS Cancer Cell Line SF-295

Compounds IV, V, XXXV, XXXVI, XXXVIII, XL, XLII, XLIV, LII and LX showed >50% inhibition.

Compounds XXXIX and XLI showed 25%-50% inhibition.

Activity Against CNS Cancer Cell Line SF-539

Compounds IV, V, XXXV, XXXVI, XXXIX, XL, XLI, XLII, XLIV, LII and LX showed >50% inhibition.

Compounds XXXVII and XXXVIII showed 25%-50% inhibition.

Activity Against CNS Cancer Cell Line SNB-19

Compounds IV, V, XXXV, XXXVI, XXXIX, XL, XLI, XLII, XLIV, LII and LX showed >50% inhibition.

Compounds VI, XXXVIII, XLIII and XLVIII showed 25%-50% inhibition.

Activity Against CNS Cancer Cell Line SNB-75

Compounds IV, V, XXIV, XXXV, XXXIX, XL, XLI, XLII, XLIV, LII and LX showed >50% inhibition.

Compounds I, III, XI, XII and XLIX showed 25%-50% inhibition.

Activity Against CNS Cancer Cell Line U251

Compounds IV, XXXVI, XXXVIII, XLIII, XLVIII, LII and LX showed >50% inhibition.

Compounds V, XX and XXXVII showed 25%-50% inhibition.

Activity Against Melanoma Cell Line LOX IMVI

Compounds XXXV, XXXVI, XXXIX, XL, XLI, XLII, XLIV, XLVIII, LII and LX showed >50% inhibition.

Compounds V and XXXVIII showed 25%-50% inhibition.

Activity Against Melanoma Cell Line MALME-3M

Compounds IV, V, XII, XXXV, XXXVI, XXXVIII, XXXIX, XL, XLI, XLII, XLIV, LII and LX showed >50% inhibition.

Compounds I, XXIV, XXXVII, XLIII and XLVIII showed 25%-50% inhibition.

Activity Against Melanoma Cell Line M14

Compounds XXXV, XXXVI, XXXIX, XL, XLI, XLII, XLIV, LII and LX showed >50% inhibition.

Compounds IV, V, XXXVIII and XLVIII showed 25%-50% inhibition.

Activity Against Melanoma Cell Line MDA-MB-435

Compounds IV, XXXV, XXXVI, XXXVIII, XXXIX, XL, XLI, XLII, XLIV, LII and LX showed >50% inhibition.

Compounds V and XXXVII showed 25%-50% inhibition.

Activity Against Melanoma Cell Line SK-MEL-2

Compounds IV, XXXV, XXXVI, XXXVIII, XXXIX, XL, XLI, XLII and LII showed >50% inhibition.

Compounds IV, XXXVII, XLIV and LX showed 25%-50% inhibition.

Activity Against Melanoma Cell Line SK-MEL-28

Compounds IV, XXXV, XXXVI, XXXVII, XXXVIII, XXXIX, XL, XLI, XLII, XLIV, LII and LX showed >50% inhibition.

Compounds V, XX, XLIII and XLIX showed 25%-50% inhibition.

Activity Against Melanoma Cell Line SK-MEL-5

Compounds IV, V, XXXV, XXXVI, XXXVII, XXXVIII, XXXIX, XL, XLI, XLII, XLIV, LII and LX showed >50% inhibition.

Activity Against Melanoma Cell Line UACC-257

Compounds IV, V, XXXV, XXXVI, XXXVIII, XXXIX, XL, XLI, XLII, XLIV and LII showed >50% inhibition.

Compounds I, XII, XXIV, XXXVII, XLIII, XLVIII and LX showed 25%-50% inhibition.

Activity Against Melanoma Cell Line UACC-62

Compounds IV, V, XXXV, XXXVI, XXXVII, XXXVIII, XXXIX, XL, XLI, XLII, XLIV, LII and LX showed showed >50% inhibition.

Compounds XXIV, XLIII and XLIX showed 25%-50% inhibition.

Activity Against Ovarian Cell Line IGROV1

Compounds XXIV, XXXV, XXXVI, XXXIX, XL, XLI, XLII, XLIV, XLVIII, LII, and LX showed >50% inhibition.

Compounds IV, V, XXXVIII, XLIII and XLIX showed 25%-50% inhibition.

Activity Against Ovarian Cell Line OVCAR-3

Compounds XXXV, XXXVI, XXXVIII, XXXIX, XL, XLI, XLII, XLIV, LII, and LX showed >50% inhibition.

Compounds IV, XXXVII, XLIII and XLVIII showed 25%-50% inhibition.

Activity Against Ovarian Cell Line OVCAR-4

Compounds V, XXXV, XXXVIII, XL, XLI, XLII, XLIV, XLVIII and LII showed >50% inhibition.

Compounds IV, XXXVI, XXXIX and LX showed 25%-50% inhibition.

Activity Against Ovarian Cell Line OVCAR-5

Compounds XXXV, XXXVI, XXXVIII, XL, XLI, XLII, XLIV, LII and LX showed >50% inhibition.

Compound XLVIII showed 25%-50% inhibition.

Activity Against Ovarian Cell Line OVCAR-8

Compounds XXXV, XXXVI, XXXVIII, XL, XLI, XLII, XLIV, LII and LX showed >50% inhibition.

Compounds XXXVII, XXXVIII and XLIII showed 25%-50% inhibition.

Activity Against Ovarian Cell Line NCI/ADR-RES

Compound LX showed >50% inhibition.

Compounds V, XXXVI, XL, XLIV and LII showed 25%-50% inhibition.

Activity Against Ovarian Cell Line SK-OV-3

Compounds XXXV, XXXVI, XXXVIII, XXXIX, XL, XLI, XLII, XLIV, LII, and LX showed >50% inhibition.

Compounds IV, V and XLVIII showed 25%-50% inhibition.

Activity Against Renal Cancer Cell Line 786-0

Compounds XXXV, XXXVI, XXXIX, XL, XLI, XLII, XLIV and LII showed >50% inhibition.

Compounds XXIV, XXXVIII, XLVIII and LX showed 25%-50% inhibition.

Activity Against Renal Cancer Cell Line A498

Compounds IV, V, XXXV, XXXVI, XXXIX, XL, XLI, XLII, XLIV, LII, and LX showed >50% inhibition.

Compounds I, XI, XV, XXXVIII and XLVIII showed 25%-50% inhibition.

Activity Against Renal Cancer Cell Line ACHN

Compounds IV, V, XXXVI, XL, XLII, XLIV, LII, and LX showed >50% inhibition.

Compounds IV and XXXV showed 25%-50% inhibition.

Activity Against Renal Cancer Cell Line CAKI-1

Compounds XXXVI, XL, XLIV, LII, and LX showed >50% inhibition.

Compounds XXXV, XXXVII, XXXIX, XLI and XLII showed 25%-50% inhibition.

Activity Against Renal Cancer Cell Line RXF 393

Compounds XXXV, XXXVI, XXXVII, XXXVIII, XXXIX, XL, XLI, XLII, XLIV, XLVIII, LII, and LX showed >50% inhibition.

Compounds IV, V, XXIV, XLIII and XLIX showed 25%-50% inhibition.

Activity Against Renal Cancer Cell Line SN12C

Compounds XXXV, XXXVI, XXXIX, XL, XLI, XLII, XLIV and LII showed >50% inhibition.

Compounds XXIV, XXXVII, XXXVIII, XLVIII and LX showed 25%-50% inhibition.

Activity Against Renal Cancer Cell Line TK-10

Compounds IV, V, XXXV, XXXVI, XXXVIII, XXXIX, XL, XLI, XLII, XLIV, XLVIII, LII, and LX showed >50% inhibition.

Compounds IV, XXXVII and XLIII showed 25%-50% inhibition.

Activity Against Renal Cancer Cell Line UO-31

Compounds XXXVI, XL, XLIV, LII and LX showed >50% inhibition.

Compounds V, XXXV, XXXVII, XXXVIII, XXXIX, XLII, XLIII and XLVIII showed 25%-50% inhibition.

Activity Against Prostate Cancer Cell Line PC-3

Compounds IV, V, XXXV, XXXVI, XL, XLI, XLII, XLIV, XLVIII, LII, and LX showed >50% inhibition.

Compounds I, XX, XXIV, XXXVII, XXXVIII and XXXIX showed 25%-50% inhibition.

Activity Against Prostate Cancer Cell Line DU-145

Compounds IV, XXXV, XXXVI, XXXIX, XL, XLI, XLII, XLIV, LII, and LX showed >50% inhibition.

Compounds V and XXXVIII showed 25%-50% inhibition.

Activity Against Breast Cancer Cell Line MCF7

Compounds IV, V, XXXV, XXXVI, XXXVIII, XXXIX, XL, XLI, XLII, XLIV, and LII showed >50% inhibition.

Compounds XXIV, XXXVII, XLVIII, XLIX and LX showed 25%-50% inhibition.

Activity Against Breast Cancer Cell Line MDA-MB-231/ATCC

Compounds V, XXXV, XXXVI, XXXIX, XL, XLI, XLII, XLIV, LII, and LX showed >50% inhibition.

Compounds IV, XXXVIII and XLVIII showed 25%-50% inhibition.

Activity Against Breast Cancer Cell Line HS 578T

Compounds IV, V, XXXV, XXXVI, XXXVII, XXXVIII, XXXIX, XL, XLI, XLII, XLIV, XLVIII and LII showed >50% inhibition.

Compounds I, XII, XX, XXIV, XLIII, XLIX and LX showed 25%-50% inhibition.

Activity Against Breast Cancer Cell Line BT-549

Compounds IV, V, XXXV, XXXVI, XXXVIII, XXXIX, XL, XLI, XLII, XLIV, and LII showed >50% inhibition.

Compounds XXIV, XXXVII, XLVIII and LX showed 25%-50% inhibition.

Activity Against Breast Cancer Cell Line T-47D

Compounds XXXV, XXXVI, XXXVII, XXXVIII, XXXIX, XL, XLI, XLII, XLIII, XLIV, XLVIII, LII, and LX showed >50% inhibition.

Compounds I, III, XI, XII, XXIV and XLIX showed 25%-50% inhibition.

Activity Against Breast Cancer Cell Line MDA-MB-468

Compounds XXXV, XXXVI, XXXVII, XXXVIII, XXXIX, XL, XLI, XLII, XLIII, XLIV, XLVIII, LII, and LX showed >50% inhibition.

Compounds I, III, XI, XII, XXIV and XLIX showed 25%-50% inhibition.

TABLE 3
shows the LC₅₀ data for selected compounds in NCI-60 Cell Five-
Dose screen.

| | | Compound Number | Compound Number |
|---|---|---|---|
| Cancer | Cell Line | (LC₅₀ < 10 μM) | (10 μM < LC₅₀ < 100 μM) |
| Leukemia | CCRF-CEM | | XXXVI |
| | SR | | XXXVI |
| Non-Small | A549/ATCC | | XXXVI, XL, XLI, XLII, |
| Cell Lung | | | XLIV |
| Cancer | EKVX | | XLI, XLII |
| | HOP-62 | XLI | XXXVI, XLII, XLIV |
| | HOP-92 | XLI, XLIV | XXXVI, XLII |
| | NCI-H226 | | XXXVI |
| | NCI-H23 | | XLII |
| | NCI-H322M | | XXXV, XLI, XLIV |
| | NCI-H460 | | IV, XXXVI, XXXIX, XL, |
| | | | XLI, XLII, XLIV |
| | NCI-H522 | XXXVI, XLI | XLII, XLIV |
| Colon Cancer | COLO 205 | XLI | XXXV, XXXVI, XLII, |
| | | | XLIV |
| | HCC-2998 | XLI | IV, XXXVI, XLII |
| | HCT-116 | XLI, XLII | XXXVI |
| | HCT-15 | | XXXVI, XLI, XLII |
| | HT29 | | XXXVI, XLI, XLII |
| | KM12 | XL, XLI, XLIV | XXXV, XXXVI, XLII |
| | SW-620 | XXXV, XL | XXXVI, XLI, XLII |
| CNS Cancer | SF-268 | XL, XLI | |
| | SF-295 | XXXVI, XLI | XLII |
| | SF-539 | XXXV, XL, XLI, | XXXVI, XLII |
| | | XLIV | |
| | SNB-19 | | XLI |
| | SNB-75 | | XLI, XLII |
| | U251 | XXXVI, XLI, XLIV | XLII |
| Melanoma | LOX IMVI | XXXVI, XLI, XLIV | XLII |
| | MALME-3M | XLI | XXXVI, XLIV |
| | M14 | XLI, XLII | |
| | MDA-MB-435 | XLI | XXXVI, XLII, XLIV |
| | SK-MEL-2 | | XXXVI, XLI, XLII, |
| | | | XLIV |
| | SK-MEL-28 | XLI | XXXVI, XLII, XLIV |
| | SK-MEL-5 | XXXV, XXXVI, XLI | XLII, XLIV |
| | UACC-257 | | XXXVI, XLI, XLII |
| | UACC-62 | | XXXVI, XLII |
| Ovarian | IGROV1 | | |
| Cancer | OVCAR-3 | XL | XXXV, XXXVI, XLI |
| | OVCAR-4 | | XLI |
| | OVCAR-5 | | XLI, XLII, XLIV |
| | OVCAR-8 | | XXXVI |
| | NCI/ADR-RES | | XXXVI |
| | SK-OV-3 | | XXXVI, XLI, XLII, |
| | | | XLIV |
| Renal Cancer | 786-0 | XLI, XLIV | |
| | A498 | XXXVI, XXXIX, XL, | IV, XLII, XLIV |
| | | XLI | |
| | ACHN | | XL, XLI, XLIV |
| | CAKI-1 | | IV, XXXVI, XLI, XLII |
| | RXF 393 | XXXVI, XL, XLI, | IV, XXXIX, XLII |
| | | XLIV | |
| | SN12C | | XLI |
| | TK-10 | | XLI |
| | UO-31 | | XLI, XLIV |
| Prostate | PC-3 | | XLI |
| Cancer | DU-145 | | IV, XXXV, XXXIX, XL, |
| | | | XLI, XLII, XLIV |
| Breast Cancer | MCF7 | XXXVI, | XLI |
| | MDA-MB-231/ATCC | XLI | XXXVI, XLII, XLIV |
| | HS 578T | | |
| | BT-549 | | XXXVI |
| | T-47D | | XLI |
| | MDA-MB-468 | XLI | XXXV, XXXVI, XLII |

The present inventive concept has been described in terms of exemplary principles and embodiments, but those skilled in the art will recognize that variations may be made and equivalents substituted for what is described without departing from the scope and spirit of the disclosure as defined by the following claims.

1. A dual inhibitor of phosphoinositide 3-kinase (PI3K) and histone deacetylase (HDAC), the dual inhibitor comprising:
a core comprising a quinazoline moiety or a quinazolin-4(3H)-one moiety;
a kinase hinge binding moiety; and
a histone deacetylase pharmacophore,
a pharmaceutically acceptable salt thereof, a prodrug thereof, or solvate thereof.

2. The dual inhibitor of claim 1, wherein the histone deacetylase pharmacophore comprises: 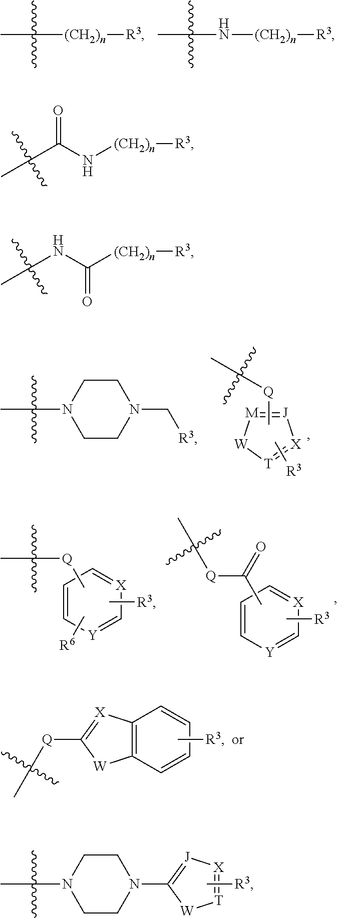
wherein in the above formulae,
at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 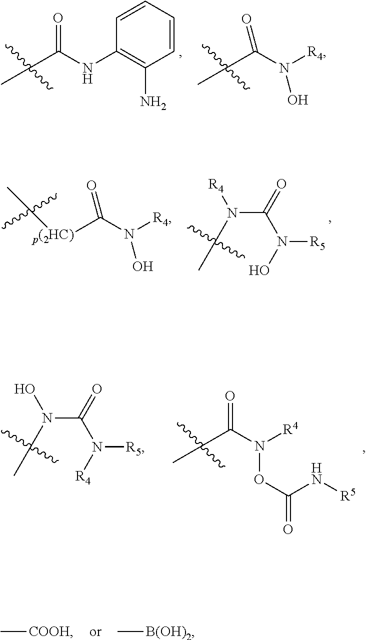
wherein R⁴ and R⁵ are each independently H or a C₁-C₅ alkyl group; and
R⁶ is H or a C₁-C₄ alkyl group.

3. The dual inhibitor of claim 1, wherein the kinase hinge binding moiety is: 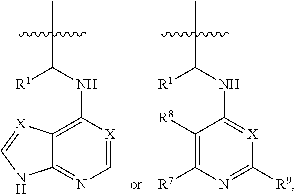
wherein R¹ is a C₁-C₅ alkyl group;
R⁷ is H, a C₁-C₅ alkyl group, a C₁-C₅ alkyl containing 1-5 fluorine atoms, a C₁-C₅ alkyl containing 1-5 deuterium atoms, or NH₂;
R⁸ is H, a C₁-C₅ alkyl group, Cl, CONH₂, or CN;
R⁹ is H, a C₁-C₅ alkyl group, a C₁-C₅ alkyl containing 1-5 fluorine atoms, a C₁-C₅ alkyl containing 1-5 deuterium atoms, or NH₂; and
X is CH or N.

4. The dual inhibitor of claim 1, wherein the core is represented by Formula 1: 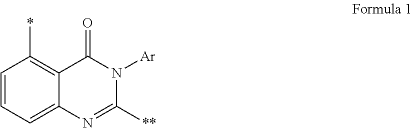
wherein Ar is an aryl or heteroaryl group unsubstituted or substituted with 1-3 C₁-C₆ alkyl groups,
“*” indicates a binding site to the histone deacetylase pharmacophore, and
“**′” indicates a binding site to the kinase hinge binding moiety.

5. The dual inhibitor of claim 4, wherein the histone deacetylase pharmacophore is:

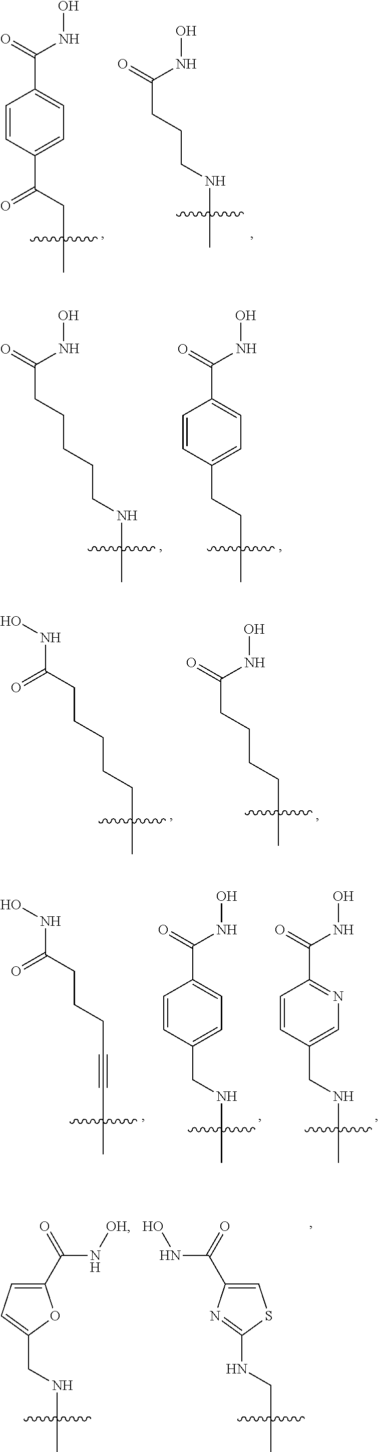

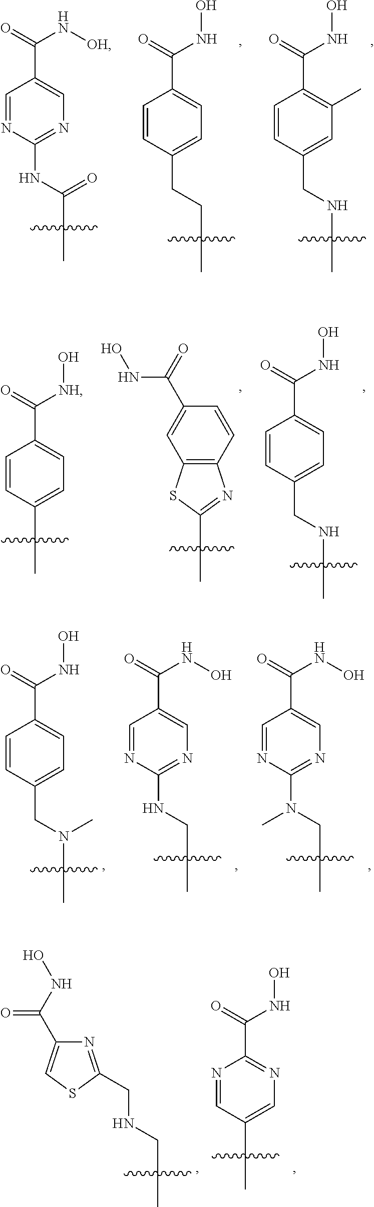

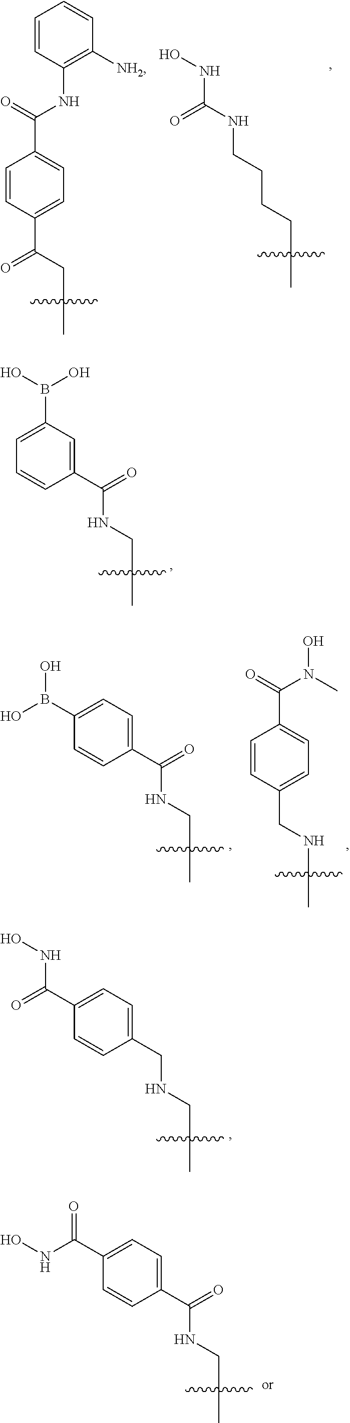

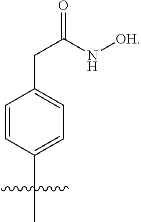 [SMILES: *c1ccc(CC(=O)NO)cc1]

6. The dual inhibitor of claim 4, wherein the kinase hinge binding moiety is: 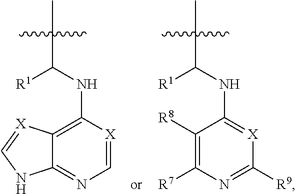
wherein R¹ is a C₁-C₅ alkyl group;
R⁷ is H, a C₁-C₅ alkyl group, a C₁-C₅ alkyl containing 1-5 fluorine atoms, a C₁-C₅ alkyl containing 1-5 deuterium atoms, or NH₂;
R⁸ is H, a C₁-C₅ alkyl group, Cl, CONH₂, or CN;
R⁹ is H, a C₁-C₅ alkyl group, a C₁-C₅ alkyl containing 1-5 fluorine atoms, a C₁-C₅ alkyl containing 1-5 deuterium atoms, or NH₂; and
X is CH or N.

7. The dual inhibitor of claim 1, wherein the core is represented by Formula 2: 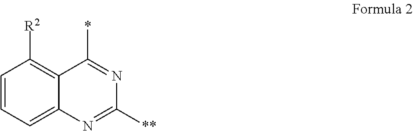
wherein
R² is hydrogen, a halogen, or a C₁-C₅ alkyl group,
“*” indicates a binding site to the histone deacetylase pharmacophore, and
“**′” indicates a binding site to the kinase hinge binding moiety.

8. The dual inhibitor of claim 7, wherein the histone deacetylase pharmacophore is:

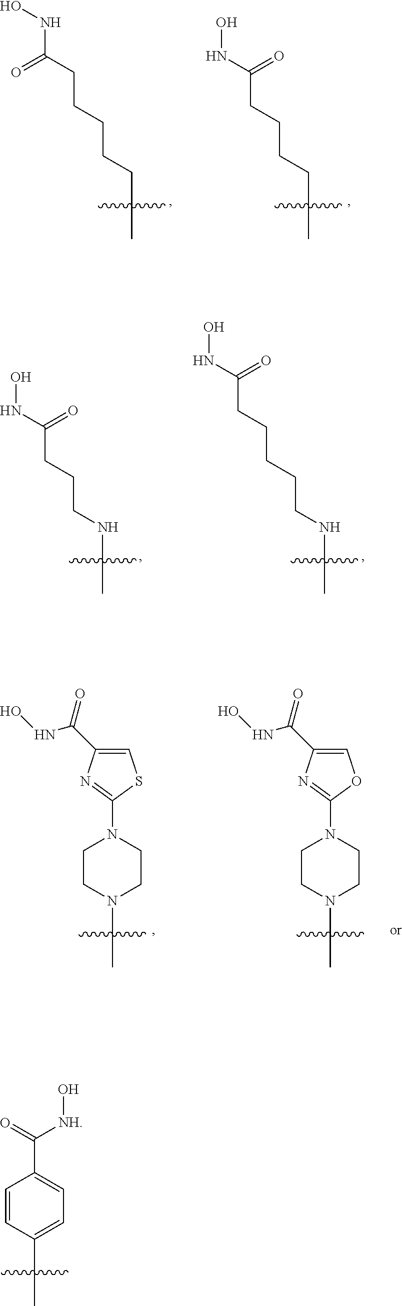

9. The dual inhibitor of claim 7, wherein the kinase hinge binding moiety is: 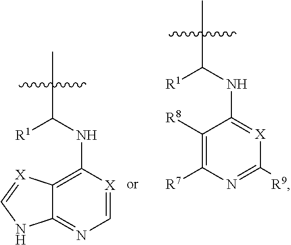
wherein R¹ is a C₁-C₅ alkyl group;
R⁷ is H, a C₁-C₅ alkyl group, a C₁-C₅ alkyl containing 1-5 fluorine atoms, a C₁-C₅ alkyl containing 1-5 deuterium atoms, or NH₂;
R⁸ is H, a C₁-C₅ alkyl group, Cl, CONH₂, or CN;
R⁹ is H, a C₁-C₅ alkyl group, a C₁-C₅ alkyl containing 1-5 fluorine atoms, a C₁-C₅ alkyl containing 1-5 deuterium atoms, or NH₂; and
X is CH or N.

10. The dual inhibitor of claim 1, represented by Formula 3: 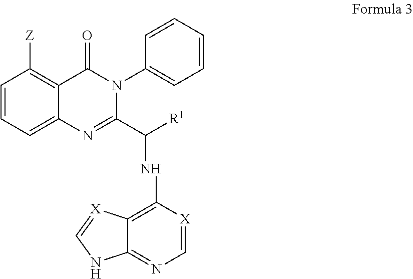
wherein, in Formula 3,
R¹ is a C₁-C₅ alkyl group;
X is CH or N; and
Z is: 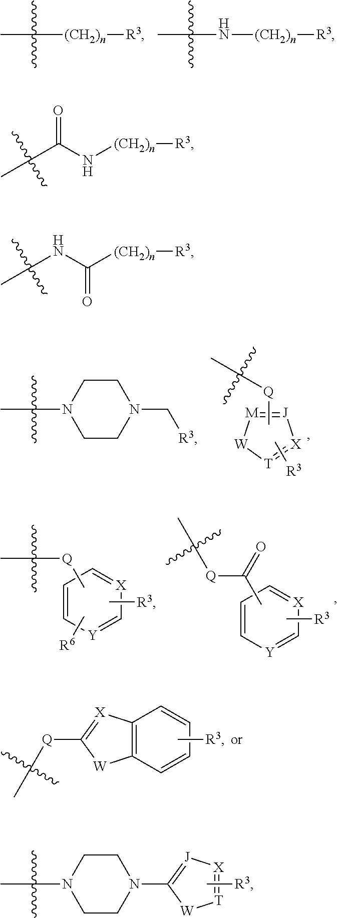
wherein, at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 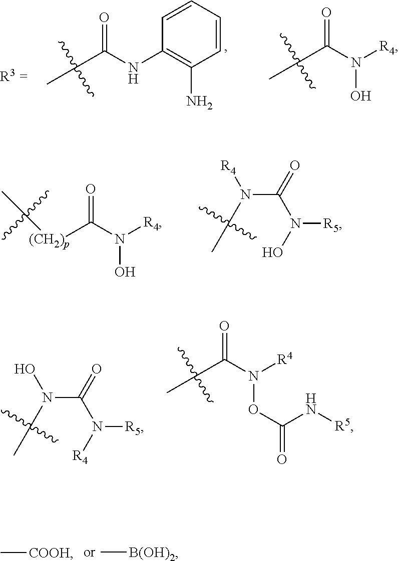
wherein R⁴ and R⁵ are each independently H or a C₁-C₅ alkyl group; and
R⁶ is H or a C₁-C₄ alkyl group.

11. The dual inhibitor of claim 1, represented by Formula 4: 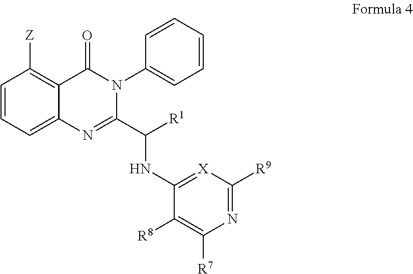
wherein, in Formula 4,
R¹ is a C₁-C₅ alkyl group;
R⁷ is H, a C₁-C₅ alkyl group, a C₁-C₅ alkyl containing 1-5 fluorine atoms, a C₁-C₅ alkyl containing 1-5 deuterium atoms, or NH₂;
R⁸ is H, a C₁-C₅ alkyl group, Cl, CONH₂, or CN;
R⁹ is H, a C₁-C₅ alkyl group, a C₁-C₅ alkyl containing 1-5 fluorine atoms, a C₁-C₅ alkyl containing 1-5 deuterium atoms, or NH₂;
X is CH or N; and
Z is 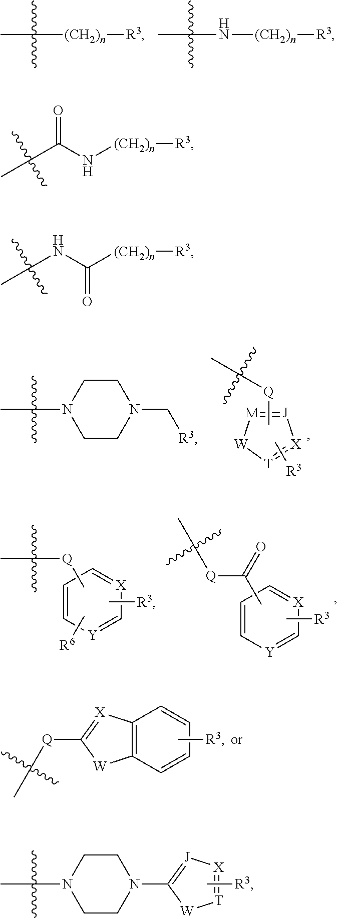
wherein, at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 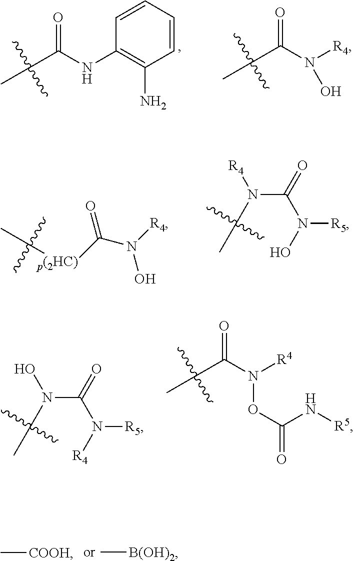
wherein R⁴ and R⁵ are each independently H or a C₁-C₅ alkyl group; and
R⁶ is H or a C₁-C₄ alkyl group.

12. The dual inhibitor of claim 1, represented by Formula 5: 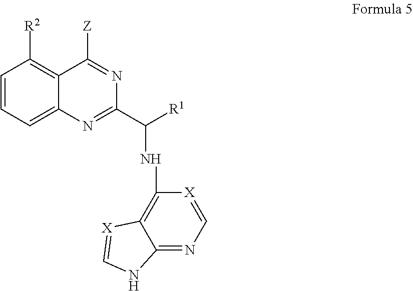
wherein, in Formula 5,
R¹ is a C₁-C₅ alkyl group;
R² is hydrogen, a halogen, or a C₁-C₅ alkyl group;
X is CH or N; and
Z is 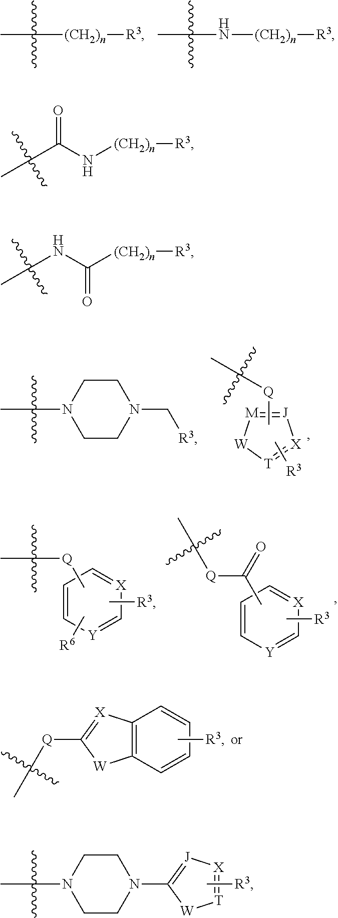
wherein in the above formulae,
at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 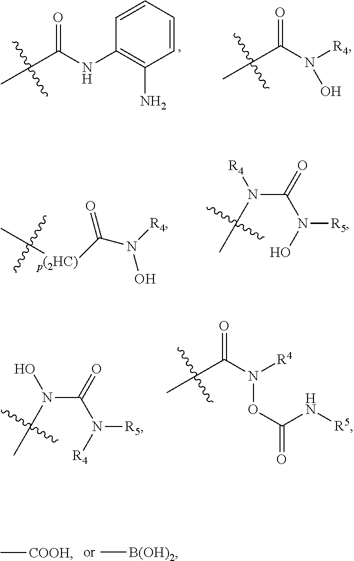
wherein R⁴ and R⁵ are each independently a C₁-C₅ alkyl group; and
R⁶ is H or a C₁-C₄ alkyl group.

13. The dual inhibitor of claim 1, represented by Formula 6: 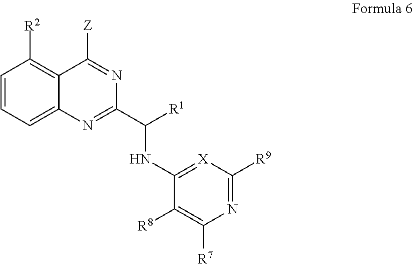
wherein, in Formula 6,
R¹ is a C₁-C₅ alkyl group,
R² is hydrogen, a halogen, or a C₁-C₅ alkyl group,
R⁷ is H, a C₁-C₅ alkyl group, a C₁-C₅ alkyl containing 1-5 fluorine atoms, a C₁-C₅ alkyl containing 1-5 deuterium atoms, or NH₂;
R⁸ is H, a C₁-C₅ alkyl group, Cl, CONH₂, or CN;
R⁹ is H, a C₁-C₅ alkyl group, a C₁-C₅ alkyl containing 1-5 fluorine atoms, a C₁-C₅ alkyl containing 1-5 deuterium atoms, or NH₂;
X is CH or N; and
Z is 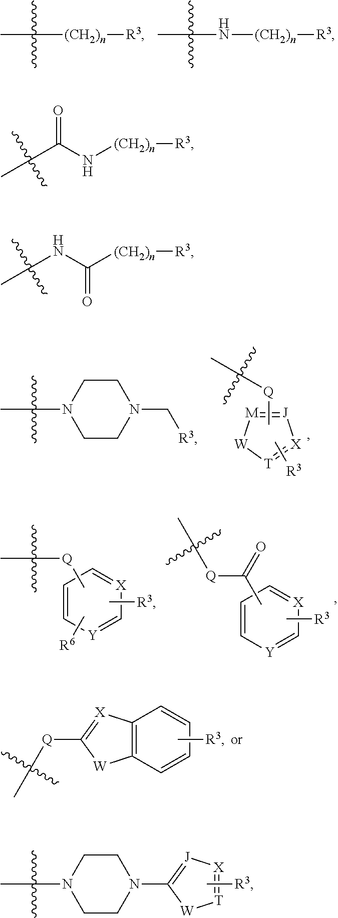
wherein in the above formulae,
at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 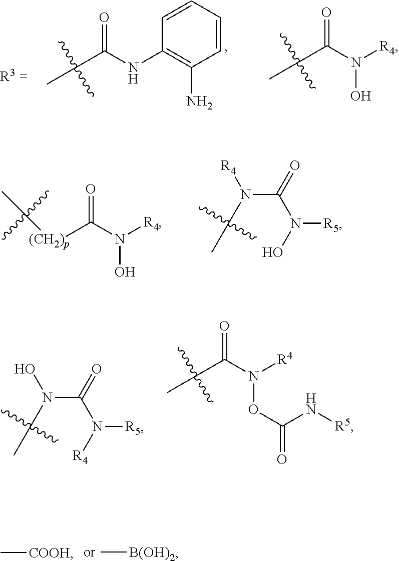
wherein R⁴ and R⁵ are each independently H or a C₁-C₅ alkyl group; and
R⁶ is H or a C₁-C₄ alkyl group.

14. The dual inhibitor of claim 1, represented by one of the following compounds:

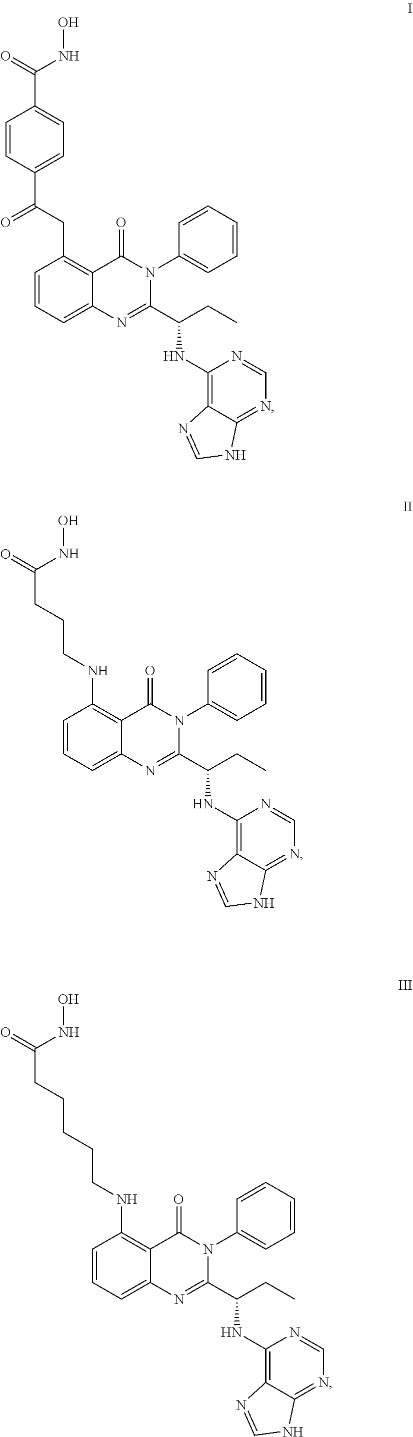 [SMILES: CC[C@H](Nc1ncnc2[nH]cnc12)c1nc2cccc(CC(=O)c3ccc(C(=O)NO)cc3)c2c(=O)n1-c1ccccc1 ; CC[C@H](Nc1ncnc2[nH]cnc12)c1nc2cccc(NCCCC(=O)NO)c2c(=O)n1-c1ccccc1 ; CC[C@H](Nc1ncnc2[nH]cnc12)c1nc2cccc(NCCCCCC(=O)NO)c2c(=O)n1-c1ccccc1]

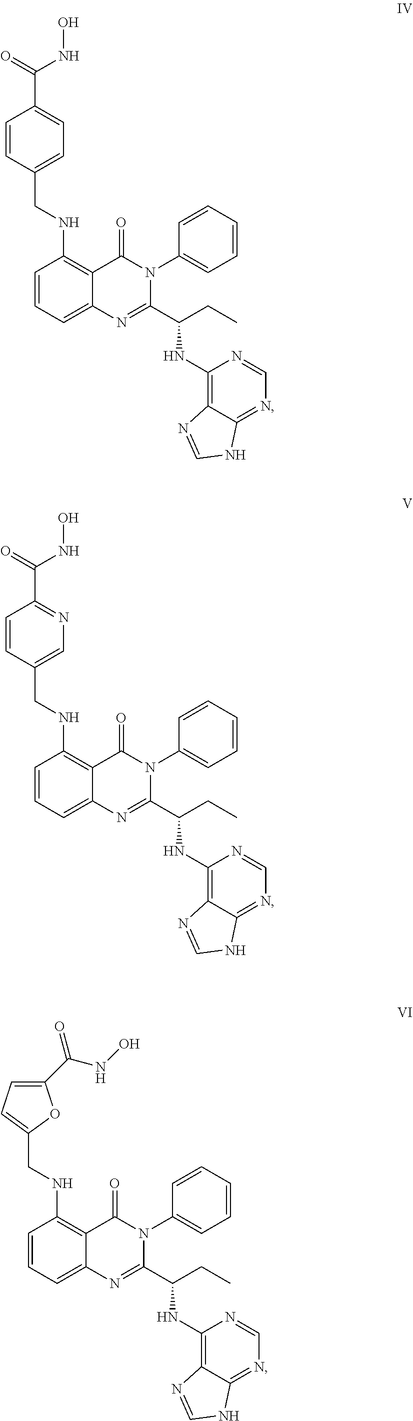 [SMILES: CC[C@H](Nc1ncnc2[nH]cnc12)c1nc2cccc(NCc3ccc(C(=O)NO)cc3)c2c(=O)n1-c1ccccc1 ; CC[C@H](Nc1ncnc2[nH]cnc12)c1nc2cccc(NCc3ccc(C(=O)NO)nc3)c2c(=O)n1-c1ccccc1 ; CC[C@H](Nc1ncnc2[nH]cnc12)c1nc2cccc(NCc3ccc(C(=O)NO)o3)c2c(=O)n1-c1ccccc1]

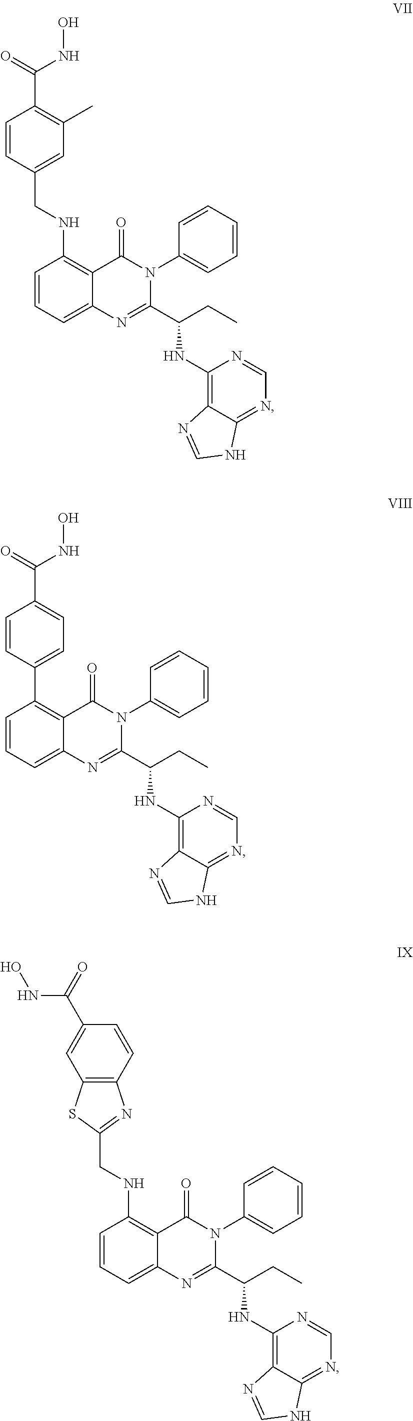 [SMILES: CC[C@H](Nc1ncnc2[nH]cnc12)c1nc2cccc(NCc3ccc(C(=O)NO)c(C)c3)c2c(=O)n1-c1ccccc1 ; CC[C@H](Nc1ncnc2[nH]cnc12)c1nc2cccc(-c3ccc(C(=O)NO)cc3)c2c(=O)n1-c1ccccc1 ; CC[C@H](Nc1ncnc2[nH]cnc12)c1nc2cccc(NCc3nc4ccc(C(=O)NO)cc4s3)c2c(=O)n1-c1ccccc1]

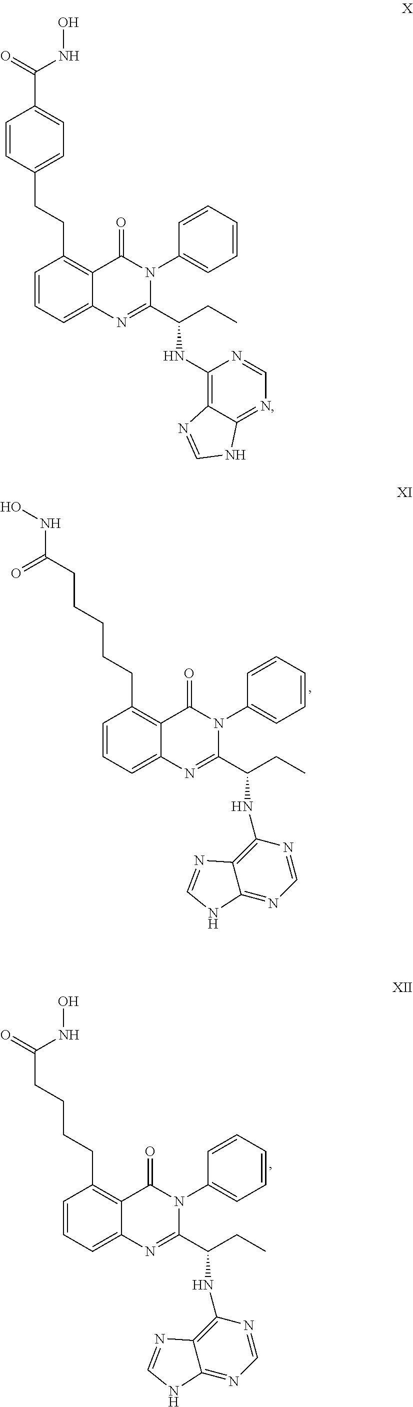 [SMILES: CC[C@H](Nc1ncnc2[nH]cnc12)c1nc2cccc(CCc3ccc(C(=O)NO)cc3)c2c(=O)n1-c1ccccc1 ; CC[C@H](Nc1ncnc2[nH]cnc12)c1nc2cccc(CCCCCC(=O)NO)c2c(=O)n1-c1ccccc1 ; CC[C@H](Nc1ncnc2[nH]cnc12)c1nc2cccc(CCCCC(=O)NO)c2c(=O)n1-c1ccccc1]

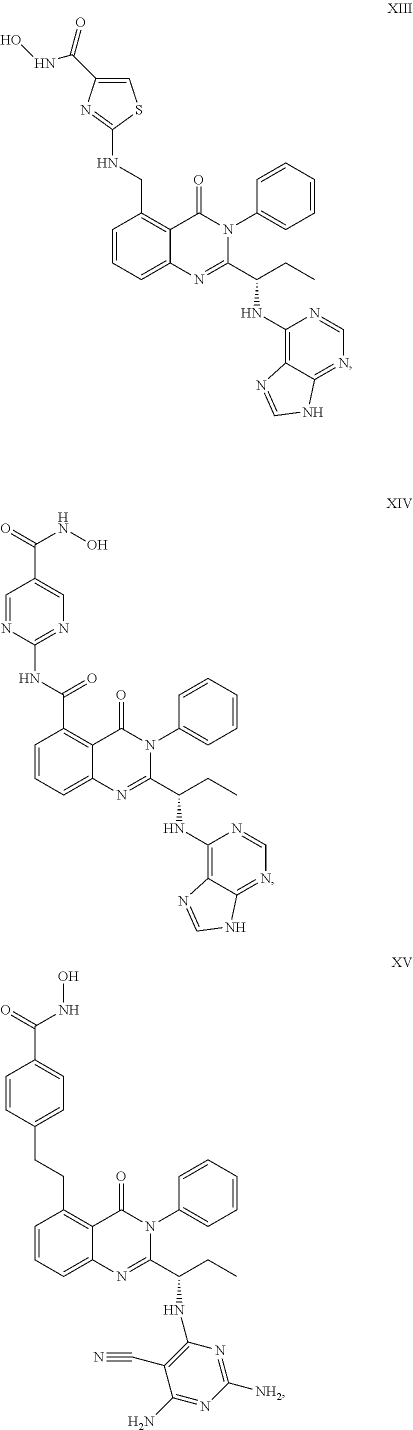 [SMILES: CC[C@H](Nc1ncnc2[nH]cnc12)c1nc2cccc(CNc3nc(C(=O)NO)cs3)c2c(=O)n1-c1ccccc1 ; CC[C@H](Nc1ncnc2[nH]cnc12)c1nc2cccc(C(=O)Nc3ncc(C(=O)NO)cn3)c2c(=O)n1-c1ccccc1 ; CC[C@H](Nc1nc(N)nc(N)c1C#N)c1nc2cccc(CCc3ccc(C(=O)NO)cc3)c2c(=O)n1-c1ccccc1]

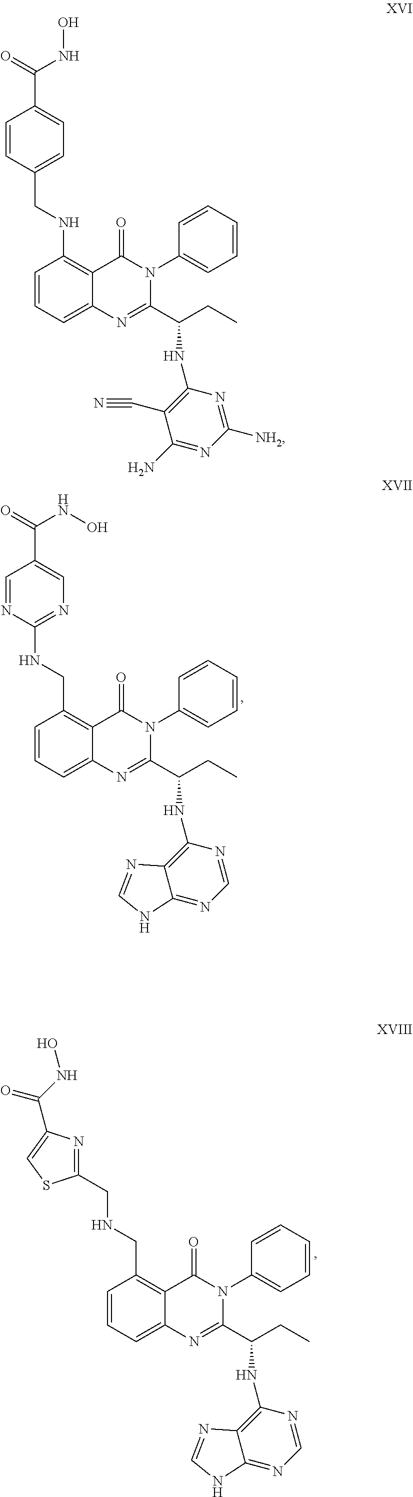 [SMILES: CC[C@H](Nc1nc(N)nc(N)c1C#N)c1nc2cccc(NCc3ccc(C(=O)NO)cc3)c2c(=O)n1-c1ccccc1 ; CC[C@H](Nc1ncnc2[nH]cnc12)c1nc2cccc(CNc3ncc(C(=O)NO)cn3)c2c(=O)n1-c1ccccc1 ; CC[C@H](Nc1ncnc2[nH]cnc12)c1nc2cccc(CNCc3nc(C(=O)NO)cs3)c2c(=O)n1-c1ccccc1]

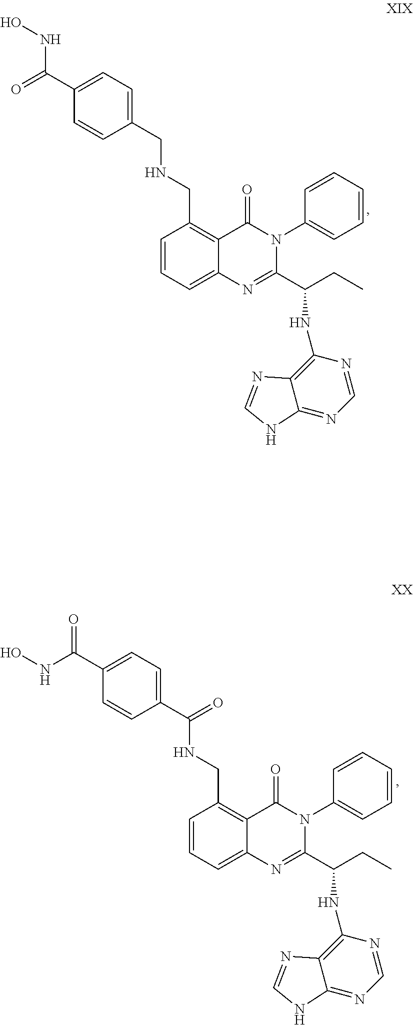 [SMILES: CC[C@H](Nc1ncnc2[nH]cnc12)c1nc2cccc(CNCc3ccc(C(=O)NO)cc3)c2c(=O)n1-c1ccccc1 ; CC[C@H](Nc1ncnc2[nH]cnc12)c1nc2cccc(CNC(=O)c3ccc(C(=O)NO)cc3)c2c(=O)n1-c1ccccc1]

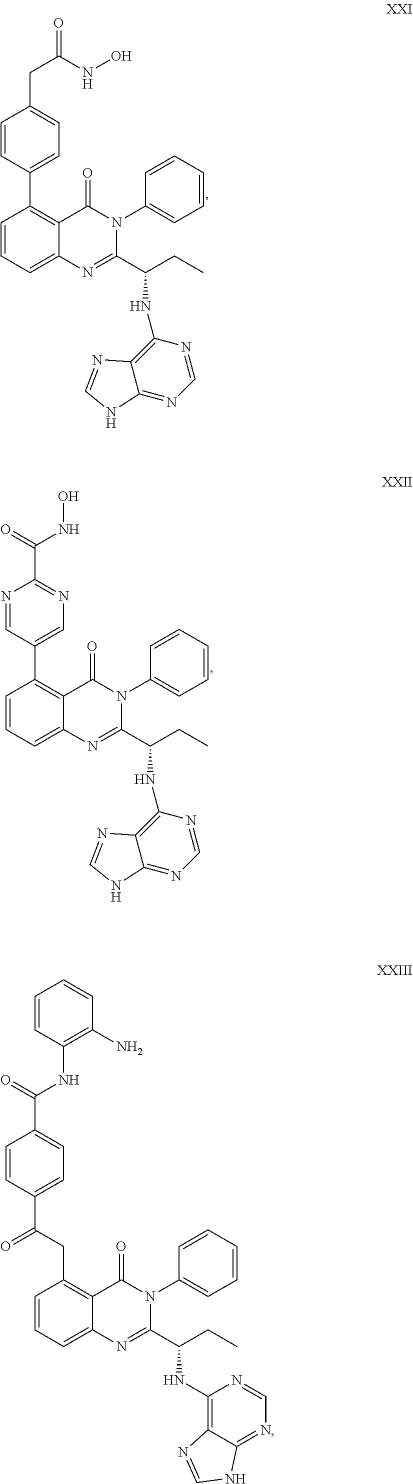 [SMILES: CC[C@H](Nc1ncnc2[nH]cnc12)c1nc2cccc(-c3ccc(CC(=O)NO)cc3)c2c(=O)n1-c1ccccc1 ; CC[C@H](Nc1ncnc2[nH]cnc12)c1nc2cccc(-c3cnc(C(=O)NO)nc3)c2c(=O)n1-c1ccccc1 ; CC[C@H](Nc1ncnc2[nH]cnc12)c1nc2cccc(CC(=O)c3ccc(C(=O)Nc4ccccc4N)cc3)c2c(=O)n1-c1ccccc1]

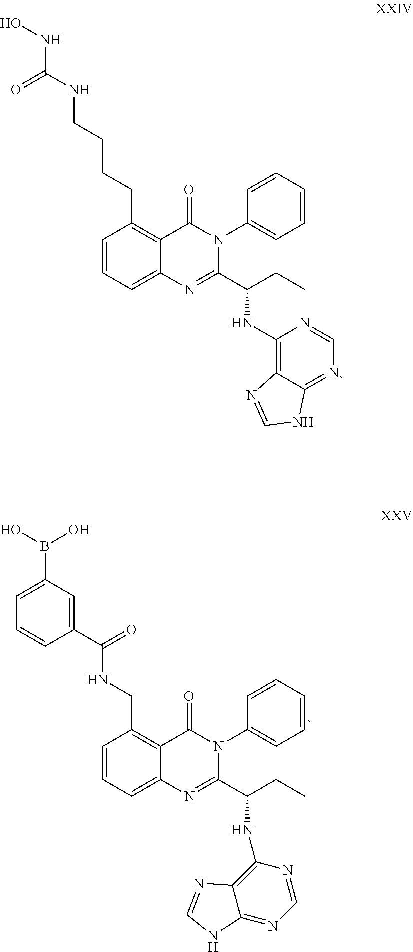 [SMILES: CC[C@H](Nc1ncnc2[nH]cnc12)c1nc2cccc(CCCCNC(=O)NO)c2c(=O)n1-c1ccccc1 ; CC[C@H](Nc1ncnc2[nH]cnc12)c1nc2cccc(CNC(=O)c3cccc(B(O)O)c3)c2c(=O)n1-c1ccccc1]

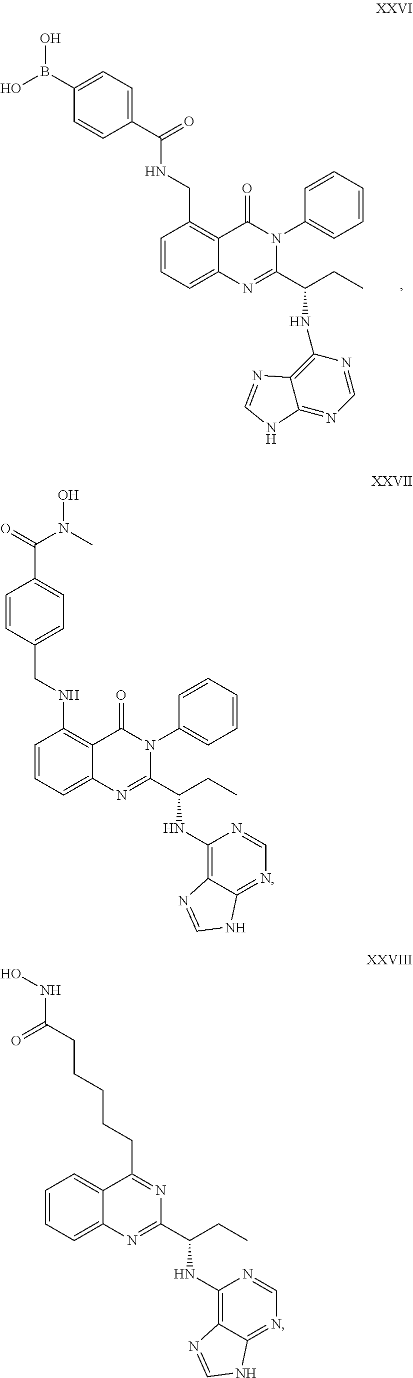 [SMILES: CC[C@H](Nc1ncnc2[nH]cnc12)c1nc2cccc(CNC(=O)c3ccc(B(O)O)cc3)c2c(=O)n1-c1ccccc1 ; CC[C@H](Nc1ncnc2[nH]cnc12)c1nc2cccc(NCc3ccc(C(=O)N(C)O)cc3)c2c(=O)n1-c1ccccc1 ; CC[C@H](Nc1ncnc2[nH]cnc12)c1nc(CCCCCC(=O)NO)c2ccccc2n1]

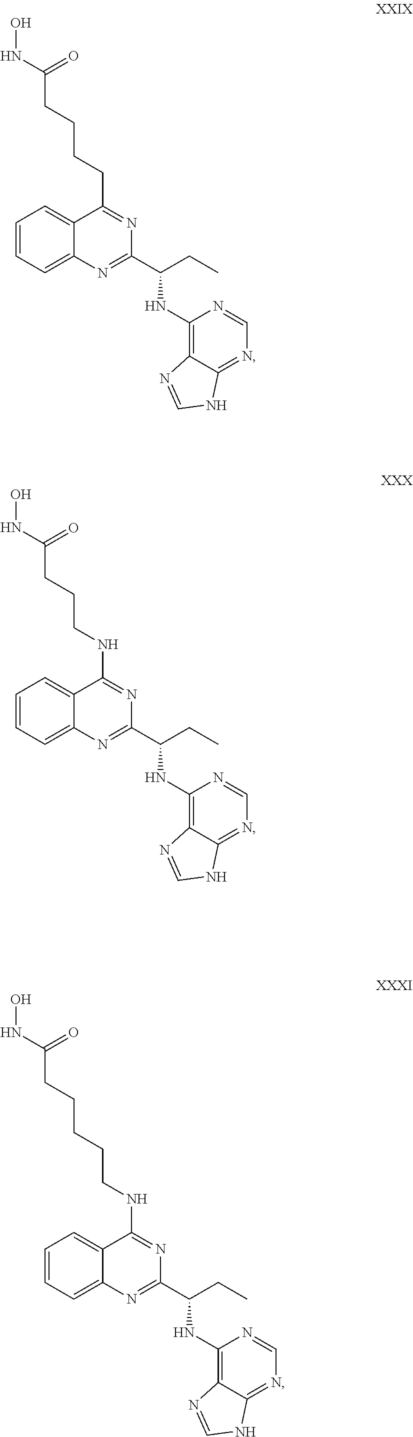 [SMILES: CC[C@H](Nc1ncnc2[nH]cnc12)c1nc(CCCCC(=O)NO)c2ccccc2n1 ; CC[C@H](Nc1ncnc2[nH]cnc12)c1nc(NCCCC(=O)NO)c2ccccc2n1 ; CC[C@H](Nc1ncnc2[nH]cnc12)c1nc(NCCCCCC(=O)NO)c2ccccc2n1]

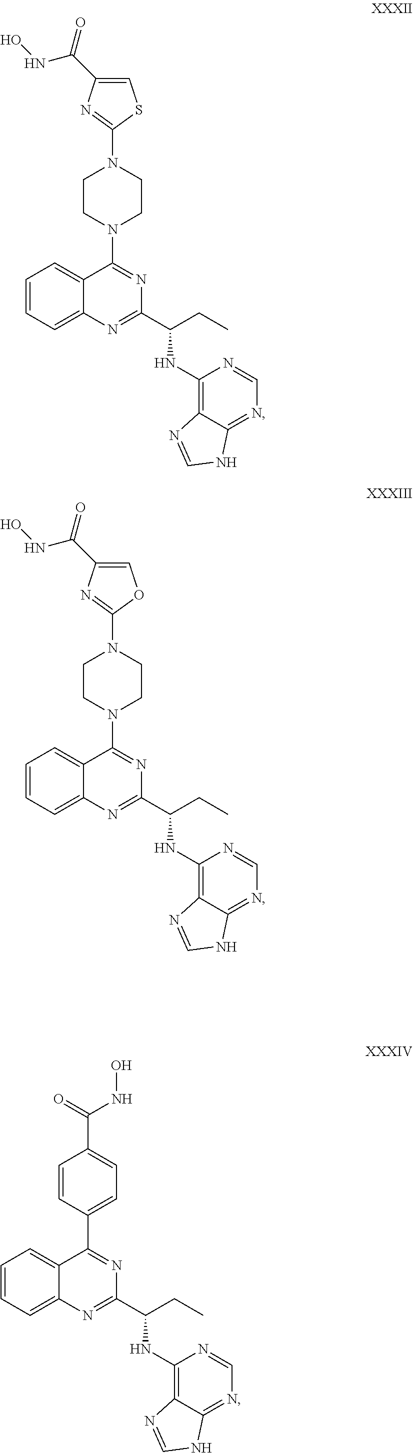 [SMILES: CC[C@H](Nc1ncnc2[nH]cnc12)c1nc(N2CCN(c3nc(C(=O)NO)cs3)CC2)c2ccccc2n1 ; CC[C@H](Nc1ncnc2[nH]cnc12)c1nc(N2CCN(c3nc(C(=O)NO)co3)CC2)c2ccccc2n1 ; CC[C@H](Nc1ncnc2[nH]cnc12)c1nc(-c2ccc(C(=O)NO)cc2)c2ccccc2n1]

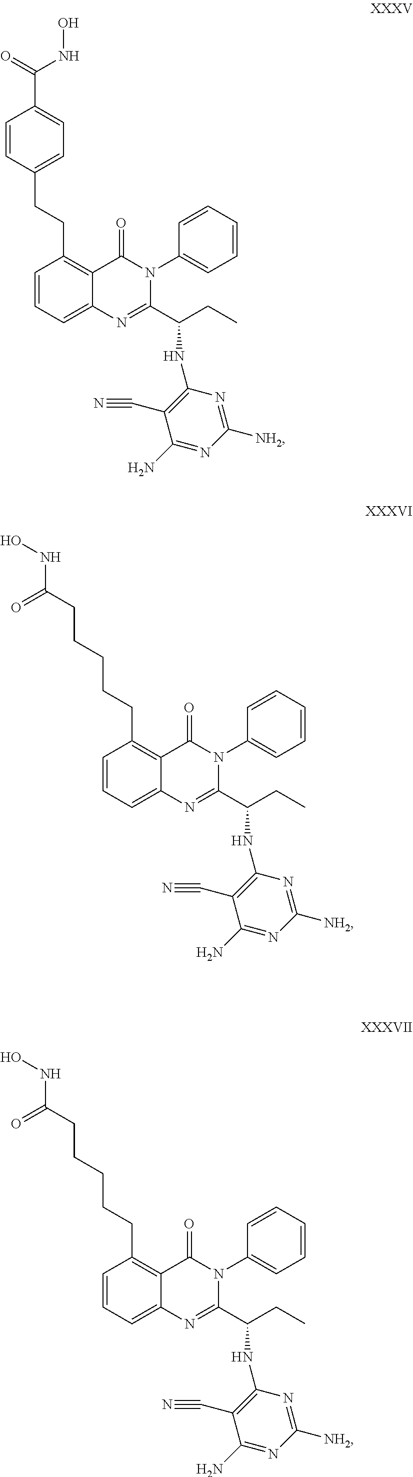 [SMILES: CC[C@H](Nc1nc(N)nc(N)c1C#N)c1nc2cccc(CCc3ccc(C(=O)NO)cc3)c2c(=O)n1-c1ccccc1 ; CC[C@H](Nc1nc(N)nc(N)c1C#N)c1nc2cccc(CCCCCC(=O)NO)c2c(=O)n1-c1ccccc1 ; CC[C@H](Nc1nc(N)nc(N)c1C#N)c1nc2cccc(CCCCCC(=O)NO)c2c(=O)n1-c1ccccc1]

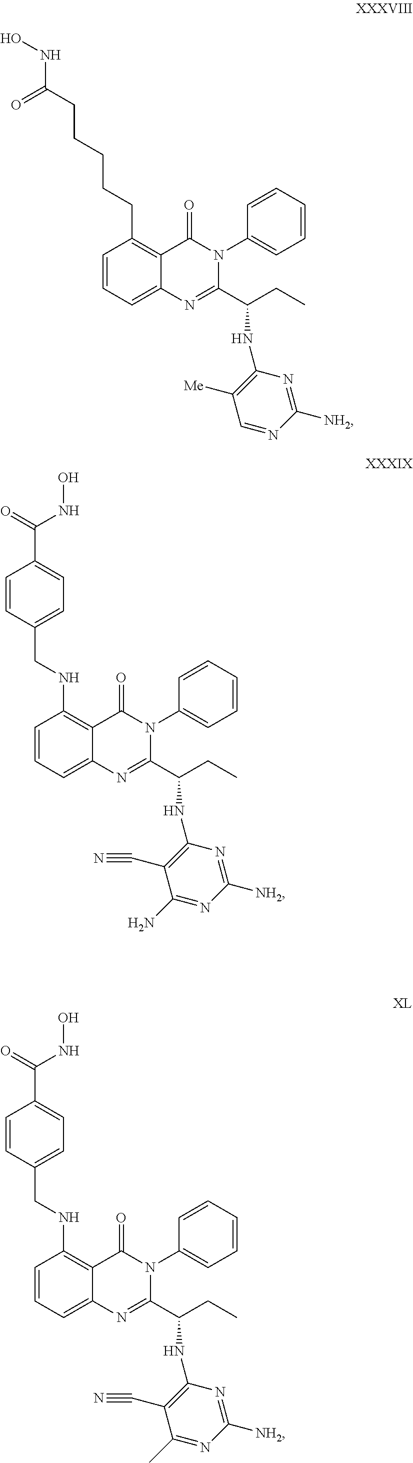 [SMILES: CC[C@H](Nc1nc(N)ncc1C)c1nc2cccc(CCCCCC(=O)NO)c2c(=O)n1-c1ccccc1 ; CC[C@H](Nc1nc(N)nc(N)c1C#N)c1nc2cccc(NCc3ccc(C(=O)NO)cc3)c2c(=O)n1-c1ccccc1 ; CC[C@H](Nc1nc(N)nc(C)c1C#N)c1nc2cccc(NCc3ccc(C(=O)NO)cc3)c2c(=O)n1-c1ccccc1]

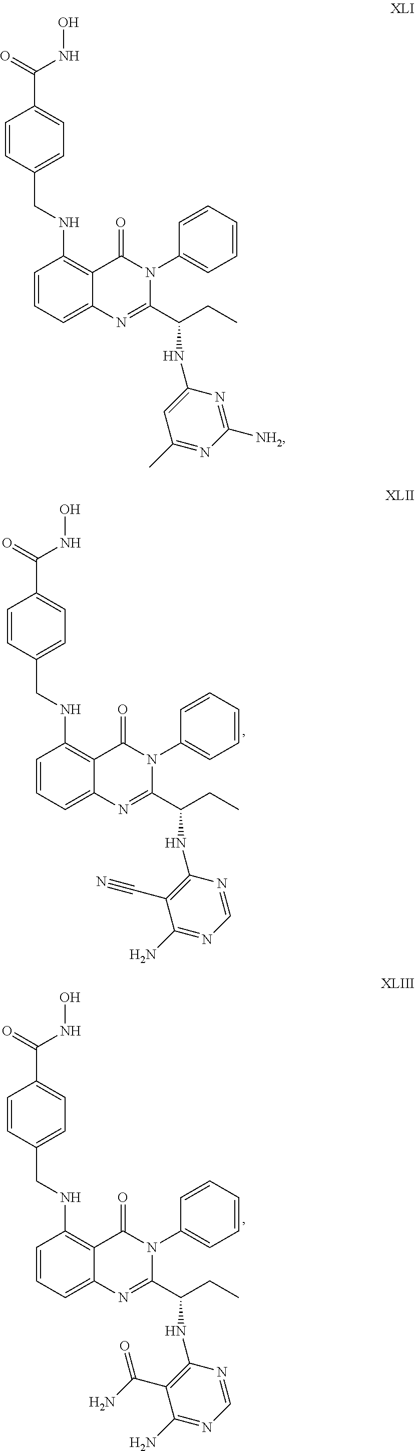 [SMILES: CC[C@H](Nc1cc(C)nc(N)n1)c1nc2cccc(NCc3ccc(C(=O)NO)cc3)c2c(=O)n1-c1ccccc1 ; CC[C@H](Nc1ncnc(N)c1C#N)c1nc2cccc(NCc3ccc(C(=O)NO)cc3)c2c(=O)n1-c1ccccc1 ; CC[C@H](Nc1ncnc(N)c1C(N)=O)c1nc2cccc(NCc3ccc(C(=O)NO)cc3)c2c(=O)n1-c1ccccc1]

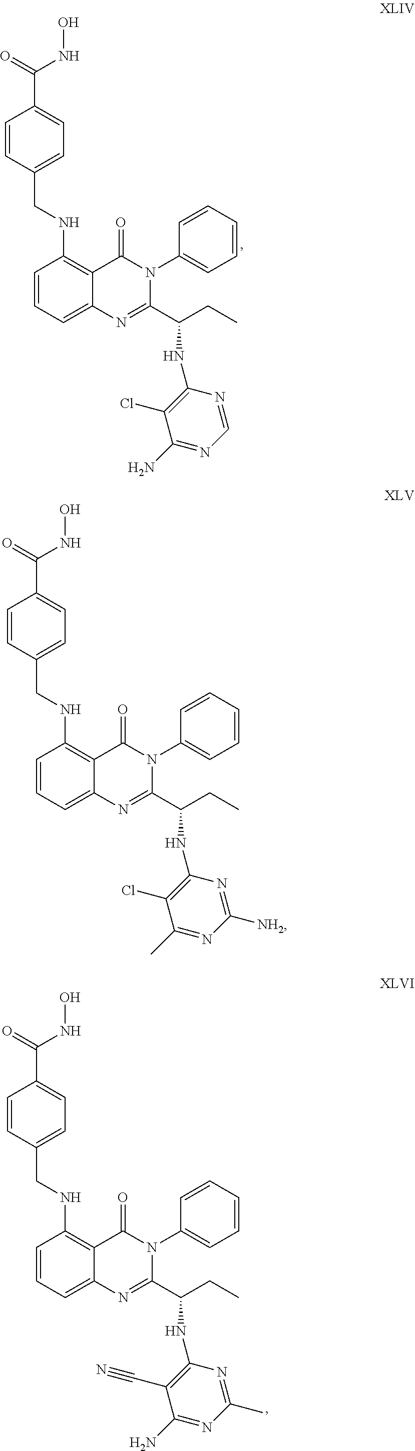 [SMILES: CC[C@H](Nc1ncnc(N)c1Cl)c1nc2cccc(NCc3ccc(C(=O)NO)cc3)c2c(=O)n1-c1ccccc1 ; CC[C@H](Nc1nc(N)nc(C)c1Cl)c1nc2cccc(NCc3ccc(C(=O)NO)cc3)c2c(=O)n1-c1ccccc1 ; CC[C@H](Nc1nc(C)nc(N)c1C#N)c1nc2cccc(NCc3ccc(C(=O)NO)cc3)c2c(=O)n1-c1ccccc1]

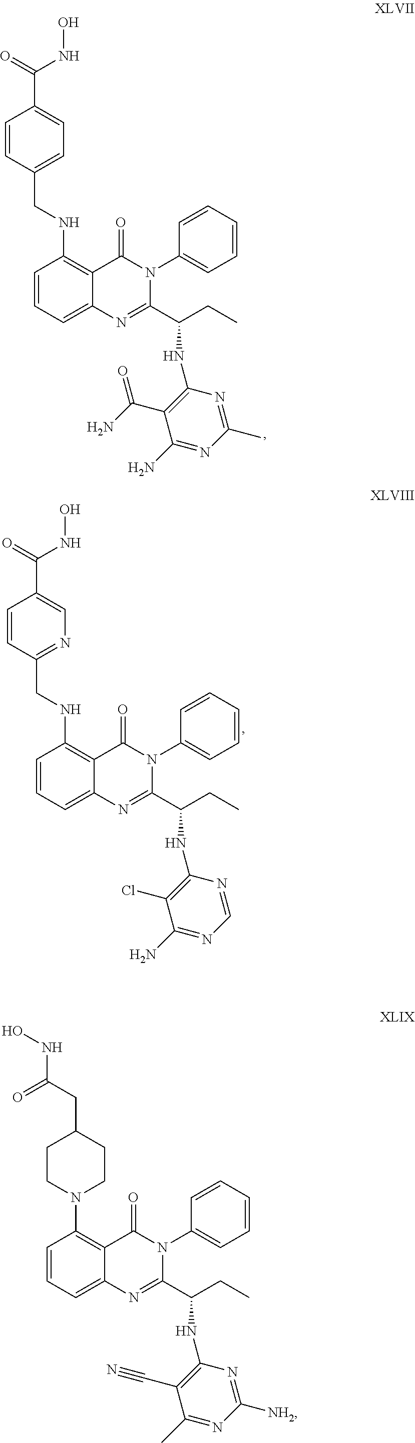 [SMILES: CC[C@H](Nc1nc(C)nc(N)c1C(N)=O)c1nc2cccc(NCc3ccc(C(=O)NO)cc3)c2c(=O)n1-c1ccccc1 ; CC[C@H](Nc1ncnc(N)c1Cl)c1nc2cccc(NCc3ccc(C(=O)NO)cn3)c2c(=O)n1-c1ccccc1 ; CC[C@H](Nc1nc(N)nc(C)c1C#N)c1nc2cccc(N3CCC(CC(=O)NO)CC3)c2c(=O)n1-c1ccccc1]

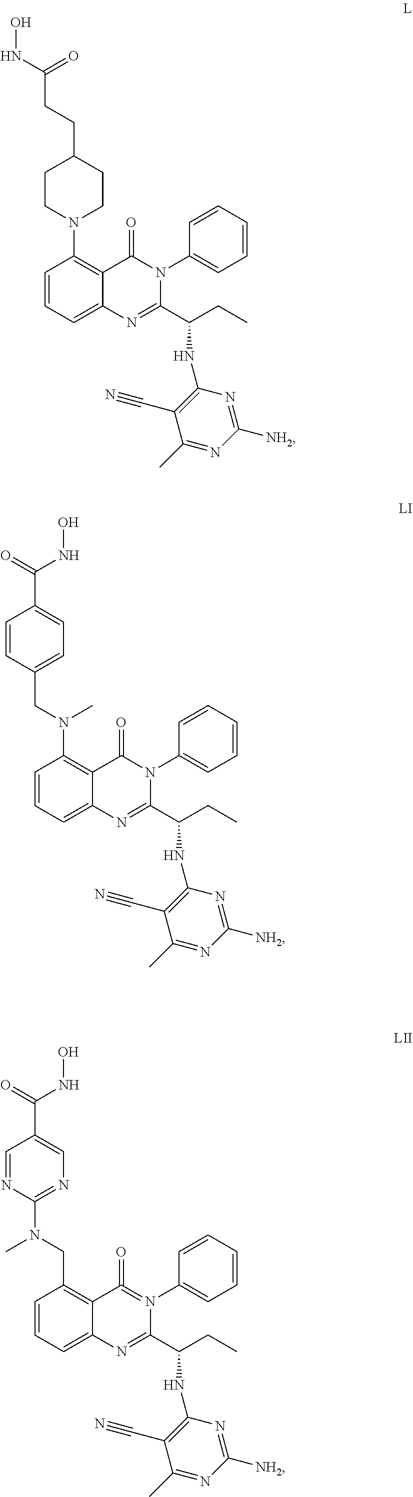

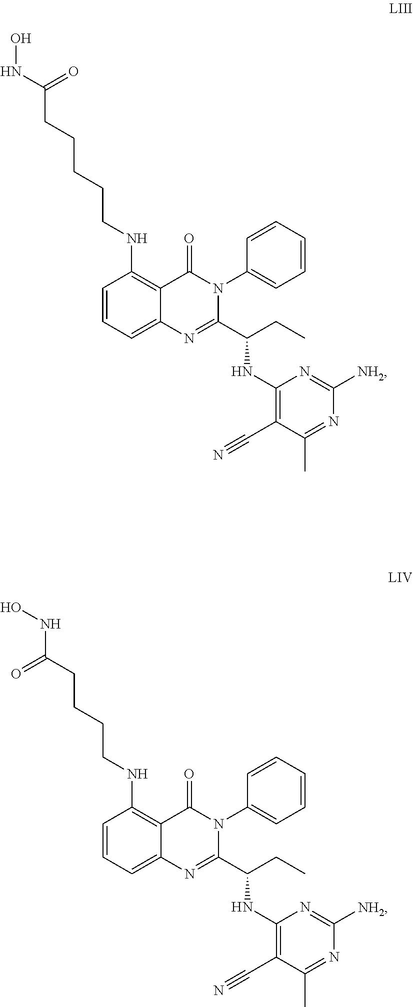 [SMILES: CC[C@H](Nc1nc(N)nc(C)c1C#N)c1nc2cccc(NCCCCCC(=O)NO)c2c(=O)n1-c1ccccc1 ; CC[C@H](Nc1nc(N)nc(C)c1C#N)c1nc2cccc(NCCCCC(=O)NO)c2c(=O)n1-c1ccccc1]

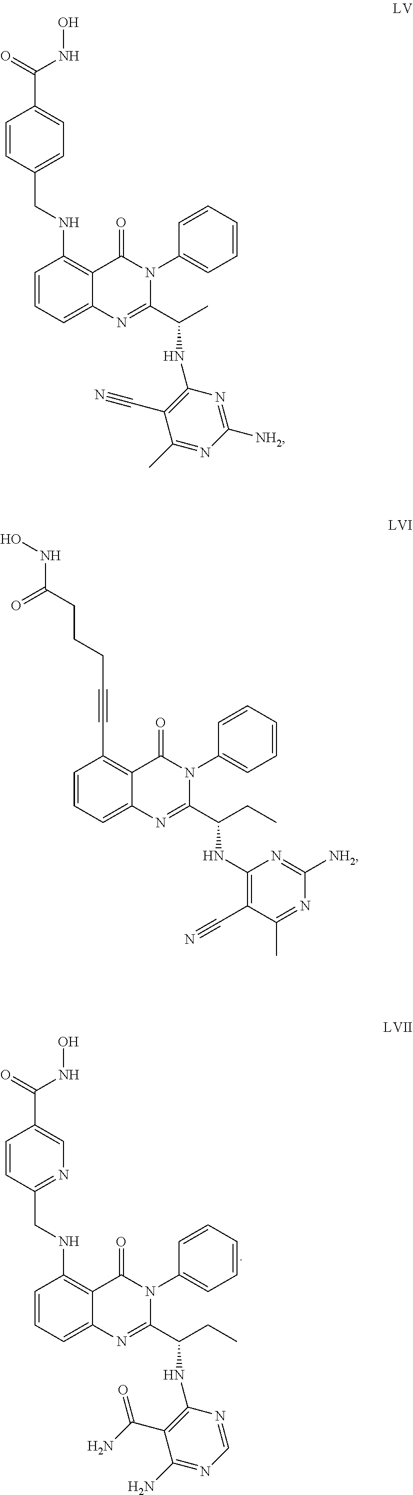 [SMILES: Cc1nc(N)nc(N[C@@H](C)c2nc3cccc(NCc4ccc(C(=O)NO)cc4)c3c(=O)n2-c2ccccc2)c1C#N ; CC[C@H](Nc1nc(N)nc(C)c1C#N)c1nc2cccc(C#CCCCC(=O)NO)c2c(=O)n1-c1ccccc1 ; CC[C@H](Nc1ncnc(N)c1C(N)=O)c1nc2cccc(NCc3ccc(C(=O)NO)cn3)c2c(=O)n1-c1ccccc1]

15. (canceled)

16. A method for treating or diagnosing cancer in a mammal, comprising administering to the mammal a pharmaceutical composition comprising an effective amount of an active agent, wherein the active agent is a dual inhibitor of phosphoinositide 3-kinase (PI3K) and histone deacetylase (HDAC), wherein the dual inhibitor comprises:
a core comprising a quinazoline moiety or a quinazolin-4(3H)-one moiety;
a kinase hinge binding moiety; and
a histone deacetylase pharmacophore,
a pharmaceutically acceptable salt thereof, a prodrug thereof, or solvate thereof.

17.-19. (canceled)

20. A compound represented by Formula 7 or Formula 8, or a pharmaceutically acceptable salt, prodrug, or solvate thereof: 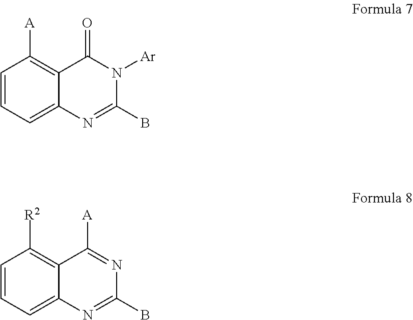
wherein
Ar is an aryl or heteroaryl group unsubstituted or substituted with 1-3 C₁-C₆ alkyl groups,
R² is hydrogen, a halogen, or a C₁-C₅ alkyl group,
A is selected from: 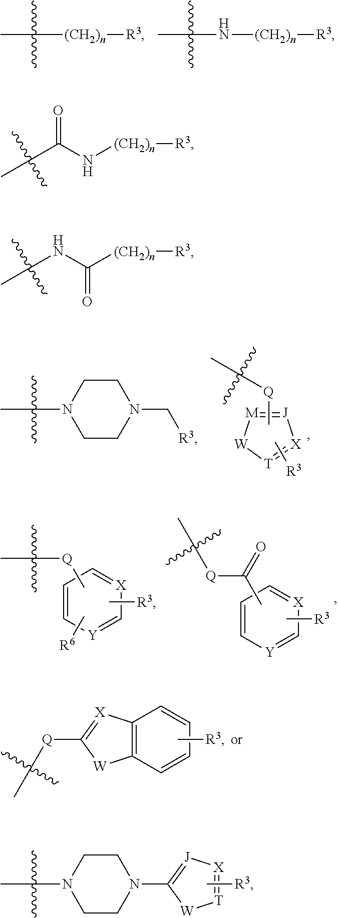
wherein in the above formulae,
at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 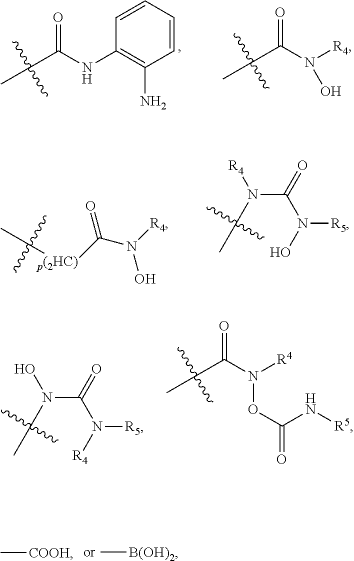
wherein R⁴ and R⁵ are each independently H or a C₁-C₅ alkyl group; and
R⁶ is H or a C₁-C₄ alkyl group, and
wherein B is selected from: 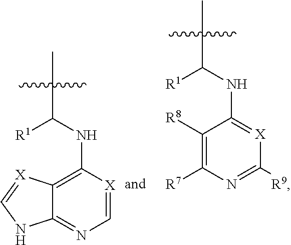
wherein R¹ is a C₁-C₅ alkyl group;
R⁷ is H, a C₁-C₅ alkyl group, a C₁-C₅ alkyl containing 1-5 fluorine atoms, a C₁-C₅ alkyl containing 1-5 deuterium atoms, or NH₂;
R⁸ is H, a C₁-C₅ alkyl group, Cl, CONH₂, or CN;
R⁹ is H, a C₁-C₅ alkyl group, a C₁-C₅ alkyl containing 1-5 fluorine atoms, a C₁-C₅ alkyl containing 1-5 deuterium atoms, or NH₂;
X is CH or N;
A is histone deacetylase pharmacophore; and
B is a kinase hinge binding moiety.

21. (canceled)

22. An inhibitor of histone deacetylase (HDAC) comprising:
a core comprising a quinazoline moiety or a quinazolin-4(3H)-one moiety; and
a histone deacetylase pharmacophore,
a pharmaceutically acceptable salt thereof, a prodrug thereof, or solvate thereof.

23. The inhibitor of claim 22, represented by Formula 9: 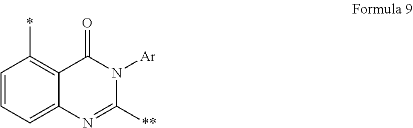
wherein
Ar is an aryl or heteroaryl group unsubstituted or substituted with 1-3 C₁-C₆ alkyl groups,
“*”, is 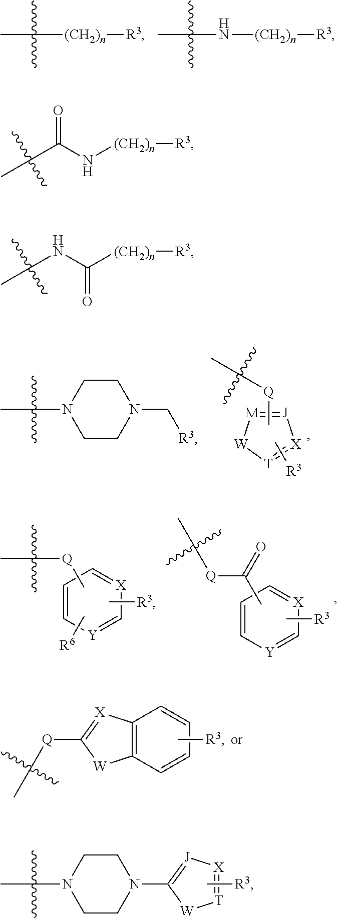
wherein in the above formulae,
at least one non-adjacent —CH₂— group is optionally replaced with —O—;
n is 1, 2, 3, 4, and 5;
J is CH or N;
M is CH or N;
W is N, O, or S;
X is CH or N;
T is CH or N;
Q is —(CH₂)ₚ—, —(CH₂)ₚNH(CH₂)ᵣ—, —NH(CH₂)ₚ— or —(CH₂)ₚNH—, wherein p and r are each independently 0, 1, 2, 3, or 5;
Y is CH or N;
R³ is 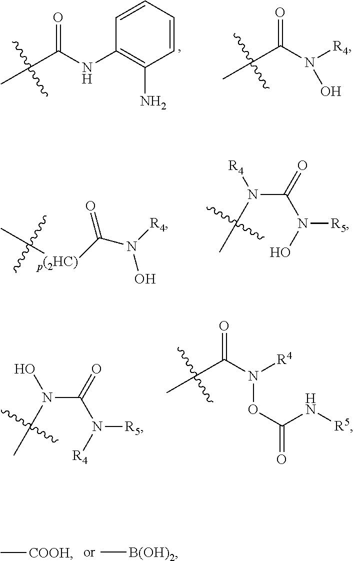
wherein R⁴ and R⁵ are independently be H or a C₁-C₅ alkyl group; and
R⁶ is H or a C₁-C₄ alkyl group, and
“**′” is H, C₁-C₆ alkyl, C₃-C₆ cycloalkyl, or aryl.

24. The inhibitor of claim 22, represented by one of the following compounds:

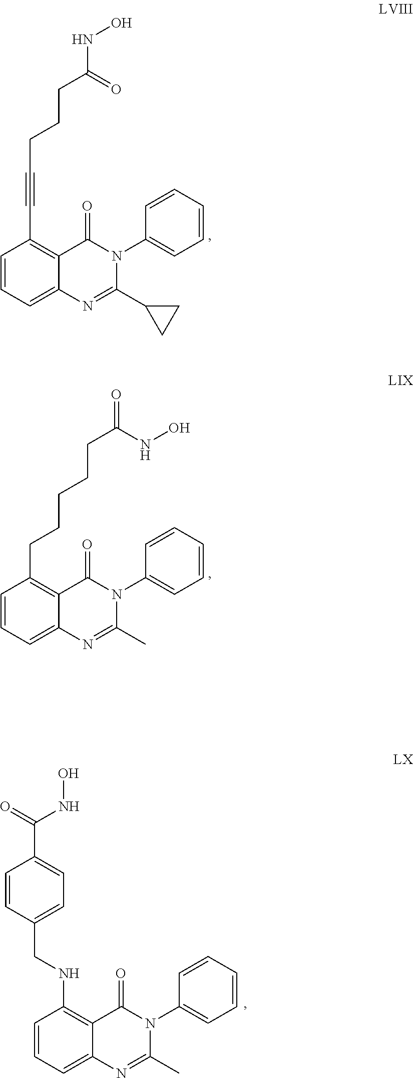 [SMILES: O=C(CCCC#Cc1cccc2nc(C3CC3)n(-c3ccccc3)c(=O)c12)NO ; Cc1nc2cccc(CCCCCC(=O)NO)c2c(=O)n1-c1ccccc1 ; Cc1nc2cccc(NCc3ccc(C(=O)NO)cc3)c2c(=O)n1-c1ccccc1]

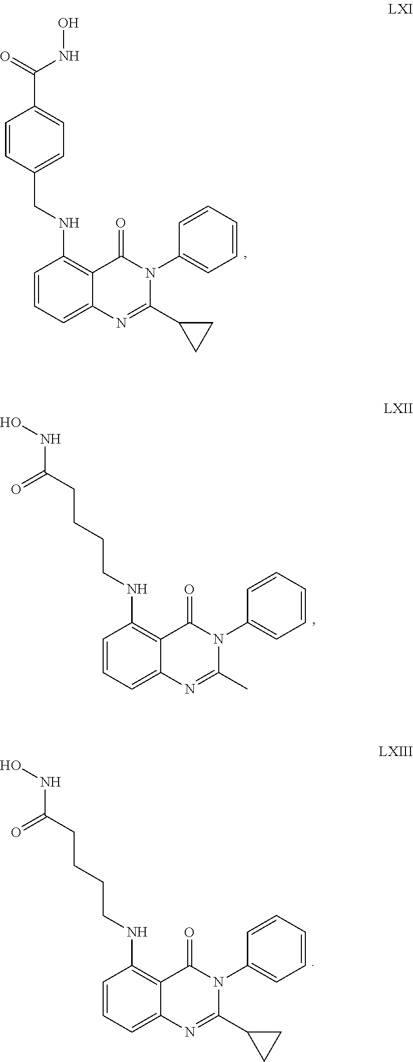 [SMILES: O=C(NO)c1ccc(CNc2cccc3nc(C4CC4)n(-c4ccccc4)c(=O)c23)cc1 ; Cc1nc2cccc(NCCCCC(=O)NO)c2c(=O)n1-c1ccccc1 ; O=C(CCCCNc1cccc2nc(C3CC3)n(-c3ccccc3)c(=O)c12)NO]

25.-27. (canceled)
